SEQUENCE LISTING (255 sequences)

SEQ ID NO: 1 - DNA, 483 bp, unknown
  >sr|IMAGp998N195554.2|cl|IMAGp998N195554|gb| BX108731|oid|234551841 BX108731 NCI_CGAP_GC6 Homo sapiens cDNA clone IMAGp998N195554 ; IMAGE2244690, mRNA sequence
tggagagctgcgggcaagctcagcctcgaaacacacacacccacacgagatgggcacggagtagtcttgaaagacattttaataaccttggctgctaaggacaacattgatagaagccgtctctggctatagataagtagatctaatactagtttggatatctttagggtttagaatctaacctcaagaataagaaatacaagtacgaattggtgatgaagatgtattcgtattgtttgggattgggaggctttgcttattttttaaaaactattgaggtaaagggttaagctgtaacatacttaattgatttcttaccgtttttggctctgttttgctatatcccctaatttgttggttgtgctaatctttgtagaaagaggtcttgtatttgctgcatcgtaatgacatgagtactactttagttggtttaagttcaaatgaatgaaacaaatatttttcctttagttgattttaccctga

SEQ ID NO: 2 - DNA, 683 bp, Homo sapiens
  >ImageID=147103 IMAGP971H0410_S1 CC FromEXP of IMAGp971H0410_s1.exp April 26, 2002 1306
caaatggtctaagattgacagtagcacagttttactctgtgaaggtaatgttcaggacaaatttcaagaaaactagaaaaccattctttacagctgaaatctttccctaaccattgttatttccacttttaagtcctcaagagatgagaaaagggaggtaaggcttccttatacatttcctgcacaatgaaacatttttcctcctccaggcaaagattcaagcagaactggcaaatatcttatcttgctcttctcaataataataatgttgttagataataaagttctatagcaatttaaccctagaatctttttgaaaagtaattctttaaagttgagaatcacagctgtctagcaagcatttccttgggcacttgaagctgtttattcactttggtctttcctcccaggggaatgaagtgaggccagtggccgggtctgctgagtccgcagcactcagactacgtgcacctctgcagcaggtgcaggcccagttgtcacccctccagaacatctccccatggattctggcggtattgactcttcagattcagaggtagggatcattctgacttattaaagagctatataaccagttaattccatctgtttgatgcttgacatccctaactagacagatgagggttgaagttagtttttggtggggtttggganggtgaaca

SEQ ID NO: 3 - DNA, 718 bp, unknown
  >sr|IMAGp998F08222.1|cl|IMAGp998F08222|gb| BX091352|oid|234517071 BX091352 Soares placenta Nb2HP Homo sapiens cDNA clone IMAGp998F08222 ; IMAGE146695, mRNA sequence.
atttttttaaaagactatccccttcatcactgtgctttgtccctttgtctaaagtcggttgttcatatatgtgggtctgttccgtagactttattcagttctattgatcttttttgatatgatgccagtaccatactctgttagtaacgatagtgttttaataatacatcttgaaattaggaagtatagagttattttgcctatagtagatcctttattttgcattttagaatcagcttaagagtttctgcaaaaatacctgcaggaattttgatggagattgcattgattctctagatcaatttggggagaattttaacaatattgagtattttgacatatgagtgcaatatgcctgtcattcattcaggtctttaatttctctcaggattgatctttttaaaagtaaaccctttattttgggataattttcatttttttcccccttcatgtcacagtttttcattttatttttccccctccatgtcacagttttgatcatggaataatttttcaagatctgagagctacagtgaaaaggaaaaaaagaaagaaagaaaacatgatactatagtccctgatgattgattttttttaatcccaatcatctgtaattcttcccagagataagaaatggttgtagtatgttatctcctaggaattttttttggaattcaaacagataaataaaatatactcatagaaaaaaaaaaaaaaa

SEQ ID NO: 4 - DNA, 461 bp, unknown
  >gi|855247|sr|IMAGp998A06220s1|cl| IMAGp998A06220|gb|R78966|R78966 yi83b03.r1 Homo sapiens cDNA clone 145805 5′.
agccaactgaacctgctaattgccagaattaggcatattcggctgatctgtcacaggaaatgctgggctggagcaggcctcccatgttttacaacctgcctacataagacataagcatctaggagcagagaatccctcctcctttcaagcagctctttcccaacagaataaagggaagtcactgagctgtgaatgcctgttctattcagatttacctcttagataagttgttccagctaccccgggaaggaggggtaaaggggatgagatgtttgaactgtaacaccctgtacatgggaggagacaacatgagttgttgatgggagcatccttccattcttacctttttgggcttttanaaaaacttttantggcagtgcagnaaaatatttgaaaattgtttttnacagggtttttgaaggccattncccaggctgctggnaaatcctggnaaaattgnt

SEQ ID NO: 5 - DNA, 305 bp, unknown
  >sr|IMAGp998D203167.2|cl|IMAGp998D203167|gb| BX101698|oid|234537773 BX101698 NCI_CGAP_GC4 Homo sapiens cDNA clone IMAGp998D203167 ; IMAGE1258339, mRNA sequence
aaccaaagctttgtgtaaaatcttgaatttatggggcgggagggtaggaaagcctgtacctgtctgtttttttcctgatccttttccctcattcctgaactgcaggagactgagcccctttgggctttggtgaccccatcactggggtgtgtttatttgatggttgattttgctgtactgggtacttcctttcccattttctaatcattttttaacacaagctgactcttcccttcccttctcctttccctgggaaaatacaatgaataaataaagacttattggtactcaaaaaaaaaaaaaaa

SEQ ID NO: 6 - DNA, 628 bp, unknown
  >sr|IMAGp998D054091.2|cl|IMAGp998D054091|gb| BX092581|oid|234519530 BX092581 NCI_CGAP_GC3 Homo sapiens cDNA clone IMAGp998D054091 ; IMAGE1613140, mRNA sequence
ataagatgggaactctcccggcacgtagtacatatcccgccgtgggtgaaagttcccgaagacctgaaagatccagaggtgttccaggtccagacgcggctgctgaaagccattttcggcccggacggatctcgaatcccttacatcgagcaggtgagcaaggccatgctcgagctgaaggctctggagtcttcagacctcaccgaggtcgtggtttacggctcctatttgtacaagctccggaccaagtggatgctccagtccatggctgagtggcaccgccagcgccaggagcgagggatgctcaaacttgccgaagccatgaatgccctcgaactaggcccttggatgaagtgaaccagtttccagccaatgcaatgaagccgggttgcagagattaggttgtggccagagctagagtgattccttaagcttgttttaaaatctgctccagcctaaagagttaagggaaaaccatttgttcccttaaagagttaagggaaaacccttggctctgagtcttgttgtgaatatttctttgatgattgttaataaaaagtgttttttcttttttcccatttttaaaaataacaataaagntttaaataagttgaaaaaaaaaaaaaaa

SEQ ID NO: 7 - DNA, 545 bp, unknown
  >gi|1526341|gb|AA046430|sr|IMAGp998K241161.1| cl|IMAGp998K241161|AA046430 zk70f12.s1 Soares_pregnant_uterus_NbHPU Homo sapiens cDNA clone IMAGE488207 3′ similar to PIRA54560 A54560 TPA-induced protein OTS-8 - mouse ;contains element PTR5 repetitive element ;, mRNA sequence
tttttcacatggttacagcatgtaatatcgatccaatggtccagtgttagttacaaatcagtttatacagttttgctccattttctatgaattttgtctttcaaataatcttttatagaatgtatattcaaaaacatgtttgaacagcaaatgtttagaggagccaagtctggtgaacggtctttccttcggttattttggcaagcggagaggtcaccgtggattctgagtgggccaggcaagtgttccacgggtcatctgctcccacgagctcagggacagggcacagagtcagaaacggtctttttttaagcacgttggcagcagggcgtaacccttcagctctttagggcgagtaccttcccgacatttttcgcataaccacaacgatgattccaccaatgaagccgatgntagtaagaccccaactatgattccaaccagggtcactgttgacaaaccatctttctcaactgttgtctgtgtgtctccatccactttctccgtggagtgactggtggccacgtttgaggctgcctcgtgcc

SEQ ID NO: 8 - DNA, 223 bp, unknown
  >gi|4682406|gb|AI631076|sr|IMAGp998J095673.1| cl|IMAGp998J095673|AI631076 tz32c05.x1 NCI_CGAP_Ut2 Homo sapiens cDNA clone IMAGE2290280 3′, mRNA sequence.
tttttttttttttttttttttttttttttttttttttttttttttttttttttttattgggaaaggtttatttaaaggtttgggggccccttttacccccccccaaaaagaaaaaaaaaaattttttttttgccgtcccaaacagggaagtcccttcaaagaggagctggcgggtgggttcaaaccccaaaagggggacccctgccagggggtttttcccggc

SEQ ID NO: 9 - DNA, 491 bp, unknown
  >sr|IMAGp998I175722.2|cl|IMAGp998I175722|gb| BX097870|oid|234530113 BX097870 NCI_CGAP_GC6 Homo sapiens cDNA clone IMAGp998I175722 ; IMAGE2309080, mRNA sequence
gcacccaggcagcctgcgcggagaaatcggatcggctaggacggcctgcagcccctgcgcgcggggaaagggaagaagtcctcccctacaaagcgagttcacaaacttggaagaagccactttcacaggatgcacagatctcaacggaaggacacagaaaacatgaaaaagcaaggaaatggggcgcctccaacggaacccagtaattctccagcagcagatccccatccaaaagaaacagaagacattttggagagagaattgaatatacatattatacagttgtgggcataatacaagcatcatttggaaaagaacagagaaaaaagatggaaggagttaccgtactgcaggaaaaaattatcaactccagtgatattattaaatacaacctaacgacacttctgaagctgaagaaatcactggatgaaatacaaagtaaactcaaaagcttcagtgatagactagaacaagaagaagaaagactctca

SEQ ID NO: 10 - DNA, 296 bp, unknown
  >gi|4682412|gb|AI631082|sr|IMAGp998J105673.1| cl|IMAGp998J105673|AI631082 tz32d05.x1 NCI_CGAP_Ut2 Homo sapiens cDNA clone IMAGE2290281 3′, mRNA sequence.
tttttttttttttttttttttttttttttttttttttttttttttttttttttttattgggaaaggtttatttaaagggttggggtccccttttaaccccccccaaaaagagaaaaaaaaattttttttttgccgttccaaacaggaaagtcctttcaaagaggagctggcgtgtgggttcaaagcccaagagggggacccctgccagggggtttttcccggcctgggccaccctgggtaggagggccacccaggccccggtttcctgggaccctgggggttttggagggagggag

SEQ ID NO: 11 - DNA, 550 bp, unknown
  >gi|4283562|gb|AI436660|sr|IMAGp998N115245.1| cl|IMAGp998N115245|AI436660 th91c06.x1 Soares_NSF_F8_9W_OT_PA_P_S1 Homo sapiens cDNA clone IMAGE2126026 3′ similar to TRP70423 P70423 CATIONIC AMINO ACID TRANSPORTER. ;, mRNA sequence.
ttttatttttctactttcttttaatatcattttttaaagttggtaagcagctagacatcatttagaagcagacgggttaaaatagacaagaaatagcaaagacacatccttcacatcgtacagaactgtattagtatccaccaccaccatcacaggggagggctagctgtcactggggtcaggagtactctccattattgtgcaggggaccagacagcatttaggtgtgacgatgtcaaactgagtggacatagagagtgccgggatcaaggtctacagttttggctctagacttgcgtgagggttggttactcttaatctcttccaggctgtgctggatcccatagccgaagtagatagcaaagccaatcagcatccagaccccaaatcgggcccaggtaccagctgtcatctgcatcataaggtaaatattcacaaagatgctcattagtgggaggagaggcaaagcaggcaccttaaagtgaaggggagtggaactctgtggctgtcttcagatgaccacaatgatcccaataatgagcagcaggag

SEQ ID NO: 12 - DNA, 618 bp, unknown
  >sr|IMAGp998J05529.1|cl|IMAGp998J05529|gb| BX091496|oid|234517359 BX091496 Soares fetal liver spleen 1NFLS Homo sapiens cDNA clone IMAGp998J05529 ; IMAGE245476, mRNA sequence.
agtcattgacggtgaatacaggtctggaagtctgaggtccaatataataaaaaatctagtcttcaccttctatcctagggatagacattaggaatatttgaggatgttgttcttggtcttcacgttcatgcattttccctctatttttataattggatcaatgaatcaataagcaaatatattttaactgcataaagataaaaataggcgttgtccaattatggaaatatatggtgaaataagaaatactattcacatttattaaaaacactgaggtcttctttctattaacaaataaatgactcagaggtattactcgcttgctgcaggatggcatatacagtaaaaataataataacgtatgtgaggtcatttgggtcaggaccatgttcatggttgtttgttttctcaagaaaaaatagtttcacacatacttacatgactaatagaatgaaaggagaaatgtgtggataaatggacagaatatttccatctttttatcttttttgctttgtactctcacaatgaacaccctcaagttgacttaagcacccttccatggtcatgaggtttctggttgcctctgtgtgttagaagcttggagaccaatgccttctg

SEQ ID NO: 13 - DNA, 713 bp, unknown
  >sr|IMAGp998A03577.1|cl|IMAGp998A03577|gb| BX102059|oid|234538498 BX102059 Soares melanocyte 2NbHM Homo sapiens cDNA clone IMAGp998A03577 ; IMAGE263690, mRNA sequence
ttaaatccagtgtggaatagctgccttaggaccttgacccacacttactttaagaaatcgttctagttcttaaatttcaatggtaaagccagaggctggtgcttgaaaagtatttacagtgctcaggcagcactttctgcagttacagattctttactgcccctttttatgtctgaactttacatactggattactcgtgtcaataggagaaggaacagtgccttgatttaacacgtccttgtaatagtcgtggtccttctcttctgggaaaggagaagaaatagtgcccctggagtcccattgccttgggaggcatatggctcgtttcagtgattccttgagaattctggtaagaagtcaaaaataagcatttaaataaaaagaatagtaaaaaccacagtaaggaaagagcttctgatccatttagtaaaataaaacatacattttataaggtagaaatattaaatagaaccatttattactctaaattctatccttatttatctctcaaaataatagaacagtgccacttttgataatttttctatttgcacacccaaaatgcctgttttaaaatatataatagagaatgaagaaatttggggaatttcagaccaacaatgtgtttatattcatgtaagtctgattttcaaatcagtgtaaattgtcagtaatttccataatacatattggcctagttctgtatgcttac

SEQ ID NO: 14 - DNA, 335 bp, unknown
  >gi|3756508|gb|AI203902|sr|IMAGp998H234408.1| cl|IMAGp998H234408|AI203902 qd71g12.x1 Soares_testis_NHT Homo sapiens cDNA clone IMAGE1734982 3′ similar to contains L1.t2 L1 repetitive element ;, mRNA sequence.
tttttttttttttttaaacataccctttattaacatctaggtaatatctgtaatattccttgctcctcatccccaagtacgctattagctgtccatccttctgggtagaagtgtattttcgttttacttgttgatttttggatgcatgctgggggaggaaagcatattgtttgtagtcaccctggcgtgctaaggtatattattccccagtaattctctcaaggtgggcatatgcaaaacataatctctaaattcttcaatactaagaaatacctttgttttacccctaaaatcaaatgccattttggctggatataggattctaggattaaagc

SEQ ID NO: 15 - DNA, 565 bp, unknown
  >sr|IMAGp998G13226.1|cl|IMAGp998G13226|gb| BX093404|oid|234521175 BX093404 Soares placenta Nb2HP Homo sapiens cDNA clone IMAGp998G13226 ; IMAGE148260, mRNA sequence
tttttttatataacagaggttatcttggaagagagatagcaaacaatttctgtgtgtctttgtgacctttaaattcaaaacaaaaaaaataagccactcttttaaaagtgctcagtctaaaaaataaacacgtagtctagaggcggcaaggcccgtgtgtgaagcctggctcttcctcggctaagctgtgtgacggtcggcaagttgcttaccttcttgaagctgcattctcctcattcttaaaataaaaatggtaataacaggaccgcctcattgagcttgtgtgaagagtaacagacaactgcagccaagtccccagaaggtgcctggcacatagtgagggcttgatatgaggtagctattatcaggacatgattcctgtaatcttcacatttttttactttgacaatcaaaaagccaatacagataattgaaaagacaagaaaagtgacatcagcaggctccttagcccaaaattcaccccctccctctctcgacccagagctccacttgagaagggcgggtggcagacacacctcttatatttaggaatattcctgcggcc

SEQ ID NO: 16 - DNA, 580 bp, unknown
  >sr|IMAGp998J23134.1|cl|IMAGp998J23134|gb| BX092461|oid|234519290 BX092461 Soares fetal liver spleen 1NFLS Homo sapiens cDNA clone IMAGp998J23134 ; IMAGE129142, mRNA sequence.
caactatttgccccagnacaactgtttttcccccttccttctatggattgaccagccagttctgctttagatggtattggatggagccctggaatggctgaaaagtccaaactggcctgacttgctcagccagcaatgcggtgcaggctcctagctggggccttagttctcctgcacctgcggctctccatatggctgcttggacttcctcatagcatggcagatggattacgagaaggagcattcccaaataaaggaccacataagctagatctctggagggctagcctcagaagtcacccagtgtcacatgggccacattttattggttacagggccagccagtttgaaggggaagagaaatatgtcgctgtttatgctgtgtccagtgctagcttgctacctgctctcccagttccagtgctcagggcagcactggcagaacagatgtacttactgagttaaaaacagcaacatccaagacaattgttaacttttaaaactgtctcccatcccagaaggtataactaaaaaactaacaataaaaataatagtaataaataataaaaaaaaaaaaaaa

SEQ ID NO: 17 - DNA, 306 bp, unknown
  >gi|4987550|gb|AI699650|sr|IMAGp998K225545.1| cl|IMAGp998K225545|AI699650 tt18f11.x1 NCI_CGAP_GC6 Homo sapiens cDNA clone IMAGE2241165 3′, mRNA sequence.
gtcacgcaccgcacgttcgtggggaacctggcgctaaaccgacagatttaacaaagggagtgatgtgtgatcacatatgtgttcttaaaagattgttatgcatgtttcatgtagaatagattagggaagatcagggaagatacagggagacaatatactcctgctgtagtagtcaagccagaaatcctgttgcctggatccagggttgtggcagtagaaaaatagatcaaaagtagataaaatttgcagtagtaattaactgtattggagcatttgaaaaggatgatttaaaaaaaaaaaaaaaaa

SEQ ID NO: 18 - DNA, 443 bp, unknown
  >gi|1525834|gb|AA045940|sr|IMAGp998N121162.2| cl|IMAGp998N121162|AA045940 zk75d06.r1 Soares_pregnant_uterus_NbHPU Homo sapiens cDNA clone IMAGE488651 5′, mRNA sequence
ggaccagcatgccggtggggtgcaggcgccatttgtcttggaagtcaatttccttcaggagacaaagcaagtttggggtggtagccttaatgcccagcaccttggattgattcccatggtctaagagggtctttttgactaaggccagcctgtggtgatcccagcagctttggagggcctgcagccacacactgactgatacatggtatgttacagggacttgtcactttggatcaggctgggcactacagggcctgatgggcaaacttgggtaatcactgggaggagggaaactgaggcccgaggcagatacctgggtttggggagggctgggtttagcccggttctctnaactacatacccccagctgttaagccttcctcttgaggggttgaggtgggcagtatacaggagccaagcagcccagggtggggtcgcagccc

SEQ ID NO: 19 - DNA, 509 bp, unknown
  >sr|IMAGp998H07676.3|cl|IMAGp998H07676|gb| BX100963|oid|234536302 BX100963 Soares_fetal_lung_NbHL19W Homo sapiens cDNA clone IMAGp998H07676 ; IMAGE301878, mRNA sequence.
gaaccctggcttggtggattttgctagatttttctgatttttaaactcctgaaaaatatcccagataactgtcatgaagctggtaactatcttcctgctggtgaccatcagcctttgtagttactctgctactgccttcctcatcaacaaagtgccccttcctgttgacaagttggcacctttacctctggacaacattcttccctttatggatccattaaagcttcttctgaaaactctgggcatttctttgagcaccttgtggaggggctaaggaagtgtgtaaatgagctgggaccagaggcttctgaagctgtgaagaaactgctggaggcgctatcacacttggtgtgacatcaagataaagagcggaggtggatggggatggaagatgatgctcctatcctccctgcctgaaacctgttctaccaattatagatcaaatgccctaaaatgtagtgacccgtgaaaaggacaaataaagcaatgaatacaaaaaaaaaaaaaaa

SEQ ID NO: 20 - DNA, 775 bp, unknown
  >sr|IMAGp998B171780.3|cl|IMAGp998B171780|gb| BX108462|oid|234551303 BX108462 Soares ovary tumor NbHOT Homo sapiens cDNA clone IMAGp998B171780 ; IMAGE725680, mRNA sequence
cctgattgtcatagacaaatcctacatgaaccctggagaccagagtccagctgattctaacaaaaccctggagaaaatggagaaacacaggaaataaaattggaacgaagaaaggttaggagagtagggaaggaacaggactgcaaaaatccttctccaccgcacagactgggaacccctcctggcctgggggaagagtttgttacctaccttactatttaaagagccttcactggttctgcatcacccgcccctggacttcttagttgtttctctagcgctgagctatctcctaactttggacctattatcagaaggtgacaagtactggctctttattcattaagctttttttttttgaaccccattctttccttctctgaaagtggtgctataagttttagaatcttttaaatacattccctgggccaacagacccacacacttagccattgaaatgtcaaattgatgtgccctagatcaacagatcaacaataccttttttttcagtgttaaggtaatggttggtttttgtgtccgctaaatatttaccttgaaaaaaagaaaagtgtgtatctagcttcttcagagatcaagtcctctggtaggaggcaaaggttctatctgcttagcaactagttaataagtggtatctgacacactctaaaccccgtgttcaaacgggggccttctggttttaggaaacttgtagaaacgaagcctgctgattgatttttttctcctttttttttttttttttttttaactttggaag

SEQ ID NO: 21 - DNA, 773 bp, unknown
  >sr|IMAGp998A2488.1|cl|IMAGp998A2488|gb| BX098995|oid|234532364 BX098995 Soares fetal liver spleen 1NFLS Homo sapiens cDNA clone IMAGp998A2488 ; IMAGE111647, mRNA sequence
atgaatgcagaacctcaagnctacctgtgtcaatatcaatgtgtcaatgaacctgggaaattctcatgtatgtgcccccagggataccaagtggtgagaagtagaacatgtcaagatataaatgagtgtgagaccacaaatgaatgccgggaggatgaaatgtgttggaattatcatggcggcttccgttgttatccacgaaatccttgtcaagatccctacattctaacaccagagaaccgatgtgtttgcccagtctcaaatgccatgtgccgagaactgccccagtcaatagtctacaaatacatgagcatccgatctgataggtctgtgccatcagacatcttccagatacaggccacaactatttatgccaacaccatcaatacttttcggattaaatctggaaatgaaaatggagagttctacctacgacaaacaagtcctgtaagtgcaatgcttgtgctcgtgaagtcattatcaggaccaagagaacatatcgtggacctggagatgctgacagtcagcagtatagggaccttccgcacaagctctgtgttaagattgacaataatagtggggccattttcattttagtcttttctaagagtcaaccacaggcatttaagtcagccaaagaatattgttaccttaaagcactattttatttatagatatatctagtgcatctacatctctatactgtacactcacccataattcaaacaattacaccatggtataaagtgggcatttaatatgtaaagattcaa

SEQ ID NO: 22 - DNA, 623 bp, unknown
  >sr|IMAGp998H01154.1|cl|IMAGp998H01154|gb| BX102894|oid|234540168 BX102894 Soares infant brain 1NIB Homo sapiens cDNA clone IMAGp998H01154 ; IMAGE32759, mRNA sequence
ttttttttttttttaaagggaaagttaatttttaatacaaatacaaaatttatttccatagaaagcaaaatttataagttaaatataaagattaaaatagtgtttgaaaagctcactatagtgctaacaaactaaatgaaaataaggctattttgcatttaaaactgaaatacctcttaaaataatttgatccccagtgtttgctctttttgaagtaaccaacttactcttaaaaaggatggctgccaagatggaaagtcttactgggttttcatgttaacctattctttggacataactatgaattttgtatacaatgcacttcatgaaaagttgtggctcccccagattgcccacaagtgtgatcttgaagtcctaaacatttgtccatgtaagcttcaaaacagcgttaactgagttattcaagtagcagtacttaaagatacaattcttgaagcagtttcaatggtttctgatccaaataatcagtttctgaacattactacttcacataatagagtccatcttcagtttcttctcactttctctttcccttttgggtttcctttttgtggcctgaggccaccagttctttgggtactatcaagatacttccatcatgg

SEQ ID NO: 23 - DNA, 531 bp, unknown
  >sr|IMAGp998J02274.1|cl|IMAGp998J02274|gb| BX107144|oid|234548669 BX107144 Soares infant brain 1NIB Homo sapiens cDNA clone IMAGp998J02274 ; IMAGE46959, mRNA sequence
tttttttttttttnctgattctattacttttattaaatagtgggtttccacacatggctttttaaataatccaggcaggagaagagaggagggcacacttggaactcccctccccacaatacgtgattatttacattttagtaattggacaatcccggctcaggaggaggttgcaagaatctgcaaaagttggagggagcgccccaggagaacaaacagcaagccttatttcccctagcccatcccccaaaaaaccatccatcccatcctagtgtctggtggtgtccggtggtgtccatcttccattccttcccaaattatggaagtaaggttcttctcaccagaataagagcacttgggataacagagtagggtcccctcacccaaaaaaaaaaaaaaaaaaaagaagccttggggtaacaacagggcnnttnncctcccccagaataaagaatcctgggctgaggcagctaagcagcttggcccaatatgggaccctaggctaggggaaagggtccccnttactaaaat

SEQ ID NO: 24 - DNA, 742 bp, unknown
  >sr|IMAGp998L10121.1|cl|IMAGp998L10121|gb| BX111578|oid|234557533 BX111578 Soares fetal liver spleen 1NFLS Homo sapiens cDNA clone IMAGp998L10121 ; IMAGE124569, mRNA sequence
acctggcctgaatctatgtactttcatcagcgcggccagtaagtttgcacagctccagtgtctgtggaagacgagagactcaggaactgtgagtgcgctgacttggagcctctcttcctatacctgtgcaacaagaataatcacaaccttaatgaccaccaatgattttacaattcttctacgttttgtgatcatgctggctttaaatatatgggtaacagtgacagtacttcgctaccggaagaccgctataaaggctgaatgatggatacattattccttcacacagtggattttgagtaactgaaccaaaggaaaaagaagctctttgctaaattaaggtcttttataaatttagtaaatcagtttataatctttaaagccaaaggtttttttagacttgaaagaaagagccacttaaattcttgtttaaaaataccaatttgcctcctccttcctcacttcgttaggttatggtagtgctcagacatctgcagtgttgagaaggccagtcactgttggaagtcatccaagaagcccattttgagccttactcttaagttctctatgaagaactacattgatttgttggctttcagaatcttttaggaaataaatcctctccaggacaaaaatgaacatgaatggagtggcattttgttccaagtcagaggtgggcacctataataaatgactagggttcactttctgggactgatgnttaattgtaacacagatacaa

SEQ ID NO: 25 - DNA, 455 bp, unknown
  >gi|4735457|gb|AI651478|sr|IMAGp998J075711.1| cl|IMAGp998J075711|AI651478 wb06c04.x1 NCI_CGAP_GC6 Homo sapiens cDNA clone IMAGE2304870 3′, mRNA sequence.
ttttttttttattttttacatatgtaattataatgaactgaaagatatatatatatatcaaaaggaacaaaaaaatgacttcaaattcagactagaattgctcaactatcaattgctactgatcaatcatctttttaataacatcaaataattaggtctaataactacatacaactaaacagctgtctaaaggagatgctgttaaagaactgttccaggtcaaaatgaccaagctgttaaattatactcaaagtaaaaaacaaacacaataaaaaatatttaaaaaaacaaacaaacacaatagtagatactgaaataattatcaatcccctgcaagtagatggatgtacttaaggagactcaaaattctcgggtgtgggtgggatacattgggaaggctgttttaacactggagccttgcatggatttcaggaggttcctgaatctactgaaat

SEQ ID NO: 26 - DNA, 704 bp, unknown
  >sr|IMAGp998N06536.1|cl|IMAGp998N06536|gb| BX103225|oid|234540830 BX103225 Soares fetal liver spleen 1NFLS Homo sapiens cDNA clone IMAGp998N06536 ; IMAGE248261, mRNA sequence
gtcttaaacaagccacggaaactgcgatattaaatgccaactacatggccaagcgattagaaacacactacagaattcttttcaggggtgcaagaggttatgtgggtcatgaatttattttggacacgagacccttcaaaaagtctgcaaatattgaggctgtggatgtggccaagagactccaggattatggatttcacgcccctaccatgtcctggcctgtggcagggaccctcatggtggagcccactgagtcggaggacaaggcagagctggacagattctgtgatgccatgatcagcattcggcaggaaattgctgacattgaggagggccgcatcgaccccagggtcaatccgctgaagatgtctccacactccctgacctgcgttacatcttcccactgggaccggccttattccagagaggtggcagcattcccactccccttcgtgaaaccagagaacaaattctggccaacgattgcccggattgatgacatatatggagatcagcacctggtttgtacctgcccacccatggaagtttatgagtctccattttctgaacaaaagagggcgtcttcttagtcctctctccctaagtttaaaggactgatttgatgcctctccccagagcatttgataagcaagaaagatttcatctcccaccccagcctcaagtaggagttttatatactgggt

SEQ ID NO: 27 - DNA, 468 bp, unknown
  >sr|IMAGp998M17826.1|cl|IMAGp998M17826|gb| BX108828|oid|234552035 BX108828 Soares_fetal_heart_NbHH19W Homo sapiens cDNA clone IMAGp998M17826 ; IMAGE359608, mRNA sequence
ttttttttttacttagtctgtgtttggttccccagttttatgctgcctttctactctgttcttgctgtctccctctttacctgagtcaacggtactgagtcctgtctctctctgatgttccccagtcttccttggtgcatgttctagctccacacactagtccttggaggaaggttgagaccaatgatttcctgttatgagtcatgaggaaactgaatcacctagaagtggaataatgtgctcagggtcaccatagcccattagtggaaggaccaggactagacctttagtcttctgaggtccagccccttaggctgtctgtcatcactgtacccaagtgatgtcactaccaaggccaaatgatggtgggctaaattttaattctcaaaagtgtaggaggctaatattgtcttctaagttccaaaagaagatgtaataaaagtctgttaccttaaaaaaaaaaaaaaa

SEQ ID NO: 28 - DNA, 756 bp, unknown
  >sr|IMAGp998P11794.1|cl|IMAGp998P11794|gb| BX093757|oid|234521882 BX093757 Soares_fetal_heart_NbHH19W Homo sapiens cDNA clone IMAGp998P11794 ; IMAGE347386, mRNA sequence
aggcctaggaagagggtcattcttaagccacacattagctgcactgcgtggctgcagccaaaacaaagaactgggtgttgagtattcatcaactaagaaccaaaatccagggcactcatatgtgaaggataagaacctcacttccttactcctccaaaaagaagtggggaaagaaccatcaaacctttcctcctgacttaccaaaccaggaaaacagcaggagagggtggctcaggacttagggacagggtatagcttagatggtggaaagcaaaggagagcaggaagttgtaaatcactggctaatgagaaaaggagacagctaactctaggatgaagctgtgactaggctggagttgcttccttgaagatgggactccttgggtatcaagacctatgccacatcacactggggctagggaagtaggtgatgccagccctcaagtctgtcttcagccagggacttgagaagttatattgggcagtggctccaatctgtggaccagtatttcagctttccctgaagatcaggcagggtgccattcattgtctttctctcctagccccctcaggaaagaaggactatatttgtactgtaccctaggggttctggaagggaaaacatggaatcaggattctatagactgataggccctatccacaagggccatgactgggaaaaggtatgggagcagaaggagaattgggatttttaagggtgcagctacgctcaccctaaacttttggtggcctggg

SEQ ID NO: 29 - DNA, 363 bp, unknown
  >gi|772089|sr|IMAGp998O08148|cl|IMAGp998O08148| gb|R18479|R18479 yf95f04.r1 Homo sapiens cDNA clone 30149 5′ similar to gbX14174 ALKALINE PHOSPHATASE, TISSUE-NONSPECIFIC ISOZYME PRECURSOR (HUMAN);.
acgtgatcatggggggtggccggaaatacatgtaccccaagaataaaactgatgtggagtatgagagtgacgagaaagccagggnaccgaggctggacggcctggacctcgttgacacctggaagagcttcaaaccgagatacaagcactcccacttcatctggaaccgcacggaactcctgacccttgacccccacaatgtggactacctattgggtctcttcgagccaggggacatgcagtacgagctgaacaggaacaacgtgacggacccgtcactcttccgagatggtggtggtggccatccagatcctgcggaagaaccccaaaggcttcttctttgctngttggaagggaggcaga

SEQ ID NO: 30 - DNA, 449 bp, unknown
  >gi|5367341|gb|AI801869|sr|IMAGp998C095623.1| cl|IMAGp998C095623|AI801869 tx28e05.x1 NCI_CGAP_Lu24 Homo sapiens cDNA clone IMAGE2270912 3′, mRNA sequence
tttgttgttgatccattcataagcatttattgagggctgactggatgcccagcactagggaaacggcagatggcacaaagggtggggtccccaaggacagggcgatttcagctcttcccttgccaaccccgggtcatccccagggctcaatcccaggctggggggagggtctgggagtgggggcagtcgtcatagggtgggtcttcctggaaatccagccgagcccgggtgcccttgcccaggtgtaggcagcctcctaggtggacatggagggagagcaggagggcgaaggctcagacttcccggcacctctgaaactcggtgtttctctacgtctaactctccttcttgctgcaacgcatttgccggggcgggcggcctctgcattcctgaactgggtcagaccaggagagaggattcctggatcctggcccagtggccaatccgcc

SEQ ID NO: 31 - DNA, 525 bp, unknown
  >gi|4969890|gb|AI692550|sr|IMAGp998O205798.1| cl|IMAGp998O205798|AI692550 wd73f10.x1 NCI_CGAP_Lu24 Homo sapiens cDNA clone IMAGE2337259 3′, mRNA sequence
atgttaaaattgcacaaacatggtgctctaccaggaaggattcgaggtagataggctcaggccacactttaaaaacaaacacacaaacaacaaaaaacgggtattctagtcatcttggggtaaaagcgggtaatgaacattcctatccccaacacatcaattgtattttttctgtaaaactcagattttcctcagtatttgtgtttttacattttatggttaatttaatggaagatgaaagggcattgcaaagttgttcaacaacagttacctcattgagtgtgtccagtagtgcaggaaatgatgtcttatctaatgatttgcttctctagaggagaaaccgagtaaatgtgctccagcaagatagactttgtgttattctatcttttattctgctaagcccaaagattacatgttggtgttcaaagtgtagcaaaaaatgatgtatatttataaatctatttataccactatatcatatgtatatatatttataaccacttaaattgtgagccaagccatgta

SEQ ID NO: 32 - DNA, 461 bp, unknown
  >gi|788859|sr|IMAGp998A10192|cl|IMAGp998A10192| gb|R33016|R33016 yh70b05.s1 Homo sapiens cDNA clone 135057 3′ similar to SPZK783.2 CE00841 TYROSINE PHOSPHATASE ;._gi|1343426|gb|G23100|G23100 human STS WI-11994
ggacaatgcttttaatggcaagtcatcacagcaggtctgcggaggtgcagggcagcgctcaggccttgctcagtttcttcttgatgaagtagctcaccagccgctgcggcctctgctggtactcgctgagcacattgcgagggctgctgatctggtccccttccaggcggttcaggagggtgacacacaccacggccgnttggaggccctagggctngcacatggcggcaaacaccgaggactccatctcgatattgcggacgccggctgcataggctgcctccagatacgcctgcttgtccttctccgtntaggagcagagagccccatccagacggccttgcccttcataggaagtccaagggtgcacatgggttttccccaccactnttggttgaactcgttcagttttgcagaacacagcaacagtttctgcaccagtttttttttaaaggnccttt

SEQ ID NO: 33 - DNA, 500 bp, unknown
  >sr|IMAGp998F20979.1|cl|IMAGp998F20979|gb| BX115560|oid|234565495 BX115560 Soares_fetal_liver_spleen_1NFLS_S1 Homo sapiens cDNA clone IMAGp998F20979 ; IMAGE418195, mRNA sequence
taatcagtgatgatactttggtgtatgttcaggactgaaaccttcttattgtgctaacttgatggtttctttaaaaataatagtcttgccataaattatgaataagacttgtttttttagccatatactcagaagaacactagagttctccaactggctacgcttagttcttgctttgccaggtagtcccacgcttactataatgttcaatttagagttctaagtataaaatatttttacttatttatttatttttgtgacagagtcttgcactgttgtccaggctggagtgcagtggtgtgatctcggctcactgcagcctccacttcctgggttcaagtgatcctcacgcctcaacctcctgagtagctgggattacaggtgtgagccatggtgcctggctaatttttttttctttttttccttttctgagacggagtctcccactgtcactccagtttgggcaacagagtgagaccctgtttaaaaaaaaaaaaaaa

SEQ ID NO: 34 - DNA, 565 bp, unknown
  >gi|4736233|gb|AI652254|sr|IMAGp998B095717.1| cl|IMAGp998B095717|AI652254 wb28c05.x1 NCI_CGAP_GC6 Homo sapiens cDNA clone IMAGE2306984 3′, mRNA sequence
cacagtatgaatgtattttattttgtgaaccgggaaggacaccaaccaaaacataattgaaacaaaagtttcacagcacaatagttaccttagtacatgtgatacacaatggactctattcttgtccacactgttccattaaagaaaaatatgacacacttactgatttcacagtggggccttgaaagtgcccacaataggctgtcaggtgagaggaaacacagaagggaggtagcttactacagaatgatcatggcctaataacatctgggtgattatatgggttgcacctcagctgtctataaactttagcgtcagcagatctgtccatggctgtctctgcttttccagttggttgggcatatctccggcctgagatggtcacccgtcagaatcctttgagtatcttgccagaattcagcagtttttctgtgcaccaggaggatgctgcagggaggctgagattctctgaagatacgtgtattgctgaaagtagacttatgattatggaatcataatttccttgtacttttatctaaaattatacatgagcccgctgaaggag

SEQ ID NO: 35 - DNA, 518 bp, unknown
  >gi|4889149|gb|AI678967|sr|IMAGp998M125582.1| cl|IMAGp998M125582|AI678967 tu60b06.x1 NCI_CGAP_Gas4 Homo sapiens cDNA clone IMAGE2255411 3′, mRNA sequence
ttttttttttttttttcttacaaatgggagttttatttgcattctaacccaagcacagatcccacatacagacttctcgtccgcactatggtacagtaacaagtgcaaaatatgctttatttcaggtacaaaaacatggcagtaggacaactttgagctcaacgcccacctccccaggacctttcacacccacctgacccatccaagggccacaccaccccgacagatactcccaccacctttagaaaagagtcacccaatctggagaaaggtgtggaggttacatctttagaaagaaatcattttaaatacatgaacattagagaacacagtaaccgtgcttccacccagcacggggaggctgcagagaggcaccccaaacagtccccttctcttctgtggttaaccaagcaagccccgcaaagcttttcccagcagagcacacccagccagcaatgaggcctcaggacagagccagcactgtgacaaaagaacttgtcccacctatctgagagcat

SEQ ID NO: 36 - DNA, 430 bp, unknown
  >gi|3801639|gb|AI219436|sr|IMAGp998M154512.1| cl|IMAGp998M154512|AI219436 qh13a08.x1 Soares_NFL_T_GBC_S1 Homo sapiens cDNA clone IMAGE1844534 3′ similar to contains Alu repetitive element;contains element MER33 repetitive element ;, mRNA sequence
tttgagacaagagtctgactttattgcctagtggagtggcatgatctctgctcactgcaacctctgtctcccgggttcaggcaattctcatgcctcaacctcctgagtagctgggattacagacacatgccaccacgcccgactataaaattgagttcttatagttcaataaagttaatttattatctctttatgctagtattatagaattttggagcaggtagtatcttagattccctacattagcagtgttatataaaagagaaataattcaagtcacatatgtaatttaaaatattttagtagccatattaaaaaagcaaaaagaaacaggtgaaattacttttgattgtacatttttttaacccagtatatcagaaatactgtgtcaacatgtaatcaatgtaaaattattagagatagtttgcac

SEQ ID NO: 37 - DNA, 493 bp, unknown
  >sr|IMAGp998M144547.2|cl|IMAGp998M144547|gb| BX107117|oid|234548615 BX107117 Soares_NhHMPu_S1 Homo sapiens cDNA clone IMAGp998M144547 ; IMAGE1857973, mRNA sequence
aggacataggctgctaatgaacagccccatctgatttaatcactgtgcaatgaccgtgaactgtacagttacctccgtgtgtcacaggaagacttttttgacttaagcacccttccatggtcatgaggtttctggttgcctctgtgtgttagaagcttggagaccaatgccttctgctgtgatcgcgttctccacagttgcctctgggtaggagcctggacactaatagaaaatggatgcacttgagccgacctaagcattcatggtcatgagatttgtggttgccactgttggttaggagcatagagatcagtaatggacacacctgggctggcagaacatcttgccttgatctggagccccatgtctgcccctctgtagaggtgcacatgggccaatgatggatgcaatgagctgatcattttgctgtgatcaaggctatgttgcctctgtgttaggccaatgtgagtaatggttcttgtcttggtttagg

SEQ ID NO: 38 - DNA, 294 bp, unknown
  >gi|4312796|gb|AI459915|sr|IMAGp998L105312.1| cl|IMAGp998L105312|AI459915 ar81h05.x1 Barstead colon HPLRB7 Homo sapiens cDNA clone IMAGE2151705 3′, mRNA sequence
tttccagtacaaaaccaatttattttacattccatcgtgggcgatgggagaagctaaataataatttagcatgcatacgtagacgtttctttttatatataaatacaatatacaaaaataaagacagtacagttttgagatttccagcagtttgaacgctcgtgacataatccccccgaccccagagacgtaagcataaacccttttttctttttccaaatactctgcagaatggcggctccagaggcggtttcaagtttcataagtcaggtaacactgtgggtttccgccttc

SEQ ID NO: 39 - DNA, 434 bp, unknown
  >sr|IMAGp998D201008.1|cl|IMAGp998D201008|gb| BX109226|oid|234552831 BX109226 Soares_fetal_liver_spleen_1NFLS_S1 Homo sapiens cDNA clone IMAGp998D201008 ; IMAGE429283, mRNA sequence
tgaagatgcagcccctggatggtcccagactctcaggacatgcccagctcaggggcttcgagccacaggcctggcctcatatggcatgagggggatctggcataggagccagactgctcttagccccctggctttgtgccaggcctggaggagggcagtcccccatggggtgccgagccaacgcctcaggaatcaggaggccagcctggtaccaaaaggagtacccagggcctggtacccaggcccactccagaatggcctctggactcaccttgagaagggggagctgctgggcctaaagcccactcctgggggtctcctgctgcttaggtccttttgggacccccacccatccaggccctttctttgcacacttcttcccccacctctatgcatcttccccccactgcggtgttcggcctgaaggtggtggg

SEQ ID NO: 40 - DNA, 513 bp, unknown
  >gi|5397100|gb|AI810534|sr|IMAGp998H065732.1| cl|IMAGp998H065732|AI810534 wb89h03.x1 NCI_CGAP_Pr28 Homo sapiens cDNA clone IMAGE2312885 3′, mRNA sequence
ataatantttaatttttttttttttttttttaacttttcgccttatttaatactgggatatggggtcagggagtttgccccacttggagagatgacttcccttggaaaacatggggtttccttctgaaggggaatcctgattccttccttaatgttggggggatccaaaagtgtggccagatggaacatctcattggcggtttctttcttgcttgcttggggcttctctgtttggccttttccccattctcctttgtgtccggcagggggagaggcggcttcgtataagggcctttttgaggtttattatttccttggccatatacttgatgctcttcattggcttgtctgggacctgccttaggttctccgaggcataaaagggccggacagcccccgagttgggggaactctgaagcttcttggtggctggaaccttggtcatcttaaaaatccttcaggttttagcctgtgcccccaagacaaggatttttccagaatcttctacttcag

SEQ ID NO: 41 - DNA, 489 bp, unknown
  >sr|IMAGp998H084295.2|cl|IMAGp998H084295|gb| BX104734|oid|234543848 BX104734 Soares_fetal_heart_NbHH19W Homo sapiens cDNA clone IMAGp998H084295 ; IMAGE1691575, mRNA sequence
ctttgtgagttcttttccctcctttaaagttgggaaaacttgattcgtggggcaggagattgtttcttcattcttctgacagcccccatctgacgcgtaactgcccattttaaggaaactcttggtgctacaaaaccctgaccagacacttggcaaatttacctctttcttcaaaagaaaaactttaagaaaatgagccaatgggcttcattctcagtcatgcccggagatcacccaggagaaataatacaaacaccaccactgtccagagagagtaaagaagcagaaagagaaagaatttgcaaccatgaggaatgttcccacctcccgacgggacgtgcatttggaaaacacagaatcagccctcagggtgcactccagccacctcagtgctctaagctcacagaagtgaaataatgtctgtgggttggcaatggctttgtgggatcatatgtcttggccaaagatgggaaaacctatgttgaagag

SEQ ID NO: 42 - DNA, 467 bp, unknown
  >sr|IMAGp998F176102.2|cl|IMAGp998F176102|gb| BX107249|oid|234548879 BX107249 NCI_CGAP_Ut1 Homo sapiens cDNA clone IMAGp998F176102 ; IMAGE2453776, mRNA sequence
ttttttttttttttaagcaagcacattataagaagcaatagatgtgccagggcatatgaacatcagatgaatggaagttatcctgtgtttttctttttaacttaaattcatcttttttgaattaaatcttaattgatacatcctgtatatgaaatttcactttatggtataaatcagaggcttcctatcctataggaaagattcatgcttgtttttcaaattcagtacaattctcccagtgatttctaggaaagaataaagaggagaggagaggtagacatcagaatgtagagaggcaatactgtgtaaaaatgaaaaacaaaaaaaaaaactttatttctattttcccaggctgcacaaatctggctttgatgaagaaactcagaagatcctgatgtctgtggaagaaaaaattccccatctgaccatttcacatggaccttggattgacgaggaatacaagatca

SEQ ID NO: 43 - DNA, 275 bp, Homo sapiens
  RZPD CloneID  IMAGp998K013431
tttttttttttttttttttttttttttttttttttccaaaagccaaaaagtttatggccagaaaagcaaacttcataaagaccccttaaagtacatcgaataggacaagcaaaataaacagaaaactttgcccaaagaaaagatggccgcggtcaggcacagtcaaattaataccaacccaaacagggtcctcgaagggtataggggttatacaacccaccctcggaaactaagggggccccattccaaaatccttggttttgggttgggggttt

SEQ ID NO: 44 - DNA, 359 bp, unknown
  >sr|IMAGp998M23174.2|cl|IMAGp998M23174|gb| BX113547|oid|234561471 BX113547 Soares infant brain 1NIB Homo sapiens cDNA clone IMAGp998M23174 ; IMAGE41558, mRNA sequence
ttttttttttttttaagcagaaaaaagagaatttttaanggcncaacaaaaaagatttacagcctacttttctacagaaatgatggaagtcanaanacaanggagngacatcttcaggtaaagtnttaaaagaaaataagngccaatttataattntataccagcaaaaatatgcttcaaaaatgaaagtaaaataaagatgtctactaggcaaacaaaacagagantttatcaccagcaaacctgtnctgaaagaaatgctaaagaattntttanacacaaggaaaatgatcccagagggaaacatgaaattatncaaagtnttgancaccancagaaacggtaaatatgngaataaa

SEQ ID NO: 45 - DNA, 713 bp, unknown
  >sr|IMAGp998C17793.3|cl|IMAGp998C17793|gb| BX089984|oid|234514327 BX089984 Soares_fetal_heart_NbHH19W Homo sapiens cDNA clone IMAGp998C17793 ; IMAGE346696, mRNA sequence
acatccgccaaatctatgacaaattcccggagaaaaagggtggactcaaggatctcttcgaacggggaccctccaatgccttttttcttgtgaagttctgggcagacctcaacaccaacatcgaggatgaaggcagctccttctatggggtctccagccagtatgagagccccgagaacatgatcatcacctgctccacgaaggtctgctctttcggcaagcaggtggtggagaaagttgagacagagtatgctcgctatgagaatggacactactcttaccgcatccaccggtccccgctctgtgagtacatgatcaacttcatccacaagctcaagcacctccctgagaagtacatgatgaacagcgtgctggagaacttcaccatcctgcaggtggtcaccaacagagacacacaggagaccttgctgtgcattgcctatgtctttgaggtgtcagccagtgagcacggggctcagcaccacatctacaggctggtgaaagaatgagagactcggggagcagggagggggggaagagacgtgtgtgcaggaaacggggacggggggaaggggaccctgcaggggcagccccctgaagtgccaagagagctgagaggagcagttgtgactctacccaggaacaaactgtgcctgaacctgaggtgcccaaccccaaataaacccaagatgctgngtaaaaaaaaaaaaaaa

SEQ ID NO: 46 - DNA, 432 bp, unknown
  >sr|IMAGp998O144918.2|cl|IMAGp998O144918|gb| BX100064|oid|234534504 BX100064 NCI_CGAP_Lym12 Homo sapiens cDNA clone IMAGp998O144918 ; IMAGE2000485, mRNA sequence
ttttttttttttttgacagtgagcaataaagtttaattaaccagtactgtgaggtcaccttcacggactagcatcccttccgtgagctgaaggactgaataagcagaagcacaggccaaacccatccctccatgctccccactgtgcagatgggctcactgaggctccggtgtacattgcttgctcagcactggatctatttcgggccaaagtgtcagcagaactgatacccacagggcatccaagtccacatccagactgggctccctctaatgccatctcttaaaccgaaccactgcctgccgatggtgagtttccaatcacgagctgcactgggggtgctggaggcctggctgggcacacatcctggtgggtatgaaggggatttggggagtggcatctagaagatcagcatgaggggcacagaggaga

SEQ ID NO: 47 - DNA, 710 bp, unknown
  >sr|IMAGp998O021010.1|cl|IMAGp998O021010|gb| BX115510|oid|234565395 BX115510 Soares_fetal_liver_spleen_1NFLS_S1 Homo sapiens cDNA clone IMAGp998O021010 ; IMAGE430297, mRNA sequence
ctggtggccttgctggatgacccaacaaatgcccatttcattgcctggacgggccggggaatggagttcaagctcattgagcctgaggaggtcgccaggctcttgggcatccagaagaaccggccagccatgaattacgacaagctgagccgctcgctccgatactattatgagaaaggcatcatgcagaaggtggctggtgagcgttacgtgtacaagtttgtgtgtgagcccgaggccctcttctctttggccttcccggacaatcagcgtccagctctcaaggctgagtttgaccggcctgtcagtgaggaggacacagtccctttgtcccacttggatgagagccccgcctacctcccagagctggctggccccgcccagccatttggccccaagggtggctactcttactagcccccagcggctgttccccctgccgcaggtgggtgctgccctgtgtacatataaatgaatctggtgttggggaaaccttcatctgaaacccacagatgtctctggggcagatccccactgtcctaccagntgccctagcccagactctgagctgctcaccggagtcattggganggaaaagtggagaaatggcaagtctagagtctcagaaactcccctggggggtttcacctgggccctggaggaattcagctcagcttcttcctaggtccaagccccccacaccttttccc

SEQ ID NO: 48 - DNA, 666 bp, unknown
  >sr|IMAGp998E201114.1|cl|IMAGp998E201114|gb| BX109581|oid|234553541 BX109581 Soares_pregnant_uterus_NbHPU Homo sapiens cDNA clone IMAGp998E201114 ; IMAGE470011, mRNA sequence
cttgggtggatgggaaagttaaatattggtctcttcaagttttttttttcttttgctttgttaccacttgtcactgtctccatgttaaaatgccaaaaatgatgtagttgttgttgcttttttccctattttccaccccagtcgctccttaccgtgactcctgcccttggagggcatgtagcagtgtctgtcctgccagtcccaaggccctgtgggaggagactggcctgcatctctctaagacttagtctgacgccacgcgcatctcttgttctgtgttcaatcagtagtccaggggagaagcttctgctacttcagagctttgctaaactaacctaatttgtccaaatcaccccaaaaccaccatctctgacgtaagcttccatgcgacagcctgatccgtttccctggacaggtctctttcctggaatgcagcccaggcacctgtgctcctggcacccttgaggtctctcctttgagccgtggtcaccgagagggttgaggacgcagcacccgaggtcccagcctttgcaggagcctccctgggcttagctggacttagatcttcggtggcctcatgtaaacgtggcagccagcctcttctagaaccctagcccagggactggagcaggaaagggaccttcaaagtgaagactgccttgtccc

SEQ ID NO: 49 - DNA, 1515 bp, unknown
  Homo sapiens|chromosomeNCBI34243429015434305291
ggctttgctaatcacatcacaagaccttgagccagggccaaaggcgcggagtggcgcgggacgtcccgggcgggggagctgactctgaggacaggaggaaaggccgggagagccgtgcgggcttgaaggcgacttagcgccacctttccgtctgctggtcgggccggccttccccagccctctggggctgggggtgcagctgcctgccgaggctccccgcccggcacagcctgggcctgttgcgtcctcccagcctcttccctccccggcagtgggggcgccgcgcccagcctttgctcgatgagggctgcaggatccggctgagaccaacccgcggcgcaggtggggctgcacagacgctgatggtgggcgcgccaccctctgttttccctcactcgccagccccgcaccccaacagcttgcgctggaaaatgcgctttgccagcgggcgccccgccttcgtccgggtacggaggggacactcccatctagcgcccagtgaggctaaagccgctgacagccccactaggaacgatctggagtctggtctagggcacttctccctccgcgttcatacaggttgccacaaaaagcttgggcctgaagctcgaaggccccggacccgctggggaaagaactaggcctgccactcagggattcgagtccctggagtgtatcctgcatttcccactactgtactcccctcatggcccagggagccagagtccagggtaaggatcaggaatgctgggctggagttcaggtcctgccccagagaaatcatgtggtcttaggcaaatcacatcatctctttggtacttgggtttccttgttaaacgaagataatattcttgctttattcatctctcacctttggcaaggtgcaaatgatatctgtgaaggtgtttaaaagacaagcaagaactaaaccatctttatgtattaaaccaaattgacaaaagcgtaagggtggagaaagcctggcttctgaactgtggccagatggacagagagccacagaatcatgagtgcagagctgtgggggcacccaagtcctggaccctaggactcacagccaagagcaggtgacctcagagggaccagtttcacttttagcacctaccctacactgaccccttgacatacctgggcaactgtggtctggtttgttctttggtgagatgaccccaaacatctcaccaaactgtgaaacagccacttccctaggcactcctattaggttagtaacaagatttcagatacagaaccagtggaaacatctgtttgaaacaacgtccaaatctggggtgagtcccagtcaacagctgtgagaacacccactttgcagattaggattctggagataacatgtggacatgttacgggacatgcttgtgtacagagtacctgctagtataaaatgtctaatgtagctgcttttatttcattttaaatggagaaaaaaagaagtgctggccatttccaatgagctgggctttgttgggccagtttcat

SEQ ID NO: 50 - DNA, 302 bp, unknown
  >gi|4851404|gb|AI671673|sr|IMAGp998G195647.1| cl|IMAGp998G195647|AI671673 ty26e10.x1 NCI_CGAP_Ut3 Homo sapiens cDNA clone IMAGE2280234 3′, mRNA sequence
ttttttttttttttttttttttttttttttttttttttttttttttttttttttttttttcaaaaaaaccggggaatttttttaaaggccggggggccccagaacccaagggggttgggggttttaaaggacccgaaaaaaaaagggtaaaacccccgaatttccccccggcccccaaaatttggggggggaaaaaaggggttttggaaaaaaaccccccgccaaacccaaccaaaaaaaaaatcgggagggggtttttttcccccttggggtttaaaaaaacccccaaaattaggggccca

SEQ ID NO: 51 - DNA, 538 bp, unknown
  >sr|IMAGp998K06166.1|cl|IMAGp998K06166|gb| BX092114|oid|234518596 BX092114 Soares infant brain 1NIB Homo sapiens cDNA clone IMAGp998K06166 ; IMAGE38228, mRNA sequence
ttttttttttttttgattaagaaagctaaatttatattaaattatcataaagtcctaaaatactgaacatagtggttaaataactccagaaagtccaatctctccagtgagtaacgttaaaaccattacacatgagcatgggagaatcgcttccattagtttaggacagagagattttgctttttacagagtaaatcagtgctcaaatagatacttcctcaaatatgtcctttctacattctgaacagcccaagtgcaataagatccttccccctttccaatcaagaaaatgccacttttctacttgctcttcctccccagacatgagtctaaggacccaaagtgctcactcctttactgcttgttaagtgtaatgtggggaggctcagaactggggctgacgctactgagagcaaggctaagggcctggtatctctctctagcagtcttagaactgactagaaaagaattgatcttgtcctttttcactttggtagctgatttctcaataccagtaaccacaggagaagnttcaact

SEQ ID NO: 52 - DNA, 680 bp, unknown
  >sr|IMAGp998D03384.3|cl|IMAGp998D03384|gb| BX111866|oid|234558109 BX111866 Soares fetal liver spleen 1NFLS Homo sapiens cDNA clone IMAGp998D03384 ; IMAGE200018, mRNA sequence
ttnggccctacgngccagtaattcggcacgagnatttgccttggctcagaaggcctctgggggccatccctgaccactaggcttcccacttagacctgctccagcagcaccacccctcgccactgcctggtcctttccttcacccttgattctgtcttcttttgtccttctccaggtcttggcagcacctgtctcctttcaccccagggcctgagcctggccagcctacaatggggatgttgtgtttctgttcaccttcgtttactatgcctgtgtcttctccaccacgctggggtctgggaggaatggacagacagaggatgagctctacccagggcctgcaggacctgcctgtagcccactctgctcgccttagcactaccactcctgccaaggaggattccatttgcagagcttcttccaggtgcccagctatacctgtgcctcggcttttctcagctggatgatggtcttcagcctctttctgtcccttctgtccctcacagcactagtatttcatgttgcacacccactcagctccgtgaacttgtgagaacacagccgattcacctgagcaggacctctgaaaccctggaccagtggtctcacatggtgctacgcctgcatgtaaacacgcctgcaaacgctgcctgccggtaaacacgcctgcaaacgctgcc

SEQ ID NO: 53 - DNA, 713 bp, unknown
  >sr|IMAGp998E01223.1|cl|IMAGp998E01223|gb| BX103782|oid|234541945 BX103782 Soares placenta Nb2HP Homo sapiens cDNA clone IMAGp998E01223 ; IMAGE147048, mRNA sequence
cacgtgtggtaccagggcaacgcgtcgctggggggacacctaacgcacgtgctggaaggcccagacaccaacaccacgatcattcagctgcagcccttgcaggagcccgagagctgggcgcgcacgcagagtggcctgcagtcctacctgctccagttccacggcctcgtgcgcctggtgcaccaggagcggaccttggcctttcctctgaccatccgctgcttcctgggctgtgagctgcctcccgagggctctagagcccatgtcttcttcgaagtggctgtgaatgggagctcctttgtgagtttccggccggagagagccttgtggcaggcagacacccaggtcacctccggagtggtcaccttcaccctgcagcagctcaatgcctacaaccgcactcggtatgaactgcgggaattcctggaggacacctgtgtgcagtatgtgcagaaacatatttcctcggaaaacacgaaagggagccaaacaagccgctcctacacttcgctggtcctgggcgtcctggtgggcagtttcatcattgctggtgtggctgtaggcatcttcctgtgcacaggtggacggcgatgttaattactctccagccccctcagaaggggctggattgatggaggctggcaagggaaagtttcagctcactgtgaagccagactccccaactgaaacaccagaaggtttggagtgacagc

SEQ ID NO: 54 - DNA, 317 bp, unknown
  >gi|4073985|gb|AI337058|sr|IMAGp998D054941.1| cl|IMAGp998D054941|AI337058 qx82g03.x1 NCI_CGAP_GC6 Homo sapiens cDNA clone IMAGE2009044 3′, mRNA sequence.
aagtcttcattaagagaggaaaagatgaaaactgcctttacatgataaattatatatgtcaaaaatctagtatcaagtttaatggaacaatgtcgcagcctgatgggttcttctttcccactgccctgaaaaaagactaatgagaccagcaggagttgcaacaaagaatgtaataatcacaggcctgctgagcaaggagaacagggagaaagttctcaaacctgtctcccccagaatttggaggctggggttttgtaaaagtactttttaaaaagtactttcccagcagggttctgggaaactggaacagatgattg

SEQ ID NO: 55 - DNA, 832 bp, unknown
  >sr|IMAGp998A17115.1|cl|IMAGp998A17115|gb| BX097937|oid|234530247 BX097937 Soares fetal liver spleen 1NFLS Homo sapiens cDNA clone IMAGp998A17115 ; IMAGE122008, mRNA sequence
acaaaggctattaacacataggaaaaaatgtatttactaagtgtcacatttctctaagatgaaagatttttactctagaaactgtgcgagcacaacacacacaatcctttctaactttatggacactaaactggagccaatagaaaagacaaaaatgaaagagacacagggtgtatatctagaacgataatgcttttgcagaaactaaagcctttttaagaaatgccagctgctgtagaccccatgagaaaagatgtcttaatcatccttatgaaaacagatgtaaacaactatatttcaactaacttcatcttcactgcatagcctcaggctagtgagtttgccaaaaccaaagggggtgaatacttccccaagattcttcctgggaggatggaaacagtgcagcccaggtcccatgggggcagctccatcccagagcatttctgatagttgaactgtaatttctactcttaagtgagatatgaagcattatccttttgttcagttgccccgggcttttgaacagaagagtaaatacagaattgaaaaagataaacactcaaccaaacaatgtgaaaacgggttctgtagtatttgtaaaaaggcccggcccaggaccactgtgagctggaaaagggagaaaggcagtgggaaaagaggtgagccgaagatcaattcgacagacagacggtgtgtatgcccctccctgtttgacttcacacacactcataactttccaaatgaaaccccacagtatagcgcatattttcgatatttttgtgaattccaaaagggaaatcacaggggctggtcgaaatattgggggaacact

SEQ ID NO: 56 - DNA, 455 bp, unknown
  >gi|4735095|gb|AI651116|sr|IMAGp998K035709.1| cl|IMAGp998K035709|AI651116 wa97e02.x1 NCI_CGAP_GC6 Homo sapiens cDNA clone IMAGE2304122 3′, mRNA sequence
ttttttttttttttttttttttttctaagggaaattattgttatttcgctctaacgattacccaagaccccaaaataaaaactaacccagcaggttttgttcacattaatggccagagtacaacccatgaatctttaaagggagaaaaatgcttcaaattttatgccctgagagtttttcttctatttgttctagcctatcattgaagcttttgagggtactttgtatttcatccaatgatttcttcagttccagaaatactcttctgttgaattttaaaacctttttctggcaggaaatcatctcatatatctccccacctcttttcctcatttctttatgttggtgttcaaaagactcttccatgttgatgaccttctttacaatcagtccggccaattctctgctcaattctctgtttgagctttcctgaatttcttttggatggggatctg

SEQ ID NO: 57 - DNA, 480 bp, unknown
  >sr|IMAGp998F13278.1|cl|IMAGp998F13278|gb| BX092118|oid|234518604 BX092118 Soares infant brain 1NIB Homo sapiens cDNA clone IMAGp998F13278 ; IMAGE48538, mRNA sequence
ttttttttttttttaaacgaaaagcaaacttctttattgtacacagtacagaatataacgcagcttggcaggatgcatacggccctgcgcaggggaaagtatttcaaatcagctggcaggttcaagcctttctgcactgtagactttccacactctggaaaagaagcaaacaaacaaaccccaaagaacccccgaaaaaaacaaaaaccatccgggaggtgcatgagtccaatgggaatgcaaccgtgatgccgctgtcctatgcccagtgacagcacaggtcacgtaagttacagcaggggaggggtagctcaagctacagaggattattgtcatattgctaagacagcataaatccattcaaaaaaaaaaaaaaaaaaatccaaaccagggtaagtaaagaaaggaaaaccaaatctatacagcatttacaacaaataaatctctagccagctgggggtaaaatatgcatctatgtat

SEQ ID NO: 58 - DNA, 786 bp, unknown
  >sr|IMAGp998B06202.4|cl|IMAGp998B06202|gb| BX092972|oid|234520310 BX092972 Soares placenta Nb2HP Homo sapiens cDNA clone IMAGp998B06202 ; IMAGE138917, mRNA sequence
ctttgtctcagagccctctgggctctctcctctgagggagggacctttctttcctcacaagggacttttttgttccattatgccttgttatgcaatgggctctacagcaccctttcccacaggtcagaaatatttccccaagacacagggaaatcggtcctagcctggggcctggggatagcttggagtcctggcccatgaacttgatccctgcccaggtgttttccgaggggcacttgaggcccagtcttttctcaaggcaggtgtaagacacctcagagggagaactgtactgctgcctctttcccacctgcctcatctcaatccttgagcggcaagtttgaagttcttctggaaccatgcaaatctgtcctcctcatgcaattccaaggagcttgctggctctgcagccacccttgggccccttccagcctgccatgaatcagatatctttcccagaatctgggcgtttctgaagttttggggagagctgttgggactcatccagtgctccagaaggtggacttgcttctggtgggttttaaaggagcctccaggagatatgcttagccaaccatgatggattttaccccagctggactcggcagctccaagtggaatccacgtgcagcttctagtctgggaaagtcacccaaccctagcagttgtcatgtgggtaacctcaggcacctctaagcctgtcctggaagaaggaccagcagcccctccagaactctgcccaggacagcaggtgcctgctggctctggtttggaagttgggggnaa

SEQ ID NO: 59 - DNA, 476 bp, unknown
  >sr|IMAGp998E1783.1|cl|IMAGp998E1783|gb| BX109827|oid|234554033 BX109827 Soares fetal liver spleen 1NFLS Homo sapiens cDNA clone IMAGp998E1783 ; IMAGE109816, mRNA sequence
ctttgccctgggtaagctgctgtagggtcagaagtaaccctttctgtgtcagttgagaatgagcctgtgtggtagctgatgtcagaggacaaagctctctgcaagggctggacacagagctgcagagtcctgaacatccctcctttcaggctgcagaagggagaggcaatgaagacagctgctccggaagcagcatcagggctcttggaggggactggtggggactcaggctgggtgcagcctccaaacagagaacggaacttaggtgtgtctctacagctaggcccagcctagcccagcccagaacaaacacccttcagagcctaaccaaagaacataagctgcaaaatgtgcacccatattttaagctgctttttcaggggataaatagtgtttggggacattgaaatggatgttctcaggttgtatttatttcggacaaataaactagagaattgtgtaaaaaaaaaaaaaaa

SEQ ID NO: 60 - DNA, 457 bp, unknown
  >sr|IMAGp998L095608.2|cl|IMAGp998L095608|gb| BX105935|oid|234546251 BX105935 NCI_CGAP_Ut3 Homo sapiens cDNA clone IMAGp998L095608 ; IMAGE2265368, mRNA sequence
ttttttttttttttaaaaataatttactttattacaaagatagtaatacaatttctagaaaatacatgcatgtacaaagattgaaataaaaaaaacactcataaatatccagatttaaatgtcagcatataccttcctgatctttttctatcatactgtatctatcaacatctaacctgcttttttaactataagtcatacattttgccatatcagtacacttttttctgtaacattatttttagagcatatcaaaccatagtttatttaaccaatccccaattaattcataattagaatatttcgtgtttttccagtataacagttctataatctttgattataacctttgtgtaaattcctataagtattgagtcaaaggagacacacatcttttcaggcattacatataaaataaatactaaactagactccaggaaagtggtatcaattcaca

SEQ ID NO: 61 - DNA, 517 bp, unknown
  >gi|1274428|sr|IMAGp998E11660s1|cl| IMAGp998E11660|gb|W02449|W02449 za47a06.r1 Soares fetal liver spleen 1NFLS Homo sapiens cDNA clone 295666 5′
tctttagagaaaaatatttttaatgtagaaaagatgaatagctgtaatgaaatacaaaccgttcctatacacagtaaacttactaagaactgctaatccttctccaggttcttagaatctgggtacagcattatagaaagtaaccacgctatcgaaaacacacacccaaaacccgatgggtggcaattgtgatagattaggaagacaaaacagattcagaagtagcagagaatactgcacattttcttggaaaatttcttaattgttcagtgctttctgacaagtttccccagcccaatttatctcttagctattctctgctacttagtcctgtcttctgacaatcatcatacggggacgatcatctgccttacagtgtaacatgtttttcctaacacagccgcattttatctggccctaagagaatagcaactaatgggctttcaatgaagtcaccatgatttaggaaagtttaaaaccncaaatttttccatggtgggtaaaacttctaattacc

SEQ ID NO: 62 - DNA, 343 bp, unknown
  >gi|2229312|gb|AA495991|sr|IMAGp998K051891.2| cl|IMAGp998K051891|AA495991 zw06e03.s1 Soares_NhHMPu_S1 Homo sapiens cDNA clone IMAGE768508 3′, mRNA sequence
ttttttttttttttttaagaaaaaatgtctattatttattacacaataattctgaccaccaacaaccaacggcgggggcgggcaggagagaagaacatcttgcttctcaacaaactttcctcccttgctttaacatttttgaggattctttcccaaacctattacacctgtattatgatggttacaaattttccaactcttccactccttccagtgcattatttttagtatcttcattaaacgggcaaaaaaaaagatcccctacttgtaataacaaaacaatgttggaaactgtcattaaatcaggataagtgaaaaacagatctgttcccagactcgcagg

SEQ ID NO: 63 - DNA, 330 bp, unknown
  >gi|1018091|sr|IMAGp998J14406|cl| IMAGp998J14406|gb|H63290|H63290 yr49d07.r1 Homo sapiens cDNA clone 208621 5′
atgttcaacctcagtccctcaaggccatacctggacccaacggtgaagaaagacgatgaggaggaggacccgctggaccagctgatctcccgctctggctgtgctgcctcccactttgcagtgcaggagtgcatggcccagcaccaggactggcggcaatgcagccacaggtgcaggcgttcaaggattgcatgagtnaacagcaggcgaggcggcaagaggagctgcagaggangcaagaacaagccggtntccaccactgagaccncaaaccacctatcnccagtagatnggcccttcaagaccagcatcctagcaagattattagag

SEQ ID NO: 64 - DNA, 430 bp, unknown
  >sr|IMAGp998B231782.3|cl|IMAGp998B231782|gb| BX089818|oid|234513990 BX089818 Soares_testis_NHT Homo sapiens cDNA clone IMAGp998B231782 ; IMAGE726454, mRNA sequence
ctcaatggcggtctggccgtgaagaccaatgagatctctgtgatccagagtgggggggttcctaccctcccggtttccttgggggccacctccgttgtgaataacgccactgtctccaagatggatggctcccagtcgggtatcagtgcagatgtggaaaaaccaagtgctactgacggcgttcccaaacaccagtttcctcacttcctggaagaaaacaagattgcggtcagctaagggagaacttgcgtggaaggagcaatgcagacacagtgaaatctctagaatctgctttgttttgtaagaactcatctcctcctgttttctttttcttactgatatgcaaatgatgtttactacgttggttgtgaccacaacctcaggcaagtgctacaatcacgattgttgctatgctgctttgcaaaaagtt

SEQ ID NO: 65 - DNA, 365 bp, unknown
  >gi|3148939|gb|AA973759|sr|IMAGp998M024021.1| cl|IMAGp998M024021|AA973759 oq16b01.s1 NCI_CGAP_GC4 Homo sapiens cDNA clone IMAGE1586473 3′ similar to contains element MSR1 repetitive element ;, mRNA sequence
tcactttgcaattctttattagaactcaagtttgtgtttgcaaccatgcaacagaaagggtaaaacaaaaaatccacatcctctccccaaccccagcttctgggcggaaatacgggctttcactttaacccctgactggctctggctccagggcctgagatgccttcataatctagtaactgggtcccgggcatcctcgcgaaaccgctgggtcactgccttgctggcagcttcgggggatcgctgggtccccgacttgttggcagcctggagagactgctgggtcccggcctcctgcacctccaccggagaaccctgtgtcccgacctcctggacttccaccgaacgctgggtcccggcctccc

SEQ ID NO: 66 - DNA, 507 bp, unknown
  >gi|5368831|gb|AI803437|sr|IMAGp998C105092.1| cl|IMAGp998C105092|AI803437 tc39f05.x1 Soares_total_fetus_Nb2HF8_9w Homo sapiens cDNA clone IMAGE2067009 3′, mRNA sequence.
tttgtttataaatggttattttattcagagtggttgctttttaatgctgctctcttaatttcctgttaaatctgtttcatctatagccatccaatattagtatttattttgttcatttaactttaaagtattcttaaattaaatctattacaaatattctactgctggtgaagctaattcagcataggcactaccacaaaaatatggaaaacatgatcaatgtattatagatgtaatctggcttcagaaacacaatactaaaaactattttagtaaattaagaaataagcattctagattgtgttataaaatccatttctaaatatacccctttttaaacctttatgcttctttaccttggaattcttaccagcgtagctagattttgtattgcaatggtaattatacttaagaatcattgtgtgttaaaatattccccacgtacagattatttttatcatcttttccctaaaataaaattctcagcatttaacctatgcatgttgg

SEQ ID NO: 67 - DNA, 638 bp, unknown
  >sr|IMAGp998I19131.1|cl|IMAGp998I19131|gb| BX110791|oid|234555961 BX110791 Soares fetal liver spleen 1NFLS Homo sapiens cDNA clone IMAGp998I19131 ; IMAGE127962, mRNA sequence
tgtggaagttgttctagaagttagatggacagtgggagcacattcgtgagtggggcttgggagagctcaaaagtggcactttgattttaggctgggcaggcagaagtctcacagtaaaatatatttcactagacgtccaaaccaacactgcattgtagtaacctcttctgaagtaactggttaaattaataactggtcccataagtcagggtcaagccacatttataacccattcgggccggccatgaagggttctgactcgacattggtatttaaatgctgttactttcccaatgcttagtagcacttgagactttatttccatcgacaaaaaccaagaaggccacagaagtagcctaacagtcctccctggtcatactcgtttcaaacaggtctgcctgtctagacatattcaggcaggaggttgcttgctgctatcttgaaaaccactggaagattcagcttcttggggacctcagagcccatattcatatggaagcccacccatataagcctcacagagcaaatcacagcaatgttgaagttgttattttcaagtgcttatttcacgacattaaaaaaaatattttttcctggtgtattaaaattaaaaataaaatcataacttttgatttaaa

SEQ ID NO: 68 - DNA, 445 bp, unknown
  >sr|IMAGp998F246080.2|cl|IMAGp998F246080|gb| BX104907|oid|234544194 BX104907 NCI_CGAP_Gas4 Homo sapiens cDNA clone IMAGp998F246080 ; IMAGE2445335, mRNA sequence
ttttttttttttttaatgcacaaaggttttaataccttggctttaatgatttttcaaggttaagaaacaaattcaaattggttggagcttcaactcagtaattacaatcacaatgcatctctgaaaggccctgcatttggaggcagagtaatctgcaaagatgatagtttttacatatgtcctgttacctacaccaatataattactacattatcttataaagacaaacagttgcttcaaactctttaaaaaatatatatataatgagtttcccaaagactcgagtctatattcaaagatgagtaaaaaaaaatccattacttccctagggtcactttcttcctttactcctgcttaaatgcaaaagctgatagtttctgatttgtagaaaaatctaaaggtttctgctttttagacaaattcaggttctcttttgctttttctt

SEQ ID NO: 69 - DNA, 464 bp, unknown
  >gi|3483725|gb|AF086380|sr|IMAGp998B03791.1| cl|IMAGp998B03791|AF086380 Homo sapiens full length insert cDNA clone ZD69C02
gtttggaaacatttaatttatacccctctcctctgtctccagaagcttctagctctggggtctggctaccgctaggaggctgagcaagccaggaagggaaggagtctgtgtggtgtgtatgtgcatgcagcctacacccacacgtgtgtaccgggggtgaatgtgtgtgagcatgtgtgtgtgcatgtaccggggaatgaaggtgaacatacacctctgtgtgtgcactgcagacacgccccagtgtgtccacatgtgtgtgcatgagtccatgtgtgcgcgtgggggggctctaactgcactttcggcccttttgctctgggggtcccacaaggcccagggcagtgcctgctcccagaatctggtgctctgaccaggccaggtggggaggctttggctggctgggcgtgtaggacggtgagagcacttctgtcttaaaggttttttctgattgaaaaaaaaaa

SEQ ID NO: 70 - DNA, 727 bp, unknown
  >sr|IMAGp998N15372.1|cl|IMAGp998N15372|gb| BX105335|oid|234545051 BX105335 Soares fetal liver spleen 1NFLS Homo sapiens cDNA clone IMAGp998N15372 ; IMAGE195662, mRNA sequence
caatccttctgccttggccccccaagtgctgggattgtgggcatgagctgctgtgcccagcctccatgttttaatatcaactctcactcctgaattcagttgctttgcccaagataggagttctctgatgcagaaattattgggctcttttagggtaagaagtttgtgtctttgtctggccacatcttgactaggtattgtctactctgaagacctttaatggcttccctctttcatctcctgagtatgtaacttgcaatgggcagctatccagtgacttgttctgagtaagtgtgttcattaatgtttatttagctctgaagcaagagtgatatactccaggacttagaatagtgcctaaagtgctgcagccaaagacagagcggaactatgaaaagtgggcttggagatggcaggagagcttgtcattgagcctggcaatttagcaaactgatgctgaggatgattgaggtgggtctacctcatctctgaaaattctggaaggaatggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaatttttttcttaggagcaagaagaaaatgtggccctaaagggggttagntg

SEQ ID NO: 71 - DNA, 383 bp, unknown
  gi|2113210|gb|AA430036|sr|IMAGp998G201924.1| cl|IMAGp998G201924|AA430036 zw65f10.s1 Soares_testis_NHT Homo sapiens cDNA clone IMAGE781099 3′, mRNA sequence
tttactacatataaaatattttattacaaaaagctccattgcatggcatttcaaaaagaacatttcctacagttttctttcttcttcttttttttttttttttgctgttacacagaagagactagttaagtgtaccattgaagactattacgcatcacgtgcctacgttcatcaggccttaagccctcaaagaacaacagcatcaggggctctgcgagaccgccacctgagcctcaccgcttagttcataaaacagccctgtgtcctttctacaaagccaggtcgtgttttctacccacagttcaggactccagcgtagacagcaaacacaaaacttgtttcctttttgaaacaaaacagtcttagtagaagcaaaacaaaca

SEQ ID NO: 72 - DNA, 428 bp, unknown
  >gi|713479|sr|IMAGp998E1983|cl|IMAGp998E1983| gb|T85127|T85127 yd31a10.r1 Homo sapiens cDNA clone 109818 5′
taagcccagaatgaagctgctgggtcatcgtagggcacttgcatgctttatgatttaatgactgcctagttgtgcacagaggcctgcccagtctgtttccaccagtatatccagtaaagggtgctgtacaccacctctgcccacacttggcctttcccactctctggtggttgccagtccacaggcgtagnagctgccttgctgttcttcaggcacatccctctgggcacccacataacagaggtgtctctgtgtgctctgcctgctgttcattcccagctaattcggggagggccgagggcaggnaaaattcgctttaaacccggggagggcaggaggtttggcaagttgaggccgaggatggncaaccatttgcggttttcagttttnggggcaaacaggaggttaggatttccntttttcaaaaa

SEQ ID NO: 73 - DNA, 379 bp, unknown
  >sr|IMAGp998C09200.1|cl|IMAGp998C09200|gb| BX105022|oid|234544425 BX105022 Soares placenta Nb2HP Homo sapiens cDNA clone IMAGp998C09200 ; IMAGE138176, mRNA sequence
tttaaatgcactgaacgtgaaagcatttctctcaccaaattagaaagttcacccaggaagcttcataaagataagagacaggaaaataaacataagacctttttaccggtgaaaggtaacacagaaaaatcaaacatgctggagtttaaattatgtccagatatcttactaaagaatacaaactctgtggaagaacggaaggatgtaaagcctcatcctaggaaggagcaagcccctctgcaagtttcaggaataaaaagtacaaaagaagactggttaaaatttgttgctacaaagaaaaggacacagaaagacagccaagagagagataatgntaattcaagactctcgaagagaagcttcagtgcagntggatttg

SEQ ID NO: 74 - DNA, 607 bp, unknown
  >gi|4074023|gb|AI337096|sr|IMAGp998I204942.1| cl|IMAGp998I204942|AI337096 qx88b10.x1 NCI_CGAP_GC6 Homo sapiens cDNA clone IMAGE2009563 3′, mRNA sequence
ccagttgttacaaatttattttctatagtggacttcacatgtaattttttccaacaaagtattcaattgaatggaagcaggaaattgaaaatcactgatattgaagatgatgtactggcggataaaggcacctgccctcctgtaatattcagtagaaattttgagatcttgaaaacatatgaaaaatgtgggctatgttatcactcctgacagtgatggctttgatggagtgggatgactttggacccatctcttggccagtgtatctgtgccagggtcaaaggagaaggcactgggtttggactcactcaatccaaattgccttgctctgagattttatgtagatcagtgaaaacttgagacttcctgaagaccagaaggaggcttccattccatctgatctcacctagatctggagcaaaggcaacatgataggtaaaatttgcacatcagatggtcaaataggaatcagataataaaaatgtggtcctcgctgtctgttcagcatggtgagtgtgtgcctgtgctccctgattcttcttggctgaggtgttgagctgatactggtgagtggcaaggagtcctttgccttctgagctttgagcac

SEQ ID NO: 75 - DNA, 495 bp, unknown
  >gi|5439998|gb|AI820919|sr|IMAGp998H171818.2| cl|IMAGp998H171818|AI820919 zu39g09.y5 Soares ovary tumor NbHOT Homo sapiens cDNA clone IMAGE740416 5′, mRNA sequence
actatcctgacgtgtgacaggtccctgatccgccccagcctgcaggccagaagcagtgttacacacttcctggaggagctaaggaaatcctcagactcctgcgtttttgttgttgctgttgatgttttttaaaaggtgtgttggcatataatttatggtaatttattttgtctgccacttgaacagtttgggggggtgaggtttcatttaaaatttgttcagagatttgtttcccatagttggattgtcaaaaccctatttccaagttcaagttaactagctttgaatgtgtcccaaaacagcttcctccatttcctgaaagtttattgatcaaagaaatgttgtcctgggtgtgttttttcaatcttctaaaaaataaaatctggaatcctgcttttttgctctactagtacctctgtcacactagtcttatcaaaaaccagttcttaagaatcaatgtaagtttattagttaatgtaaatntctcatcctcga

SEQ ID NO: 76 - DNA, 719 bp, unknown
  >gi|4435381|gb|AI521246|sr|IMAGp998P175394.1| cl|IMAGp998P175394|AI521246 to66g09.x1 NCI_CGAP_Gas4 Homo sapiens cDNA clone IMAGE2183296 3′ similar to gbU02570 ALU CLASS C WARNING ENTRY (HUMAN);, mRNA sequence.
aacttattaaaaagagttttatttagcacttggttctgttggctgggaaattcaggatcaggcagctgtatctggtgggttctcatgactgcctcatgctgcatcaaaacatcgtagagaaacagaaggggaccgagtttgtgcaaacaaaaagcgcaaaatagaagaggcagcattgttttataacaactcactctcttgggaactaaccattcccaggagaacccagtctcagtgtcaagagaaagatgttaatccatcttagcaacctaattacctcttaaaaataccatctcccaacactattacattggcaattgaactacaacatgagttttggaggggccaaacaacatgcaaaccatagcagcaggtttaaaggtaaaaggatgggagaaaagtacacagaaagatattctggagaaaaggcacagagtgagccaaggcacaaggtaaaatgtttgactggttagagacgctagattattttgtgtggctaagacaaaacaaagctggtggggagtggcaggaggtaagtctagaatggcacaatggggccagactgcgaatgcaaacaagtatgtgatcttttatttctaaagaaatgtagtatgattagcacacacacacacaaaaattattggccgggcgcagtggccatgcctgttatccagcactttgggaggccgagagggtggacatgaggtcagagttcagac

SEQ ID NO: 77 - DNA, 420 bp, unknown
  >gi|4834825|gb|AI670051|sr|IMAGp998F105821.1| cl|IMAGp998F105821|AI670051 we64d05.x1 Soares_thymus_NHFTh Homo sapiens cDNA clone IMAGE2345865 3′, mRNA sequence
ttttttttttttttttttttttttttttttttttttttttttttttttttttttttttttttttttttttttggggttttaaaaaaaaaaagggtttttccaggggggaaagggaggggtaaacccgggcccccaaatgggggcccgttcccccgggtaaaaaaaagcccccggaaaaaagggtccccattgggtttaaaccccctaaaaaattttttggccccccccccccccgggggccttttggaaaaaaaaatttggctaatatggcaaaaaaccccaaccctttttcccgccccctttaaaaaaaccggcccctaccggggggccggcccccccccaaaaggggggaaattccaaatgggttgggtaaacccccccctttaaaaaaaggggaaaccggggccaaaaaaggaaaaa

SEQ ID NO: 78 - DNA, 708 bp, unknown
  >sr|IMAGp998G01671.1|cl|IMAGp998G01671|gb| BX109981|oid|234554341 BX109981 Soares_fetal_lung_NbHL19W Homo sapiens cDNA clone IMAGp998G01671 ; IMAGE299928, mRNA sequence
catctgcaatagtatggctaagacgctgcagacagccctgcagccccctgactggctgcagggccactacctggtggtgcggtacgaggacctggtgggagaccccgtcaagacactacggagagtgtacgattttgtgggactgttggtgagccccgaaatggagcagtttgccctgaacatgaccagtggctcgggctcctcctccaagcctttcgtggtatctgcacgcaatgccacgcaggccgccaatgcctggcggaccgccctcaccttccagcagatcaaacaggtggaggagttttgctaccagcccatggccgtcctgggctatgagcgggtcaacagccctgaggaggtcaaagacctcagcaagaccctgcttcggaagccccgtctctaaaaggggttcccaggagacctgattccctgtggtgatacctataaagaggatcgtagtgtgtttaaataaacacagtccagactcaaacggaggaagcccacatattctattatagatatataaataatcacacacacacttgctgtcaatgttttgagtcagtgcatttcaaggaacagccacaaaatacacacccctaagaaaaggcaagacttgaacgttctgaccaggtgccccctcttcttctttgncttctcttggcctctttctcctatttcttaccctgncctccacctgcctttcnt

SEQ ID NO: 79 - DNA, 398 bp, unknown
  >gi|4687900|gb|AI636570|sr|IMAGp998D125539.1| cl|IMAGp998D125539|AI636570 ts91h06.x1 NCI_CGAP_GC6 Homo sapiens cDNA clone IMAGE2238683 3′, mRNA sequence
tttttttttttttttttaggggaagagaagcaaggcccctcagagacacctttattcttgctctttctcctttcaagacatggttgctccctaggcagtcacatttctctggtgatggagagaaaggagggcagttggagattgaggggacagcatgggatagccgagacatgctgatagctggaggggaggctgcctgggacctgaagaagcagtgatgtcccagctccagtcatctgaactcccctaataggcctctccatctctccagacattagatcttgtagagaaagaaactgctagaatccaaatcttttcacacagtgaggcccccaaacagcccagccttcacacacaagccagcctgcttctcctgactgacttgatcaatacctcaa

SEQ ID NO: 80 - DNA, 615 bp, unknown
  >gi|1123701|sr|IMAGp998C22571|cl| IMAGp998C22571|gb|H99033|H99033 yx11f11.s1 Homo sapiens cDNA clone 261453 3′
ntttttaaagttggtctctgctttttattatcatgtgctgacaagtctaaacaatgtcaatgctaaaatatttcttcttgggcagaccattcctaccttctcggtaccacaccgtaccacaagtagcttacccattcctctagtgatagacatttgggttgtttccagcatgggtttattagaaagagcactgtagccgggcatggtggttcatgcctgttaagtgagctgaggtaaaatatattaatacatgaaatctatcattttaaccatttttatgtgtacaattcagtgcattaagtacattcacaatgttatgcaaccaccaccactctccatttccagaacatcttcatcatcccagagaatctgtttgcagcctcactccctattccanccttcccctagccctggcaacctctaccttcttggcnctgaaattggcctattcnagatgcctcatctaaatgggatcaaattggccctttgnggctggctaattcattaacaggtttcaaggtcatccacgtggagctggcacagacttatgcagcccaaaccanccctacggatcaatccccggagaaggggcaggccagcagatccctggcaccg

SEQ ID NO: 81 - DNA, 759 bp, unknown
  >sr|IMAGp998M24260.1|cl|IMAGp998M24260|gb| BX097294|oid|234528960 BX097294 Soares breast 3NbHBst Homo sapiens cDNA clone IMAGp998M24260 ; IMAGE161471, mRNA sequence
tgcacaggatcccaagtcatgctgtagagccggattcctagacccagggctctgcactctcaaggctggccccatgtgctcaagggggtctaatgtttgggctccaaactaaccatctcggagctgggctcctcatttactgccaaaccctcagcttatgtagctagaaagggccctggagtgagaaagcctggattttcaaattgatgctcccctactgactagctgtgccactctgggcaaatgctcttccttgagcctgtttccacacctgtaaagtggggatgatgatcctatctcactgcttttgtgaggattacaggaaagcacctgtcctggctctgtacctggcacgtagtaggtgctcagttcatgctggtttccttcctgcctttagtagggacctgctctgtgctcacacctcggctgcatgcaccctgctgtgacggaggctagtgtggaagaggtcctgtcctcagggaattaactgtcttattgggagacaacaactgtcctccttggaacacccaagaaaccatgcaaagcagtggacaacacagaacacgccctcctcctcgctgcctgcagctccaatctgattctgcttgggaatgggcggagcacgtgggctgcttaactgctgtataggacaagccccttacccctctctgggcccatgaattcctggcttggtttatgttctgatttgacacactgattttaatcttcgaatcatgacactgagtgcagaggaggtgg

SEQ ID NO: 82 - DNA, 751 bp, unknown
  >sr|IMAGp998N11579.1|cl|IMAGp998N11579|gb| BX118177|oid|234570730 BX118177 Soares melanocyte 2NbHM Homo sapiens cDNA clone IMAGp998N11579 ; IMAGE264778, mRNA sequence
gtttctttggattttacagaggtctgatgcctactattttaggaatggctccatatgcaggtgtttcattttttacttttggtaccttgaagagtgttgggctttcccatgctcctacccttcttggcagaccttcatcagacaatcctaatgtcttagttttcaaaactcatgtaaacttactttgtggtggtgttgctggagcaatagcgcagacaatatcctacccatttgatgtgactcgtcggcgaatgcaattaggaactgttctgccggaatttgaaaagtgccttaccatgcgggatactatgaagtatgtctatggacaccatggaattcgaaaaggactctatcgtggtttatctcttaattacattcgctgtattccctctcaagcagtggcttttacaacatacgaacttatgaagcagttttttcacctcaactaaaaaaaaattatggttggtttttcttaatacattctcagagggagaaatgaaacattactataattgtggggggaacattacttgaatggggatatttaccctgtcacaagagccactggtattttagtacttgattattttttctttagtcacaaatcagaactgcttaccatactttttgatgccaaacattataccttagaacattgaagaaaatattcctaagctgatgctggctaaaccgctttaaagntttatttggaagtagaaactagctttaaaacggggttcaagangttgcc

SEQ ID NO: 83 - DNA, 401 bp, Homo sapiens
  >ImageID=36807 IMAGP971H10113_S1 CC   FromEXP of IMAGp971H10113_s1.exp April 26, 2002 1308
agagaaaaatatttttaatgtagaaaagatgaatagctgtaatgaaatacaaaccgttcctatacacagtaaacttactaagaactgctaatccttctccaggttcttagaatctgggtacagcattatagaaagtaaccacgctatgaaaacacacacccaaaacccgatgggtggcaattgtgatagattaggaagacaaaacagattcagaagtagcagagaatactgcacattttcttggaaaatttcttaattgttcagtgctttctgacaagtttccccagcccaatttatctcttagctattctctgctacttagtcctgtcttctgacaatcatcatatggggacgatcatctgccttacagtgtaacatgtttttcctaacacagccgcatt

SEQ ID NO: 84 - DNA, 406 bp, unknown
  >gi|900602|sr|IMAGp998N06288s1|cl| IMAGp998N06288|gb|H29692|H29692 ym61d03.r1 Homo sapiens cDNA clone 52960 5′
tcagtccttccccaggtgggccaacccacagggctgcttgagtgtcttcacaatatggcgctcggcttccccccagagcaagagattcaagggcccagggtaaaagccaacgtgttatttttatccctagcctcagaattcacacgccgttgcctccaccatgctctggtttgatacagcccagctctgattggaaggggctggggctgcccgtgctgactcttcaaaggcatcccatcctgcagatggtgttcacagggagagtttgtgggggccggcactccctcatctactggggctcattctggaagaaggtccagaagaattgggagaccccttgccccttcacccaaactttgggaggttggcagggtgaacagcaggccaagttcaggttccaagacag

SEQ ID NO: 85 - DNA, 646 bp, unknown
  >sr|IMAGp998M18194.1|cl|IMAGp998M18194|gb| BX114372|oid|234563121 BX114372 Soares placenta Nb2HP Homo sapiens cDNA clone IMAGp998M18194 ; IMAGE136121, mRNA sequence
ctttattnagtatnatatatgtggtcggcaagaaatattaataataaatgttgaaaacactattcccattaagaaataagtggatcaaagtgttacccttaatgatcatatagacactgataaataatagtcaactttattactgtggttcaaaccaagtggaggttgtatgttttgtctagaaaatatataaactacaaaaagttaataaacacctaagtgaatgagtaaattaaatgtggggagtaagattcaattaagataaagtcctaattttcttttaatcaaatgctctgctttgtactccttagaatggatgcactggagctgatttaaacattctttggtggtcacaagatgtatagctgcctctgtatgtttggcgcatggaaaaaaatatgagtttttgtggacatacactctttatttgaaaagtatgaaatataatccaagaaagccagccatggctcagtgataaatatggaattgatatgaaacattatattgattaatcacctttgaggatgattaataaaagagagaaagtgattctatatagagatgcaggactatttcaggctcattcatgatctaggtttttgtgcttcatggttattttattatcatcaataaaaaaaaaaaaaaa

SEQ ID NO: 86 - DNA, 437 bp, unknown
  >sr|IMAGp998J155722.2|cl|IMAGp998J155722|gb| BX112114|oid|234558605 BX112114 NCI_CGAP_GC6 Homo sapiens cDNA clone IMAGp998J155722 ; IMAGE2309102, mRNA sequence
agtaacttcattggcttctactcagtagatgccaatgttgaccctcctcccagtgtgacgatcaaacatttcttcagactttggcaaatttcatctgtggggggcaacattgtccccagttgagaaccactgaggtacaacatggactgaagaggagcatgtgttaaaaacgtgtgaattatttatcctgtagtatcttactgatgttcagataaagaaaaagcaaaacaaaagacctaaaactaggctcttcccccacggggtcaggttcatgtgtatagttgcatttaattgtattacgattctgggttgttgatgggctgatcatactgtgttttctccgaaaaaacaaagaattgttcatgagtacttactaaatgaataagtgaaatgacaaatgaataaagaaacacataaaggagaaaaaaaaaaaaaaa

SEQ ID NO: 87 - DNA, 236 bp, unknown
  >sr|IMAGp998B21790.1|cl|IMAGp998B21790|gb| BX114745|oid|234563867 BX114745 Soares_fetal_heart_NbHH19W Homo sapiens cDNA clone IMAGp998B21790 ; IMAGE345524, mRNA sequence
agaagtatctgcacaattagaaaagtcctcagtaagctttttcttggagggtacactttcttcactgtccctattcctagacctggggcttgagctgaggatgggacgatgtgcccagggagggacccaccagagcacaagagaaggtggctacctgggggtgtcccagggactctgtcagtgccttcagcccaccagcaggagcttggagtttggggagtggggatgagtncgnc

SEQ ID NO: 88 - DNA, 506 bp, unknown
  >sr|IMAGp998J09792.1|cl|IMAGp998J09792|gb| BX088778|oid|234511653 BX088778 Soares_fetal_heart_NbHH19W Homo sapiens cDNA clone IMAGp998J09792 ; IMAGE346472, mRNA sequence
acccacagggctgcttgagtgtcttcacaatatggcgctcggcttccccccagagcaagagattcaagggcccagggtaaaagccaacgtgttatttttatccctagcctcagaattcacacgccgttgcctccaccatgctctggtttgatacagcccagctctgattggaaggggctggggctgcccgtgctgactcttcaaaggcatcccatcctgcagatggtgttcacagggagagtttgtgggggccggcactccctcatctactggggctcattctggaagaaggtccagaagaattggagacccctgcccctcacccaaactttggaggtggcagggtgaacagcaggccaagttcaggtcccaagacaggccaaggccagtgcggtttcccttccactgcctcagtttacctgtattcagaagacagtctaggaagagttgagcagagttccctctaaaagagtagggagctgataacagtccccaagccctcctct

SEQ ID NO: 89 - DNA, 371 bp, unknown
  >sr|IMAGp998H175550.2|cl|IMAGp998H175550|gb| BX114820|oid|234564017 BX114820 NCI_CGAP_GC6 Homo sapiens cDNA clone IMAGp998H175550 ; IMAGE2243008, mRNA sequence
acagccaggtgggaaggactccttggcaaaactccaaccagcctgtacactgggaggaatgtgcactgggatggagccatagaagtttgtgtcgtttgcagtggggaggagcctggtccctcctcttcctgtgaggaacctggaattcaatctgtgaggttgttctggagatgttctggggagactgcattaaacacagcttcgcaccattgaataaactcagcaacaagccaatgcataaaagtaatctatgcttcaggtcacagaagcttcaaggggaaaaaaacagaatactctagggccattgttcacaaactcatctgaaaacatcctggaaaaattttcccaaacacatgaaaaaaaaaaaaaaa

SEQ ID NO: 90 - DNA, 784 bp, unknown
  >sr|IMAGp998E24312.1|cl|IMAGp998E24312|gb| BX099422|oid|234533220 BX099422 Soares adult brain N2b5HB55Y Homo sapiens cDNA clone IMAGp998E24312 ; IMAGE172415, mRNA sequence
cttctggaggggtgtgggactagagctgggtgtcacgtgaacccttcctggtagggtgacccccttcccctggagggggttttagagctgccgcctctgctccctctaacctctttggaggcagggttgggggtattgtcattcaaggccttttttttgtctgctccctccccgaccctgtgccctcttctggaggtttctcgtctgggagagtccctcccagcagtccctccacctccataaggacacactggacaaaactcccgcagctcttcaggaatgaccgatgcctacctgtggggttcagttgcccatagtttgaggccttctctcctcccttaccaccgctctggatcatgttactagttccgtcttttgtgtggccttgggccagcttccttgataccttgaagatgggcttcttgtgagtccccagggagaaagggacaagagctaagatttttgcatcagcccttctggcagaaggtgtggtaggggccatttgttttttttagtggacttgggatttgtggtgtaatcatatcattaatgatccagggtgtgggaaaaatggaggtccttgaagtggctgaatctcattgtatttaagacactgtcagttgccagatgtaggcttatttttggagatgtctaggagaggaaaaagctaccaatcatactcttgatatccgtctggctgtgtgaggcacccctacctcatggggggtgtcttgggattgatgaactgtggaacctgcctcctgcgctccccaaagcttattaa

SEQ ID NO: 91 - DNA, 579 bp, unknown
  >sr|IMAGp998J081153.1|cl|IMAGp998J081153|gb| BX100023|oid|234534422 BX100023 Soares_pregnant_uterus_NbHPU Homo sapiens cDNA clone IMAGp998J081153 ; IMAGE485095, mRNA sequence
ttgctacaggtccctgagnatgcaatcagattaagcgtagcaagcattgccaatgggaaagtcaaaataatttattttttttccctttccccctaccccatccccagccaagaatttcttttcaagatatcgtcatcattcttaaacaacattcttaacccccagctggggtccccattttaatagatgtcattgcttcaagtctaacggcgccgggaggcctgtttgagggaaaacattagtttgaaaaatccccgttcccttcatccactgcccttgttctccacgtgggagtgtgcttgtggcccctcagaaagatagtctgctggctcctaggggttggggtgggggacacacctttttctcaggaagaggtgatggcaatgtaaaacatctaagcaaagttttaaatgaaaaaaaggaaacacatttaaacatcctgataatggagggaaggggggcacatttacacatagcccagaacttgtagaattctgcatagtgaatgtatattgaattagtctcctgccttatacattcaggaggaataaatttccataatgtaaaaaaaaaaaaaaa

SEQ ID NO: 92 - DNA, 628 bp, unknown
  >sr|IMAGp998G18196.1|cl|IMAGp998G18196|gb| BX091497|oid|234517361 BX091497 Soares placenta Nb2HP Homo sapiens cDNA clone IMAGp998G18196 ; IMAGE136745, mRNA sequence
tatgttgaagtctttcagtgttcagctgaggagatgaactttgtgttaatggggggcactttaaatcgaaatggcttatccccaccgccatgtaagttaccatgtaagtttttcttgggtcttggcgctattctacgctatatgctggtaggtgcttaagctgctttcgtaactttctgtgcccctggttctttctgaggcaggtgaggtggttatataaggctctcccatctgtaatcagtagtacctggtaatcatttaagaccttggttgtggacacccataattagagatgctacaaatacctcctttcaaattatattcctgcttttccagtgataagcacttgatttctgaagcttagtgtgctccagataagcactgactctgggaggcaacctggctatatagataattttagtacgcatttaaaggcaaaataaaagtatggctggattctttttattttattttttaaaacgtattaacctttaagaagggcagtttgatgcacatgttgcatttaactttttgagcatcaagtatttttttaacatttttgagcatttctgttgctctccaggatatctggaatgagcaagtgagcaaaaacaaaaaaaaaaaaaaa

SEQ ID NO: 93 - DNA, 260 bp, unknown
  >gi|2106587|gb|AA426115|sr|IMAGp998F241862.1| cl|IMAGp998F241862|AA426115 zv52d12.s1 Soares_testis_NHT Homo sapiens cDNA clone IMAGE757271 3′, mRNA sequence
tttttttccaactggttagagaggcaagtgatttattctccggctcatgtttctatatggaggcattttccaagactaacaatcctggcttctctcttttggctttccttgtcctccgtggggcaaagttatctggaaagtctggaagggaccaggcacacgcagctcagcctttgctgctctcccctctcttcacacttgtgttatctggagggagatggaggcaggtgtccgtagccgggtcctgagcccctcgtccc

SEQ ID NO: 94 - DNA, 723 bp, unknown
  >sr|IMAGp998B09229.1|cl|IMAGp998B09229|gb| BX112820|oid|234560017 BX112820 Soares placenta Nb2HP Homo sapiens cDNA clone IMAGp998B09229 ; IMAGE149288, mRNA sequence
tatttcttgtacttggcctgctccttttcttcgaggatttgttaattatctaaaagtcagaaacatgtctgaaagtatggcagctgctcatagaacaaggtatttcttcttattgtagagactgcatcaacccgacattcctttcttataccaatgtgaaatttccagatcatctgtaaacctacaactttaatagaagactactaataacagaagacaaattagtgaagaaaagactgagtttcgaaattgaatggcagggtggtttttgcttacaagccatttctgttcattctttaagtatctatatttcatttgttttgcacatatgcatatgtgcccatttaagatatttgcatatacttgatagaaaccataaagttgtagcagttaagtccagtcacatttggttaatcagtgtttgatataattgaaagagttgagtggataaacagtcttccagcttgtaaatgccattgacttctgacctgacatttagtataataaaaatgaaattcttaaccatgtcaaatgatttagtttctggctcttagactcatctggcagttctacacatgaaacatcttttgttatataaggtgtattgaaacctgcagtgctgattattagaaaggatttgtcagatttttgaacatgatatttacattattatttaggaaaactcttcctgtaaataaccatgcataacttactttctgcaat

SEQ ID NO: 95 - DNA, 450 bp, unknown
  >gi|757038|sr|IMAGp998E11127|cl| IMAGp998E11127|gb|R06418|R06418 yf09a06.s1 Homo sapiens cDNA clone 126322 3′ similar to SPI1BC_HUMAN P29466 INTERLEUKIN-1 BETA CONVERTASE PRECURSOR
tttgttngtcttcacttttattgaaatacaaaatgttaaatatgcaagcngtactaatganggtgctccttgaagttnattaaggagggctgggctgcttgtggcttccattttcaattgccaggaaagaggtagaaatatcttgtcatggacagncgttctatggtgggcatttgagcttnggccctncgagttncaaatgattgctgtaccttccgaaatacttcctctaggtggcagcaccaagantatttctgggaagcatgtgatgagtcgtgtgatgatgatagagcccattgtgctgtctctccngggncacgtgtgtgtgggcgttgaaggaggcagtanagccaatgaagtcccttctccacgtggggnctttgttaaacagcatcttcccctagggtgctcagancacttttnaagacgccacttnccagggtgctggga

SEQ ID NO: 96 - DNA, 725 bp, unknown
  >sr|IMAGp998N02421.1|cl|IMAGp998N02421|gb| BX110042|oid|234554463 BX110042 Soares fetal liver spleen 1NFLS Homo sapiens cDNA clone IMAGp998N02421 ; IMAGE214465, mRNA sequence
tttgagtggaacaaagtgacctcttgcatccataacatcttaagcgggcagaggtggattgagcactatggagagattgtcatcaagaacctgcatgatgattcctgctactgcaaagtgaattttataaaggcaaaatactggagcactaatgcccatgagattgaaggcacagtgtttgacaggagtggaaaagcggttcatcggctgtttgggaaatggcatgaaagcatctactgtggcggcggctcctcttctgcctgtgtatggagagcaaatcctatgccgaaaggctacgagcaatactatagcttcacacagtttgcgctggaattaaatgaaatggatccatcatcaaagtctttattgccacctactgacactcgatttaggccagaccagaggtttctagaggaagggaacttagaagaagctgaaatacaaaagcagaggattgaacaactgcagagagaaaggcggcgggtcttagaagaaaatcatgtggagcaccagcctcggtttttcaggaaatccgacgatgactcttgggtgagcaacggcacctatttggaacttagaaaagatcttggtttttccaaactggaccatcctgtcttatggtgaaaaagtaaagaagaaagataacattagtgtatttctcctgtgcttgccttctgaagtggcacaaacctgtgtttatatatttaaaagatactctaggatga

SEQ ID NO: 97 - DNA, 335 bp, unknown
  >gi|5637283|gb|AI917428|sr|IMAGp998B145536.1| cl|IMAGp998B145536|AI917428 ts79d07.x1 NCI_CGAP_GC6 Homo sapiens cDNA clone IMAGE2237485 3′ similar to contains element MER22 repetitive element ;, mRNA sequence
ttttttttttttttttttttttttttttttttttttttttttttttttttttttttttttttttttttttttaaaaaggggccccttaaattttccttgggaaaaaacttttcaaaaatccctttatttttcccaattcctttggggggaaaccggggggggggggggaaaaaaccaaaaaccaatttttttaaacccccggccccccaaaaaagccttttggttcaaaaccccaaaaaaaagtttttggtctttttggggccccccccgggggggggggggaacccaaccggccaccccgggggaaaatttaaaaaacccccccccggggggaa

SEQ ID NO: 98 - DNA, 464 bp, unknown
  >gi|3179587|gb|AA993042|sr|IMAGp998C144120.1| cl|IMAGp998C144120|AA993042 ot92f07.s1 Soares_total_fetus_Nb2HF8_9w Homo sapiens cDNA clone IMAGE1624261 3′, mRNA sequence
tttcctttattttattcactgacatctctcagacctagaccagtgcctggcatatcctaggcatacattatattgaatgcatgaatttaaacatttaataattttaatttcatagttgtcttttaatatttgagatttttaccagatcttgcacattctgttgctactagaaaagccccttttctatgggcttgaaggatagaaaaatcttagcctgacttcactaaccagtataaatccagtggccccaggaaggctgggaacatcctatgtgaccatccttgattttggaccctaaaacgaaagctcaacttcacactcatctggaaacatctaatatcttttcaaacacatgtattttgttcccccaactagacatgaaacacattgacagaaattgcacaaagtattcagcacagtcctgaatacttcataaagacataaaacctggcttttgatggact

SEQ ID NO: 99 - DNA, 232 bp, unknown
  >gi|2583451|gb|AA651799|sr|IMAGp998M232973.1| cl|IMAGp998M232973|AA651799 ns19a12.s1 NCI_CGAP_GCB1 Homo sapiens cDNA clone IMAGE1184062 3′ similar to contains Alu repetitive element;contains LTR5.b3 LTR5 repetitive element ;, mRNA sequence.
ctttgcacaaatttaggatcaattttgttaggaaagcaagcattgaagtgtaggccgatgcgggaggatcacttggggtcaggaattcaagatcagtgtgggaaacataccaagagcccatctatacaaaaaacatttttaattagctgggcatggtggtgttcacctgtagacccaattacttggaaagctgagacaggaggattacttgaaccatgattgcaccactgca

SEQ ID NO: 100 - DNA, 680 bp, unknown
  >sr|IMAGp998D051943.3|cl|IMAGp998D051943|gb| BX103410|oid|234541200 BX103410 Soares_total_fetus_Nb2HF8_9w Homo sapiens cDNA clone IMAGp998D051943 ; IMAGE788308, mRNA sequence
ataaggatggatgaatttagacagataattcaaaggtncatgttaaaagaactggatactttttgtcactggaacgaacttgatcagttcatcaataagtttttcctaaacggngtctttatacatgatcagaagctcttcactgactttgttaatgatgttaaagattatcttagaaacatgaaggaatatgaagtagataatgatggagtatttgagaagttggatgaatatatatatagacacttctttggtcacactttttcccctccatatggacccaggtcggtttacataaaaccgtgtcattacagtagtttgtaacatttgtagattggatagcatttttatgatttgatgagtttcttgtaaggttaccgtttctaagagttgtgctttatggccactgagagaattcagaataaattgaaagatggagtctaaaaattattagctgttacaaatggaacatttcattataacgtgatcactttgacttgagcaaatggtttaatttttatcttaaaaatcagttaagaatatataaaatcctactttggccaagtttgtttcttttcattatagtttatatgaaaagatcaccttaagtgaaattattttcctttaatcttttatgnatttattcacttttggaagctaggaatgagcaacacaaattt

SEQ ID NO: 101 - DNA, 402 bp, unknown
  >gi|2538624|gb|AA626237|sr|IMAGp998M031831.1| cl|IMAGp998M031831|AA626237 zu93a02.s1 Soares_testis_NHT Homo sapiens cDNA clone IMAGE745514 3′ similar to SWPOL_MLVMO P03355 POL POLYPROTEIN ;, mRNA sequence.
tttaaagtcttctcctggttagcctcctagaggaggggctctttctttccttctctttgtggcttagccataagctagaaatttgggatctaaatgcagcagaaccttgctgcccttaggaactctcttatttgacaccaggtggttggagtaggaagtgcacaaacagattgcttttgtttactactaagccatctttcctcttggcttatataaaggcttaaatactggagacttttagagcagatttgagcttttttctgacactttatgtcctgcctttcacagcaggtgcagggagtcttgggtttctcctgggtgcagtagcccttggctggagggtttttttttataccatggccttgaccattctccttattttatttatttattttttggggc

SEQ ID NO: 102 - DNA, 593 bp, unknown
  >sr|IMAGp998K243513.1|cl|IMAGp998K243513|gb| BX089019|oid|234512315 BX089019 Soares_testis_NHT Homo sapiens cDNA clone IMAGp998K243513 ; IMAGE1391375, mRNA sequence
agaggatgttaaagtctctctttgataaacttaaattatgaaatagcccagttgttcagggtcacttctattgactcaatttgtgtgagactttgcactcactatctcgccggccggactgcatgcccagggtcagtagtcagcagagctaaaggtgcccgaatttagcacagctggctgtggtgcctgacatagggcagtaccgggggattcttcagaggtcagccaagtcggtaacattctgtgaagcagattaaatacagtaaaatcaaacaaacggaggtctttggaagtgatgctgggcccaggcagtgaaaatcaagaaaagaggagaaagaccaagacctggatgtcctggacgacctgacccaaaaaggggcacaaagcagcagagcttcaccaggtgactgggtggcttctttgtcctcattaaatgtacatctttgcatgaaaaggaccttgactggtctggactttaacaaagtaaaatatgaagaaacgtaaaattatgcttctgaaaaatatactgaaataaaattttgtaaatagctaaaagaaaaataaataaatataataacaaaaaaaaaaaaaaa

SEQ ID NO: 103 - DNA, 671 bp, unknown
  >gi|4511234|gb|AI560893|sr|IMAGp998B165468.1| cl|IMAGp998B165468|AI560893 tq41d08.x1 NCI_CGAP_Ut1 Homo sapiens cDNA clone IMAGE2211375 3′ similar to gbM20560 ANNEXIN III (HUMAN);, mRNA sequence.
ttaacaaaagcaacaatttttattatcttgctttatatttaatggattagaactataaagattcttaactttgaaagcagaaatataagttggatagtagttgcagatctttaataccattttcaatttcatttatgagctgctacattataaatgagatgctctaaaataataatcgcttttgttgttgttgttatagaacaatgaaaattcctgttaggaacacaagttgctgtttatatttgcttgttctcttaaatagtatgagaagaagtaaggtggagctgttggaaaagcccatcgtggacctttggagattatcttcttggttcagtcatctccaccacagatttttaagagtgtgatttcatagtctccagaagtatccgatttaattgctgaatatagggaatagccataatgcttcttgaactctgttcgaatgtccaaaaggtcaatttctgatctggacaccattattcggttcagagtaaactcatcagttccaatacccttcaaggctcgatgcagtctttcggctaaaaaaggccggcgtgttctcacacaattaactatggcccacagtagtcttcaaaatgccagatatttctctttatgctggccacatgtcctnttggctgatattctgattcatcaaatgtagttttaat

SEQ ID NO: 104 - DNA, 467 bp, unknown
  >sr|IMAGp998G024171.2|cl|IMAGp998G024171|gb| BX089765|oid|234513883 BX089765 Soares_testis_NHT Homo sapiens cDNA clone IMAGp998G024171 ; IMAGE1643929, mRNA sequence
tatgccaggtactttgctttctttgtattaactcttattcctcacaacaattctgaagtattactgtaactaccaacatgttatagatgaggaaacaaagagaagttaagcaacttgctccagtgacgcagctggtaagcagcagagctgggattaaaacccaggcattctgattccaccacctacacacttagccattccgccccactgagaatgaaaatgtcaaaaaatactaggccagtaagtgttagcaaggcaagaaatttccacaccaaaggtgagtatgttgtttggaaagattgggatggaaatgagtaaagtatgtttctttttaaaagtcttttagtaagatttaaagctctatatgtaagaagcagatgaaaataccaagtgaatttttagatttgatgttatgtaaggttttgttttgttaattaaaatttggtcattttaaaaaaaaaaaaaaa

SEQ ID NO: 105 - DNA, 510 bp, unknown
  >sr|IMAGp998K15174.1|cl|IMAGp998K15174|gb| BX094991|oid|234524351 BX094991 Soares infant brain 1NIB Homo sapiens cDNA clone IMAGp998K15174 ; IMAGE41539, mRNA sequence.
ttttttttttttttatctctattagtgggtttattgacataactggacataacaatgtataacatttattcatctcccactgaaagacttcccagtgaaactaaagtagaacacagatccaggcattaggcattttcagtgatgttctatggctgaaaaggtgggtggtgaggagaaccagaggccttgtccatacttgcaatgggactcaatattaccctgctgggcaaaagaaatagctaggagatgcaatagtgctatgattgaaaaaagccaggagcacaaaaggtgccagatagctccctggttgaaaataagagcaaacaacactactgctctcatgtaccagtatgctctcaatctaaaataagccctggcagcaaactaagaatgacagtctatttcctgtggtttattaagttttggccccgcagacgagactgccagaaaagcagctgtagttttaagtggtctggacatttactcatttgaacataaacaacgatcagc

SEQ ID NO: 106 - DNA, 268 bp, unknown
  >sr|IMAGp998I04624.1|cl|IMAGp998I04624|gb| BX116135|oid|234566646 BX116135 Soares_multiple_sclerosis_2NbHMSP Homo sapiens cDNA clone IMAGp998I04624 ; IMAGE281931, mRNA sequence
tctgcagttctcagcctggtcctttcttctgtcgagatcaggtctgcttcaaatacttctgccggagctgctggcactggcggcacagcatggagggcctgcgccaccacagccccctgatgcggaaccagaagaaccgagattccagctagaggagctggccttgcccagtggcctgtggcgcccaaagctggcaggtcaggcaagcagcctgtaccaccctgccactggcgaccagggagctggcttcccaaggacaagggaaaat

SEQ ID NO: 107 - DNA, 828 bp, unknown
  >sr|IMAGp998M24235.1|cl|IMAGp998M24235|gb| BX116782|oid|234567940 BX116782 Soares placenta Nb2HP Homo sapiens cDNA clone IMAGp998M24235 ; IMAGE151871, mRNA sequence
ttacccagggtttttggagtctctggatgatttttacattcttagcagtggattgatattgctgcagaccacaaacagtgtgtttaataaaaccctgctaaagcaggtaatacccgagactctcctgtcctggcaaagagtccgtgtggccaatatgatggcagatagtggcaagaggtgggcagacatcttttcaaaatacaactctggcacctataacaatcaatacatggttctggacctgaagaaagtaaagctgaaccacagtcttgacaaaggcactctgtacattgtggagcaaattcctacatatgtagaatattctgaacaaactgatgttctacggaaaggatattggccctcctacaatgttcctttccatgaaaaaatctacaactggagtggctatccactgttagttcagaagctgggcttggactactcttatgatttagctccacgagccaaaattttccggcgtgaccaagggaaagtgactgatacggcatccatgaaatatatcatgcgatacaacaattataagaaggatccttacagtagaggtgacccctgtaataccatctgctgccgtgaggacctgaactcacctaacccaagtcctggaggttgttatgacacaaaggtggcagatatctacctagcatctcagtacacatcctatgccataagtggtcccacagtacaaggtggcctccctgtntttcgctgggaccgtttcaacaaaactctacatcagggcatggcagaggtctacaactttgattttattaccatgaaaccaattttgaaacttgatataaaatgaag

SEQ ID NO: 108 - DNA, 565 bp, unknown
  >sr|IMAGp998F16280.1|cl|IMAGp998F16280|gb| BX102345|oid|234539070 BX102345 Soares infant brain 1NIB Homo sapiens cDNA clone IMAGp998F16280 ; IMAGE49499, mRNA sequence
ttttttttttttttacaaataagttttaccttttattgtattccattatacaattttacatagtaccattattttaagcaataattaagcaaattgaactactgtttttgagctctacttttcagcatatagatttacctaaaaaagtacaaaccaagcacagtggtgcaattttccaggcactttcaagttgcttatttacaaatatgtaagcaccctgttggaacacatgaaaattttggtgtcgagaagaaacactccatgtatatcaaatctttaaattttaacatttgcacagctccaaatattcctgagaaaggtcagtctcattgtcacgtacatctgcatttcctcacacttcagcgtatcttggggaaccataacctcggtcacttgatgtaccactaattgcacaaaatacatgtaatgtgttgctcatccagggagaatgcattgtgtcttcttgtaatatcgacttaagtcttcaaagctgctcatggataatgtcagataattcttttaccatgtctctgaaatttgactttagcccttcatggtactcaac

SEQ ID NO: 109 - DNA, 453 bp, unknown
  >gi|1290893|sr|IMAGp998B23677s1|cl| IMAGp998B23677|gb|W16493|W16493 zb15c12.r1 Soares fetal lung NbHL19W Homo sapiens cDNA clone 302134 5′
gagaaactgtgtgtgaggggaagaggcctgtttcgctgtcgggtctctagttcttgcacgctctttaagagtctgcactggaggaactcctgccattaccagctcccttcttgcagaagggagggggaaacatacatttattcatgccagtctgttgcatgcaggctttttggcttcctaccttgcaacaaaataattgcaccaactccttagtgccgattccgcccacagagagtcctggagccacagtcttttttgctttgcattgtaggagagggactaagtgctagagactatgtcgctntcctgagctaccgagagcgctcgtgaactggaatcaactgcttcagggaaaaagaaaaaaaaaaaaaaaaagacttgcctgggagncgcgagaaacttgcattggaanttcanaaccagcattcgagaaactcctctctactttancac

SEQ ID NO: 110 - DNA, 490 bp, unknown
  >sr|IMAGp998N204778.2|cl|IMAGp998N204778|gb| BX092450|oid|234519268 BX092450 NCI_CGAP_GC4 Homo sapiens cDNA clone IMAGp998N204778 ; IMAGE1946707, mRNA sequence
cctttggccttttataaaaaggtatttataattcgttgataaaaaccaatatttgaagctgttgattaccaacaaaaaagtttcacactctttatagtgcttcaggatacaactttttcagggccttattagagaaaaactgattcacatgttattcttctaaagctaaattattaaaatgtaggttgaaagattggattgattgtgaatctacactaaagatatggttccaggcagacctccttagagacctccaggcagacctccttactgtcttcagtgatgtttaatttcattactgttactgggtgccaagtgtctttcatttggaagtgaacttactccagttattgaatgtttatcatatcataatttgcatttttccaacattgaaggtattttaaaaagtcactaagagattcattcttttattattcaactacaaaaaaatagtgtaatgatcagaatttaaaatgtgtgattctttttc

SEQ ID NO: 111 - DNA, 493 bp, Homo sapiens
  >ImageID=1309431 IMAGP971F12104_S1 CC FromEXP of IMAGp971F12104_s1.exp April 26, 2002 1257
agtttttttttagaatttagagcattatgagactattgtagaaattttaactaccaggacctacaggatggagagtgggggaggggagaagaccagaagagaggcaggaaaacagatgcctttgctgatgtcctttgtagtataaaacttaacagcaccacagcacctaatcattttctcaggtgtggctttgagaaataaacgttttatgttaaaaatgaaaatacttcacaatatgagacagctggcatttgaatacttttcccagtatgtttgtaaacattttggattcatatttcatattaaaatggacatttagtaggattattagatgaaaagggtcccccaggggatatctaatatcaaattatctgcaggacatcttctccagaaagcagactgactgtaatatgaactaattttcccccctttgagttgcttgcaagaatctctgagatctgacactgatgggctacagtaatcagcagttctt

SEQ ID NO: 112 - DNA, 496 bp, unknown
  >sr|IMAGp998I144113.2|cl|IMAGp998I144113|gb| BX093451|oid|234521269 BX093451 Soares_testis_NHT Homo sapiens cDNA clone IMAGp998I144113 ; IMAGE1621717, mRNA sequence
cggctacatagctgtgactacngggggtgcagtctctggggaccaccactaccgaggctgggtaccatgattatcgcggaacacctgcccattaatcttgaccctgttaaaacaataactgcctatccagatatgccacacttctgcctgctgttggcaaccctcctggactaggctgctcttgttaatcacatggatgttgctgattactctgagtgcaaaccttttcactgttccagagaggagcctgacaaccacattctccttctcaagatgtgaaaggcattgtacagaagaagatgagaatcacagtaaaccaaacaaaggaaatgcagaagagatattttcagctgaatattggaaagtaagattccctgcgagcaggatctcaggcaacgtcaatgaacaacatgaagcctgacacattgtaggtgccccgtaaatattggttgagtaactgaataaacgtatttagtaacttaaaaaaaaaaaaaaa

SEQ ID NO: 113 - DNA, 496 bp, unknown; identical to SEQ ID NO: 112
  >sr|IMAGp998I144113.2|cl|IMAGp998I144113|gb| BX093451|oid|234521269 BX093451 Soares_testis_NHT Homo sapiens cDNA clone IMAGp998I144113 ; IMAGE1621717, mRNA sequence

SEQ ID NO: 114 - DNA, 392 bp, unknown
  >sr|IMAGp998N045486.2|cl|IMAGp998N045486|gb| BX099882|oid|234534140 BX099882 NCI_CGAP_Ov23 Homo sapiens cDNA clone IMAGp998N045486 ; IMAGE2218563, mRNA sequence
ttttttttttttttatagcttaaattttcaactccaaaattttatgattttattattctagttagccaaatatttttcagagtccacggatgtctctgtggacattccagaaaactaagagtctattaaactccaaatcttgatctaaccattttacactgtcaaattatttattccttacagtttttgataataaaatggtcgaggattttgagaacatgtgggcagcagattcctgcctctaattgggattcgtgatgccagggcaggaaaatatcacttcccagtccctgccagtgaaaactcaaaaaaagctgttcagctcatttcatcatgctgtcactaccctctttctaaaaactgcaaaactcttttcttggaaatcattatcc

SEQ ID NO: 115 - DNA, 615 bp, unknown
  >sr|IMAGp998I16328.1|cl|IMAGp998I16328|gb| BX104592|oid|234543564 BX104592 Soares adult brain N2b5HB55Y Homo sapiens cDNA clone IMAGp998I16328 ; IMAGE178647, mRNA sequence
tttttgtgggatcctgcagtaaaattgacttttttgtgtctttgggagatttaaattgcgctaacagtgttgcgcaaaaatgagttcatgccatttaacatattgtattttaattattaactgtattaatttactatgaaatggacatccttttaactaaaatggaattgaacattgcagttttcaaatatttttccttgttgggtctggaaaaggaattctactttgatctgcatagaaaattttgatacaattttttgaaagttcttaggtgaaacatttacccattaaaaaggaagcagaaatactgagacatgaaaggcattatcaactaactctagactctagaacccattctagcatatctcacgtgcaatttttaaaaataagttaataattcatctcatatcaacaaaagcctttgaaacatgggttttcactagatatcacctagtgctaagataaaaaccaaaacaatatcagaattacatttatgctctaaatttgtagttgtccattgttgtgcttagtaaatgtgtgtcattaatgctgtattctcctagctattatggaaacttgnttaaataaagatatggatataaaaaaaaaaaaaaa

SEQ ID NO: 116 - DNA, 508 bp, unknown
  >sr|IMAGp998C14658.1|cl|IMAGp998C14658|gb| BX106304|oid|234546989 BX106304 Soares fetal liver spleen 1NFLS Homo sapiens cDNA clone IMAGp998C14658 ; IMAGE294853, mRNA sequence
tctagtatttatatgaatctgtgagagataatttttttggtctcactgcaatgaaccaaaagcggcctgagtttggtcttttaattgtagccntgtattgaaggcatctttttgaccaactcttgttggttctgtcttgaaccattgttacgtcactgtgctgtaattagtatagctaaatcttttccttccttgctcctcccccagcccaccccgtcttcccttaacattttttcagggggggttgggagtggtttcattttagtgtgagtggatgttttgatagttgtaaggaaaaaatgcatttcagacacatttcacacatgagctattttcttacacagtatgtcttattgttaataagaatgtaatttcatgatacaagtatttaataccttttttaagtcagtaaatattctagccatcaataaattgcagtagagtattaagaggggacaggatgagtttactcttaaaattaatcagtatcaaactaagtctactgtgt

SEQ ID NO: 117 - DNA, 707 bp, Homo sapiens
  >ImageID=1704376 IMAGP971I1573_S1 CC FromEXP of IMAGp971I1573_s1.exp April 26, 2002 1315
ccacctttaaattcctgcaatttatgctgcaaccaacatcttagtgtttgctctctccgggaagaggccaggtgggggagaacatacggtccagagcctgagatcgcctggctggggtgcaggcaagcaggaacccagtctagatagaatgcagtgttctctaaagctatggatggtttcagtgaaatgtctgtgtctgacaatcgtcacgcaagagtacttactgtcttcccttgtacatacattgtctgtatttagcttttcctagtattgtgaggtttcaaataggggttccatgtaaatactctttctgctactgaaatgccggcccccttgagtatgcatcacccccacagatggttttgcctttctctatgctaacactttttgagccactgacgtgcagatcggtggttagtttcttgggcttttattttgcaattttatatatatacacatattatatatatatatatatttatgggttggttttgtatagttttctgtttgaatctctgagcaccaaagctgccctttgattttcacgagaactttccaaagccacttcctgagccctcctgctgctttggtgtctgacggtgatgctgcggtggcatctctgcccgtgaatttctgtatgtgtgtgctgttacctcacctgcagacatgaccctagtaaatctaacgtgcttggcaaaaaaaaaaaa

SEQ ID NO: 118 - DNA, 390 bp, Homo sapiens
  >ImageID=2328917 IMAGP971B0496_S1 CC FromEXP of IMAGp971B0496_s1.exp April 26, 2002 1237
tgagttgtccaatcagttttcaattgatactgttggtagtnatggagctgttaaatgtaaaggcctgaaaatggactatcaagttggtgtcactatagacctgagcagttttaacattactagaattgtgacatttacccctttttatatgattaaaaacaaaagcaaataccatatatcagtggctgaagaaggaaatgataaatggctctctcttgatttggagcaggtgggtagatgaatttcaaaaatatacctctttggattctattttctccttgaagntagatacctaaagtcaccatgtaatgntttaactctccccttactccaattttgttttagnttcatgaatctttattgaatatttgngccctgctctagatagat

SEQ ID NO: 119 - DNA, 385 bp, unknown
  >sr|IMAGp998D01196.1|cl|IMAGp998D01196|gb| BX106181|oid|234546743 BX106181 Soares placenta Nb2HP Homo sapiens cDNA clone IMAGp998D01196 ; IMAGE136656, mRNA sequence
tttgntttcttatnttgtataatcaggttaatgatttattttttgactaaatgtgcaatttcttatcactagataactttcagtatcagtggtggttacttattacttaaatcagaggaaggattttataaagattaataaatttaattttaccaataaatattcccataatttagaaaaggatgtcgacttgctaatttcagaaataattattcatttttaaaaagccccttttaaagcatctacttgaagattggtataattttcataaaatgtctttttttttagtgtcccaaagatatcttagataaactattttgaagntcagatttcagatgaggcaacattttcttgagataattacccaagcttcatccatgttgaa

SEQ ID NO: 120 - DNA, 501 bp, unknown
  >sr|IMAGp998B2070.1|cl|IMAGp998B2070|gb| BX112318|oid|234559013 BX112318 Soares infant brain 1NIB Homo sapiens cDNA clone IMAGp998B2070 ; IMAGE22170, mRNA sequence
ttttttttttttttaacatgtgtcaaagnattttattaaactaattcaatttgttagggaaatggtaagatgttacaactggttaagaggagcattaaaagtgcagacatatattcaggccatcaagaatgaggaaatgaatttgctaaattgatgcaacatcaatcaatagccagggcctgtcattcacagacaatcactccaccacacgcacacatacattcctacggtttgtccacaacttccagagctgaagagtagctcaaggacatcaaagggcagaggcccctaggaagggctgagccacaatgaagcagtgctccttcactcccgatttccatggctaccaccgtttttctctggcaaatcagagacgaattttaaagactgagtaaatctataaaagcgattaatcacagaagttcaatgtctgataaattattccaaaagtgttcctgggcactgagactcaccaagcatacattctgaaatcaaattcta

SEQ ID NO: 121 - DNA, 764 bp, unknown
  >sr|IMAGp998I17258.1|cl|IMAGp998I17258|gb| BX109425|oid|234553229 BX109425 Soares breast 3NbHBst Homo sapiens cDNA clone IMAGp998I17258 ; IMAGE160600, mRNA sequence
gtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataaaattgngatctctctcttcattctattctttttctctcaagagttccatttaatggaaataaaaacggtataattaataattctctaggggggaaaaatgaagcaaatctcattggatatttt

SEQ ID NO: 122 - DNA, 457 bp, unknown
  >gi|776508|sr|IMAGp998M04179|cl|IMAGp998M04179| gb|R21727|R21727 yh21b02.s1 Homo sapiens cDNA clone 130347 3′
ttttttgtggttccaactntttttatactgaaatatgtctgagctgatccaaaaaagcgttgggttgggggagaaggaaatgatggaagtggggtgatctcagagctccatgttagcaactctgggagtntgggccggtccatctgtctgtctgcccaccctggcaggacaatcaggcctaggaggtctggaggagctcgccgagctgggcggaggaggcaggccagtcccatgctggaggggaccccacggccccttctacagggcaggagagtcctcagctgaagggcttcaccagcttcatggcagcatagttcgggaaggcntagatgaagatggccagggaacagcagcagnctgaagaggatgatnaagaggatgatggcccaccngaaacgccgccacaggatgaactttcatggtcttgtattgggnaagtaaaccacaggaaggangt

SEQ ID NO: 123 - DNA, 376 bp, unknown
  >gi|5631530|gb|AI911675|sr|IMAGp998K175764.1| cl|IMAGp998K175764|AI911675 wc71e09.x1 NCI_CGAP_Pan1 Homo sapiens cDNA clone IMAGE2324104 3′ similar to contains element MER22 repetitive element ;, mRNA sequence
ttgacacaaaatatgttatttattgtcaatttctgcaaagacacatttaagcaacaaaatatacatttgtcaaccttttagaatttgttttgtacattaacaaatacaatgtgctaccatagcaataaattaaatgtacattatttacattacataggcagttgggatctacgaatcacctcctcactaagaggaaagaaagcaaagtcgtcgtcacatggagcatcacttccaagccacagttcttaggttcgaccacacaggggaatctgcacactcacacgccagcagtccccgagctggagtgtccaaggggaacacccaaaacaggagagagctgcacgcatgctaaggagcaggaagccggggggggnng

SEQ ID NO: 124 - DNA, 501 bp, unknown
  >sr|IMAGp998J11128.1|cl|IMAGp998J11128|gb| BX090520|oid|234515407 BX090520 Soares fetal liver spleen 1NFLS Homo sapiens cDNA clone IMAGp998J11128 ; IMAGE126826, mRNA sequence
gatacagtgtgacattttaatacatttacatattgtgtaatgatcaaattaggataattagcttatgtatcacttcaaaaacataatttctttgtgatgagaacattcaaaatcttctagtattttgaaatacataatgcaatattgttaaccacaatcaccctactgtgcaatagaacaccagagtttactcctcctttccatctgtagcttcagacccattgaccaatctctccccttcgcccccaccaccccccccccgctgtcaccactctcctcccctccccagcaagaggcttgaatcttgaaggagcacatcagatagaaagagggaaatcaactgggacatggtttgttggaagtggggtggaatctggtttgtttacagtttcagagtttgaggcatcaaaagtgtatgtcattgcccaaaagtatttaacagctttctatgttttaggaattcactagaaataaggcatgctttttaaaaaaaaaaaaaaa

SEQ ID NO: 125 - DNA, 293 bp, unknown
  gi|4628611|gb|AI619485|sr|IMAGp998H195650.1| cl|IMAGp998H195650|AI619485 ty38g10.x1 NCI_CGAP_Ut2 Homo sapiens cDNA clone IMAGE2281410 3′ similar to contains MSR1.t1 MSR1 repetitive element ;, mRNA sequence
tttttttttttttttttttttttttttttttttttttttttttttttttttttttttttttttttggggaaaagggattaaaaaacctttttcccaaaaaggaaggccccccccccccccaaatccctttgggacaagaaccgaccccccccggaaaccccaaccattgtccccccctcccacccctggtctttcccaaggaaaaaaggccattcaaaaatggggggggggggttagagggggggggccccttccctttaaatgggggaaaaaaaaaaaagcccccgggtggc

SEQ ID NO: 126 - DNA, 519 bp, unknown
  gi|5111779|gb|AI743491|sr|IMAGp998P025858.1| cl|IMAGp998P025858|AI743491 wf63h01.x2 Soares_NFL_T_GBC_S1 Homo sapiens cDNA clone IMAGE2360305 3′, mRNA sequence
tttaaactccacactgtaaccaggagtcttttcaaagcaatatcctggtcttgtctggcattcccttggtctaccttggaggcatttatccaggtgagaattgtggttaatgaagctccataaaacccacacaaagggtcaaccctgcattatttggtaaagtgatggggttaccataaccagcagcacaaagcacagcacttagacctgtgccagagagcttaactgtacaatgtgggccaccctagccatgataaaacaatgactgatgaagactaactcgacaaagaattgtttgagagtcacgcattctacccacttacctactctgtattttattctggcgttaaactggactgtcagaaacaaaatgctttggcttcactacccaacccaactagtctcccaacacaaccccatcccgctaagaaaactgctatcactgagtcatgtttttttgtcatgccctcaccttccagcaagggttgctgtaagaaacagcctcgagaatactaaggc

SEQ ID NO: 127 - DNA, 370 bp, unknown
  >gi|846987|sr|IMAGp998M03208|cl|IMAGp998M03208| gb|R72955|R72955 yi38a02.r1 Homo sapiens cDNA clone 141482 5′.
cgagataaagctttcaggtacaagctgaaggtggggtgtctaacaactaaaaactatcactaaatctcaaagagaaagttcttgcaaaatatgtaaagttcacaaggtgcagacattttccttctttaggcttttatctaaggaagggctatgaaactgggcccatctgtatacaggctcaaatttacgtttttaaaggaagaatctatgcagctgaggcttattgcaggaaatacttcatttgtatgtaaatattattgtaaataaatagggggctgtatatttttgaagttgttgatctgcacttgaaataggaagtctctaggggtcntgcctataaaccataaatgtttccccggnaattagttgg

SEQ ID NO: 128 - DNA, 283 bp, unknown
  >gi|3483811|gb|AF086466|sr|IMAGp998H07795.1|cl| IMAGp998H07795|AF086466 Homo sapiens full length insert cDNA clone ZD86G04
caagactccatctcaagaaaaaaaaaaaaattagctgggtgtggtggcacatgcctgtagtcccagctacttgggaggctgaggtgaaaggatcgcttgggcctgggaagcggtgattgcaacgctgcacatcagcctgggtgaccaagcgggactctgtctcagaacaaaacaaaacaaaaatacatattcccagatcctggaatgtcctgggaagctatatgtgtgtcatgccctcccagctgcacctgaacaatgagcatagcaaacatcaaaaaaaaaa

SEQ ID NO: 129 - DNA, 451 bp, unknown
  >gi|2183927|gb|AA459020|sr|IMAGp998A112011.2| cl|IMAGp998A112011|AA459020 aa26a06.r1 NCI_CGAP_GCB1 Homo sapiens cDNA clone IMAGE814354 5′, mRNA sequence
agaatccctgtgcaattctgtaaaagaataccaggttctctggcatgtagaagtgaacttgggcaggtggaaatggttaacaaagaattctaggagtagagaataaggtctgaatcctcttaggacagttgggagttacaaaaaattaacaaacagaaacattgagattggtgctttagaacttggagtaaattaagaataaagggaagtagacacctttaaaaaacaaagaaaaagaacttggtgagtaaaaatagaaaagacaaattatggagtaggaaataaaaactcaccagaaaagttgtcttttacctcctaagggaaaagctaaaggaattagagttgttttgtctaaagaaaggcaagggaagactcaatttgttgttgtcagttattaacaatttatataaagaatagtgtactatttcttttctgtctgcagagattgagc

SEQ ID NO: 130 - DNA, 424 bp, unknown
  >sr|IMAGp998A205772.2|cl|IMAGp998A205772|gb| BX097589|oid|234529551 BX097589 NCI_CGAP_Co3 Homo sapiens cDNA clone IMAGp998A205772 ; IMAGE2326939, mRNA sequence
agtgaaatagagaaagcgggcctggcatccccagtaggaggaagacgctgttggtaaagagccgctgaaggcagcacccaagaaacgacaattgctggacagcgacgaggaacaggaagaagatgagggcaggaacagagcaccagagttgggagctccaggaatccaaaagaagaaacgataccagattgaggtgggtcgcagcaccagatggggagctttatgacatgacaggcgggagttttgtaccacttttagaagataccagatgcctctgtcctcattttgtttcctatgatcatcctctagataccagatagaggcccattcctgcatttgagatagggttggaatatagtactctagatgatgaatgtgtttgctttgtgtttggtattaaacatgtaagattagaagttctcan

SEQ ID NO: 131 - DNA, 472 bp, unknown
  >sr|IMAGp998I09280.1|cl|IMAGp998I09280|gb| BX091207|oid|234516781 BX091207 Soares infant brain 1NIB Homo sapiens cDNA clone IMAGp998I09280 ; IMAGE49203, mRNA sequence
ttttttttttttttaaaaatatataangttttattgtcaaaaatagacaaactttaatttcctttaacaggaatattaatttaacagccttccataagccatcaccattttgtaagcataacaggcaagagagtcaaagataactgttagtggaaaaaggacaacagttctacatcgatgcccaanaagccttgcccagtcagnggtgacaactccaggacagcggcagaaacacagngaacctttggagcttaacaatagacatgcaaaacaacattgatttatcttggcccaattctataaagattggctttgtagtatctttccaagcatttgaagagtttagtttgttagaacactggctaatttgaacagngacattttaggtcacttatagtatcagtaacaggatcacacctgtttttcagtaacaccaggatgaatacatctgtccctacgccggtctccagca

SEQ ID NO: 132 - DNA, 666 bp, unknown
  >sr|IMAGp998J24653.1|cl|IMAGp998J24653|gb| BX098770|oid|234531914 BX098770 Soares fetal liver spleen 1NFLS Homo sapiens cDNA clone IMAGp998J24653 ; IMAGE293111, mRNA sequence
ttctttggatgtagacacttttcaaagaccaatgagctgctgcagaagtctggcaagaagcccattgactggaaggagctgtgatcatcagctgaggggtggcctttgagaagctgctgttaacgtatttgccagttacgaagttccactgaaaattttcctattaattcttaagtactctgcataagggggaaaagcttccagaaagcagccatgaaccaggctgtccaggaatggcagctgtatccaaccacaaacaacaaaggctaccctttgaccaaatgtctttctctgcaacatggcttcggcctaaaatatgcagaagacagatgaggtcaaatactcagttggctctctttatctcccttgcctttatggtgaaacaggggagatgtgcacctttcaggcacagccctagtttggcgcctgctgctccttggttttgcctggttagactttcagtgacagatgttggggtgtttttgcttagaaaggtccccttgtctcagccttgcagggcaggcatgccagtctctgccagttccactgcccccttgatctttgaaggagtcctcaggcccctcgcagcataaggatgttttgcaactttccagaatctggcccagaaattagggctcaatttcctgattgtagtagaggttaaga

SEQ ID NO: 133 - DNA, 430 bp, unknown
  >gi|2103286|gb|AA424316|sr|IMAGp998N161887.1| cl|IMAGp998N161887|AA424316 zv90d08.r1 Soares_NhHMPu_S1 Homo sapiens cDNA clone IMAGE767055 5′, mRNA sequence
caagaaaataagaaaggtagaaacaggaggacccaccgaatttgagagggcacccaggagtgtctgagctgcattacctctagaaacctcaaacaagtagaaacttgcctagacaataactggaaaaacaaatgcaatatacatgaacttttttcaaagcattatgtggatgtttacaatggtgggaaattcagctgagttcaccaatataaattaaatccatgagtaactttcctaataggctttttttcctaataccaccacctaacagagaacacaggtgaatgcagatgttcactttagcagacttaatgtttcctatgagatttcactgtacaggtttgtctttcttctttgcctgagaaataaaaatgtcatttgccatattgccatctaaaggagaaaaactgcatcagcaaagccattgtat

SEQ ID NO: 134 - DNA, 2698 bp, Homo sapiens
  >TXNIP (HUGO ID) THIOREDOXIN INTERACTING PROTEIN; UPREGULATED BY 1,25-DIHYDROXYVITAMIN D-3. [Source RefSeq (NM_006472)]
gcttagtgtaaccagcggcgtatattttttaggcgccttttcgaaaacctagtagttaatattcatttgtttaaatcttattttatttttaagctcaaactgcttaagaataccttaattccttaaagtgaaataattttttgcaaaggggtttcctcgatttggagctttttttttcttccaccgtcatttctaactcttaaaaccaactcagttccatcatggtgatgttcaagaagatcaagtcttttgaggtggtctttaacgaccctgaaaaggtgtacggcagtggcgagaaggtggctggccgggtgatagtggaggtgtgtgaagttactcgtgtcaaagccgttaggatcctggcttgcggagtggctaaagtgctttggatgcagggatcccagcagtgcaaacagacttcggagtacctgcgctatgaagacacgcttcttctggaagaccagccaacaggtgagaatgagatggtgatcatgagacctggaaacaaatatgagtacaagttcggctttgagcttcctcaggggcctctgggaacatccttcaaaggaaaatatgggtgtgtagactactgggtgaaggcttttcttgaccgcccgagccagccaactcaagagacaaagaaaaactttgaagtagtggatctggtggatgtcaatacccctgatttaatggcacctgtgtctgctaaaaaagaaaagaaagtttcctgcatgttcattcctgatgggcgggtgtctgtctctgctcgaattgacagaaaaggattctgtgaaggtgatgagatttccatccatgctgactttgagaatacatgttcccgaattgtggtccccaaagctgccattgtggcccgccacacttaccttgccaatggccagaccaaggtgctgactcagaagttgtcatcagtcagaggcaatcatattatctcagggacatgcgcatcatggcgtggcaagagccttcgggttcagaagatcaggccttctatcctgggctgcaacatccttcgagttgaatattccttactgatctatgttagcgttcctggatccaagaaggtcatccttgacctgcccctggtaattggcagcagatcaggtctaagcagcagaacatccagcatggccagccgaaccagctctgagatgagttgggtagatctgaacatccctgataccccagaagctcctccctgctatatggatgtcattcctgaagatcaccgattggagagcccaaccactcctctgctagatgacatggatggctctcaagacagccctatctttatgtatgcccctgagttcaagttcatgccaccaccgacttatactgaggtggatccctgcatcctcaacaacaatgtgcagtgagcatgtggaagaaaagaagcagctttacctacttgtttctttttgtctctcttcctggacactcactttttcagagactcaacagtctctgcaatggagtgtgggtccaccttagcctctgacttcctaatgtaggaggtggtcagcaggcaatctcctgggccttaaaggatgcggactcatcctcagccagcgcccatgttgtgatacaggggtgtttgttggatgggtttaaaaataactagaaaaactcaggcccatccattttctcagatctccttgaaaattgaggccttttcgatagtttcgggtcaggtaaaaatggcctcctggcgtaagcttttcaaggttttttggaggctttttgtaaattgtgataggaactttggaccttgaacttatgtatcatgtggagaagagccaatttaacaaactaggaagatgaaaagggaaattgtggccaaaactttgggaaaaggaggttcttaaaatcagtgtttcccctttgtgcacttgtagaaaaaaaagaaaaaccttctagagctgatttgatggacaatggagagagctttccctgtgattataaaaaaggaagctagctgctctacggtcatctttgcttaagagtatactttaacctggcttttaaagcagtagtaactgccccaccaaaggtcttaaaagccatttttggagcctattgcactgtgttctcctactgcaaatattttcatatgggaggatggttttctcttcatgtaagtccttggaattgattctaaggtgatgttcttagcactttaattcctgtcaaattttttgttctccccttctgccatcttaaatgtaagctgaaactggtctactgtgtctctagggttaagccaaaagacaaaaaaaattttactacttttgagattgccccaatgtacagaattatataattctaacgcttaaatcatgtgaaagggttgctgctgtcagccttgcccactgtgacttcaaacccaaggaggaactcttgatcaagatgccgaaccctgtgttcagaacctccaaatactgccatgagaaactagagggcaggtcttcataaaagccctttgaacccccttcctgccctgtgttaggagatagggatattggcccctcactgcagctgccagcacttggtcagtcactctcagccatagcactttgttcactgtcctgtgtcagagcactgagctccacccttttctgagagttattacagccagaaagtgtgggctgaagatggttggtttcatgtttttgtatta

SEQ ID NO: 135 - DNA, 4609 bp, Homo sapiens
  CALD1 (HUGO ID) CALDESMON (CDM). [Source SWISSPROT (Q05682)]
ttgcagaatcgcctaccagaggaatgacgatgatgaagaggaggcagcccgggaacggcgccgccgagcccgacaggaacggctgcggcagaagcaggaggaagaatccttgggacaggtgaccgaccaggtggaggtgaatgcccagaacagtgtgcctgacgaggaggccaagacaaccaccacaaacactcaagtggaaggggatgatgaggccgcattcctggagcgcctggctcggcgtgaggaaagacgccaaaaacgccttcaggaggctctggagcggcagaaggagttcgacccaacaataacagatgcaagtctgtcgctcccaagcagaagaatgcaaaatgacacagcagaaaatgaaactaccgagaaggaagaaaaaagtgaaagtcgccaagaaagatacgagatagaggaaacagaaacagtcaccaagtcctaccagaagaatgattggagggatgctgaagaaaacaagaaagaagacaaggaaaaggaggaggaggaagaggagaagccaaagcgagggagcattggagaaaatcaggtagaggtgatggtggaagagaaaacaactgaaagccaggaggaaacagtggtaatgtcattaaaaaatgggcagatcagttcagaagagcctaaacaagaggaggagagggaacaaggttcagatgagatttcccatcatgaaaagatggaagaggaagacaaggaaagagctgaggcagagagggcaaggttggaagcagaagaaagagaaagaattaaagccgagcaagacaaaaagatagcagatgaacgagcaagaattgaagcagaagaaaaagcagctgcccaagaaagagaaaggagagaggcagaagagagggaaaggatgagggaggaagagaaaagggcagcagaggagaggcagaggataaaggaggaagagaaaagggcagcagaggagaggcagaggataaaggaggaagagaaaagggcagcagaggagaggcagaggataaaagaggaagagaaaagggcagcagaggagaggcaaagggccagggcagaggaggaagagaaggctaaggtagaagagcagaaacgtaacaagcagctagaagagaaaaaacatgccatgcaagagacaaagataaaaggggaaaaggtagaacagaaaatagaagggaaatgggtaaatgaaaagaaagcacaagaagataaacttcagacagctgtcctaaagaaacagggagaagagaagggaactaaagtgcaagctaaaagagaaaagctccaagaagacaagcctaccttcaaaaaagaagagatcaaagatgaaaagattaaaaaggacaaagaacccaaagaagaagttaagagcttcatggatcgaaagaagggatttacagaagttaagtcgcagaatggagaattcatgacccacaaacttaaacatactgagaatactttcagccgccctggagggagggccagcgtggacaccaaggaggctgagggcgccccccaggtggaagccggcaaaaggctggaggagcttcgtcgtcgtcgcggggagaccgagagcgaagagttcgagaagctcaaacagaagcagcaggaggcggctttggagctggaggaactcaagaaaaagagggaggagagaaggaaggtcctggaggaggaagagcagaggaggaagcaggaggaagccgatcgaaaactcagagaggaggaagagaagaggaggctaaaggaagagattgaaaggcgaagagcagaagctgctgagaaacgccagaagatgccagaagatggcttgtcagatgacaagaaaccattcaagtgtttcactcctaaaggttcatctctcaagatagaagagcgagcagaatttttgaataagtctgtgcagaaaagcagtggtgtcaaatcgacccatcaagcagcaatagtctccaagattgacagcagactggagcagtataccagtgcaattgagggaacaaaaagcgcaaaacctacaaagccggcagcctcggatcttcctgttcctgctgaaggtgtacgcaacatcaagagtatgtgggagaaagggaatgtgttttcatcccccactgcagcaggcacaccaaataaggaaactgctggcttgaaggtaggggtttctagccgcatcaatgaatggctaactaaaaccccagatggaaacaagtcacctgctcccaaaccttctgacttgagaccaggagacgtatccagcaagcggaacctctgggaaaagcaatctgtggataaggtcacttcccccactaaggtttgagacagttccagaaagaacccaagctcaagacgcaggacgagctcagttgtagagggctaattcgctctgttttgtatttatgttgatttactaaattgggttcattatcttttatttttcaatatcccagtaaacccatgtatattatcactatatttaataatcacagtctagagatgttcatggtaaaagtactgcctttgcacaggagcctgtttctaaagaaacccatgctgtgaaatagagacttttctactgatcatcataactctgtatctgagcagtgataccaaccacatctgaagtcaacagaagatccaagtttaaaattgcctgcggaatgtgtgcagtatctagaaaaatgaaccgtagtttttgtttttttaaatacagaagtcatgttgtttctgcactttataataaagcatggaagaaattatcttagtaggcaattgtaacactttttgaaagtaacccatttcagatttgaaatactgcaataatggttgtctttaaaaaaaaaaaagaaatgtactgttaaggtattactttttttcatgctgatgattcatatctaaattacattattatgttagctgacagtggtactgattttttaggttggttgttttgtggatttctttagtagtgatagtagcctgaaccacattttagataactcaattatgtatgtatgtgcatacacatatacaaacacactaatggtagaatgcttttttatgtgctagactattatatttagtagtatgtcattgtaactagccaatatcacagcttttgaaaaattaaaaaatcacactatattaatatttcatatttgccaaaagaaacatggcagataggtctcaatatgttttcaatgcctgatgacctataagaagaaagtattgaaaagaagagagattagaactgttagaaggagttgaaattttctaaaagacatagtatttagtttataattaaatgcattcttgaagtccagtgtgaattttattaatgctatcatctcgaccaagctcaaagcctacttattagaaacaatgaagttcacaataggtcataaggtctcttccttttctaaaattgaaagacaagaaatttagtgccaatattgtacagacagaaattccatgtatgagtctcaacaaagactacctttggctaaatgtctagaagcagagaagtaaagtgagcaaaatccagtgttgaggagtcatgacagtactttgatctttatatactctgaagcatttcttcaaacttttctacttttatttgtcattgatacctgtagtaagttgacaatgtggtgaaatttcaaaattatatgtaacttctactagttttactttctcccccaagtcttttttaactcatgatttttacacacacaatccagaacttattatatagcctctaagtctttattcttcacagtagataatgaaagagtcctccagtgtcttggcaaaatgttctagtatagctggatacatacagtggagttctataaactcatatctcagtggacttaaccaaaattgtgttagtctcaattcctaccacactgagggagcctcccaaataactattttcttatctgcagtattcctccagaagagctaaccagggcagggctggcatgagaagtgacatctgcgttacaaagtctatcttcctcataagtctgtaaagagcaattgaatcttctagctttagcaaacctaagccaaaggaaggaaagccacgaagaatgcagaagtcaaaccctcatgacaaagtaggcacaagtctacaataagctaaatcagaatttacaaatacaagtgtcccaggtagcattgactcccgtcattggagtgaaatggatcaaagtttgaattaaggcctatggtaaggtaacattgctttgttgtacttttgaacaagagctcctcctgatcactattacatatttttctagaaaatctaaagttcagaagagaatgtatcactgctgacttttattccaatatttggatggagtaagttttagggtagaattttgttcagtttggatttaatcttttgaaaagtaaattccttgtttactggtttgactataattctctgttatctttacgaggtaaaactgcaagctgactagcatgttctgtgaatctgccattcctaaaaattttataaacacttgatacttttcactgataatggatcgctccaataaacatatattgtgaaaatgcatccacaataaatggaattccttcctgc

SEQ ID NO: 136 - DNA, 1501 bp, Homo sapiens
  >CD74 (HUGO ID) HLA CLASS II HISTOCOMPATIBILITY ANTIGEN, GAMMA CHAIN (HLA-DR ANTIGENS ASSOCIATED INVARIANT CHAIN) (IA ANTIGEN-ASSOCIATED INVARIANT CHAIN) (II) (P33) (CD74 ANTIGEN). [Source SWISSPROT (P04233)]
cagggtcccagatgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagtactgaccaagtgccaggaagaggtcagccacatccctgctgtccacccgggttcattcaggcccaagtgcgacgagaacggcaactatctgccactccagtgctatgggagcatcggctactgctggtgtgtcttccccaacggcacggaggtccccaacaccagaagccgcgggcaccataactgcagtgagtcactggaactggaggacccgtcttctgggctgggtgtgaccaagcaggatctgggcccagtccccatgtgagagcagcagaggcggtcttcaacatcctgccagccccacacagctacagctttcttgctcccttcagcccccagcccctcccccatctcccaccctgtacctcatcccatgagaccctggtgcctggctctttcgtcacccttggacaagacaaaccaagtcggaacagcagataacaatgcagcaaggccctgctgcccaatctccatctgtcaacaggggcgtgaggtcccaggaagtggccaaaagctagacagatccccgttcctgacatcacagcagcctccaacacaaggctccaagacctaggctcatggacgagatgggaaggcacagggagaagggataaccctacacccagaccccaggctggacatgctgactgtcctctcccctccagcctttggccttggcttttctagcctatttacctgcaggctgagccactctcttccctttccccagcatcactccccaaggaagagccaatgttttccacccataatcctttctgccgacccctagttccctctgctcagccaagcttgttatcagctttcagggccatggttcacattagaataaaaggtagtaattag

SEQ ID NO: 137 - DNA, 4009 bp, Homo sapiens
  >C7 (HUGO ID) COMPLEMENT COMPONENT C7 PRECURSOR. [Source SWISSPROT (P10643)]
cagggagaggcagagaggcaggcagcctgctgggctcttcctgctgttgaaaacttacccggcccttacagaggaaatcttcctcctctcttctgccctgaatgttttcccaaacatgaaggtgataagcttattcattttggtgggatttataggagagttccaaagtttttcaagtgcctcctctccagtcaactgccagtgggacttctatgccccttggtcagaatgcaatggctgtaccaagactcagactcgcaggcggtcagttgctgtgtatgggcagtatggaggccagccttgtgttggaaatgcttttgaaacacagtcctgtgaacctacaagaggatgtccaacagaggagggatgtggagagcgtttcaggtgcttttcaggtcagtgcatcagcaaatcattggtttgcaatggggattctgactgtgatgaagacagtgctgatgaagacagatgtgaggactcagaaaggagaccttcctgtgatatcgataaacctcctcctaacatagaacttactggaaatggttacaatgaactcactggccagtttaggaacagagtcatcaataccaaaagttttggtggtcaatgtagaaaggtgtttagtggggatggaaaagatttctacaggctgagtggaaatgtcctgtcctatacattccaggtgaaaataaataatgattttaattatgaattttacaatagtacttggtcttatgtaaaacatacgtcgacagaacacacatcatctagtcggaagcgctccttttttagatcttcatcatcttcttcacgcagttatacttcacataccaatgaaatccataaaggaaagagttaccaactgctggttgttgagaacactgttgaagtggctcagttcattaataacaatccagaatttttacaacttgctgagccattctggaaggagctttcccacctcccctctctgtatgactacagtgcctaccgaagattaatcgaccagtacgggacacattatctgcaatctgggtcgttaggaggagaatacagagttctattttatgtggactcagaaaaattaaaacaaaatgattttaattcagtcgaagaaaagaaatgtaaatcctcaggttggcattttgtcgttaaattttcaagtcatggatgcaaggaactggaaaacgctttaaaagctgcttcaggaacccagaacaatgtattgcgaggagaaccgttcatcagagggggaggtgcaggcttcatatctggccttagttacctagagctggacaatcctgctggaaacaaaaggcgatattctgcctgggcagaatctgtgactaatcttcctcaagtcataaaacaaaagctgacacctttatatgagctggtaaaggaagtaccttgtgcctctgtgaaaaaactatacctgaaatgggctcttgaagagtatctggatgaatttgacccctgtcattgccggccttgtcaaaatggtggtttggctactgttgaggggacccattgtctgtgccattgcaaaccgtacacatttggtgcggcgtgtgagcaaggagtcctcgtagggaatcaagcaggaggggttgatggaggttggagttgctggtcctcttggagcccctgtgtccaagggaagaaaacaagaagccgtgaatgcaataacccacctcccagtgggggtgggagatcctgcgttggagaaacgacagaaagcacacaatgcgaagatgaggagctggagcacttgaggttgcttgaaccacattgctttcctttgtctttggttccaacagaattctgtccatcacctcctgccttgaaagatggatttgttcaagatgaaggtacaatgtttcctgtggggaaaaatgtagtgtacacttgcaatgaaggatactctcttattggaaacccagtggccagatgtggagaagatttacggtggcttgttggggaaatgcattgtcagaaaattgcctgtgttctacctgtactgatggatggcatacagagtcacccccaaaaacctttctacacagttggtgagaaggtgactgtttcctgttcaggtggcatgtccttagaaggtccttcagcatttctctgtggctccagccttaagtggagtcctgagatgaagaatgcccgctgtgtacaaaaagaaaatccgttaacacaggcagtgcctaaatgtcagcgctgggagaaactgcagaattcaagatgtgtttgtaaaatgccctacgaatgtggaccttccttggatgtatgtgctcaagatgagagaagcaaaaggatactgcctctgacagtttgcaagatgcatgttctccactgtcagggtagaaattacacccttactggtagggacagctgtactctgcctgcctcagctgagaaagcttgtggtgcctgcccactgtggggaaaatgtgatgctgagagcagcaaatgtgtctgccgagaagcatcggagtgcgaggaagaagggtttagcatttgtgtggaagtgaacggcaaggagcagacgatgtctgagtgtgaggcgggcgctctgagatgcagagggcagagcatctctgtcaccagcataaggccttgtgctgcggaaacccagtaggctcctggaggccctggtcagcttgcttggaatccagcaggcagctggggctgagtgaaaacatctgcacaactgggcactggacagcttttccttcttctccagtgtctaccttcctcctcaactcccagccatctgtataaacacaatcctttgttctcccaaatctgaatcgaattactcttttgcctcctttttaatgtcagtaaggatatgagcctttgcacaggctggctgcgtgttcttgaaataggtgttaccttctctgggccttggttttttaaaatctgtaaaattagaggattgcactagagaaacttgaatgctccattcaggcctatcattttattaagtatgattgacacagcccatgggccagaacacactctacaaaatgactaggataacagaaagaacgtgatctcctgattagagagggtggttttcctcaatggaaccaaatataaagaggacttgaacaaaaatgacagatacaaactatttctatcctgagtagtaatctcacacttcatcctatagagtcaaccaccacagataggaattccttattctttttttaatttttttaagacagagtctcactttgttgcccaggctggagcgcagtggggtgatctcatctccctgcaacctccgcctcctgggttcaagcgattcttgtgcctcagcttcccaagcagctgggattacaggtgcccgccaccacgcccagctaatttttgcatttttagtagagatggggtttcaccatgttggccacgctcgtctccaactcctgacctcaggtaatccgcctgccttggcctcccaaagtgctgggattacagacatgaaccaccacgcctggctggaatacttactcttgtcgggagattgaaccactaaaatgttagagcagaattcattatgctgtggtcacaggggtgtcttgtctgagaacaaatacaattcagtcttctctttggggttttagtatgtgtcaaacataggactggaagtttgcccctgttcttttttcttttgaaagaacatcagttcatgcctgaggcatgagtgactgtgcatttgagaatagttttccctattctgtggatacagtcccagagttttcagggagtacacaggtagattagtttgaagcattgaccttttatttattccttatttctctttcatcaaaacaaaacagcagctgtgggaggagaaatgagagggcttaaatgaaatttaaaataagctatattatacaaatactatctctgtattgttctgaccctggtaaatatatttcaaaacttcagatgacaaggattagaacactcattaaagatgctattcttc

SEQ ID NO: 138 - DNA, 2240 bp, Homo sapiens
  >CXCL16 (HUGO ID) SMALL INDUCIBLE CYTOKINE B16 (TRANSMEMBRANE CHEMOKINE CXCL16) (SR- PSOX) (SCAVENGER RECEPTOR FOR PHOSPHATIDYLSERINE AND OXIDIZED LOW DENSITY LIPOPROTEIN). [Source SWISSPROT (Q9H2A7)]
catgcagcgcggctgggtcccgcggcgcccggatcggggaagtgaaagtgcctcggaggaggagggccggtccggcagtgcagccgcctcacaggtcggcggacgggccaggcgggcggcctcctgaaccgaaccgaatcggctcctcgggccgtcgtcctcccgcccctcctcgcccgccgccggagttttctttcggtttcttccaagattcctggccttccctcgacggagccgggcccagtgcgggggcgcagggcgcgggagctccacctcctcggctttccctgcgtccagaggctggcatggcgcgggccgagtactgagcgcacggtcggggcacagcagggccggggggtgcagctggctcgcgcctcctctccggccgccgtctcctccggtccccggcgaaagccattgagacaccagctggacgtcacgcgccggagcatgtctgggagtcagagcgaggtggctccatccccgcagagtccgcggagccccgagatgggacgggacttgcggcccgggtcccgcgtgctcctgctcctgcttctgctcctgctggtgtacctgactcagccaggcaatggcaacgagggcagcgtcactggaagttgttattgtggtaaaagaatttcttccgactccccgccatcggttcagttcatgaatcgtctccggaaacacctgagagcttaccatcggtgtctatactacacgaggttccagctcctttcctggagcgtgtgtgggggcaacaaggacccatgggttcaggaattgatgagctgtcttgatctcaaagaatgtggacatgcttactcggggattgtggcccaccagaagcatttacttcctaccagccccccaatttctcaggcctcagagggggcatcttcagatatccacacccctgcccagatgctcctgtccaccttgcagtccactcagcgccccaccctcccagtaggatcactgtcctcggacaaagagctcactcgtcccaatgaaaccaccattcacactgcgggccacagtctggcagctgggcctgaggctggggagaaccagaagcagccggaaaaaaatgctggtcccacagccaggacatcagccacagtgccagtcctgtgcctcctggccatcatcttcatcctcaccgcagccctttcctatgtgctgtgcaagaggaggagggggcagtcaccgcagtcctctccagatctgccggttcattatatacctgtggcacctgactctaatacctgagccaagaatggaagcttgtgaggagacggactctatgttgcccaggctgttatggaactcctgagtcaagtgatcctcccaccttggcctctgaaggtgcgaggattataggcgtcacctaccacatccagcctacacgtatttgttaatatctaacataggactaaccagccactgccctctcttaggcccctcatttaaaaacggttatactataaaatctgcttttcacactgggtgataataacttggacaaattctatgtgtattttgttttgttttgctttgctttgttttgagacggagtctcgctctgtcatccaggctggagtgcagtggcatgatctcggctcactgcaacccccatctcccaggttcaagcgattctcctgcctcctcctgagtagctgggactacaggtgctcaccaccacacccggctaattttttgtatttttagtagagacggggtttcaccatgttgaccaggctggtctcgaactcctgacctggtgatctgcccacccaggcctcccaaagtgctgggattaaaggtgtgagccaccatgcctggccctatgtgtgttttttaactactaaaaattatttttgtaatgattgagtcttctttatggaaacaactggcctcagcccttgcgcccttactgtgattcctggcttcattttttgctgatggttccccctcgtcccaaatctctctcccagtacaccagttgttcctcccccacctcagccctctcctgcatcctcctgtacccgcagcgaaggcctgggctttcccaccctccctccttagcaggtgccgtgctgggacaccatacgggttggtttcacctcctcagtcccttgcctaccccagtgagagtctgatcttgtttttattgttattgcttttattattattgcttttattatcattaaaactctagttcttgttttgtctctccg

SEQ ID NO: 139 - DNA, 4234 bp, Homo sapiens
  CYBRD1 (HUGO ID) CYTOCHROME B REDUCTASE 1; DUODENAL CYTOCHROME B. [Source RefSeq (NM_024843)]
ggccactacccagagggctgccgccgcctctccaagttcttgtggcccccgcggtgcggagtatggggcgctgatggccatggagggctactggcgcttcctggcgctgctggggtcggcactgctcgtcggcttcctgtcggtgatcttcgccctcgtctgggtcctccactaccgagaggggcttggctgggatgggagcgcactagagtttaactggcacccagtgctcatggtcaccggcttcgtcttcatccagggcatcgccatcatcgtctacagactgccgtggacctggaaatgcagcaagctcctgatgaaatccatccatgcagggttaaatgcagttgctgccattcttgcaattatctctgtggtggccgtgtttgagaaccacaatgttaacaatatagccaatatgtacagtctgcacagctgggttggactgatagctgtcatatgctatttgttacagcttctttcaggtttttcagtctttctgcttccatgggctccgctttctctccgagcatttctcatgcccatacatgtttattctggaattgtcatctttggaacagtgattgcaacagcacttatgggattgacagagaaactgattttttccctgagagatcctgcatacagtacattcccgccagaaggtgttttcgtaaatacgcttggccttctgatcctggtgttcggggccctcattttttggatagtcaccagaccgcaatggaaacgtcctaaggagccaaattctaccattcttcatccaaatggaggcactgaacagggagcaagaggttccatgccagcctactctggcaacaacatggacaaatcagattcagagttaaacagtgaagtagcagcaaggaaaagaaacttagctctggatgaggctgggcagagatctaccatgtaaaatgttgtagagatagagccatataacgtcacgtttcaaaactagctctacagttttgcttctcctattagccatatgataattgggctatgtagtatcaatatttactttaatcacaaaggatggtttcttgaaataatttgtattgattgaggcctatgaactgacctgaattggaaaggatgtgattaatataaataatagcagatataaattgtggttatgttacctttatcttgttgaggaccacaacattagcacggtgccttgtgcagaatagatactcaatatgtgaatatgtgtctactagtagttaattggataaactggcagcatccctggcctgttgtcatgcagtcatttcctgttaattctgggagacaatgatttcacaactagcgggaagcagtcctaaaagtttaaaatccgataaggaatatctgggacagggtttagatcatgactctacacagataccatgatgagagtatattaaagaaatttaggaaagcacctggttcctttctccccatgcctgccttctgctccctccccagctggtttgggctcaaattgtccctggagactagggtttatgttagggtattgatagattagagcaggtggttgaagagatcttctctggtcagacttggaagaatttccaaaactgaagttagccccaagacttccctagggttgatgtactttatgatccagatgctaaacttcttagaatgaaaatatgcttcaacacttaagtagcatacactgccctacaaacctcagagagcacttttccccaagttcttgtttttatttttgaaagtactcacacagcacttactatgctccaaacactcctctaagcactttacacatattagctcattcagtccccagacagacgggatgaagtaggtattgttactgttcccattttacaggtgagagatttgaagcctggggaggctagtaactcaccccaaggtcacacggctcatacatggtgggactgagactcagatgcaggcagtctggcacctcagtctggattctaaccatttcactaagctatttttgtcttgtactactttgacccacccctgaataaacctcaattgctggagtggggtgtagttattaaagggatgctttttaccttttgctgtttgctgtggcagattccccagataaccaaggaaaaggggccacccatacctggaaataggccatagggcccctactactgccaacaagccatggcctaccttgacacttgtttgatcttaaaattgtgtcttggtaacaaaagatttggacaggcatatctgtagctttcaagttaattaattgcaatatttttttcttcaggattttagctgctgaacaactttcagtttggagctaaaagagacctgtctcatggtctgcccttccctggggcaatagctagggtctttcctgatttttatggaattttaggggatattttgagctttgggttctcagtagtgaattgagacttggaggtgacttttcatgtttggagtatcatctctgtctgggatctgggctgacaaattaaaacctagagtagtgcttatgctgaaatgatacttttcattttttggttgatttttttgccttcccttcaattttaaactgaagcattttaatatgggtagaaactctacaccaaatacactaaacattttggtgcttagtggatttctttttaggtaactggtacttacttccaaagactgaatacaagccacactccatcatatcccttaaacttcatgaaaaaccattcaagatccccttgctgcaacactgttctcttcttctctactaaattctatttccaaaattggtaatagagccagaaggatccccagtacccagccctctgcctggcacaaagtggtagcacaattaaattcagtatgggtggagcatggtacagtcttggtgccatagaaggagtagttgcatagtcacacatcatttgataagttggatgttccattacatagaggaacacaaaattccagggtttttggaggaagggattagatagtgactaagccgccagaattgaggtggccattcctttttgtataggctaagaaacaggttatcagtgaaaagttaattatggctttggcactagaatagcactgttgcaaagtatttaagcaccccccatctcagccctttattttatctttcatgtgggctaatgtgaggataatcttacagatattataggaatttcttttctatctttatgaaaacaacgtatataaaatatatctagaaaacctttgtttgagactcttatttaatgggcttttgattctaatgataattgtacctttatctttcaaaagctgatatttcctacctaagcatctcccgagaaaaatatctcattaaaaagcccataaataataggggagaagaaagccttaggtatcaattccaaaacagtgattgaaatttcccaaaataattatggcttctgtcatctccagagataatctggcttggtttaccccataatctaatttcagaaaagaaagctttattttaacactcatctgaatcaacattaaagccttttctctcaaagcgtttattgagaaactcaaatgaatatactttttgaattactgtcatcaaaagtgtacggcttcctgtgctgcttgtgtcaaatggaacctgccctctaaagcactttctttcctttacttgcgtggtttcatgtaagctgtgctgtttagaaacaacatctcagactttacaaagaaatgacaaagaaggcaattgcactttttaagggatatcgacaagcagtttctgttttctaaaggacaaaatacagagtgtgtgtcatttttaattagattctttcccctgctgagttggaaattccagtgcagcactgattgaccacagttgccaatctaaaagcacaaagacagaagtaaagctttatgctaattttatttcaatatgatagaaaatttatcttggtatgtccttttttagataactccagcaggaaactgtaactgctatgtctttaggaaaatgtagaagaaagaacattattgttctttaattcctacaaggtactcgaaaaccttaagtgaaaaagatttctatctttttatcttagcgcatttatggaaaaaatattaactatcctgaatattttataattttgtaggaaaaatatgcatctattttttcttgacttcttttatatagtaataaaagttattttgg

SEQ ID NO: 140 - DNA, 606 bp, Homo sapiens
  >IFITM1 (HUGO ID) INTERFERON-INDUCED TRANSMEMBRANE PROTEIN 1 (INTERFERON-INDUCED PROTEIN 17) (INTERFERON-INDUCIBLE PROTEIN 9-27) (LEU-13 ANTIGEN) (CD225 ANTIGEN). [Source SWISSPROT (P13164)]
cccttcgctccacgcagaaaaccacacttctcaaaccttcactcaacacttccttccccaaagccagaagatgcacaaggaggaacatgaggtggctgtgctggggccaccccccagcaccatccttccaaggtccaccgtgatcaacatccacagcgagacctccgtgcccgaccatgtcgtctggtccctgttcaacaccctcttcttgaactggtgctgtctgggcttcatagcattcgcctactccgtgaagtctagggacaggaagatggttggcgacgtgaccggggcccaggcctatgcctccaccgccaagtgcctgaacatctgggccctgattctgggcatcctcatgaccattggattcatcctgttactggtattcggctctgtgacagtctaccatattatgttacagataatacaggaaaaacggggttactagtagccgcccatagcctgcaacctttgcactccactgtgcaatgctggccctgcacgctggggctgttgcccctgcccccttggtcctgcccctagatacagcagtttatacccacacacctgtctacagtgtcattcaataaagtgcacgtgcttgtg

SEQ ID NO: 141 - DNA, 3423 bp, Homo sapiens
  SALL4 (HUGO ID) SAL-LIKE PROTEIN 4 (ZINC FINGER PROTEIN SALL4). [Source SWISSPROT (Q9UJQ4)]
aactccaggaatttgtggcggagagggcaaataactgcggctctcccggcgccccgatgctcgcaccatgtcgaggcgcaagcaggcgaaaccccagcacatcaactcggaggaggaccagggcgagcagcagccgcagcagcagaccccggagtttgcagatgcggccccagcggcgcccgcggcgggggagctgggtgctccagtgaaccacccagggaatgacgaggtggcgagtgaggatgaagccacagtaaagcggcttcgtcgggaggagacgcacgtctgtgagaaatgctgtgcggagttcttcagcatctctgagttcctggaacataagaaaaattgcactaaaaatccacctgtcctcatcatgaatgacagcgaggggcctgtgccttcagaagacttctccggagctgtactgagccaccagcccaccagtcccggcagtaaggactgtcacagggagaatggcggcagctcagaggacatgaaggagaagccggatgcggagtctgtggtgtacctaaagacagagacagccctgccacccaccccccaggacataagctatttagccaaaggcaaagtggccaacactaatgtgaccttgcaggcactacggggcaccaaggtggcggtgaatcagcggagcgcggatgcactccctgcccccgtgcctggtgccaacagcatcccgtgggtcctcgagcagatcttgtgtctgcagcagcagcagctacagcagatccagctcaccgagcagatccgcatccaggtgaacatgtgggcctcccacgccctccactcaagcggggcaggggccgacactctgaagaccttgggcagccacatgtctcagcaggtttctgcagctgtggctttgctcagccagaaagctggaagccaaggtctgtctctggatgccttgaaacaagccaagctacctcacgccaacatcccttctgccaccagctccctgtccccagggctggcacccttcactctgaagccggatgggacccgggtgctcccgaacgtcatgtcccgcctcccgagcgctttgcttcctcaggccccgggctcggtgctcttccagagccctttctccactgtggcgctagacacatccaagaaagggaaggggaagccaccgaacatctccgcggtggatgtcaaacccaaagacgaggcggccctctacaagcacaagtgtaagtactgtagcaaggtttttgggactgatagctccttgcagatccacctccgctcccacactggagagagacccttcgtgtgctctgtctgtggtcatcgcttcaccaccaagggcaacctcaaggtgcactttcaccgacatccccaggtgaaggcaaacccccagctgtttgccgagttccaggacaaagtggcggccggcaatggcatcccctatgcactctctgtacctgaccccatagatgaaccgagtctttctttagacagcaaacctgtccttgtaaccacctctgtagggctacctcagaatctttcttcggggactaatcccaaggacctcacgggtggctccttgcccggtgacctgcagcctgggccttctccagaaagtgagggtggacccacactccctggggtgggaccaaactataattccccaagggctggtggcttccaagggagtgggacccctgagccagggtcagagaccctgaaattgcagcagttggtggagaacattgacaaggccaccactgatcccaacgaatgtctcatttgccaccgagtcttaagctgtcagagctccctcaagatgcattatcgcacccacaccggggagagaccgttccagtgtaagatctgtggccgagccttttctaccaaaggtaacctgaagacacaccttggggttcaccgaaccaacacatccattaagacgcagcattcgtgccccatctgccagaagaagttcactaatgccgtgatgctgcagcaacatattcggatgcacatgggcggtcagattcccaacacgcccctgccagagaatccctgtgactttacgggttctgagccaatgaccgtgggtgagaacggcagcaccggcgctatctgccatgatgatgtcatcgaaagcatcgatgtagaggaagtcagctcccaggaggctcccagcagctcctccaaggtccccacgcctcttcccagcatccactcggcatcacccacgctagggtttgccatgatggcttccttagatgccccagggaaagtgggtcctgccccttttaacctgcagcgccagggcagcagagaaaacggttccgtggagagcgatggcttgaccaacgactcatcctcgctgatgggagaccaggagtatcagagccgaagcccagatatcctggaaaccacatccttccaggcactctccccggccaatagtcaagccgaaagcatcaagtcaaagtctcccgatgctgggagcaaagcagagagctccgagaacagccgcactgagatggaaggtcggagcagtctcccttccacgtttatccgagccccgccgacctatgtcaaggttgaagttcctggcacatttgtgggaccctcgacattgtccccagggatgacccctttgttagcagcccagccacgccgacaggccaagcaacatggctgcacacggtgtgggaagaacttctcgtctgctagcgctcttcagatccacgagcggactcacactggagagaagccttttgtgtgcaacatttgtgggcgagcttttaccaccaaaggcaacttaaaggttcactacatgacacacggggcgaacaataactcagcccgccgtggaaggaagttggccatcgagaacaccatggctctgttaggtacggacggaaaaagagtctcagaaatctttcccaaggaaatcctggccccttcagtgaatgtggaccctgttgtgtggaaccagtacaccagcatgctcaatggcggtctggccgtgaagaccaatgagatctctgtgatccagagtgggggggttcctaccctcccggtttccttgggggccacctccgttgtgaataacgccactgtctccaagatggatggctcccagtcgggtatcagtgcagatgtggaaaaaccaagtgctactgacggcgttcccaaacaccagtttcctcacttcctggaagaaaacaagattgcggtcagctaagggagaacttgcgtggaaggagcaatgcagacacagtgaaatctctagaatctgctttgttttgtaagaactcatctcctcctgttttctttttcttactgatatgcaaatgatgtttactacgttggttgtgaccacaacctcaggcaagtgctacaatcacgattgttgctatgctgctttgcaaaaagttg

SEQ ID NO: 142 - DNA, 2412 bp, Homo sapiens
  TRA1 (HUGO ID) ENDOPLASMIN PRECURSOR (94 KDA GLUCOSE-REGULATED PROTEIN) (GRP94) (GP96 HOMOLOG) (TUMOR REJECTION ANTIGEN 1). [Source SWISSPROT (P14625)]
atgagggccctgtgggtgctgggcctctgctgcgtcctgctgaccttcgggtcggtcagagctgacgatgaagttgatgtggatggtacagtagaagaggatctgggtaaaagtagagaaggatcaaggacggatgatgaagtagtacagagagaggaagaagctattcagttggatggattaaatgcatcacaaataagagaacttagagagaagtcggaaaagtttgccttccaagccgaagttaacagaatgatgaaacttatcatcaattcattgtataaaaataaagagattttcctgagagaactgatttcaaatgcttctgatgctttagataagataaggctaatatcactgactgatgaaaatgctctttctggaaatgaggaactaacagtcaaaattaagtgtgataaggagaagaacctgctgcatgtcacagacaccggtgtaggaatgaccagagaagagttggttaaaaaccttggtaccatagccaaatctgggacaagcgagtttttaaacaaaatgactgaagcacaggaagatggccagtcaacttctgaattgattggccagtttggtgtcggtttctattccgccttccttgtagcagataaggttattgtcacttcaaaacacaacaacgatacccagcacatctgggagtctgactccaatgaattttctgtaattgctgacccaagaggaaacactctaggacggggaacgacaattacccttgtcttaaaagaagaagcatctgattaccttgaattggatacaattaaaaatctcgtcaaaaaatattcacagttcataaactttcctatttatgtatggagcagcaagactgaaactgttgaggagcccatggaggaagaagaagcagccaaagaagagaaagaagaatctgatgatgaagctgcagtagaggaagaagaagaagaaaagaaaccaaagactaaaaaagttgaaaaaactgtctgggactgggaacttatgaatgatatcaaaccaatatggcagagaccatcaaaagaagtagaagaagatgaatacaaagctttctacaaatcattttcaaaggaaagtgatgaccccatggcttatattcactttactgctgaaggggaagttaccttcaaatcaattttatttgtacccacatctgctccacgtggtctgtttgacgaatatggatctaaaaagagcgattacattaagctctatgtgcgccgtgtattcatcacagacgacttccatgatatgatgcctaaatacctcaattttgtcaagggtgtggtggactcagatgatctccccttgaatgtttcccgcgagactcttcagcaacataaactgcttaaggtgattaggaagaagcttgttcgtaaaacgctggacatgatcaagaagattgctgatgataaatacaatgatactttttggaaagaatttggtaccaacatcaagcttggtgtgattgaagaccactcgaatcgaacacgtcttgctaaacttcttaggttccagtcttctcatcatccaactgacattactagcctagaccagtatgtggaaagaatgaaggaaaaacaagacaaaatctacttcatggctgggtccagcagaaaagaggctgaatcttctccatttgttgagcgacttctgaaaaagggctatgaagttatttacctcacagaacctgtggatgaatactgtattcaggcccttcccgaatttgatgggaagaggttccagaatgttgccaaggaaggagtgaagttcgatgaaagtgagaaaactaaggagagtcgtgaagcagttgagaaagaatttgagcctctgctgaattggatgaaagataaagcccttaaggacaagattgaaaaggctgtggtgtctcagcgcctgacagaatctccgtgtgctttggtggccagccagtacggatggtctggcaacatggagagaatcatgaaagcacaagcgtaccaaacgggcaaggacatctctacaaattactatgcgagtcagaagaaaacatttgaaattaatcccagacacccgctgatcagagacatgcttcgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcccgaagaagaacctgaagagacagcagaagacacaacagaagacacagagcaagacgaagatgaagaaatggatgtgggaacagatgaagaagaagaaacagcaaaggaatctacagctgaaaaagatgaattgtaa

SEQ ID NO: 143 - DNA, 4757 bp, Homo sapiens
  >COL1A2 (HUGO ID) ALPHA 2 TYPE I COLLAGEN; COLLAGEN I, ALPHA-2 POLYPEPTIDE; COLLAGEN OF SKIN, TENDON AND BONE, ALPHA-2 CHAIN. [Source RefSeq (NM_000089)]
atgctcagctttgtggatacgcggactttgttgctgcttgcagtaaccttatgcctagcaacatgccaatgtaagtgccttcagcttgtttgggggagactgggtagagagggtccaccaggccccccaggcagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtccccctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccaggaattctttgtggacattggcccagtctgtttcaaataaatgaactcaatctaaattaaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatggaaaatgtcaacctttgtaagaaaaccaaaataaaaattgaaaaataaaaaccataaacatttgcaccacttgtggcttttgaatatcttccacagagggaagtttaaaacccaaacttccaaaggtttaaactacctcaaaacactttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaattaaaagtatgcagattatttgcccaaatcttcttcagattcagcatttgttctttgccagtctcattttcatcttcttccatggttccacagaagctttgtttcttgggcaagcagaaaaattaaattgtacctattttgtatatgtgagatgtttaaataaattgtgaaaaaaatgaaataaagcatgtttggttttccaaaagaacatat

SEQ ID NO: 144 - DNA, 1934 bp, Homo sapiens
  HDAC3 (HUGO ID) HISTONE DEACETYLASE 3 (HD3) (RPD3-2). [Source SWISSPROT (O15379)]
gcggcggccgcgggcggcgggcggcggaggtgcggggcctgctcccgccggcaccatggccaagaccgtggcctatttctacgaccccgacgtgggcaacttccactacggagctggacaccctatgaagccccatcgcctggcattgacccatagcctggtcctgcattacggtctctataagaagatgatcgtcttcaagccataccaggcctcccaacatgacatgtgccgcttccactccgaggactacattgacttcctgcagagagtcagccccaccaatatgcaaggcttcaccaagagtcttaatgccttcaacgtaggcgatgactgcccagtgtttcccgggctctttgagttctgctcgcgttacacaggcgcatctctgcaaggagcaacccagctgaacaacaagatctgtgatattgccattaactgggctggtggtctgcaccatgccaagaagtttgaggcctctggcttctgctatgtcaacgacattgtgattggcatcctggagctgctcaagtaccaccctcgggtgctctacattgacattgacatccaccatggtgacggggttcaagaagctttctacctcactgaccgggtcatgacggtgtccttccacaaatacggaaattacttcttccctggcacaggtgacatgtatgaagtcggggcagagagtggccgctactactgtctgaacgtgcccctgcgggatggcattgatgaccagagttacaagcaccttttccagccggttatcaaccaggtagtggacttctaccaacccacgtgcattgtgctccagtgtggagctgactctctgggctgtgatcgattgggctgctttaacctcagcatccgagggcatggggaatgcgttgaatatgtcaagagcttcaatatccctctactcgtgctgggtggtggtggttatactgtccgaaatgttgcccgctgctggacatatgagacatcgctgctggtagaagaggccattagtgaggagcttccctatagtgaatacttcgagtactttgccccagacttcacacttcatccagatgtcagcacccgcatcgagaatcagaactcacgccagtatctggaccagatccgccagacaatctttgaaaacctgaagatgctgaaccatgcacctagtgtccagattcatgacgtgcctgcagacctcctgacctatgacaggactgatgaggctgatgcagaggagaggggtcctgaggagaactatagcaggccagaggcacccaatgagttctatgatggagaccatgacaatgacaaggaaagcgatgtggagatttaagagtggcttgggatgctgtgtcccaaggaatttcttttcacctcttggttgggctggagggaaaaggagtggctcctagagtcctgggggtcaccccagggcttttgctgactctgggaaagagtctggagaccacatttggttctcgaaccatctacctgcttttcctctctctcccaaggcctgacaatggtacctattagggatgagatacagacaaggatagctatctgggacattattggcagtgggccctggaggccagtccctagccccccttgccccttatttcttccctgcttccctcgaacccagagatttttgagggatgaacgggtagacaaggactgagattgcctctgacttcctcctcccctgggttctgacttcttcctccccttgcttccagggaagatgaagagagagagatttggaaggggctctggctccctaacacctgaatcccagatgatgggaagtatgttttcaagtgtggggaggatatgaaaatgttctgttctcacttttggctttatgtccattttaccactgtttttatccaataaactaagtcggtattttttgtaccttt

SEQ ID NO: 145 - DNA, 1235 bp, Homo sapiens
  >HLA-DRA (HUGO ID) HLA CLASS II HISTOCOMPATIBILITY ANTIGEN, DR ALPHA CHAIN PRECURSOR. [Source SWISSPROT (P01903)]
attcttgtctgttctgcctcactcccgagctctactgactcccaacagagcgcccaagaagaaaatggccataagtggagtccctgtgctaggatttttcatcatagctgtgctgatgagcgctcaggaatcatgggctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacgaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatcttcatcatcaagggattgcgcaaaagcaatgcagcagaacgcagggggcctctgtaaggcacatggaggtgatggtgtttcttagagagaagatcactgaagaaacttctgctttaatggctttacaaagctggcaatattacaatccttgacctcagtgaaagcagtcatcttcagcattttccagccctatagccaccccaagagtggttatgcctcctcgattgctccatactctaacatctagctggcttccctgtctattgccttttcctgtatctattttcctctatttcctatcattttattatcaccatgcaatgcctctggaataaaacatacaggagtctgtctctgctatggaatgccccatggggcatctcttgtgtacttattgtttaaggtttcctcaaactgtgatttttctgaacacaataaactattttgaagatcttgggtggaa

SEQ ID NO: 146 - DNA, 5918 bp, Homo sapiens
  >OPA1 (HUGO ID) DYNAMIN-LIKE 120 KDA PROTEIN, MITOCHONDRIAL PRECURSOR (OPTIC ATROPHY 1 GENE PROTEIN). [Source SWISSPROT (O60313)]
cacgggggctcccgcgtggccgtctcggcgcctgcgtgacctccccgccggcgggatgtggcgactacgtcgggccgctgtggcctgtgaggtctgccagtctttagtgaaacacagctctggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttgagaaaattagaaaagcccttcctagttcagaagaccttgtaaagttagcaccagactttgacaagattgttgaaagccttagcttattgaaggacttttttacctcaggtcacaaattggttagtgaagtcataggagcttctgacctacttctcttgttaggttctccggaagaaacggcgtttagagcaacagatcgtggatctgaaagtgacaagcattttagaaaggtgtcagacaaagagaaaattgaccaacttcaggaagaacttctgcacactcagttgaagtatcagagaatcttggaacgattagaaaaggagaacaaagaattgagaaaattagtattgcagaaagatgacaaaggcattcatcatagaaagcttaagaaatctttgattgacatgtattctgaagttcttgatgttctctctgattatgatgccagttataatacgcaagatcatctgccacgggttgttgtggttggagatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataaattaaaatcgtactcataatcagctctgcatacatctgaagaacaaaaacatcaacgtcttttgtccagcctctttttcttctgctgttccacctttctaaacatacaataaagtcatgggataaaaataatcgatgtatgttacgggcgctttaaccatcagctgcctctcgaatggaagaacagtggtaatggattaacatcctattttgttgtactaaagtgacaaatcggaataatataattggtatggccattaggttcagtccttgaagataagaaacttgttctctgtttgttgtcttatttgtggtggcactcgtttaatggattaactgaggttgctcaatgttcagtttcttttccagaaatacaatgctaggtgttttgaaataaaacttatatagcaattgtttaaagttatcaattgtatataaaatcacagtagcctgctaaatcattgtatgtgtctgtagtattctattcccagaaactatttgaccatgataattcagtttatattcaccacatgaaagaaaaatgggtaacagaagaacccttaaaacaggttaatttggattgtaacgttcagtgaaagaaatttcaacccttcatagccagcgaagaaatttgccttggaagccaagtcagtaccagcttacctatttgattcagttgctgttttctcactctctatatccatttgaaattgatttattttagatgttgtatacttacgttaggctttctgttaatagtggtttttctcctgttgacagagccaccggattatgacacaggatgaggaagattaaggataatcaattgactaatttcatttagaatattatcaaacatttcaactaggtatcagaaaaaggctttctttcataagactattttaaatagaaattatttcaacaattaaagtaatgttgaccatccccctctcagctgaataaagaaaaatttagttcaatttattgcaatttaattacaatactaccttcacaacattttcatgtgttttaaataaatattttttaattggctaaaggacattcaagcaaagaaatgctttctttacttaaaatgtctatctcatttgctgccttttcactaagcctttactttgttaataaaagtgtccattgtgtgatgtttttgattttacagtttgctaaatcttattttcttggagttgctttttggtaacagccccattgctactccccattttattgttttacatcaatgcatgcttcgttgtgatccctcaagatgtaacacttggtatgctcggttgaggatatgaaaaaatacttccgaaaccaggaattcaatgtatgtttgttttatactgtttgataagaaaagtaggtccagccttaagcagcacagatgcgctggtagatgcatagtcaggaactttttttatttcttttaggtctagggacaggagtgaatagaaagggaggagagctctattatgttctatacacagattaggagatgaccttactgggtacacccctctaaccagtgcttacaggttaatgcatgttaatgaatatttttgcagttgtaaagcataacaattacaactacacatctatttctaaagaataaaacaggaccatatttatttacttctgtcaactatagaaagaaagaccttcagctgtatttccacagatttctcccaaggaaaaggctaatattagtcactactgttatcacatccctttgtataagttttaaaaagagatggagggagatcttcatttctttgaggagatcagtattgtaacgtatgtgaatagatgataacaattaatattactaaaagtcccacatgagagtcctgacgccctctccatgccccacagtaatgtggcttctttcatgggtttttttttcttctttttagctgatctcatcctaagcatgctttatttttccttgaaagctaggtatttatcaactgcagatgttattgaaagaaaataaaattcagtctcaagagtaaaccctgtgtcttgtgtctgtagttcaaaagtcagaaatgattctaatttaaacaaaaagatactaaatatacagaagttaaattcgaactagccacagaatcatttgtttttatgtcagaatttgcaaagagtggagtggacaaagctctgtatggaagactgaacaactgtaaatagatgatatccaaacttaatttggctaggacttcaattttaaaaatcagtgtacctaggcagtgcacagcacgaaataagtggcccttgcagcttccccgtttaacccactgtgctatagttgcgggtggaacagtcaacctttctagtagtttatgatattgccctctttgtattcccattttctacagttttttccgcagacttctttctgcaaattattcagcctccaaatgcaaatgaatgatataaaaataagtagggaacatggcagagagtggtgcttcccagcctcacaatgtgggaatttgacataggatgagagtcagagtataggtttaaaagataaaatctttagttaataattttgtatttatttattctagatgtatgtatctgaggaaagaaatctggtatttttgctttccaataaaggggatcaaagtaatggtttttctctcagttctctaagctggtctatgttatagctctagcagtatggaaatgtgctttaaaatatgcttaccttttgaatgatcatggctatatgttgttgagatatttgaaacttaccttgttttcacttgtgcactgtgaatgaactttgtattatttttttaaaaccttcacattacgtgtagatattattgcaacttatattttgcctgagcttgatcaaaggtcatttgtgtagatgagtaattaaaaaatatttaaatcac

SEQ ID NO: 147 - DNA, 16396 bp, Homo sapiens
  BPAG1 (HUGO ID) BULLOUS PEMPHIGOID ANTIGEN 1 ISOFORMS 1/2/3/4/5/8 (230 KDA BULLOUS PEMPHIGOID ANTIGEN) (BPA) (HEMIDESMOSOMAL PLAQUE PROTEIN) (DYSTONIA MUSCULORUM PROTEIN) (FRAGMENT). [Source SWISSPROT (Q03001)]
cagctgccacttttcaccgttagaagtagagctttttccagacctcctaccttttagtctactttgaaaggtgaaagaaagaacatcgtttcaggaataaaaatgcacagtagtagttatagttaccgtagcagtgattctgtgtttagtaacactaccagcactcgaaccagtcttgattcaaatgaaaatcttctcttggttcattgtggtccaacactgatcaactcttgcattagcttcggcagtgaatcctttgatggacacaggttagaaatgttgcaacagattgccaacagagttcagagggacagtgtcatctgtgaagacaaactgattcttgctggaaatgctcttcagtctgattctaaaagattagaatcaggagtgcagtttcagaatgaagcagaaattgctgggtatatacttgaatgtgagaaccttttacgccagcatgtaattgatgtacagattcttattgatggaaaatactaccaggcagatcaattggtacagagggttgcaaaactgcgtgacgaaattatggccttaaggaacgaatgttcttctgtgtacagcaaaggacgcatactgacaacagaacagacaaagctcatgatatcaggaatcactcaaagtttaaactcaggatttgcacagaccttacaccctagtctgacctcagggctgacccagagtttaacaccttccctaacctcttctagtatgacttctggcctgtcatcagggatgacttcccgcctgactccatctgtcactccagcttatacacctggtttcccatcaggattagttccaaatttcagttcaggagtagagccaaattcattgcaaactttgaagttgatgcagatccgaaaaccccttctaaagtcttctttgctggatcaaaatttaacagaagaagaaatcaatatgaaatttgttcaggatcttttgaattgggttgatgagatgcaggtacaactggaccgcactgagtggggctcagatttgccaagtgttgaaagccatttagaaaatcataaaaatgttcatagagctattgaagaatttgaatctagtctcaaagaagctaaaatcagtgagattcaaatgacagcacctcttaaactgacttatgcagaaaagttgcacagattagagagtcagtatgcaaaactcttgaatacatccaggaatcaagaacggcaccttgatacactccataattttgtaagtcgtgcgactaatgaacttatttggttgaatgaaaaagaagaggaggaagttgcttatgactggagtgagagaaacaccaacatagctaggaaaaaagattatcatgctgaattaatgagagaacttgatcaaaaggaagaaaatattaaatcagttcaggagatagcagagcagctacttctagaaaatcatccagcccggttaactattgaggcctacagagcggcaatgcagacgcagtggagctggatcttacagctctgccagtgtgtggagcagcacataaaggagaacacagcgtatttcgagtttttcaatgatgccaaagaagctactgattacttaaggaatctaaaagatgccattcagcggaagtacagctgtgatagatcaagcagcattcacaagctagaagaccttgttcaggaatcaatggaagagaaagaagaacttctgcagtacaaaagcactatagcaaacctaatgggaaaagcaaaaacaataattcaactgaagccaaggaattctgactgtccactcaaaacttctattccgatcaaagctatctgtgactacagacaaattgagataaccatttacaaagacgatgaatgtgttttggcgaataactctcatcgtgctaaatggaaggtcattagtcctactgggaatgaggctatggtcccatctgtgtgcttcaccgttcctccaccaaacaaagaagcggtggaccttgccaacagaattgagcaacagtatcagaatgtcctgactctttggcatgagtctcacataaacatgaagagtgtagtatcctggcattatctcatcaatgaaattgatagaattcgagctagcaatgtggcttcaataaagacaatgctacctggtgaacatcagcaagttctaagtaatctacaatctcgttttgaagattttctggaagatagccaggaatcccaagtcttttcaggctcagatataacacaactggaaaaggaggttaatgtatgtaagcagtattatcaagaacttcttaaatctgcagaaagagaggagcaagaggaatcagtttataatctctacatctctgaagttcgaaacattagacttcggttagagaactgtgaagatcggctgattagacagattcgaactcccctggaaagagatgatttgcatgaaagtgtgttcagaatcacagaacaggagaaactaaagaaagagctggaacgacttaaagatgatttgggaacaatcacaaataagtgtgaggagtttttcagtcaagcagcagcctcttcatcagtccctaccctacgatcagagcttaatgtggtccttcagaacatgaaccaagtctattctatgtcttccacttacatagataagttgaaaactgttaacttggtgttaaaaaacactcaagctgcagaagccctcgtaaaactctatgaaactaaactgtgtgaagaagaagcagttatagctgacaagaataatattgagaatctaataagtactttaaagcaatggagatctgaagtagatgaaaagagacaggtattccatgccttagaggatgagttgcagaaagctaaagccatcagtgatgaaatgtttaaaacgtataaagaacgggaccttgattttgactggcacaaagaaaaagcagatcaattagttgaaaggtggcaaaatgttcatgtgcagattgacaacaggttacgggacttagagggcattggcaaatcactgaagtactacagagacacttaccatcctttagatgattggatccagcaggttgaaactactcagagaaagattcaggaaaatcagcctgaaaatagtaaaaccctagccacacagttgaatcaacagaagatgctggtgtccgaaatagaaatgaaacagagcaaaatggacgagtgtcaaaaatatgcagaacagtactcagctacagtgaaggactatgaattacaaacaatgacctaccgggccatggtagattcacaacaaaaatctccagtgaaacgccgaagaatgcagagttcagcagatctcattattcaagagttcatggacctaaggactcgatatactgccctggtcactctcatgacacaatatattaaatttgctggtgattcattgaagaggctggaagaggaggagaagtcactggaagaagaaaagaaagaacatgttgaaaaagccaaagaattgcaaaaatgggtatcaaatatcagcaagacattgaaagatgcagagaaggctggaaaacctcccttctctaagcaaaagatttccagtgaggaaatatcaaccaagaaggaacaattatcggaagcacttcaaactatacagctttttttggcaaaacatggagacaaaatgacagatgaagaaagaaatgaattggaaaaacaagtgaaaactcttcaggaaagttataatttattattcagtgaatctctgaaacagctacaagaatcacaaacctcaggagatgtaaaggtagaggagaagattgtagcagagcgtcagcaggagtataaagagaaactccaagggatatgtgatcttcttacccaaactgaaaaccgcctaattggtcaccaagaagccttcatgattggagatggcacagttgagttaaagaaatatcagtccaagcaagaggaattacagaaagatatgcaaggaagtgcacaggcattggctgaagtagtgaaaaacacagagaacttcttaaaagagaatggtgaaaagctgtcacaggaagataaggctttgattgaacagaaacttaatgaagctaagataaagtgtgaacagcttaatttaaaagcagaacagtctaaaaaggagctggataaagttgtgacaacagcaatcaaggaagaaactgaaaaggtagcagcagtgaagcagctggaagagagcaaaaccaaaatagaaaaccttttggactggttgtcaaatgttgacaaagactcagaaagggcagggacaaaacacaaacaggtaatcgaacagaacgggacccattttcaagaaggtgatggcaagtcagcaattggagaagaggatgaagttaatggtaacctgttggagactgatgttgatgggcaagttggaaccactcaggagaatctgaatcagcaatatcagaaagttaaggcccaacacgagaagatcatctctcagcaccaagcagtgatcatagccactcagtctgcacaagtgttgcttgagaaacagggtcagtatctctctcctgaagaaaaagagaaactgcaaaagaacatgaaggaactgaaagtgcattatgaaactgcactggctgagtcagagaagaaaatgaagttaactcactctctgcaggaagaattagaaaagtttgatgctgactataccgagtttgagcactggctacagcagtcagaacaagaacttgaaaacctggaggcaggtgcagatgacatcaatggtttaatgaccaaattgaagaggcagaagagtttctcagaagacgttatttctcacaaaggtgacttgagatacatcacaatttctggaaacagagtgttggaagctgccaaatcttgcagcaagagagacggtggcaaggttgatacttctgcaacccacagagaagtgcagcgcaagttggatcatgctactgatcggttcaggtctctctactctaagtgcaatgttcttggaaataatcttaaagatctggtggataaatatcaacactatgaagatgcttcatgtggacttcttgctggactccaggcctgtgaggccacagcgagcaaacacttatctgaacctattgcggtggaccccaaaaatcttcaaaggcaattagaagagaccaaggctttgcaaggacagatatcaagtcagcaggttgctgtagagaaactgaagaaaacggctgaagtgcttttagatgccaggggatctttacttccagccaagaatgatatccagaaaacacttgatgacattgttgggagatatgaggatctgtccaagtctgttaatgaacgaaatgaaaaactccagataaccttgacccgttcactgagtgtgcaggatggcttggatgaaatgctggactggatgggaaatgtggaaagttctctgaaagaacaaggtcaagtgcctttaaactctactgctcttcaggatattatcagtaaaaacattatgttggaacaggatattgcaggtcgtcagagcagtataaatgccatgaatgaaaaagtgaagaaatttatggaaacaacagatccatccactgcatcctcactgcaagccaaaatgaaagacttgtctgctcggttttcagaagcaagtcataaacacaaagaaactcttgccaaaatggaggagctaaaaaccaaagtagaactgtttgagaacctctcagaaaagctccagacatttttagaaacaaaaactcaggctcttactgaagtagatgtgccaggaaaagatgttactgaattgtctcagtatatgcaggaatcaacttctgaatttttagaacacaagaaacatcttgaagttttgcatagtctcttgaaggagatatccagccatggcttaccaagtgataaggctttggtccttgaaaaaacaaataatttgtcaaagaaattcaaggaaatggaggataccatcaaagaaaaaaaagaagctgtaacttcttgtcaagaacagttggatgctttccaagttcttgttaaatcattgaagtcatggataaaagaaacaacaaaaaaagttcccattgtacagccttcttttggtgcagaggatttaggaaaatctttggaagacactaagaaattacaagaaaagtggagtttaaaaacaccagagattcagaaagtaaacaacagtgggatctcactatgtaacttaataagtgctgtgaccacccctgcaaaggcaatagcagcagttaaatcaggtggagcagtattaaatggtgaaggaacagccacaaatactgaggaattttgggcaaataaaggtttaacatccattaaaaaggacatgactgacataagtcatggttatgaagatcttggcctcttactcaaggacaaaatagcggaactgaacactaaactctccaaattgcaaaaggctcaggaagaatcaagtgcaatgatgcagtggttacagaaaatgaacaaaactgcaacaaaatggcagcagacacctgcacctacagatactgaagctgtgaagactcaagttgagcagaataagtcgtttgaggcagaactgaagcagaatgtaaacaaagtacaggagttgaaagacaaattgacagagctgttggaggagaacccagatactcctgaggcccccagatggaaacagatgttgacagaaatagattctaagtggcaagaactcaatcaattaacaattgatagacaacaaaaactggaagaatcctccaataatctaacccagttccagactgtagaggcccaattgaaacagtggcttgtggaaaaagaacttatggtcagtgttcttgggcccttgtcaattgacccaaatatgctaaacacacaaaggcagcaggtgcagattttgctgcaagaattcgccactcggaaacctcaatatgaacagctgacagcagctggtcagggcattctgagcaggcctggagaagacccttctttacgtgggattgtgaaagagcaactggcagctgtgacccaaaaatgggatagcctaacagggcaattgagtgacagatgtgactggattgaccaagccattgttaaaagcacacagtatcaaagcctgctgagaagcctttctgataaactgagtgacttggataataaactcagcagcagtctggctgtgagcacgcaccctgatgctatgaaccaacagttggaaacagcccaaaaaatgaagcaggagatacagcaggaaaagaagcagataaaagtggcccaggcactctgtgaggatttgtcagcactggttaaagaagagtacttgaaagcagaacttagtaggcaactagaaggcatcttaaaatcatttaaggatgttgaacagaaagcagagaatcatgtccagcaccttcagtcggcctgtgcaagctctcatcaatttcagcaaatgtctagagattttcaggcttggctggatacaaagaaagaagagcaaaacaaatctcatccaatatctgccaaactcgatgtcttggagtcattaattaaagatcataaagactttagtaaaactttgaccgctcagtctcatatgtatgaaaaaaccattgcagaaggtgaaaatctgttattaaaaacacaagggtctgagaaggcagccttacagttacagcttaatacaattaaaaccaattgggatacatttaataagcaggtgaaagaaagagaaaacaagttaaaagagtcattggaaaaagcccttaagtataaagagcaagtagagactctctggccatggatagacaaatgccaaaacaacctggaggaaataaaattttgcttggatcctgctgaaggagagaattctattgccaagttaaagtctctgcagaaggaaatggaccaacactttggtatggtagaattactgaacaacacagccaatagcttgctcagtgtctgtgagatagataaagaagttgttacagatgagaataagtcactgatccagaaggtggacatggtcactgaacaacttcacagtaagaaattctgtctggagaacatgactcagaagtttaaagaatttcaagaagtttccaaagaatctaaaaggcagcttcagtgtgcaaaggagcagctagatatccatgattcgctgggatcccaggcttacagtaacaaatacctgaccatgttgcaaactcagcagaaatcacttcaggccttgaagcatcaggtagatttggctaaaagacttgcacaggaccttgtggtagaggcctcagactcaaagggaacctctgatgttttattacaagtggaaaccatagctcaagagcatagtacactaagtcagcaggttgatgaaaagtgttctttcttagaaaccaagcttcagggcattgggcatttccagaataccattcgagaaatgttttctcagttcgcagagtttgatgatgaactggatagcatggctccagtggggagagatgcagaaacattgcaaaagcaaaaggaaactataaaagcctttctaaagaaactagaagccctcatggcaagcaatgacaatgccaataaaacctgcaagatgatgttagccacagaagaaacctctcctgaccttgttggaatcaaaagggacttggaggccttaagcaaacaatgcaacaagttactggaccgagcccaagccagagaagagcaggttgaagggacaattaagcgccttgaagaattttacagcaaattgaaagaattttctattctgctccagaaagccgaagaacatgaagagtcacaaggtcctgttggtatggaaacggagacaattaatcagcagcttaacatgttcaaggtattccagaaagaagagattgaacccttgcaaggtaaacagcaagatgtaaactggttaggtcaaggccttattcagagtgctgccaaaagcactagcactcagggcttggagcatgacctggatgatgtcaatgcacggtggaagactctcaataagaaggtggctcagcgagcagcccagctgcaggaggccttgctgcactgtgggaggttccaggatgccctggagtccctgctcagctggatggtggacactgaggagcttgtggccaatcagaagcccccgtcggctgagttcaaagtggtaaaggcccagatacaagaacaaaagcttctccagagattgttggatgaccgaaaatctacggtggaggtaatcaaacgagaaggagaaaaaattgctacaacagcagagcccgcagataaagtgaagattttgaaacagctcagtctcttggatagcagatgggaggcattgcttaataaagctgaaacaaggaatcgtcagttggaaggtatctcggtggtagcacagcaatttcatgaaaccttagaaccactgaacgagtggcttacaaccatagaaaagaggctggtgaattgtgaacccataggaacccaagcatctaaacttgaggaacaaattgcacagcacaaagccttagaagatgacatcatcaatcacaataaacatttacaccaggctgttagcattggccagtccttaaaggttttgagctccagggaggataaagatatggtgcagagtaaattagacttctctcaagtatggtacattgagattcaagagaaaagtcacagcaggtctgagctcctccagcaggccttatgtaatgctaagatttttggggaagatgaagttgaactgatgaactggctgaatgaagtgcatgacaaactgagcaagctctcagtccaggattacagcactgaggggctatggaagcagcagtctgaacttcgggttctgcaagaggacatcttactcaggaaacaaaatgtagatcaggctttactaaatggtttagaactacttaaacaaaccacaggtgatgaagttttaataattcaagataaattggaagccattaaagcaaggtacaaagacattactaaactgagcactgatgtggccaagactctggaacaggcgctgcagcttgcaaggcggctgcactccacacacgaagagctgtgtacctggctggacaaagtggaggtggaattactttcatatgaaactcaggttctgaaaggagaagaagcaagtcaagcacaaatgagaccaaaggaactgaaaaaggaagctaagaacaacaaagccttactggactcccttaatgaagtgagcagtgctttgctggaactggtaccatggagggcaagagaaggacttgagaaaatggtagctgaggacaatgagcgctaccgattagtgagcgacaccatcactcagaaggtggaggagatcgatgcagccattctgcgatcacagcagtttgaccaagcagctgatgctgagttatcctggattactgaaacagaaaaaaaattgatgtctctgggtgacatcaggcttgagcaagaccagacttctgctcagcttcaagttcaaaagacattcaccatggagattttgagacacaaggatattattgatgaccttgttaaatctgggcataaaatcatgaccgcatgcagtgaagaggaaaagcaatcaatgaagaaaaaactggacaaggtactgaagaactatgataccatctgccagattaattcagaaaggtatctgcagctggaacgggcacagtccctggttaaccaattctgggaaacatatgaagaactttggccatggctgacagaaacacaatcaatcatctctcagcttcccgccccagcccttgaatatgaaactctaaggcagcagcaggaagaacatcggcaactgcgtgagttgatagctgaacacaagcctcatatagataagatgaacaaaactgggccacagttactggaattgagccctggggaaggcttttctatccaagagaagtatgtggcagccgacaccctttacagtcaaattaaagaagatgtcaaaaagcgtgctgtggcactggatgaagccatttctcaatcaactcagttccatgacaagatagatcagatccttgagagcctggaacgcatcgtggaacgtctgaggcagccaccctctatctctgcagaggttgagaagatcaaggaacagatcagtgaaaataagaatgtgtcagtagacatggaaaagctacagccgttgtatgaaactcttaaacagaggggagaggaaatgattgctagatctggggggactgataaagacatatctgccaaagctgttcaggataagcttgaccaaatggttttcatttgggagaacatacacacactggtggaagagagggaagccaaactactggatgtgatggagctagcagaaaagttctggtgtgatcacatgtcattgatagttaccattaaagatactcaagatttcatccgggacctggaagatcctggaattgatccttcagtagtaaaacaacagcaagaagcagcagagaccataagggaagaaatagatggactacaggaggagctggatatagttattaacctaggttctgaactcattgcggcatgtggggagcctgataaacccattgtcaagaagagtatagatgagttaaattcagcatgggattctctaaataaagcttggaaagaccggattgacaaacttgaggaggcaatgcaggctgccgttcagtaccaggatggactgcaggcggtatttgactgggtagatattgcaggtggtaaattagcttcaatgtctccaattggaacagatctcgaaactgtcaagcagcagattgaagagctaaagcaatttaagtctgaggcctatcaacagcagatagaaatggaaagactgaatcatcaagcagagcttttgctaaagaaagtaacagaagagagtgacaaacacactgttcaagacccattaatggaactgaaattgatatgggatagcctggaggagagaatcatcaacagacagcataaactggagggtgctctattagccttgggtcagttccaacatgccctggatgagctcctggcatggctgacacacaccgagggcttgctaagtgagcagaaacctgttggaggagaccctaaagccattgaaattgaacttgccaagcatcatgtgctccaaaatgatgtattagcccatcagtccacagtggaagccgttaataaagcaggaaatgatctaattgaatcaagtgcaggagaagaagcaagcaaccttcagaacaagctagaggttttaaatcaacgctggcaaaatgttttggaaaaaacagaacaaaggaagcagcagctggatggtgccttgcgccaggccaaagggttccatggcgaaattgaggatttgcagcagtggctgactgacacggagcgtcatctgttggcatctaaaccgctgggaggtttaccggaaacagccaaggagcagcttaatgtccatatggaagtctgtgctgcctttgaagctaaagaagaaacatataagagtctgatgcagaaaggccagcagatgcttgcaagatgcccaaaatctgcagagacaaatattgaccaagacataaataacttgaaagaaaaatgggaatcggtggaaaccaaactcaatgaaaggaaaactaaactggaagaggctctcaacttggcaatggagttccacaattctctccaagacttcatcaactggcttactcaggctgaacagaccctaaatgtagcttctcggccaagtctcatcttggacacagtcttatttcaaattgacgaacacaaggtttttgccaatgaagtaaattctcatcgtgagcagataatagagctggacaaaactggaacccacctaaaatattttagtcagaaacaagatgttgttctaatcaagaatctacttatcagtgtacaaagtcgatgggaaaaagtggttcaacggttggtagagagaggaagatctttggatgatgcaaggaagagagccaagcagttccatgaagcttggagtaaacttatggagtggctagaagagtcagaaaagtctttggattctgaactggaaatcgcaaatgatccagacaaaataaaaacacaacttgcacaacataaggagtttcagaaatcactcggagccaagcattctgtctacgacaccaccaacaggactggacgttctctgaaggagaaaacctccctggctgatgacaacctgaaactggatgacatgctgagtgaactcagagacaaatgggataccatatgtggaaaatctgtggaaagacaaaacaaattggaggaagccctgttattttctggacaattcacagatgccctacaggctctcattgattggttatatagagttgaaccccagctggcagaagaccagcctgttcatggagacattgatttggtgatgaatctgatcgataatcacaaggccttccaaaaagagttggggaagaggaccagcagtgtgcaggccctgaagcgctcagcccgagaactcatagaaggcagtcgggatgactcctcctgggtcaaggtccagatgcaggaattaagcacacgctgggagaccgtgtgtgcactttctatatcaaagcaaacacggttagaagcagccctgcgtcaggcagaggaattccactcggtggtacatgccctcttggagtggctggctgaggcggagcaaaccctgcgtttccatggtgtcctcccagatgatgaggatgctctccggactctcattgatcagcataaagaattcatgaagaaactggaagaaaagagagctgaactaaataaagccaccactatgggcgacaccgttttggctatctgccaccccgactccatcactaccattaagcactggataacaatcatccgggcgaggtttgaggaggtgctggcctgggcaaagcaacatcagcagagattagcaagtgctctggctgggcttattgccaaacaggaattgttggaagctttgctggcttggttgcaatgggctgaaactacacttactgataaggataaagaagtcatcccccaggagatcgaagaggtgaaagcactcattgcagaacaccagaccttcatggaggaaatgaccagaaaacagcctgatgttgataaagtaacgaagacctataagaggagagctgctgatccttcctcattacaatcccatattccagtcttggataagggacgagcaggaagaaaacgctttccagcatcaagcttgtatccctctgggtcacagacacaaattgaaaccaaaaatcctagggtaaacttactggtgagcaaatggcagcaagtctggctcctggcgttggaaagaaggaggaaactcaatgatgccttggacagactagaggagctgagggaatttgctaactttgattttgatatctggcgcaaaaaatacatgcgatggatgaatcacaagaaatctcgagtgatggacttcttcaggagaattgataaagaccaggatgggaaaataacgcggcaggaatttattgatggaattctttcctcaaagtttccaaccagtcgcttggagatgagcgcagttgcagacatctttgacagagatggcgatggatatattgactactatgaatttgtagcagcccttcacccaaataaagatgcatataaacctatcacagatgccgacaaaatcgaagatgaggtgacaaggcaggtagctaagtgtaaatgtgcaaagcgatttcaagttgagcagattggtgataataaatacagggtaaaggttctttctttcttcactgaacagtttggagactcccagcaactgcgactggtccggatcctgcggagtactgtgatggttcgtgttggaggtggatggatggcacttgatgagttcttagtgaaaaatgatccttgcagggccaaaggaaggacaaacatggaactgcgtgagaagttcattttagcagatggtgccagccagggtatggctgctttccgaccccgaggccgaagatcccggccatcatcacgaggcgcttcacccaacagatccacttctgtgtccagtcaggctgcgcaggcggcctccccacaggtccctgccaccaccacacccaagattctccatcctttaacacgcaattatggtaaaccatggttgacaaacagcaaaatgtcaactccttgtaaagcagcagagtgctcagactttcccgtgccatctgcagagggaacgccaatacaaggaagcaagcttcgacttccaggatatttatcagggaaaggcttccactctggggaggacagtggcttgataacaactgcagctgccagagtccgaacacagtttgctgattccaagaagactcccagccgaccaggaagtcgagctggaagcaaagctggcagcagggccagcagccgccgaggcagtgatgcatcagactttgacatttcagaaatccagtccgtgtgctcagatgtggaaactgtcccccagacacacagacctacaccccgagcaggttctcggccatccacagcgaagccttcaaaaatccccacgccccagaggaaatcacctgccagcaaattggacaagtcctcaaagagatagtgcaattggttctaccaaggcccttccttgagcatttattatttaagtttgaacgatgtaaaatatggtgtagaaattcttgtgaaatattgcaagaggcgagtttaaaattctgcagatggccttatttgtgtatttgtctttttattttatctgtataattttttttgtcagatattctggggttaaagtcacatcatatgtgaggaggaaaagtttaacatgaactaacatttctgcactgtaacgtgccgggcacacactaaactcagttactgtacctacaggtaagtctacatcctctctgacagccacagcactacatcaatccctgacgttagggatacctcatgacattttcctgtttttatggaaactctgagaagctgaatgatacatgcaggggatattttttgagatgatttaaatgtaaaccaaaagatggaagacaaaaagacaaacacacccacacgcagtctttgcagtatctgacagagaactcacaggaagttacttcaagcacttgccagtactatgatattcaagtaccttgcagcatttctctgccattgctttcaatgaggccagaggcatcctggatattagacctattatactgtaagaatataagtataaagtgcgttcatatacatgtgaggttttcttttgcttgagtggacagtagcacctgtatcattgaactcattttgtatcagagcaattttgcttgcagaaagctatgaaataaaacacgtcccttaactgc

SEQ ID NO: 148 - DNA, 1751 bp, Homo sapiens
  >DCN (HUGO ID) DECORIN PRECURSOR (BONE PROTEOGLYCAN II) (PG-S2) (PG40). [Source SWISSPROT (P07585)]
tttaactgtgctatggagtagaagcaggaggttttcaacctagtcacagagcagcacctaccccctcctcctttccacacctgcaaactcttttacttgggctgaatatttagtgtaattacatctcagctttgagggctcctgtggcaaattcccggattaaaaggttccctggttgtgaaaatacatgagataaatcatgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaattctcaagaaagccctcatttttataacctggcaaaatcttgttaatgtcattgctaaaaaataaataaaagctagatactggaaacctaactgcaatgtggatgttttacccacatgacttattatgcataaagccaaatttccagtttaagtaattgcctacaataaaaagaaattttgcctgccattttcagaatcatcttttgaagctttctgttgatgttaactgagctactagagatattcttatttcactaaatgtaaaatttggagtaaatatatatgtcaatatttagtaaagcttttcttttttaatttccaggaaaaaataaaaagagtatgagtcttctgtaattcattgagcagttagctcatttgagataaagtcaaatgccaaacactagctctgtattaatccccatcattactggtaaagcctcatttgaatgtgtgaattcaatacaggctatg

SEQ ID NO: 149 - DNA, 870 bp, Homo sapiens
  >CD47 (HUGO ID) LEUKOCYTE SURFACE ANTIGEN CD47 PRECURSOR (ANTIGENIC SURFACE DETERMINANT PROTEIN OA3) (INTEGRIN ASSOCIATED PROTEIN) (IAP) (MER6). [Source SWISSPROT (Q08722)]
ggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctagggtaagt

SEQ ID NO: 150 - DNA, 941 bp, Homo sapiens
  beta2-microglobulin
tcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggagggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttg

SEQ ID NO: 151 - DNA, 484 bp, Homo sapiens
  >RPL27 (HUGO ID) 60S RIBOSOMAL PROTEIN L27. [Source SWISSPROT (P08526)]
tccttctttcctttttgctggtagggccgggtggttgctgcagaaatgggcaagttcatgaaacctgggaaggtggtgcttgtcctggctggacgctactccggacgcaaagctgtcatcgtgaagaacattgatgatggcacctcagatcgcccctacagccatgctctggtggctggaattgaccgctacccccgcaaagtgacagctgccatgggcaagaagaagatcgccaagagatcaaagataaaatcttttgtgaaagtgtataactacaatcacctaatgcccacaaggtactctgtggatatccccttggacaaaactgtcgtcaataaggatgtcttcagagatcctgctcttaaacgcaaggcccgacgggaggccaaggtcaagtttgaagagagatacaagacaggcaagaacaagtggttcttccagaaactgcggttttagatgctttgttttgatcattaaaaattat

SEQ ID NO: 152 - DNA, 605 bp, Homo sapiens
  RPS16 (HUGO ID) 40S RIBOSOMAL PROTEIN S16. [Source SWISSPROT (P17008)]
aaggcgcctgcgcagaccctgaaaagcggccagggtggcccctagctttccttttccggttgcggcgccgcgcggtgaggttgtctagtccacgctcggagccatgccgtccaagggcccgctgcagtctgtgcaggtcttcggacgcaagaagacagcgacagctgtggcgcactgcaaacgcggcaatggtctcatcaaggtgaacgggcggcccctggagatgattgagccgcgcacgctacagtacaagctgctggagccagttctgcttctcggcaaggagcgatttgctggtgtagacatccgtgtccgtgtaaagggtggtggtcacgtggcccagatttatgctatccgtcagtccatctccaaagccctggtggcctattaccagaaatatgtggatgaggcttccaagaaggagatcaaagacatcctcatccagtatgaccggaccctgctggtagctgaccctcgtcgctgcgagtccaaaaagtttggaggccctggtgcccgcgctcgctaccagaaatcctaccgataagcccatcgtgactcaaaactcacttgtataataaacagtttttgagggattttaaagtttc

SEQ ID NO: 153 - DNA, 2054 bp, Homo sapiens
  NM_018479 (RefSeq ID) UNCHARACTERIZED HYPOTHALAMUS PROTEIN HCDASE. [Source RefSeq (NM_018479)]
agtcaccaccgtggcctgcgacgaaatggcgaaaagtcttttgaagacagcctctctgtctggaaggacaaaattgctacatcaaacaggattgtcactttatagtacatcccatggattttatgaggaagaagtgaaaaaaacacttcagcagtttcctggtggatccattgaccttcagaaggaagacaatggcattggcattcttactctgaacaatccaagtagaatgaatgccttttcaggtgttatgatgctacaacttctggaaaaagtaattgaattggaaaattggacagaggggaaaggcctcattgtccgtggggcaaaaaatactttctcttcaggatctgatctgaatgctgtgaaatcactaggaactccagaggcaattttttcttcaatatacagacttcctttaataagtgttgcgctggttcaaggttgggcattgggtggaggagcagaatttactacagcatgtgatttcaggttaatgactccagagagtaagatcagattcgtccacaaagagatgggcataataccaagctggggtggcaccacccggctagttgaaataatcggaagtagacaagctctcaaagtgttgagtggggcccttaaactggattcaaaaaatgctctaaacataggaatggttgaagaggtcttgcagtcttcagatgaaactaaatctctagaagaggcacaagaatggctaaagcaattcatccaagggccaccggaagtaattagagctttgaaaaaatctgtttgttcaggcagagagctatatttggaggaagcattacagaacgaaagagatcttttaggaacagtttggggtgggcctgcaaatttagaggctattgctaagaaaggaaaatttaataaataattggtttttcgtgtggatgtactccaagtaaagctccagtgactaatatgtataaatgttaaatgatattaaatatgaacatcagaattactttgaaggctactattaatatgcagacttacttttaatcatttgaatatctgaactcatttacctcatttcttgccaattactcacttgggtatttactgcgtaatctggaacatttagctaaaatatacacttttggcttaaaaattattgctgtcaattccaataataattcttagcttataaccaaagagcagtgtttaaaaggagagcttctatacaaaacctattcctggcgttacttttcatacaatttttgttctgttttacctggaaataatttaccaaaataactgagtgttgctgctaaagaacaaaagtggggaggtatcagggaacaagaaaacaagaaagggtatgatcaatcattttcttctgctccaaacagctggagtaaaattcatgggaaatggcccttcatttaaaaaaagatgtacctcactacccactacaaatttggaactttgttcttttcaataattagttttctattgtaaattacctactaaacagtggtagccatgacatggaaagtcaactgattctacaattggacattcatttgtgtgccctggaatttccaactagtaataaacaactactgttgatgtagttttaaaccacttgaagggactcatgaagcatcctgcaacataaatttgcatttttacatcagatttcttttttttcctgaaaaacaactaaccttctaacaactatctttcaaaagtaaatgtaataaaaatgcacaacataaaatgtttatgatcccagcaatacactttttaaaaaatgtgaaagtcaaagaattaagttctagttctgactcatcacaagaggtcaaaagtatttgctactgtaacattcaattcacatttgagaatcatggtaaaaataacttgcatttgccttaccatcatgatcctactgttgagttaggaaaatatggttagacagactcacattactttttttcagaggtaaactctagattactgtgtcaacccaatactatttggccatagatgtaaaaactaccaaataaaagtggattttgtggtct

SEQ ID NO: 154 - DNA, 170 bp, Homo sapiens
  >OIC1AJ617288
gtaaagcgtatttggttattcctttgtgttctcaattttctggacagtaagatgtgcaagaagtgtttgtattctcatggggcatgtggaacaggtaactatacccccaacaactcccaccccatctcttcatcttgcacacacaacaccaatcaccatcatgtctttcc

SEQ ID NO: 155 - DNA, 2037 bp, Homo sapiens
  >Chr 5 clone (EST)ENSESTT00000023592
cactaggagggaggatgctacgatttcagacctcagctattaaatagtaaatctggaaaactagtggataaatttgccatctccattctatgttattaagaccaagatagtgtactggttttcttttaaatcacattccattatgtcatttaaaatgtcacaaaacccacacgaagcatctcatttacactaagatgcttatttagctaacaccactaatgataaatgctgttacaaatatgcatagtcagatgatagtaaccacatacagaaaaaaaatttgaacaagtatttatttcttaaaatttacttaagggattagagctaatatataatagaacatttaatataacatttggagttatgtcaacataaaaatagctgtggttacaattagcacatgcaattcactgcaaaggtaaaaatacatgctatactctagacaagccttccaaatgaagttagagtagatggggtaaaacagcaagtgaacatgaaaggattgcacttagaagaaagtgggacatagctaggatataaaagaaacatacctaatgctagtcagtcactgcattgtcctactagcaaattgcacatttatttttagagtatattcaatacacatacatattgagactagagaattttcaaatatctacctttgaaatatccctttgtttctaacacatcacattatggtattaatgtaacagcacttaaaacctgtagttatcattcaaagcttgtgttcaagagataaaagaacatcattcagacaaagcctgtgttcaagaaataaaaaaaacatctatcactatcggccttaaatgcatctcatcacacgacccatcaactttaatgaaatgggtaatgaagttaaactaaccagtttgtacaaactattgactgagaaaaagcccttaaaaatttaggaacaaatgagcaacagaagagctcaccctgagctgccaccttttaatttttaatgcaatgtatatgagaatatcatgctaatgcacatactagtagcatgtatgatggaaaaaaataggttaatgcaaaagataatacgcttggctttgtaaagttttgttttgacttacatcatctgaattctgtttatcgagtctgaaaaggaactgctgccaaggaccagtccaaagaagaattaggctgctaatcctaaaacacttgccattttatgactccatattcttttaatttacagaaaagtacaaattttttagaaagtaatcttatcagcacaatataatctaacaatacctgtaatagaaagctatcccagtaaattttttttgaacaattgaacttttgtcttatgcagcttataactttgttattttattcatctcaacaaaataaaactttattgaaacaaaagtgtatggtttaggaatatgctcttaataacaactgagcaaaattagaatgatgtggactataacagaatgaaggaagatctctcctgcttgatgtacttggtcaatatatgttagtaaacagaaatattttagggaattctatgattattctacttctacatttacatgtcatggttttaactagttaaatgggtagaaaaaagacatctttaataattgatttctatttctcaaactagatgctctactttaaatatgagctttaatgaatgttttgtaacattttaaagttgtaacatttctaagtatactccactaacttcacaataagagctagggtcactggatgacttcatgtggtaaaatcccttatcaaggaataaggctgaaatttaaatgaacaattaggtagagaagaggtatgaacacagaacatgtgaatgaaatttgctttttttccttgctgctgttcccgagtgccgactgatccatagtaaaaaaggacaagattaaaattttaggttttaatggtacaaagtgtgaagttttatagttttctaataattattcagtattatccctttcagagatgagggtaggataccacagacatcagtaactgacaagttataat

SEQ ID NO: 156 - DNA, 228 bp, Homo sapiens
  >Chromosome 16 clone (EST) No ESTs or genes
cattctgttagtgcagagctacaaatcttatttgttacatagctcaaagacatcttccacaggaagaaaaatacctgccattagtaaattcttaaccggtatgaacctatgcttctattttaaagaggttagtgcacatatgccttctaaatatctcataaaaaaccaccgtagcacatttaaaatcctggtattattacttaataccaccctacaacacaaaaatca

SEQ ID NO: 157 - DNA, 166 bp, Homo sapiens
  >Chromosome-1 clone hypothetical protein XP_211586
gacatgtctcactgtttgtatttctctctcctgtcaaagtgaatgatatattcttgaaaacccccaatttcttgaaatgggtgttctgttcattcatgggcaggggtcaggattgaagttcatatatgaaacaactggggatgtagataggaaagaatgtctgctg

SEQ ID NO: 158 - DNA, 536 bp, unknown
  >gi|5446595|gb|AI825924|sr|IMAGp998F065401.1| cl|IMAGp998F065401|AI825924 to92d03.x1 NCI_CGAP_Gas4 Homo sapiens cDNA clone IMAGE2185733 3′, mRNA sequence.
cacttttttcatttttttttattaatcaaatgatacaaaacaaccatcattctgtcaatgcccaagcacccagctggtcctctccccacatgtcacactctcctcagcctctcccccaaccctgctctccctcctcccctgccctagcccagggacagagtctaggaggagcctggggcagagctggaggcaggaagagagcactggacagacagctatggtttggattggggaagagattaggaagtaggttcttaaagacccttttttagtaccagatatccagccatattcccagctccattattcaaatcatttcccatagcccagctcctctctgttctccccctactaccaattctttggctcttacacaatttttatccctcaaatattcatccctggcccaaccagtcccctgagcctccctctggtggagactcctccacccatgagctccccagagcatccaagacagagtgcacagagacctgnggaaggaagctgaactttgcagagatgaggacaggtgcagg

SEQ ID NO: 159 - DNA, 699 bp, unknown
  >gi|4534249|gb|AI570875|sr|IMAGp998J065499.1| cl|IMAGp998J065499|AI570875 tr68d03.x1 NCI_CGAP_Pan1 Homo sapiens cDNA clone IMAGE2223461 3′ similar to gbM35252 TUMOR-ASSOCIATED ANTIGEN CO-029 (HUMAN);, mRNA sequence
ttcaccaacgtaaacagttacttttattttgagtaaaaatacacattcatatcattttcccttatatccctcaaatcttaaatgtgttcaatatgtctagaagatatctgtggtctagctagccgagacattttaaaaagacagctgctcctgacttatatagcacttacatatttaaatttacaaagccaaagcaacattttaaaggggtttgactgacgatagcttgatgcatccacagattcatttgttcccgatctggcaatacaggaccatagaaaacaccaaacccagtatctcaataactgccagtccaaatgctattccaataactataatcaaattttttgccaagaagtcttttatgaaagaaatacaggtctctttgtaaacttgttttccattatagctttggcatggtctctgcttatctagacaggcacataattcaggataggttgaaaattatttcccaatcagcagctccattgaccaaaccgcagcatttaaactcttcttgaaacancattatggctcncctggaatgtnttcacttttccctgtggcgctcaaaaaagccttgtgttcatatgagagtcatttccacaatcgatcagacttagattgaaanncagtnctatggtatcctgcncncccttcncggggagcagatagaggcaagcctttgaaaacacaaca

SEQ ID NO: 160 - DNA, 717 bp, unknown
  >sr|IMAGp998A05535.2|cl|IMAGp998A05535|gb| BX114906|oid|234564189 BX114906 Soares fetal liver spleen 1NFLS Homo sapiens cDNA clone IMAGp998A05535 ; IMAGE247564, mRNA sequence
ctttatcttgagtttcatgtcttagggttggagaagggaccaagcctcccctgaaagctgaggggtttatggtcaagaagaaagagtaactatggccacctttaggtcaccagccaataatactgcttctctatcctgttctgctgtgacatttgtcccagcaggtgttccagtctgtcctacagtagtgagaccacctgatatggtgaggaaggcaggtaattgatcttactccatcatatcatcacatctatttccaccccccgcccttggagacaaggtctcactccatcatttaggccggaatgcagtggtgcaatcatagctcattgtagtctcaaacacctgggcccaagtgatcctcactgtagtctcccaagtagctaggactacaggcacatgccaccacacctggctaattaaaaaaaattttttttgcacagagatgggggtcttgatatgttgcccaggctggtctggaactcctggccacaagtgatcctcccacctcagcctcccaaagcactgggattactgttgtgagccaccatgaccagccccatcacttcttttgtaggtttgtatttgtttttatttttgacaaagacttttatttcacggtgacttcttcacaagtttcctcagatgctagtctgaacaaaggagagcagggaactagaaatgtagactttctgntttttncagctatggcatcac

SEQ ID NO: 161 - DNA, 600 bp, unknown
  >gi|1382358|sr|IMAGp998B19790|cl| IMAGp998B19790|gb|W72422|W72422 zd65c10.s1 Soares fetal heart NbHH19W Homo sapiens cDNA clone 345522 3′ similar to SWSC13_YEAST Q04491 PROTEIN TRANSPORT PROTEIN SEC13. [1]
tataagcaatatgactttatttagttcctttggaaacaaaaccccccaaataatgcctgaacccaaaggtacataaaaatgacccaaaatagatttgaacatcacttgtagtttcttcctcgtaacatgagtgctttcagctggacagtagattacaaagcatctccgatcacgttaaggcagatgatcaatctgtggctgcatctgtaactcctcctgggaaaataatcctgttggagttgggggctcttcccagttgtctggttagttggcccaggaaggggaagtcctggagctggcgggtggggagccaggccccacctgtcttgtcactgctccgttctgctggccctctgtcacttgatgctgatacggagccctggcccttgttgacatcactgatgcacacccactgcccatcaactgactccttccacagggtcaccttattgtctccaccagagacagccaggatgttggctgtgatggacaagctcacatgccacaccacatcgttgaacttgtgcaanatttaggggaccacgtattgcttgaggcatcatcacaggtccaaatgaacacacgaacatcctgggagca

SEQ ID NO: 162 - DNA, 202 bp, unknown
  >gi|4648553|gb|AI623622|sr|IMAGp998M105514.1| cl|IMAGp998M105514|AI623622 ts22b05.x1 NCI_CGAP_Pan1 Homo sapiens cDNA clone IMAGE2229297 3′, mRNA sequence.
tttttttttttttttttttttttttttttttttttttttttttttttttttttttttttttttttggaaaaaaagggctttgtttggtccctttttaaccccccgaatttggttttcccccattttggaggtttttttttttcccccccccccccccccggttttaaaccgggggcgcccccccttttttttttttttttgg

SEQ ID NO: 163 - DNA, 407 bp, unknown
  >gi|4984008|gb|AI696108|sr|IMAGp998O095541.1| cl|IMAGp998O095541|AI696108 tt03e05.x1 NCI_CGAP_GC6 Homo sapiens cDNA clone IMAGE2239712 3′ similar to contains L1.t3 L1 repetitive element ;, mRNA sequence
gagacataacaatatttaatgtgtatgtgcctgacaacagagtataaaaatatgtgaggcaaaacccatagaaatatgaggagaaataaatgcatacagtatcataattgacttcaacactccaacagaaatggacagatccagtaggcagaaaatctgtatgtgcatagctaaaattaaccataccatcaatcaaatagatataatggacatctactgactacttcatccaacaacagcagaatacatatttttctcaagctcacatgaaacattcaacaaaatagactgtattttgggccttcagaatcaccttaaacacattttaaacataaaaattacataatgtctgggtgtctgaccacaatggacttgaactagaaatcaataacagaaaaatagctgag

SEQ ID NO: 164 - DNA, 380 bp, unknown
  >gi|2139170|gb|AA434256|sr|IMAGp998D121896.1| cl|IMAGp998D121896|AA434256 zw24h06.s1 Soares ovary tumor NbHOT Homo sapiens cDNA clone IMAGE770267 3′, mRNA sequence
cgaatgaattgatcagtctttattgatgattattacatttttcatttgcctggaacttaccacaccctaaagtattctggctctccatattttctgttcataagaatatgcagagcagttgattatcttctgggtcctttagccctttagctctcctgagggtagcagaggactccctggttttcctttggatggaagtttcctgactttctacttctggaaccagggccagtgtccttgacatgggcttcctccaggatgaaatcggacaactgcccaatgacagcaagtaaagaaagtatactcgcctataatcccagcactttgggaggccaaggcgggtggatcacgaggtcagctggataacatactggctaacg

SEQ ID NO: 165 - DNA, 479 bp, unknown
  >sr|IMAGp998H235725.2|cl|IMAGp998H235725|gb| BX112057|oid|234558491 BX112057 NCI_CGAP_GC6 Homo sapiens cDNA clone IMAGp998H235725 ; IMAGE2310214, mRNA sequence
ccgatcgcctcggaagccccgtagaccatcacggacgccgagcttccagtaactcacagtgaaagcttgtttaatgaagctagcttcaatgctgtgagctgctctatagacgcccatgtaggaggaaactcaaggggcccccagccactagccattgaggaactgaggccctcaatccaacaacccacaaggaactgaatcctgccaacaaccactgagtgagcctggaagcggatcctgcccagctgaaactcgagaagattgtagctctgctgccctgaccactattctaactgcacccttgagagagaccctcagccagctaatctgtacctggattcctaacctggagaaactgaaatagcaacgcatgttgatgtaagtgctaagtttgggagtaatttgttatgtaacaataaataactaataatacttattatttaggcaataaaaccatagaaaataaaaaaaaaaaaaaa

SEQ ID NO: 166 - DNA, 716 bp, unknown
  >sr|IMAGp998P03372.1|cl|IMAGp998P03372|gb| BX118749|oid|234571874 BX118749 Soares fetal liver spleen 1NFLS Homo sapiens cDNA clone IMAGp998P03372 ; IMAGE195698, mRNA sequence
tttttttttagagttttagaggaatataatgtatgaaagaaaattccaataacataatgtacaaaaatgttgtcacatacagaagagcaaaaatctacgtattggggactattgctgtgggaagaagggcctgatcttcatactcatcttcctcattgagtaccatgaccccttccaattcatccatactacaccatcatctgtgccatgctttgccatgtcccaggtgtactgtccaccccagtagtatctgccgtttggattggctgcatgacatctattataccaccatccaccaccgtcttctttagaacactgttttctgggatctgatgttaacctgaaagaaaatgtgtttggcgttattagaaacagaactacggtggtcaagttacgttttgcagattacccataaaagactgctacactcgtgtgcacagtagtctttcctttgggtgctatacatagagtcacactaatacaccaaagacctcctgtgccctatagcaagaaagagagataactgggcctttgtggccaatccatctagggactgtgtttgttactttatactggaataatagcttttacaacccttagtatcagagaaccctggaaatgaaaatgaaagttttcacacacactttagaatatagagatataggtatttatctaaatgctcattattcagaaagtaactgtgctcaaattctagt

SEQ ID NO: 167 - DNA, 747 bp, unknown
  >sr|IMAGp998B071870.3|cl|IMAGp998B071870|gb| BX095683|oid|234525737 BX095683 Soares_total_fetus_Nb2HF8_9w Homo sapiens cDNA clone IMAGp998B071870 ; IMAGE760230, mRNA sequence
ccgcatcctagccgccgactcacacaaggcaggtgggtgaggaaatccagagttgccatggagaaaattccagtgtcagcattcttgctccttgtggccctctcctacactctggccagagataccacagtcaaacctggagccaaaaaggacacaaaggactctcgacccaaactgccccagaccctctccagaggttggggtgaccaactcatctggactcagacatatgaagaagctctatataaatccaagacaagcaacaaacccttgatgattattcatcacttggatgagtgcccacacagtcaagctttaaagaaagtgtttgctgaaaataaagaaatccagaaattggcagagcagtttgtcctcctcaatctggtttatgaaacaactgacaaacacctttctcctgatggccagtatgtccccaggattatgtttgttgacccatctctgacagttagagccgatatcactggaagatattcaaatcgtctctatgcttacgaacctgcagatacagctctgttgcttgacaacatgaagaaagctctcaagttgctgaagactgaattgtaaagaaaaaaaatctccaagcccttctgtctgtcaggccttgagacttgaaaccagaagaagtgtgagaagactggctagtgtggaagcatagtgaacacactgattaggttatggtttaatgttacaacaactattttttaagaaaaacaagntttagaaatt

SEQ ID NO: 168 - DNA, 435 bp, unknown
  >gi|1425401|sr|IMAGp998D08822|cl| IMAGp998D08822|gb|W95494|W95494 ze02h04.s1 Soares fetal heart NbHH19W Homo sapiens cDNA clone 357847 3′
taatatgcaaaccatttattgaactatttactattttaaaatatcattcttaaaagaggccacaaaaattataacattcagaaattttaggtaaactccaaaagtaagctttttttttttttggtagtcaacttgtaccaagtttagcagcaagaggatacttccttagagactttcagtggacttaaactcagtttccgctggtgctatgtaaagcatccacgatggttttattgtactctgcaatctgcttggtcacatttttcttaattggctggtaatcactctcttgactcttggttgctatgaattctttcatcgcaaaattattttggctcaaggtgttgccactttctctccaaatttgtaagctgagaatggtggtcncattttcttggcaattgtgtttttagtggcctcatactctatgtnttt

SEQ ID NO: 169 - DNA, 410 bp, unknown
  >gi|1423675|sr|IMAGp998I08822|cl| IMAGp998I08822|gb|W94545|W94545 ze04b04.s1 Soares fetal heart NbHH19W Homo sapiens cDNA clone 357967 3′
cccaacgttttcaagttttatttttaaaatatattacaacatcacattaaaaacagtagaggaaaacaagcaaacatgatagaattacttttttccaactcaatagtacttagctttacctagaattttcatacagttgatgctaacttcttctgaaccctttctgactagtattttaccttatgtattctttatagtatttccatataatctttaccgagacctattcagagcataacatagacagggcagcacaagcaactgacacctttgttggtactacagtgttttttcaaccagtttatgagagaatgaaaagaatgaaagtgaaaattttacttaacttagggttatgtagctctaggtaatctcttcagaactcccctggcttcaccttctcaagttaatca

SEQ ID NO: 170 - DNA, 132 bp, unknown
  >ImageID=357848 IMAGP971C0935_S1 CC FromEXP of IMAGp971C0935_s1.exp April 26, 2002 1243
caagcatcaccctgngaggttcctgagggattnctcataaatgagggctgcacattgcctgttctgcttcgangtattcaataccgctcagtattttaaatgaagcngattctaagatttggntggngatca

SEQ ID NO: 171 - DNA, 390 bp, Homo sapiens
  >sr|IMAGp998K045001.2|cl|IMAGp998K045001|gb| BX109164|oid|234552707 BX109164 Barstead prostate BPH HPLRB4 1 Homo sapiens cDNA clone IMAGp998K045001 ; IMAGE2032251, mRNA sequence.
atccacccagtctctcaagaccaggaacgagaaaaaagaagtatcagtgacagcgatgaattagcttcagggttttttgtgttcccttacccatatccatttcgcccacttccaccaattccatttccaagatttccatggtttagacgtaattttcctattccaatacctgaatctgcccctacaactccccttcctagcgaaaagtaaacaagaaggaaaagtcacgataaacctggtcacctgaaattgaaattgagccacttccttgaagaatcaaaattcctgttaataaaagaaaaacaaatgtaattgaaatagcacacagcattctctagtcaatatctttagtgatcttctttaataaacatgaaagcaaaaaaaaaaaaa

SEQ ID NO: 172 - DNA, 322 bp, unknown
  >gi|5366932|gb|AI801460|sr|IMAGp998K125400.1| cl|IMAGp998K125400|AI801460 to89f06.x1 NCI_CGAP_Gas4 Homo sapiens cDNA clone IMAGE2185475 3′ similar to contains element MER22 repetitive element ;, mRNA sequence
ttttttttttttttttttttttttttttttttttttttttttttttttttttttttttttttttttttttttttttttttttttttttttttttaaaaaaaaaaggggccccctgggggggaaacaaatttggttccccccccggccccctgaaacccaatttttaaaactgggtttttttaaaaaaaagggggggggcccaaaaacccaaccccaaaagggggggtgccttttgccccccccccccccggtttttccccctttttttgggttaaaanncaagggcccccaaaaaaaagggggggggggatttttttttttt

SEQ ID NO: 173 - DNA, 460 bp, unknown
  >gi|2069924|gb|AA410759|sr|IMAGp998K151776.2| cl|IMAGp998K151776|AA410759 zt35e08.r1 Soares ovary tumor NbHOT Homo sapiens cDNA clone IMAGE724358 5′ similar to gbY00503 KERATIN, TYPE I CYTOSKELETAL 19 (HUMAN);, mRNA sequence
cccttggaccataaatttttattggcaggtcaggagaagagccgggggtaagggtcccttccttcccatccctctacccagaagacaccctccaaaggacagcagaagccccagagcctgctgcctcagaggaccttggaggcagacaaattgttgtagtgatcttcctgtccctcgagcaggctgcggtaggtggcaatctcctgctccagccgcgacttgatgtccatgagccgctggtactcctgattctgccgctcactatcagctcgcacatcgcccagctgggcttcaataccgctgatcagcgcctggatatgcgccacgtgggctccaaagcgcgcctccgtttctgccagtgtgtcttccaaggcagctttcatgctcagctgtgactgcagctcaatctcaagaccctgaagggtgcgccgcaggtcagtaacctcggacctgctcatct

SEQ ID NO: 174 - DNA, 797 bp, unknown
  >gi|5054911|gb|AI733798|sr|IMAGp998I164581.2| cl|IMAGp998I164581|AI733798 qk35b08.x5 NCI_CGAP_Co8 Homo sapiens cDNA clone IMAGE1870935 3′ similar to gbX02761_cds1 FIBRONECTIN PRECURSOR (HUMAN);, mRNA sequence
ttttttttttggggaatggaaatcttttattaaaacagttgtctttccacagtagtaaagctttggcacatacagtataaaaaataatcacccaccataattataccaaattcctcttatcaactgcatactaagtgttttcaatacaattttttccgtataaaaatactgggaaaaattgataaataacaggtaagagaaagatatttctaggcaattactaggatcatttggaaaaagtgagtactgtggatatttaaaatatcacagtaacaagatcatgcttgttcctacagtattgcgggccagacacttaagtgaaagcagaagtgtttgggtgactttcctacttaaaattttggtcatatcatttcaaaacatttgcatcttggttggctgcatatgctttcctattgatcccaaaccaaatcttagaatcacttcatttaaaatactgagcggtattgaatacttcgaagcagaacaggcaatgtgcagccctcatttatgagaaaaccctcaggaaactcccagggtgatgcttggagaagctgtgagtgagctgaagctggagaacttcctccagagcaaagggcttaagaaagaaagaagaactcctangctggtctgctaccattcactcagtttaaaatgatcctgcaganagacaatcttgtccttttggaattggaaagagattacctctcggaatcctctctgtcagcctgacatctaaaggcatgaagcactcaatttggcaataaaacaaatagggttggtctctgatggatctctga

SEQ ID NO: 175 - DNA, 408 bp, unknown
  >sr|IMAGp998F044825.2|cl|IMAGp998F044825|gb| BX106284|oid|234546949 BX106284 NCI_CGAP_Ov23 Homo sapiens cDNA clone IMAGp998F044825 ; IMAGE1964547, mRNA sequence
ttttttttttttttaaacatcagattttctatttttattaaaaactcacaaatttattcaacatgttttctttcatacagngaatggtctaatatgcactggaggtcacacaagcttaggtttattagaacaataaaagacatatgagaaatttaatatataaagaaaaagtagcagctgttgactgcatatttgaccataaaatttaaatattttggacttttattttaaagacacaaaaataaaacctgtgngggtctatataagtcatattaacaattccatgaatgttcaacaggacaaaaaaattagcaaagatgtttttttttaaatcttgnaacacttttttttttttttttttttaacactttctcaggntgctggggccaggcccctttacagnatttg

SEQ ID NO: 176 - DNA, 293 bp, unknown
  >gi|5593910|gb|AI888746|sr|IMAGp998L206082.1| cl|IMAGp998L206082|AI888746 wn22h10.x1 NCI_CGAP_Gas4 Homo sapiens cDNA clone IMAGE2446243 3′ similar to contains element MSR1 repetitive element ;, mRNA sequence.
tttttttttttttttttttttttttttttttttttttttttttttttttttgggttttttttttttttaaaaaagggcttaaatttaaaattctcaaaaaaaacaaaaaaaaaaaaaccccttgggtccaagccaaaaccccgggaaaacccccctaaaacagggcggggggccctacaaaaaaaacaaaaaagggccaagggaaatttaaaacatttaaatccttaaaggtccaccaggaaaaagggggccccccccccgggaaatcattttaaaaaaaaacaccgggcatt

SEQ ID NO: 177 - DNA, 404 bp, unknown
  >sr|IMAGp998D085368.2|cl|IMAGp998D085368|gb| BX107055|oid|234548491 BX107055 Barstead colon HPLRB7 Homo sapiens cDNA clone IMAGp998D085368 ; IMAGE2173015, mRNA sequence.
ctagtaatnactagtaatattactagtgaattcactagtaatgccagagccaacctggacttcctgtcattttcacaatcttggggctgatgaagaagggggtggggggagatctacaggagctcagcatggcatctgtgatattcctgcccaacatacatgacagaatccaatcatggaaaagcaccaaaaaacccaagttgaaggacaagtctaccaagtagctggccaggtaatcttcacaaaggtccgtgtcatgaaagacaaagcagcagaaactttcaaattagaggggacagctaaatgcaacacatggtcttggattgttttgttataaaggacattatcatgaagtctgaataaagtctgtagatgagatgagagaattccctgtttttaggaaa

SEQ ID NO: 178 - DNA, 270 bp, unknown
  >sr|IMAGp998D20611.1|cl|IMAGp998D20611|gb| BX115533|oid|234565441 BX115533 Soares_multiple_sclerosis_2NbHMSP Homo sapiens cDNA clone IMAGp998D20611 ; IMAGE276835, mRNA sequence
atattaacataattatgttggagagaagaaaaacccacacttagaagtcaggtttcttttaaaactccttgttacaaatagaatcaatgattatatctcagacataataatgggtcaaactcacaaaatcagtgctacaaaatacagtatttttccatcacttttagttgtaatgtacactatgtacagaaagataatactacctattttaaactaaatatagatgaacaaacagcttgttaaaaaccagaatcagggcttataaanagn

SEQ ID NO: 179 - DNA, 669 bp, unknown
  >sr|IMAGp998B14742.3|cl|IMAGp998B14742|gb| BX100503|oid|234535382 BX100503 Soares_fetal_heart_NbHH19W Homo sapiens cDNA clone IMAGp998B14742 ; IMAGE327085, mRNA sequence
tagtgttttcttaaaataagactgatttaaagcttatctagcagatagttttaaacacagattaaagaaaactagattgggtacaaatgtttggcttcccaagtgagaggccacatgtacatcaacagtactttccaacttaacactacgacccccattttgatggtgacagaattggaaacgggggacagtgatgagagcatcaccctcctgtcacttacagatggtcccttgggactggatttttcctgggagaccattgaccccaagaaccctgaaaacatgatatgcggtagcagaatggattagattgctgttagctttctggtaggtgaggggacctgaaaagaacagggccaccagcatttcaagggaacagccattccaattttcagaggttgattctcttggaattatttgacctgaggccataggaaagcaaagggttaaaaaagtcacgtttgacatgccagcctggggagctggacattggatacccgtgtgagcctcgtggaacaccataatcaaatgtgaaaattagagttgattaattaaagnaaaagaatttaagtctttatttctaaactgatgcatgaggacccccagtgagcaatattcccccaaggcaaatcaaccaaaagcaaaactgttttgaaaaaaaaaaaaaaa

SEQ ID NO: 180 - DNA, 711 bp, unknown
  >sr|IMAGp998B07407.1|cl|IMAGp998B07407|gb| BX111875|oid|234558127 BX111875 Soares fetal liver spleen 1NFLS Homo sapiens cDNA clone IMAGp998B07407 ; IMAGE208806, mRNA sequence
ctcattcttggcaggatggcttctcatcgtctgctcctcctctgccttgctggactggtatttgtgtctgaggctggccctacgggcaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatgagggacttctcctccagtggaactgaaggacgagggatgggatttcatgtaaccaagagtattccatttttactaaagcagtgttttcacctcatatgctatgttagaagtccaggcagagacaataaaacattcctgtgaaaggcacttttcattccactttaacttgattttttaaattcccttattgtcccttccaaaaaaaagagaatcaaaantttacaaagaatcaaaggaattctagaaagtatct

SEQ ID NO: 181 - DNA, 474 bp, unknown
  >sr|IMAGp998C16522.3|cl|IMAGp998C16522|gb| BX097461|oid|234529295 BX097461 Soares fetal liver spleen 1NFLS Homo sapiens cDNA clone IMAGp998C16522 ; IMAGE242631, mRNA sequence
aggacagagacgctggctaggccgccctccccactgttaccaacatgaagctgctcgcagcaactgtgctactcctcaccatctgcagccttgaaggagctttggttcggagacaggcaaaggagccatgtgtggagagcctggtttctcagtacttccagaccgtgactgactatggcaaggacctgatggagaaggtcaagagcccagagcttcaggccgaggccaagtcttactttgaaaagtcaaaggagcagctgacacccctgatcaagaaggctggaacggaactggttaacttcttgagctatttcgtggaacttggaacacagcctgccacccagtgaagtgtccagaccattgtcttccaaccccagctggcctctagaacacccactggccagtcctagagctcctgtccctacccactctttgctacaataaatgctgaatgaatccaaaaaaaaaaaaaaa

SEQ ID NO: 182 - DNA, 462 bp, unknown
  >gi|4739393|gb|AI655414|sr|IMAGp998O055540.1| cl|IMAGp998O055540|AI655414 ts98e03.x1 NCI_CGAP_GC6 Homo sapiens cDNA clone IMAGE2239324 3′ similar to gbS70057 GASTRIN/CHOLECYSTOKININ TYPE B RECEPTOR (HUMAN);, mRNA sequence.
ttcaggaggaagccaaacaatttttatttcttgaacagtttgggtaaggaaggagagggcagggccacggtccttaactggaaagggatgaagaaccttttcagtgcagaacagcctgttggtcagaggtatgagattaggcctgacggtattttcagtgctgattgggaagggtcactgtgaggtcaaactaggagcatcccagagtcagtcctgtgccaggctccatagtctctaaaacaatcagtgccattaatctatgtgtgtgagaggcaggtcagcccttgtcagagttcccactgtgtagaaatagttcactgctagggacgctgtgtctctccaaggtcagtgtcatgtttctatgggccaggctcagtcccatatcagaggcatgcctcctttcccatgtactgctccattcttattcctcttgtgtcaggtaagctaccttttcctgtgcgt

SEQ ID NO: 183 - DNA, 466 bp, unknown
  >sr|IMAGp998A244944.2|cl|IMAGp998A244944|gb| BX090980|oid|234516327 BX090980 NCI_CGAP_GC6 Homo sapiens cDNA clone IMAGp998A244944 ; IMAGE2010143, mRNA sequence
atggcttacatattcaaaaggttggatttgggaagcaatgctaagcagtggaatggacatcgacatagagagatcagctccacacttatactctgccactcaacttccccatgtgacttgaggatcactctaactccaaaacatagcaagctcgcggaacatcagggttcatgcaaagtattccaaggagccccttgaagcaacagaatggattgctcttctatggtggaatggcaccctggatgattaaaaccgtagcagcaaacaaaacctccatcaagtaagaattcagagtgtgagatatcacgcacagccacgcgtggatctttatatacgtgtcaatgtgtttgattgtatttttgctttcaaagtatgtattgagcatttcttctaggtcctcagtaacatctttttttaaaaaaaataaatgcttagggaattgttttatattaaaaaaaaaaaaaaa

SEQ ID NO: 184 - DNA, 16966 bp, unknown
  >Homosapiens chromosomeNCBI34474736257747532221
agcttttctcttctgtcaaccccacacgcctttggcacaatgaagtgggtaacctttatttcccttctttttctctttagctcggcttattccaggggtgtgtttcgtcgagatgcacgtaagaaatccatttttctattgttcaacttttattctattttcccagtaaaataaagttttagtaaactctgcatctttaaagaattattttggcatttatttctaaaatggcatagtattttgtatttgtgaagtcttacaaggttatcttattaataaaattcaaacatcctaggtaaaaaaaaaaaaaggtcagaattgtttagtgactgtaattttcttttgcgcactaaggaaagtgcaaagtaacttagagtgactgaaacttcacagaatagggttgaagattgaattcataactatcccaaagacctatccattgcactatgctttatttaaaaaccacaaaacctgtgctgttgatctcataaatagaacttgtatttatatttattttcattttagtctgtcttcttggttgctgttgatagacactaaaagagtattagatattatctaagtttgaatataaggctataaatatttaataatttttaaaatagtattcttggtaattgaattattcttctgtttaaaggcagaagaaataattgaacatcatcctgagtttttctgtaggaatcagagcccaatattttgaaacaaatgcataatctaagtcaaatggaaagaaatataaaaagtaacattattacttcttgttttcttcagtatttaacaatccttttttttcttcccttgcccagacaagagtgaggttgctcatcggtttaaagatttgggagaagaaaatttcaaagccttgtaagttaaaatattgatgaatcaaatttaatgtttctaatagtgttgtttattattctaaagtgcttatatttccttgtcatcagggttcagattctaaaacagtgctgcctcgtagagttttctgcgttgaggaagatattctgtatctgggctatccaataaggtagtcactggtcacatggctattgagtacttcaaatatgacaagtgcaactgagaaacaaaaacttaaattgtatttaattgtagttaatttgaatgtatatagtcacatgtggctaatggctactgtattggacagtacagctctggaacttgcttggtggaaaggactttaatataggtttcctttggtggcttacccactaaatcttctttacatagcaagcattcctgtgcttagttgggaatatttaatttttttttttttttaagacagggtctcgctctgtcgcccaggctggagtgcagtggcgcaatctcggctcactgcaaactccgcctcccgggttcacgccattctcctgcctcagcctcccgagtagctgggactacaggcgcccgccatcacgcccggctaatcttttgtatttttagtagagatggggtttcaccgtgtgccaggatggtctcaatctcctgacatcgtgatctgcccacctcggcctcccaaagtgctgggattacaggagtgagccaccgcgcccggcctatttaaatgttttttaatctagtaaaaaatgagaaaattgtttttttaaaagtctacctaatcctacaggctaattaaagacgtgtgtggggatcaggtgcggtggttcacacctgtaatcccagcactttggaaggctgatgcaggaggattgcttgagcccaggagttcaagaccagcctgggcaagtctctttaaaaaaaacaaaacaaacaaacaaaaaaattaggcatggtggcacatgcctgtagtcctagctacttaggaggctgacgtaggaggatcgtttggacctgagaggtcaaggctacagtgagccatgattgtgccactgcactccagcctgggtgacagagtgagactctgtctcaaaaaagaaaaaggaaatctgtggggtttgttttagttttaagtaattctaaggactttaaaaatgcctagtcttgacaattagatctatttggcatacaatttgcttgcttaatctatgtgtgtgcatagatctactgacacacgcatacatataaacattagggaactaccattctctttgcgtaggaagccacatatgcctatctaggcctcagatcatacctgatatgaataggctttctggataatggtgaagaagatgtataaaagatagaacctatacccatacatgatttgttctctagcgtagcaacctgttacatattaaagttttattatactacatttttctacatcctttgtttcagggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttgtaagtacattctaattgtggagattctttcttctgtttgaagtaatcccaagcatttcaaaggaattttttttaagttttctcaattattattaagtgtcctgatttgtaagaaacactaaaaagttgctcatagactgataagccattgtttcttttgtgatagagatgctttagctatgtccacagttttaaaatcatttctttattgagaccaaacacaacagtcatggtgtatttaaatggcaatttgtcatttataaacacctctttttaaaatttgaggtttggtttctttttgtagaggctaatagggatatgatagcatgtatttatttatttatttatcttattttattatagtaagaacccttaacatgagatctaccctgttatatttttaagtgtacaatccattattgttaactacgggtacactgttgtatagcttactcatcttgctgtattaaaactttgtgcccattgattagtaacccctcgtttcgtcctcccccagccactggcaaccagcattatactctttgattctatgagtttgactactttagctaccttatataagtggtattatgtactgtttatctttttatgactgacttatttcccttagcatagtgcattcaaagtccaaccatgttgttgcctattgcagaatttccttcttttcaaggctgaataatattccagtgcatgtgtgtaccacattttctttatccattaatttgttgattgatagacatttaggttggttttctacatcttgactatcatgaatagtgttgcaatgaacacaggagagctactatctcttagagatgatatcatggtttttatcatcagaaaacacccactgatttctatgctaattttgttacctgggtggaataatagtacagctatatattcctcattttagatatctttgtatttctacatacaataaaaaagcagagtacttagtcatgttgaagaactttaaacttttagtatttccagatcaatcttcaaaacaaggacaggtttatctttctctcaccactcaatctatatatacctcttgtgggcaaggccagtttttatcactggagcctttcccctttttattatgtacctctccctcacagcagagtcaggactttaactttacacaatactatggctctacatatgaaatcttaaaaatacataaaaattaataaattctgtctagagtagtatattttccctggggttacagttactttcataataaaaattagagataaggaaaggactcatttattggaaagtgattttaggtaacatttctggaagaaaaatgtctatatcttaatagtcacttaatatatgatggattgtgttactcctcagttttcaatggcatatactaaaacatggccctctaaaaagggggcaaatgaaatgagaaactctctgaatgtttttctcccctaggtgaattcacctgctgcttagaagcttattttctcttgatttctgttataatgattgctcttaccctttagttttaagtttcaaaataggagtcatataactttccttaaagctattgactgtctttttgtcctgttttattcaccatgagttatagtgtgacagttaattcttatgaaaattatatagagatggttaaatcatcagaaactgtaaacctcgattgggaggggaagcggatttttaaatgatttcctgaccaagcttaaccagtatattaaatcctttgtactgttctttggctataaagaaaaaaggtactgtccagcaactgaaacctgctttcttccatttagcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaagtaagtaatcagatgtttatagttcaaaattaaaaagcatggagtaactccataggccaacactctataaaaattaccataacaaaaatattttcaacattaagacttggaagttttgttatgatgattttttaaagaagtagtatttgataccacaaaattctacacagcaaaaaatatgatcaaagatattttgaagtttattgaaacaggatacaatctttctgaaaaatttaagatagacaaattatttaatgtattacgaagatatgtatatatggttgttataattgatttcgttttagtcagcaacattatattgccaaaatttaaccatttatgcacacacacacacacacacacacacttaacccttttttccacatacttaaagaatgacagagacaagaccatcatgtgcaaattgagcttaattggttaattagatatctttggaatttggaggttctggggagaatgtcgattacaattatttctgtaatattgtctgctatagaaaagtgactgtttttctttttcaaaatttagatacttatatgaaattgccagaagacatccttacttttatgccccggaactccttttctttgctaaaaggtataaagctgcttttacagaatgttgccaagctgctgataaagctgcctgcctgttgccaaaggtattatgcaaaagaatagaaaaaaagagttcattatccaacctgattttgtccattttgtggctagatttagggaacctgagtgtctgatacaaactttccgacatggtcaaaaaagccttccttttatctgtcttgaaaatctttcatctttgaaggcctacactctcgtttcttcttttaagatttgccaatgatgatctgtcagaggtaatcactgtgcatgtgtttaaagatttcaccactttttatggtggtgatcactatagtgaaatactgaaacttgtttgtcaaattgcacagcaaggggccacagttcttgtttatcttttcatgataatttttagtagggagggaattcaaagtagagaattttactgcatctagatgcctgagttcatgcattcattccataaatatatattatggaatgctttattttcttttctgaggagtttactgatgttggtggaggagagactgaaatgaattatacacaaaatttaaaaattagcaaaattgcagcccctgggatattagcgtactctttctctgacttttctcccacttttaaggctctttttcctggcaatgtttccagttggtttctaactacatagggaattccgctgtgaccagaatgatcgaatgatctttccttttcttagagagcaaaatcattattcgctaaagggagtacttgggaatttaggcataaattatgccttcaaaatttaatttggcacagtctcatctgagcttatggaggggtgtttcatgtagaatttttcttctaattttcatcaaattattcctttttgtagctcgatgaacttcgggatgaagggaaggcttcgtctgccaaacagagactcaagtgtgccagtctccaaaaatttggagaaagagctttcaaagcatggtaaatacttttaaacatagttggcatctttataacgatgtaaatgataatgcttcagtgacaaattgtacatttttatgtattttgcaaagtgctgtcaaatacatttctttggttgtctaacaggtagaactctaatagaggtaaaaatcagaatatcaatgacaatttgacattatttttaatcttttcttttctaaatagttgaataatttagaggacgctgtcctttttgtcctaaaaaaagggacagatatttaagttctatttatttataaaatcttggactcttattctaatggttcattatttttatagagctgtaggcatggttctttatttaattttttaaagttatttttaatttttgtggatacagagtaggtatacatatttacggggtatatgagatattttgatataagtatacaacatatataatccctttatttaattttatcttccccccaatgatctaaaactatttgcttgtccttttatgtcttatagttaaattcagtcaccaactaagttgaagttacttcttatttttgcatagctccagctctgatcttcatctcatgtttttgcctgagcctctgttttcatattacttagttggttctgggagcatactttaatagccgagtcaagaaaaatactagctgccccgtcacccacactcctcacctgctagtcaacagcaaatcaacacaacaggaaataaaatgaaaataatagacattatgcatgctctctagaaactgtcaattgaactgtatttgctcatcattcctaccatctacaccaccaaaatcaaccaaatttatgaaaaaaaacagccccaacataaaattatacacagataaacaggctatgattggttttgggaaagaagtcacctttacctgatttaggcaactgtgaaatgactagagaatgaagaaaattagacgtttacatcttgtcatagagtttgaagatagtgctggatctttctttttataagtaagatcaataaaaactccctcattctgtagaagttatgatttcttttctaagagacctttagaagtcagaaaaaatgtgtttcaattgagaaaaaagataactggagtttgtgtagtacttcccagattataaaatgcttttgtatgtattatctaatttaatcctcaaaacttcttcaatttagcatgttgtcatgacactgcagaggctgaagctcagagaggctgagccctctgctaacaagtcctactgctaacaagtgataaagccagagctggaagtcacatctggactccaaacctgatgcttctcagcctgttgccccttttagagttcctttttaatttctgcttttatgacttgctagatttctacctaccacacacactcttaaatggataattctgccctaaggataagtgattaccatttggttcagaactagaactaatgaattttaaaaattatttctgtatgtccattttgaattttcttatgagaaatagtatttgcctagtgttttcatataaaatatcgcatgataataccattttgattggcgattttctttttagggcagtagctcgcctgagccagagatttcccaaagctgagtttgcagaagtttccaagttagtgacagatcttaccaaagtccacacggaatgctgccatggagatctgcttgaatgtgctgatgacagggtaaagagtcgtcgatatgctttttggtagcttgcatgctcaagttggtagaatggatgcgtttggtatcattggtgatagctgacagtgggttgagattgtcttctgtgctttcgtctgtcctatcttcaatctttccctgcctatggtggtggtacctttctgtttttaacctggctataaattaccagataaacccattcactgatttgtaactcctttcagtcatgctctaactgtaaatgaaggcttaaactgaagtagaacagttacaaggttttacttggcagaacatcttgcaaggtagatgtctaagaagatttttttttctttttttaagacagagtttcgctcttgtttcccaggctggggtgcaatggtgtgatcttggctcagcgcaacctctgcctcctgggttcaagtgattctcatgcctcagcctcccaagtagctgggattacaggcatgcgccaccacacctggctaattttgtatttttagtagaggcggggtttcaccatattgtccagactggtctcgaactcctgacctcaggtgatccacccgccttggcctcccaaagtgctgggattacaggcatgagccaccttgcccagcctaagaagattttttgagggaggtaggtggacttggagaaggtcactacttgaagagatttttggaaatgatgtatttttcttctctatattccttcccttaattaactctgtttgttagatgtgcaaatatttggaatgatatctcttttctcaaaacttataatattttctttctccctttcttcaagattaaacttatgggcaaatactagaatcctaatctctcatggcactttctggaaaatttaaggcggttattttatatatgtaagcagggcctatgactatgatcttgactcatttttcaaaaatcttctatattttatttagttatttggtttcaaaaggcctgcacttaattttgggggattatttggaaaaacagcattgagttttaatgaaaaaaacttaaatgccctaacagtagaaacataaaattaataaataactgagctgagcacctgctactgattagtctattttaattaagtgggaatgtttttgtagtcctatctacatctccaggtttaggagcaaacagagtatgttcatagaaggaatatgtgtatggtcttagaatacaatgaatatgttctgccaacttaataaaggtctgaggagaaagtgtagcaatgtcaattcgtgttgaacaatttccaccaacttacttataggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtaagtagataagaaattattcttttatagctttggcatgacctcacaacttaggaggatagcctaggcttttctgtggagttgctacaatttccctgctgcccagaatgtttcttcatccttccctttcccaggctttaacaatttttgaaatagttaattagttgaatacattgtcataaaataatacatgttcatggcaaagctcaacattccttactccttaggggtatttctgaaaatacgtctagaaacattttgtgtatatataaattatgtatacttcagtcattcattccaagtgtatttcttgaacatctataatatatgtgtgtgactatgtattgcctgtctatctaactaatctaatctaatctagtctatctatctaatctatgcaatgatagcaaagaagtataaaaagaaatatagagtctgacaccaggtgctttatatttggtgaaaagaccagaagttcagtataatggcaatatggtaggcaactcaattacaaaataaatgtttacatattgtcagaagttgtggtgataaactgcatttttgttgttggattatgataatgcactaaataatatttcctaaaattatgtaccctacaagatttcactcatacagagaagaaagagaatattttaagaacatatctctgcccatctatttatcagaatccttttgagatgtagtttaaatcaaacaaaatgttaataaaaataacaagtatcattcatcaaagacttcatatgtgccaagcagtgtgtgctttgtgtagattatgtcatatagttctcataatccaccttccgagacagatactatttattttttgagacagagttttactcttgttgcccaggctggagtgcaatggtgccatctcggctcaccacaacctccgcctcccaggttcaagcgattctcctgcctcagcctcctgggattacaggcatgcaccaccatgcctggctaattttgtatttttagtagagatggggtttcaccatgttggtcagactggtctcaaactcctgacctctggtgatatgcctgcctcagcctcctaaagtgctgggattacaggcatgagccactgtgcccagccgacagatactattattatttccattctaccgagaaggagactaaggctctgatcatttaaataagttgcctaaggtgatgcagtgatataagtagcagagctaggaattgagccttggtaactttaactctggaccccaagtccttagctactaagctttactgcatggggtttagtcaaattaagacttttggaatatgagttacttttgagattagctttgtgatattttttgtgctcatttgtccaacaaagtctattttattttcatcttaattaggtttttgtatgaatatgcaagaaggcatcctgattactctgtcgtgctgctgctgagacttgccaagacatatgaaaccactctagagaagtgctgtgccgctgcagatcctcatgaatgctatgccaaagtggtaggtttattgttggaaaaaaatgtagttctttgactgatgattccaataatgagaaagaaaaataatgcaagaatgtaaaatgatatacagtgcaatttagatcttttcttgagatggtttcaattctggaatcttaaacatgaaagaaaaagtagccttagaatgattaacaaaatttagactagttagaatagaaagatctgaatagagcaatctctaaaaaattttgatctttttttctctttttcacaatcctgagaacaaaaaaaaattaaatttaaatgttaattagaagatatttaacttagatgtaaagtgagttaacctgattccaggattaatcaagtactagaattagtatcttatggcaaattatagaacctatccctttagaatattttcaaatctttttgaggatgtttaggaatagttttacaagaaattaagttaggagaggaaatctgttctggaggatttttagggttcccactagcatatgtaatggtttctgaactattcagaatcagagaaaactcatttttcctgctttcaagaagctactgtatgccaggcaccatgcacaaacaatgaccaacgtaaaatctctcattttggagagcctggaatctaactggaaaggtgaactaataataataatatgtacaatcatagccatcatttattaaacttttattatatgcaaggcactgtttaatttcattagcttacctggtttacagagcagctctatgagatgagtgccatctttgcccctattttagggataaggattctgaaatgtggagatggtaagtaaaattgcacaactgaagaatgagttacatgacttggctcaaatactggtcattgaactccagagcctgaatattcttaaccacttacatgatgcaagctcaccaaataaatagttcgaatgtattgtgacagagcggcattgatattcatctattcatgtggctttgagtaggaagaagaaaggatatcattctgaccagaggggtgaaaaacaacctgcatctgatcctgaggcataatactattaacacaattcttttatgtttcagttcgatgaatttaaacctcttgtggaagagcctcagaatttaatcaaacaaaattgtgagctttttgagcagcttggagagtacaaattccagaatgcgtaagtaatttttattgactgattttttttatcaatttgtaattatttaagacttaatatatgagccacctagcatagaacttttaagaatgaaaatacattgcatatttctaatcactctttgtcaagaaagataggagaggagagataaaatagttgatggggtggagaggtctatatttgaatgtagtctaaaaattgttctcttaagattggaagtatgtaggctgggagggtaaataccaaatcttggtatatcagaactgagcatgtcccttgaaggttaagaaatagttaatgggcaaatagagcatggcaatattttgtagagcagcaagtagtaggccttgaatagatgtcgctcaaaaagtaatatgtaagctgaacacaaaaatgtaacaaatgaatttagatacatatttgaatattaaattcaggttgtttgggagatgcacctagtctttgatggttaaacctttccctccatagaagagacagagacagaatggcttgctggactaatgtcccaattcaatagagtcttatctatgaaggttaaaaacaagaagagacatattatacagtagatatttattgtgtggctcatacacatggtgctcttctgattatggattttagagataataacagtgaacaagacatagtttctttcctcgagtagattaaagtcatacattgacttttaatggtgactggcattcttaatacatgattattatatattaggtaccatgtcagattaattataatactttactacttttaatttaacccttgaactatccctattgagtcagatatatttccttccattttctacttgtatctttcaagtttagcatatgctgatacatatgaagctctctccaggttttattgaaagaagaaattaataaatttattaatgtcactgaattaggcaactcactttcccaagattatgcaagtggtacaggtggaactcaaagccaagtttaactagttgttcaggagaatgttttctaccctccactaacccactactctgcagatggagataatatgatgaatggaacatagcaacatcttagttgattccggccaagtgttctctgttttatctactatgttagacagtttcttgccttgctgaaaacacatgacttctttttttcaggctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatgtgagtctttaaaaaaatataataaattaataatgaaaaaattttacctttagatattgataatgctagctttcataagcagaaggaagtaatgtgtgtgtgtgcatgtttgtgtgcatgtgtgtgtgcatgcacgtgtgtgtatgtgtgatattggcagtcaaggccccgaggatgataattttttttttttttttgagacggagtctcgctttgttgtccaggctggagtgcagtggtgccatctcggctcactgcaacctccgcctcccaggttcaagccattctcctgcctcagcctcccaagtagctgggactacaggtgcatgccaccatgcctggctaattttttgtatttttagtagaaaattttcagcttcacctcttttgaatttctgctctcctgcctgttctttagctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaacgtgaggagtatttcattactgcatgtgtttgtagtcttgatagcaagaactgtcaattcaagctagcaactttttcctgaagtagtgattatatttcttagaggaaagtattggagtgttgcccttattatgctgataagagtacccagaataaaatgaataactttttaaagacaaaatcctctgttataatattgctaaaattattcagagtaatattgtggattaaagccacaatagaataacatgttaggccatattcagtagaaaaagatgaacaattaactgataaatttgtgcacatggcaaattagttaatgggaaccataggagaatttatttctagatgtaaataattattttaagtttgccctatggtggccccacacatgagacaaacccccaagatgtgacttttgagaatgagacttggataaaaaacatgtagaaatgcaagccctgaagctcaactccctattgctatcacaggggttataattgcataaaatttagctatagaaagttgctgtcatctcttgtgggctgtaatcatcgtctaggcttaagagtaatattgcaaaacctgtcatgcccacacaaatctctccctggcattgttgtctttgcagatgtcagtgaaagagaaccagcagctcccatgagtttggatagccttattttctatagcctccccactattagctttgaagggagcaaagtttaagaaccaaatataaagtttctcatctttatagatgagaaaaattttaaataaagtccaagataattaaatttttaaggatcatttttagctctttaatagcaataaaactcaatatgacataatatggcacttccaaaatctgaataatatataattgcaatgacatacttcttttcagagatttactgaaaagaaatttgttgacactacataacgtgatgagtggtttatactgattgtttcagttggtcttcccaccaactccatgaaagtggattttattatcctcatcatgcagatgagaatattgagacttatagcggtatgcctgagccccaaagtactcagagttgcctggctccaagatttataatcttaaatgatgggactaccatccttactctctccatttttctatacgtgagtaatgttttttctgttttttttttttctttttccattcaaactcagtgcacttgttgagctcgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttataacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatct

SEQ ID NO: 185 - DNA, 364 bp, unknown
  >gi|4292568|gb|AI433922|sr|IMAGp998K105256.1| cl|IMAGp998K105256|AI433922 ti11f05.x1 NCI_CGAP_Pan1 Homo sapiens cDNA clone IMAGE2130177 3′ similar to TRQ08805 Q08805 SALIVARY PROLINE-RICH PROTEIN L ;, mRNA sequence
ttttttttttttttttttttttttttttttttttttttttttttttttttttttttttttttttttttttttttttttttttttttttttttttggggcggtcccaaaaccaacccttttaattggaaaaaaaggggaaaccccccccccccgggggggggggggggaaaaaaaaaaaaaacccccttttcaaaaaaaggcggggggggaaccaattccccccccggggaaaccccccccccccaaaaccctggaagttccctgggaaaaaaaaaaaaaaaagggggggggggaaaaaaggggggacccccccaaacccctttgcccccccccccaaggggggggaaaaaccccccggtccccc

SEQ ID NO: 186 - DNA, 474 bp, Homo sapiens
  >ImageID=1752104 IMAGP971B2475_S1_M13-RP CC FromEXP of IMAGp971B2475_s1_M13-RP.exp April 26, 2002 1241
catcatcatccccactgtggacaggtcgcagtaagaacaggagagtcacatcattctaataatggtctcagagatacatcacaatgactcccctgggcagaaagaagggataagagtcacatcacctgtgggctaggcccagagatgtcactcttacttctgtgggcatgtctcaggctggagaggagaatcacattacctaaacactggaccaagaaatacgtcacagtctttctcatgggcaaagtccaggacctcggcaaaacctgagtcctgtcctctcgctttcctccccgtgcacagcgagcttcaccacttgctccgcaccttctccatcaactactacctgtccctgtgatctgtccagtcgcccaactaaagtgctcagcaggtcagcagcgcggcgagcttctatgcaggcgtggggtctggggctcctggatctctgtgtcccatttcacagagattgccacc

SEQ ID NO: 187 - DNA, 667 bp, unknown
  >sr|IMAGp998G193982.2|cl|IMAGp998G193982|gb| BX090135|oid|234514632 BX090135 NCI_CGAP_GC4 Homo sapiens cDNA clone IMAGp998G193982 ; IMAGE1571370, mRNA sequence
ctcagcccacctgcacccaggtgaaataaacagccatgtagctcacacaacgcctgtttggtggtctcttcacacggacgcacatgaaatttggtgccctgactcggatcgggggacctcccttgggagatcaatcccctgtcctcctgttctttgctccctgagaaagatccacctatgacttcaggtcctcagactgaccagcccaaggaacatctcaccaattttaaatcagcggccaggcgttcctccagaacctcctcccacaggaacttgctacacgtgctggaaatctggcaactggaccaaggaatgcccgcagcccgggattcctaagccgcatcccatctgtgtgggaccccacggaaaatcggactgttcaactcacctggcagccactcccagagcccctggaactctggcccaaggctctctgactccttcccagatcttctcggcctagcggttgaagactgacactgcccgatcacctcggaagccccctagaccatcacggacgccgagcttcagatacgcgcatgaaaggaagattaagaaaaacaaaaaaatgtaaatttactggacactgaatatatactaagtactgcttaagtgatttacatgtcaagattaaaatattttgctttactttaaaaaaaaaaaaaaa

SEQ ID NO: 188 - DNA, 444 bp, unknown
  >gi|5340943|gb|AI793227|sr|IMAGp998P194992.3| cl|IMAGp998P194992|AI793227 qz35g10.x5 NCI_CGAP_Kid11 Homo sapiens cDNA clone IMAGE2028930 3′, mRNA sequence
tgtgcttcagtgatttattgagctttaacttcgggccagggcagcagtaggcgccgctgagaaaagatgaatgaacaagtccccaaaccaaaaataagcagcccactcctccaaaataaacacccactcccagcattaagtttaaattacaatcactcagggctcaggactttcttgttggcagcagctgttggcttcatagctttgaccattctgccacctctctgggatcccacagcccatctttgagtgtctgttttcagttcaagacagctagctcatgccagcactggcagaggtgagtccccaactgcacacaggcacctactggtataggtctggcagatttgagttcggatagagttgccatcacttcctcgctgtctggagctgcacgcgttaatatgccattagagccccagtttcttcatatgccagtagcac

SEQ ID NO: 189 - DNA, 476 bp, unknown
  >gi|5110011|gb|AI741723|sr|IMAGp998F015873.1| cl|IMAGp998F015873|AI741723 wg22c01.x1 Soares_NSF_F8_9W_OT_PA_P_S1 Homo sapiens cDNA clone IMAGE2365824 3′, mRNA sequence
tttttttttttttttttttgcatttaaatatgggctcaagtagtaggacttggctgagtttattactctcaagagtcaaacagaaagagaaggagtgaggaaaccaagaaacagtgggggtcaggctgggagtagaggaagtactagaacttgggttggcagagttcatctttgtagttcaagaagtctgaagttttacagttatttgcatcacctggatagaagactttcttggcttgcaaagctttctgccacatccataaagatcttgtttttatttacttgctggttttctttatttctttatttcatgaggctgaatagaaatgaaggaaacttggttggtgattctagaagctgctctctgggatgatctccttgttaatgatcccctatggattcatctttgatgggcaataaagaattgctttcttgagggacgcacagcttcctcagacctcttcatgctactgcag

SEQ ID NO: 190 - DNA, 417 bp, unknown
  >gi|3930893|gb|AI288116|sr|IMAGp998A054888.1| cl|IMAGp998A054888|AI288116 qv88a03.x1 NCI_CGAP_Ut2 Homo sapiens cDNA clone IMAGE1988620 3′ similar to gbJ03075 PROTEIN KINASE C SUBSTRATE, 80 KD PROTEIN, HEAVY CHAIN (HUMAN);, mRNA sequence
tttttttttttttttttttttttttttttttttttttttttttttttttttttttttttttttttttttttttttttttttggggggggggaaaaaaataaatggatggggggccaagggaaaaacatccacaaaccccccaaaaaagggggggcagaaacagggctgggggggggggggcaaaacaccaagggggggggcccaaggggggaaacccccctaacccatttttaaagggggggggggcccggaaaagggaaccaccccccccggggccccaaaaaaggggaaaaaaccccaaaaagggcctgccccccaaaaagagggcaaaaaaaccacaggcagccccaaagggggggggggacacgactgaaggggcgggtttaaaccctttttaaggggttttgcgcccaacca

SEQ ID NO: 191 - DNA, 488 bp, unknown
  >sr|IMAGp998A04528.1|cl|IMAGp998A04528|gb| BX101034|oid|234536444 BX101034 Soares fetal liver spleen 1NFLS Homo sapiens cDNA clone IMAGp998A04528 ; IMAGE244875, mRNA sequence.
aaatatgtgattttctctaatgtaacttcctcttaggatgtttcaccaaggaaatattgagagagaagtcggccaggtaggatgaacacaggcaatgactgcgcagagtggattaaaggcaaaagagagaagagtccaggaaggggcggggagaagcctgggtggctcagcatcctccacgggctgcgccgtctgctcggggctgagctggcgggagcagtttgcgtgtttgggttttttaattgagatgaaattcaaataacctaaaaatcaatcacttgaaagtgaacaatcagcggcatttagtacatccagaaagttgtgtaggcaccacctctgtcacgttctggaacattctgtcatcaccccgtgaagcaatcatttcccctcccgtcttcctcctcccctggcaactgctgatcgactttgtgtctctgttgtctaaaataggttttccctgttctggacatttcatataaatggaatca

SEQ ID NO: 192 - DNA, 526 bp, unknown
  >sr|IMAGp998H22207.1|cl|IMAGp998H22207|gb| BX096159|oid|234526691 BX096159 Soares placenta Nb2HP Homo sapiens cDNA clone IMAGp998H22207 ; IMAGE140997, mRNA sequence
ctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcggaggacgcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgttaaaaaaaaaaaaaaa

SEQ ID NO: 193 - DNA, 755 bp, Homo sapiens
  >ImageID=451838 IMAGP971M0340_S1 CC FromEXP of IMAGp971M0340_s1.exp April 26, 2002 1337
agaaacatgtatttcagcaaacccatccagtgtaccacgtaaaacctggtgggccagtaaatctcctgacaataaacctgtttggtatggtcttgatatgaacagagggtctcagttcgcttatggagaccaccaatcacctaatacagccattactcagatgacttttttgcgccttttatcaaaagaagcctcccagaacatcacttacatctgtaaaaacagtgtaggatacatggacgatcaagctaagaacctcaaaaaagctgtggttctcaaaggggcaaatgacttagatatcaaagcagagggaaatattagattccggtatatcgttcttcaagacacttgctctaagcggaatggaaatgtgggcaagactgtctttgaatatagaacacagaatgtggcacgcttgcccatcatagatcttgctcctgtggatgttggcggcacagaccaggaattcggcgttgaaattgggccagtttgttttgtgtaaagtaagccaagacacatcgacaatgagcaccaccatcaatgaccaccgccattcacaagaactttgactgtttgaagntgatcctgagactcttgaagtaatggctgatcctgcatcagcattgtatatatggtcttaagtgcctggcctccttatccttcagaatatttattttacttacaatcctcaagntttaattgantttaaatatttttcaatacaacaggttaggttttaagatgaccaatgacaa

SEQ ID NO: 194 - DNA, 467 bp, unknown
  >gi|4740154|gb|AI656175|sr|IMAGp998K055550.1| cl|IMAGp998K055550|AI656175 tt38e03.x1 NCI_CGAP_GC6 Homo sapiens cDNA clone IMAGE2243068 3′, mRNA sequence
actttgtgtcattcagtttagagcaatttaatctttttttctccaaatccatttttgaagctgagtttaacttttgcaacccatggcaaatcttaaatgccctcatttaccaatctttaccaaactcctatttaagcctctaaaagtcaatactggccatcagacccaaatttcagaagacaatagtgaaaaattacttacgtttaatctccagtcgtgtcccttggccgaaggtgatccacagtgttaacttaattactttcccctaaacaaaaatctcttttcgctgttaatatcactaacctgaccgatgcagagaaaatctacaattgagatgcctcacttaactggctagcgcttggctgttccttaagatgaactacttttctatcccttactcatctgactttttgaaagaatctggtactcttttgaattgacctgagctcatatctcaaacacaaaaa

SEQ ID NO: 195 - DNA, 474 bp, unknown
  >sr|IMAGp998A065260.2|cl|IMAGp998A065260|gb| BX119512|oid|234573224 BX119512 NCI_CGAP_Kid11 Homo sapiens cDNA clone IMAGp998A065260 ; IMAGE2131469, mRNA sequence
tcttaattttttttttttaattggaaaactcgatctctaggaggaaagaaaaaaaaggcctgggtcccagaaaactggttgtagccctagcttcttaatttattggccatgtgactatagactatttgcttacttgctctggtcctcagttttctggtctgcagaatgggtatcttgatgtgtgttgttcttctcctccacgatatgaaaccaaaggcctttatagcttgttttctgaggctccttccaggtgaaaatcctataactcccaataaaacagctggagaggctgtctctactgccttttctgtctacccaggagtttccttggttgatgggaagctgtttaagaactgaaatgagagagaagaggcagacagacccaaccgctacagctcctatcagaaagggcagcgcccgtggaattgtaggtagcctgttactggctcagagaacaagcaggcctacttatgt

SEQ ID NO: 196 - DNA, 498 bp, unknown
  >sr|IMAGp998J076121.2|cl|IMAGp998J076121|gb| BX096830|oid|234528032 BX096830 NCI_CGAP_Pr22 Homo sapiens cDNA clone IMAGp998J076121 ; IMAGE2461158, mRNA sequence
gcccccaccttcccatccagacagccgagctcgctccggaacttgtcatctccaacgacaaaaggagcttttgccactgactcggccgtgtcctgacacctccagaacgcaggtgctggcgcccgttctgcctgggaccccgggaacctctcctgccggaagccggacggcagggatgggccccaacttcgccctgcccacttgacttcaccaaatcccttcctggagactgaacctggtgctcaggagcgaaggactgtgaacttgtggcctgaagagccagagctagctctggccaccagctgggcgacgtcaccctgctcccaccccacccccaagttctaaggtcttttcagagcgtggaggtgtggaaggagtggctgctctccaaactatgccaaggcggcggcagagctggtcttctggtctccttggagaaaggttctgttgccctgatttatgaactctataatagagtatataggttttgtacctttt

SEQ ID NO: 197 - DNA, 584 bp, unknown
  >sr|IMAGp998L10662.1|cl|IMAGp998L10662|gb| BX102297|oid|234538974 BX102297 Soares fetal liver spleen 1NFLS Homo sapiens cDNA clone IMAGp998L10662 ; IMAGE296601, mRNA sequence
tccgatatggcctcaatgaagtgaagtgcaaagggaatgagtcttccttgtgggattgtcctgccagacgctggggccatagtgagtgtgggcacaaggaagacgctgcagtgaattgcacagatatttcagtgcagaaaaccccacaaaaagccacaacaggtcgctcatcccgtcagtcatcctttattgcagtcgggatccttggggttgttctgttggccattttcgtcgcattattcttcttgactaaaaagcgaagacagagacagcggcttgcagtttcctcaagaggagagaacttagtccaccaaattcaataccgggagatgaattcttgcctgaatgcagatgatctggacctaatgaattcctcaggaggccattctgagccacactgaaaaggaaaatgggaatttataacccagtgagttcagcctttaagataccttgatgaagacctggactattgaatggagcagaaattcacctctctcactgactattacagntgcatttttatggagttcttcttctnctatgattcctaanactgctgctgaatttatnaaaattaancttgn

SEQ ID NO: 198 - DNA, 732 bp, Homo sapiens
  >ImageID=114388 IMAGP971C043_S1 CC   FromEXP of IMAGp971C043_s1.exp April 26, 2002 1242
aaattccctctggggatctatgagctaatgagtttcttcatgcttatcccatgtgatgttgggaatccagactacagggcaaagtagatactttaactccattttacagataagagtgaggttcagagagatgattgagttagtgaggactgagcctggcctcaaactcgagttgactacaagcacctcttctctgcacagtgttactcatgaaagagacttccctgcaagacctgggccttttacttcaaagggcaaagagacttatttttcctttcatgcctttccatggcccactctttcattctggtttcaggttgccagtctggctgcagtcctctgtcattggtcggtaacaccctgggtcatattccacacttaaagatggaggtaattgcttcaagatgtggccctttaaggagagaataggttgaaaggcaaattctttttttcagctttagtcagagcctgagttttggaaaaaggaaatgatgagttgctccgagatataaactttctttcatctcttagaagaagtatttgtccttctgaaaaggttagtggcagttttgtccattgattttttttctaaacctttattacccagtttcatggtccatcacagtaagtaaaacaaaccaccctcccccagaccacctccctccttatttgcacagggaaccttcaaacaattgggagcagaagcagaatgatgcctgtgaagcctttgcc

SEQ ID NO: 199 - DNA, 528 bp, unknown
  >sr|IMAGp998G06783.2|cl|IMAGp998G06783|gb| BX118770|oid|234571916 BX118770 Soares_fetal_heart_NbHH19W Homo sapiens cDNA clone IMAGp998G06783 ; IMAGE342941, mRNA sequence
tcttatactaatttatacaaagatattaaggccctgttcattaagaaattgttcccttcccctgtgttcaatgtttgtaaagattgttctgtgtaaatatgtctttataataaacagttaaaagctgacagttcgcccttactcttggaggtcatgttcaggaggggcattcctttcccctgggggtcatgggtgtccccatgcccacatattgcacgtgcagggaggtaagtgcctgcatcccaaatcggttctaggtcaactggcctcaaactgatttgccatgagctcacaaaatgaatccctatgcttaatgaccaggtcacataaaatccagcccacttacaggttttctggcatctgtttgggtgtcctaatttttttggcagtgtcatttgaagaatttttttaaagcagtttatttaagaacatactgantaaatgcaggatcgctactaaaaantgctttgtatccttggtgggtgtcttctgctattntatctacttttgaacactntcaggactttt

SEQ ID NO: 200 - DNA, 489 bp, unknown
  >sr|IMAGp998K08276.1|cl|IMAGp998K08276|gb| BX090262|oid|234514887 BX090262 Soares infant brain 1NIB Homo sapiens cDNA clone IMAGp998K08276 ; IMAGE47615, mRNA sequence
ttttttttttttttctggaaggttctcaggtctttatttgctctctcaaattccaggaattgacttatttaattaatccatcaacctctcatagcaaatatttgagaaaacaaatttatattcagattcttattttcagtagggaagtaagaagttgcagctcagtgcacgtaaagttgagacagagatggagacatccagccccacctctctggaacaagaaagatgactggggaggaaacacaggtcagcatgggaacaggggtcacagtggacacaagggtgggctgtctctccacctcctcacattatgctaacagggacgcagacacattcaggtgcctttgcagaaagagatgccagaggctcttgaagtcacaaaggggaggcgtgaagaaatcctgcatctcagtccctcacaagacagctgtctcaggcttttcaagctgtgagagacacatcagagccctgggcactgtcgctggacgc

SEQ ID NO: 201 - DNA, 364 bp, unknown
  >gi|2224139|gb|AA494352|sr|IMAGp998F152230.1| cl|IMAGp998F152230|AA494352 ne27c08.s1 NCI_CGAP_Co3 Homo sapiens cDNA clone IMAGE898574 3′ similar to contains element LTR5 LTR5 repetitive element ;, mRNA sequence.
ctttcactcattggtttattcccgcactccttccctcactcattcattcattgactcttgcaggtagtgcacaccaagggtgtgaaggggtgaggtgtctggttctctgctcagacctctgcaagtgcccgcagaggtgttctctgctcagacctctgcaagtgcccgcagaggtgactgggcggcctccacttctggcctggccttgcacccctgtgctcttgggggccatggctgggcagggcaggtgtccccctctgcacaggcccagtgtgtctcagcctcagagctggcagggcagggcaggcatcccctctgcactggcccagtgtctctcagcctcagaaccagatgtgagaagacg

SEQ ID NO: 202 - DNA, 3159 bp, Homo sapiens
  >ENST00000322244 Coding sequence
atggaaggatccgagcctgtggccgcccatcagggggaagaggcgtcctgttcttcctgggggactggcagcacaaataaaaatttgcccattatgtcaacagcatctgtggaaatcgatgatgcattgtatagtcgacagaggtacgttcttggagacacagcaatgcagaagatggccaagtcccatgttttcttaagtgggatgggtggtcttggtttggaaattgcaaagaatcttgttcttgcagggattaaggcagttacaattcatgatacagaaaaatgccaagcatgggatctaggaaccaacttctttctcagtgaagatgatgttgttaataagagaaacagggctgaagctgtacttaaacatattgcagaactaaatccatacgttcatgtcacatcatcttctgttcctttcaatgagaccacagatctctcctttttagataaataccagtgtgtagtattgactgagatgaaacttccattgcagaagaagatcaatgacttttgccgttctcagtgccctccaattaagtttatcagtgcagatgtacatggaatttggtcaaggttattttgtgatttcggtgatgaatttgaagttttagatacaacaggagaagaaccaaaagaaattttcatttcaaacataacgcaagcaaatcctggcattgttacttgccttgaaaatcatcctcacaaactggagacaggacaattcctaacatttcgagaaattaatggaatgacaggtttaaatggatctatacaacaaataacggtgatatcgccattttcttttagtattggtgacaccacagaactggaaccatatttacatggaggcatagctgtccaagttaagactcctaaaacagttttttttgaatcactggagaggcagttaaaacatccaaagtgccttattgtggattttagcaaccctgaggcacctttagagattcacacagctatgcttgccttggaccagtttcaggagaaatacagtcgcaagccaaatgttggatgccaacaagattcagaagaactgttgaaactagcaacatctataagtgaaaccttggaagagaagcctgatgtaaatgctgacattgtgcattggctctcttggactgcccaaggctttttatctccacttgctgcagcagtaggaggtgttgccagccaagaagtattgaaagctgtaacaggaaaattttctcctttgtgccagtggttatatcttgaagcagcagatattgttgaatcactaggcaaacctgaatgtgaagaatttctcccacgaggagatagatatgatgccttaagagcttgcattggagacactttgtgtcagaaactgcaaaatttaaacatcttcttagtagggtgtggagccataggctgtgaaatgttgaaaaattttgctttacttggtgttggcacaagcaaagagaaaggaatgattacagttacagatcctgacttgatagagaaatccaacttaaatagacagttcctatttcgtcctcatcacatacagaaacctaaaagctacactgctgctgatgctactctgaaaataaattctcaaataaagatagatgcacacctgaacaaagtatgtccaaccactgagaccatttacaatgatgagttctatactaaacaagatgtaattattacagcattagataatgtggaagccaggagatacgtagacagtcgttgcttagcaaatctaaggcctcttttagattctggaacaatgggcactaagggacacactgaagttattgtaccgcatttgactgagtcttacaatagtcatcgggatcccccagaagaggaaataccattttgtactctaaaatcctttccagctgctattgaacataccatacagtgggcaagagataagtttgaaagttccttttcccacaaaccttcattgtttaacaaattttggcaaacctattcatctgcagaagaagtcttacagaagatacagagtggacacagtttagaaggctgttttcaagttataaagttacttagcagaagacctagaaattggtcccagtgtgtagaattagcaagattaaagtttgaaaaatattttaaccataaggctcttcagcttcttcactgtttccctctggacatacgattaaaagatggcagtttattttggcagtcaccaaagaggccaccctctccaataaaatttgatttaaatgagcctttgcacctcagtttccttcagaatgctgcaaaactatatgctacagtatattgtattccatttgcagaagaggacttatcagcagatgccctcttgaatattctttcagaagtaaagattcaggaattcaagccttccaataaggttgttcaaacagatgaaactgcaaggaaaccagaccatgttcctattagcagtgaagatgagaggaatgcaattttccaactagaaaaggctattttatctaatgaagccaccaaaagtgaccttcagatggcagtgctttcatttgaaaaagatgatgatcataatggacacatagatttcatcacagctgcatcaaatcttcgtgccaaaatgtacagcattgaaccagctgaccgtttcaaaacaaagcgcatagctggtaaaattatacctgctatagcaacaaccactgctacagtttctggcttggttgccttggagatgatcaaagtaactggtggctatccatttgaagcttacaaaaattgttttcttaacttagccattccaattgtagtatttacagagacaactgaagtaaggaaaactaaaatcagaaatggaatatcatttacaatttgggatcgatggaccgtacatggaaaagaagatttcaccctcttggatttcataaatgcagtcaaagagaagtatggaattgagccaacaatggtggtacagggagtcaaaatgctttatgttcctgtaatgcctggtcatgcaaaaagattgaagttaacaatgcataaacttgtaaaacctactactgaaaagaaatatgtggatcttactgtgtcatttgctccagacattgatggagatgaagatttgccgggacctccagtaagatactacttcagtcatgacactgattaa

SEQ ID NO: 203 - DNA, 166 bp, unknown
  >gi|2524282|gb|AA620343|sr|IMAGp998A022575.1| cl|IMAGp998A022575|AA620343 af07b01.s1 Soares_testis_NHT Homo sapiens cDNA clone IMAGE1030921 3′, mRNA sequence.
ttttggtagctattgaatcagggccacacatttaattgatattatgatcaagatgttcaaggcaaaaaatactattacttatttaatgtggaacaagtctagtctttctcttgagctcccacctgctggttaggaggcaacaatgttatttggatcctgtttagag

SEQ ID NO: 204 - DNA, 408 bp, unknown
  >sr|IMAGp998E122300.2|cl|IMAGp998E122300|gb| BX095446|oid|234525261 BX095446 NCI_CGAP_Co3 Homo sapiens cDNA clone IMAGp998E122300 ; IMAGE925427, mRNA sequence
aaatatgtataaatgtgggcaccgtcatttgtctagaaaaatctatagtaaattgttttgttttcctttcattgcaaagtctcatacttgttcttccaaaatcagttctatgagttgctttaaaattagcctgggacgtccaagaagtgttaagacatctgcagtaactggggcagccagtccctggaggccaactcagggaacaacaaactccttcgccaagccgtccacacatgccagctgatggtctaggacccaagtatgtgccaagagtgaggtgtcttacttctaaggagaggcaagcagtttgctatgcaagcgggtcgctggccctggacctaaagatcttctctctaactccatattctttaaataaacttgtcttttatgaggaaaaaaaaaaaaaaa

SEQ ID NO: 205 - DNA, 398 bp, unknown
  >gi|879189|sr|IMAGp998C12278|cl|IMAGp998C12278| gb|H14369|H14369 ym18f06.s1 Homo sapiens cDNA clone 48417 3′.
ttttttttttttgccttttcatttgtctttttattttacatttaaaaagtggttattgctctaagtggatcagaggtaaggacctctcccctagggagccttggncttgcagccccattcagcagggatggaagtcacaagacaatgagtggagcctcatgccctcccatgaggaagcccttagtattgctgacatctgccctttatcctgtctctcctccccagtgctgtcacacttgggcaaagcagagtggtggcagacccagccttgagagctcttgtaggaccggaaggaaggggcggtcattaggtgatgggcttctgggctctctgggcttgggngaccaatgctcttattcattncattttcaacaaatatttaactggaggtaccgggc

SEQ ID NO: 206 - DNA, 654 bp, unknown
  >sr|IMAGp998E191782.3|cl|IMAGp998E191782|gb| BX110507|oid|234555393 BX110507 Soares_testis_NHT Homo sapiens cDNA clone IMAGp998E191782 ; IMAGE726522, mRNA sequence
caaattcaacccaccacataggccctcccgaggccacctctgcgtcatctttgtctacagattctgtgtccgtcttcctctcctcattgactctgcatgaaggggatttctggtggagtaagggtagcagctcttgccgcacgtgcagagagagaggaacttccagtgcccgctatggagccacagcccacagcatgggggaagtccccatccagaggcagtgctgcagctagaggtagctccagagtcctccgggccctgcaccgatacagccaaagaccagcaaagcgacaagctgccagacctcatgccacctgccgtagccactgggctcagccctggagctgagagcatcgctggagatagacgtggcagagaagaggttgcgagcatggccccagccagcagctcccacgctgcccctagtcctgggcatggagcgagccttggtgtcagagaccagggtgtgcagtctgagctcctctaccttactaaagagaggcctcttttatttaccagagccacagccctgctgcctcaggaccttttcattctgccggtgctggggctgtctatctgcaagctggaggtgttgagagcaggaaaagggaggctgtgaggaaggggttcgggcagctcctcctgctctcagag

SEQ ID NO: 207 - DNA, 417 bp, unknown
  >sr|IMAGp998O124335.2|cl|IMAGp998O124335|gb| BX099272|oid|234532920 BX099272 Soares_fetal_heart_NbHH19W Homo sapiens cDNA clone IMAGp998O124335 ; IMAGE1707107, mRNA sequence
agcatggccccagccctggnacaagnacacgttctgccagggcaaagctgatgggctctatcccaatcctcgggaacggtccagcttctacagctgtgcagcggggcggctgttccagcaaagctgcccgacaggcctggtgttcagcaactcctgcaaatgctgcacctggaattgagtcgctaaagcccctccagtcccagctttgaggctgggcccaggatcactctacagcctgcctcctgggttttccctgggggccgcaatctggctcctgcaggcctttctgtggtcttcctttatccaggctttctgctctcagccttgccttccttttttctgggtctcctgggctgcccctttcacttgcaaaataaatctttggtttgtgcccctcttcccaaaaaaaaaaaaaaa

SEQ ID NO: 208 - DNA, 767 bp, unknown
  >sr|IMAGp998K102640.2|cl|IMAGp998K102640|gb| BX098917|oid|234532208 BX098917 Soares_testis_NHT Homo sapiens cDNA clone IMAGp998K102640 ; IMAGE1056129, mRNA sequence
cgcaagnataatggattgtcaaaaaggaggcaatatggcaaacggaagttgtttggacctgaagtcagaagaatcaagttcagctcactttgattctgatgggatctaccaaagcatgtttgttgatatgatcaatccaaaagagaaggtgagactgatgaacaggaggactggagtcttgtagtaggaggagtaaatgggattcatatctcacatcagcaccttatttatttgacactgctgatactgttaatttaactactaaagtctggatgtcctggcatggcagacaaaagtttcattcattatccagtgaaacactgaaatgaaattaggatgagcaagtcactgagttctttggaacgggaatcctcacggttctcctacagtcctaagaatgcttacaaaacaacgggcttatatcaatgccagatctggaagccatgggctaacttgacaagcccactggatttgactttagcttcttgctctagccagaggttaccaacacctaaacacaaaatagactatctgctcctgactgctgcagaactaaagagctgactttattcttgagttctttattcagagtcccagaaaattatttctaaaatgcatgctgggataactttcaaaatcaggatttggtttactttgcaacagatattaccacacaaaaaatattaaattttagaaaacaagacaaatcatctttaacttaatgccctagtcttccttaacaaaagcaaagacacaaanttaagaag

SEQ ID NO: 209 - DNA, 468 bp, unknown
  >gi|4735332|gb|AI651353|sr|IMAGp998F035711.1| cl|IMAGp998F035711|AI651353 wb05c02.x1 NCI_CGAP_GC6 Homo sapiens cDNA clone IMAGE2304770 3′ similar to contains Alu repetitive element;contains element TAR1 repetitive element ;, mRNA sequence
cttctttttgcttctcttttctttctttctttctttctttctttctttctttctttctttttctttttctctcttcccctctttctttcctgccttcctgcctttcttcttttcttctttcctcccttcctcccttccttctttcctcccgcctcagcctcccaaagtgctgggatgactggcgggaggcaccatgcctgcttggcccaaagagaccctcttggaaagtgagacgcagagagcgccttccagtgatctcattgactgatttagagacggcatctcgctccgtcaccccggcagtggtgccgtcgtaactcactccttgcagcgtggacgctcctggactcgagcgatccttccacctcagcctccagagtacagagcctgggaccgcgggcacgcgccactgtgcccacaccgtttttaattgtttttttttcccccgagacagagtttcactctcgt

SEQ ID NO: 210 - DNA, 726 bp, unknown
  >sr|IMAGp998O184348.2|cl|IMAGp998O184348|gb| BX105424|oid|234545229 BX105424 Soares_pregnant_uterus_NbHPU Homo sapiens cDNA clone IMAGp998O184348 ; IMAGE1712105, mRNA sequence.
acggattttattaaaacctatgttattttttgttgctaatcatttgacacttaatcgaaacagaataagttactgtggaaaaaaggatgatccaactcctgaggattgccgcctgtgtgtctttaattgattatctttgatagttagagttatttttcagatttttccccctcatactggattgtgatgctctgatatgagcatgcaatgactgatacagaaataggctggagcttcggtgtgtgcttttgctatacagcttacagttctaatgcctgatgtagagatgatgaatttctcctttttaaacctatcttctgtttactccttttgcattatgaaggccctcttcaatgatgtatgtttctgatggaatcagaaaatttcacactagaaaagtgtacctaacttgattcagtgtgagctcttgggacagggaggggagccaggggtagagcttaaagaagctggctctgtaagagttgtatatcaaagtaccttttgctacaaggtgcatatagaagtctaagataagaaatgtaccctctatactttatttttcatgcccctatcatgtttggtgtgccttaaatcttacctttaaatgaagactctatggattgtttacaaatgaagntttacactaggaccagatcgtccatacatgattggtcttctacaataataatgttcaattctttcattttaaatctttctttanggtca

SEQ ID NO: 211 - DNA, 391 bp, unknown
  >gi|1512724|sr|IMAGp998B201153|cl| IMAGp998B201153|gb|AA037588|AA037588 zk36d10.r1 Soares pregnant uterus NbHPU Homo sapiens cDNA clone 484915 5′
ccaaattgctcaacagtatgtatgtttagaggggttagactccttttgaaaatctggatatctaaccacctacttaaatctgttngatagtgtcanaccacccccacccttgatcctcccacccccaagaaaaagaaaagatgctcctgttaaatgaaggctctttnggggtangtgctaggtatactcttggctagatggattcagctcagaatttntgactttttaaagctaagtccttngtagtcacatggttgacttgaaaggatgaggtggtgtgtgcatttatgacgcttntcctacccccctganagaaacttcattggccgccntggttactcaccttgaagagagtaccatccattntacgtgaaaaacacatctgtcaccc

SEQ ID NO: 212 - DNA, 658 bp, unknown
  >sr|IMAGp998P08567.1|cl|IMAGp998P08567|gb| BX099307|oid|234532990 BX099307 Soares_placenta_8to9weeks_2NbHP8to9W Homo sapiens cDNA clone IMAGp998P08567 ; IMAGE260215, mRNA sequence
tctcgtctcacatttgcactctctccttcccacattctgtgctcagctcactcagtggtcagcgtttcctgcacactttacctctcatgtgcgtttcccggcctgatgttgtggtggtgtgcggcgtgctcactctctccctcatgaacacccacccacctcgtttccgcagcccctgcgtgctgctccagaggtgggtgggaggtgagctgggggctccttgggccctcatcggtcatggtctcgtcccattccacaccatttgtttctctgtctccccatcctactccaaggatgccggcatcaccctgagggctcccccttgggaatggggtagtgaggccccagacttcacccccagcccactgctaaaatctgttttctgacagatgggttttggggagtcgcctgctgcactacatgagaaagggactcccatttgcccttccctttctcctacagtcccttttgtcttgtctgtcctggctgtctgtgtgtgtgccattctctggacttcagagccccctgagccagtcctcccttcccagcctccctttgggcctncctaactccacctaggctgncagggaccggagtcagntggttcaaggncatnggnagctctgnctncaagtctacccttnccttccggactnct

SEQ ID NO: 213 - DNA, 548 bp, unknown
  >sr|IMAGp998A191006.1|cl|IMAGp998A191006|gb| BX118017|oid|234570410 BX118017 Soares_fetal_liver_spleen_1NFLS_S1 Homo sapiens cDNA clone IMAGp998A191006 ; IMAGE428442, mRNA sequence
cagatctctaccagctgctccagagactcttcaaaagccactcatctctggagggattgctcaaagccctgagccaggctagcacagatcctaaggaatcaacatctcccgagaaacgtgacatgcatgacttctttgtgggacttatgggcaagaggagcgtccagccagactctcctacggatgtgaatcaagagaacgtccccagctttggcatcctcaagtatcccccgagagcagaataggtactccacttccggactcctggactgcattaggaagacctctttccctgtcccaatccccaggtgcgcacgctcctgttaccctttctcttccctgttcttgtaacattcttgtgctttgactccttctccatcttttctacctgaccctggtgtggaaactgcatagtgaatatccccaaccccaatgggcattgactgtagaataccctagagttcctgtagtgtcctacattaaaaatataatgtctctctctattcctcaacaataaaggatttttgcatatgaaaaaaaaaaaaaaa

SEQ ID NO: 214 - DNA, 528 bp, unknown
  >sr|IMAGp998B02152.1|cl|IMAGp998B02152|gb| BX110160|oid|234554699 BX110160 Soares infant brain 1NIB Homo sapiens cDNA clone IMAGp998B02152 ; IMAGE31971, mRNA sequence
ttttttttttttttgaagggacaccgatttattgagtagcagagctgagggaaggcgtaggatggctccagcttccggtcagtggctacatggtcagttccatgatggcgttgacgatgtcactgtggttgtctctcagagcccgcacggccttggccctggacacattggcctgcgccatcaccagctcaatgtcacgcagttccagccccgcctcgtccacctcttcctcctcctcctcttcctcttccttgcactccagcctcacccggggcctgggtgctgactcagggaccaaggctgagggctctgagggcaccttaaacttctcagctgcggctttgtgcacttgctgggacaggtcctcaatcttggcctcgccaaagaccacataagtgtctgaggctgggctcttgaagacatcaggcttggcgatgacaaagaggatgttcttggacttctggatggtgatcctggtgactccctgaatctgccgcaagcccagctttgacattgcctttcgggcct

SEQ ID NO: 215 - DNA, 2341 bp, unknown
  >Homo sapiens|chromosomeNCBI34764716658647189981
gcagattctccaggcccagcatctgcctcaccgtggccccccacaagccaagcgcctgcctttcagcagcctctacacacccagctcctgccacccaatggctctttaggccaagctcatacctcacgatgatttttccaggcccaacttttgtctcatggcaaccttccctggccaagtttccacctatttcctggcagcctggacaggcccaggtcctgccacacactggcctctctacgcccagctcatgcctcacagtggcctctccaggcccagctcctgtcccgggacatcatctccaggcccaaaacttcctcaagtcggcctctccaggcccagttgctgcctcccggcattctctccaggcctagctcttcctcctggctgtatctacaagaccaactcctgcctcacaacaaccttttatggctcagctcctgcccaactactgccggcctttgtaggcccaaaacttcctcaagtcaagctctttaggcccaccttctgccttgcagtggcctgtacagacccagctctggcttgagaacagcctctgcaggccctgctcttgcctcttagctccctctccaggcccatctcttgcctcacagtggcttccgtgggccaagttcccgcctgcctcccagcagcctcaacaggcctagctcctccctcacaatggcttgtttaggtccagttgatgcctctggcaacctgtccaggcccagctcctgcctcacactggcctctctaggccgaggtcctttctcatactggcctgtttaggcccagctcattcctcttgtcatctctccaggcccagcttttgcctgttgttggcctctacctcacagtgcaccttccagtcccacctcttgcctcaccatggcctcctctgaccaggttcctgcctttcggcagcctctacaggcctagctgctgcctcccaatggcctttgtaggccacgctcatgcctcactgtggcctttccaggcctagctttcgctttttggccactccaggcccagaacttcccccagtcagcctctccaggcccagctcttcctcccagcaacctctgcaggcccaaatcatcctcaaattggcctcttctttcccagctcctgcctcctggtggcctctgaagacccaaatcgtcctccagttggtttttccaggcccagctcctgccttttggtggcctctccaggtgcaaaacttcctcccatcagcctgtccaggcccagctcatgcctcttggtggccttctcaggccctgcttttgacttggtggcctcttcaggcccagaacttgaactcaagtcagcctctccaggcccagctcctgccttcttaaggtctgtacaggcccagcctctacctcacagcggactctccacacccagctcttgcctcactgtagcctccccagtccaaaactcctgccttttggcagcttcgacaagcccagctcctgcctttcaatgacctctttaggccccgctcattccttacaacggcctttccaggcccagtttttcccttttggcggcctctccaggcccagaacttcctcaagtcggcctctttaggcccagttgctgcctcctggcatcctctgcaggccgagctcttcctccctgctgtgtctacaggcccaactcctgcctcacaacaacctccttggactcagcttctgcccagctcctggtggcctttgtaggctcaaaattttctcaaatcaagctctccaggcctactgtcagcctcgtggcagcctaaacaggcccagctcctgcctgacaatggcctctccaggcttttctcctgcctcgcagcaggctttccaggcccagctcttgcctcatggtggccttccccggccatgttcctatctgacttctggcagcctcaaccggcccagcttctgcctcacactggcctctctaggcccagctcctttttcacagtggcctcactaggcccatctcctacctcagatctgcctcccaagacccagctcctgtctcatggtggtctctcttacaccagctcctgcctcacaatggcctcgtctggcccatcttctgcctcacagtggccactcaaggcccatcttttgcctcatggtagcctcttctggttttgctcttgcctcacagttgcctcttccagatccagctttaagcctttgatggtcaacagcatcaaggagcctaaagcttccctggactctcatttgttcactttacagcagagtgccttagcaaaaactgtctcttaaccttgagagtggatttctgacaaatcgatagtaaattctgcctgtgtggtttc

SEQ ID NO: 216 - DNA, 407 bp, unknown
  >gi|944178|gb|R85772|sr|IMAGp998I12605.2|cl| IMAGp998I12605|R85772 yq23e06.s1 Soares retina N2b4HR Homo sapiens cDNA clone IMAGE274643 3′, mRNA sequence.
gacacaattccaatgtgtgaacatttcagttgtcatgtttcattcaacaacaccagtctcccaacaaactggctcaaatttcagttaccttggtattcagatgagtaactgcatctcagtttcttcagtccacatattacaatgtgaataacagatgtacattgtgatcagtgaccggtcacgtcacttccttcaaagtctgtcaatgattggtcattgcccttctgttatttggctcacacacagacagcaaaacatgtgttgccttcctgtctctcagcagtagacagatactatagaccaaataatttaaccattaaagcactacaactattgggataatgctcttgggtcaccctaattctcttgggggattgaaattaaaatgtgaaaatttgctttatacc

SEQ ID NO: 217 - DNA, 585 bp, unknown
  >gi|4373025|gb|AI479857|sr|IMAGp998F015343.1| cl|IMAGp998F015343|AI479857 tm70c01.x1 NCI_CGAP_Brn25 Homo sapiens cDNA clone IMAGE2163456 3′ similar to TRQ13231 Q13231 CHITOTRIOSIDASE PRECURSOR. ;, mRNA sequence.
cattcagtaactatttattggatgtctaatacatgcttagtatggtctctggtcatgcaacgaatgttaattcccctgtctctttgaaggaaggactaatggctggacactaatattaaaagagtcagtggtattcaataaaagggcttgggagagtagtgctcttgtcatgaaatagcacttcagtctggaaatcctctaagtgatttctgcactcagttcctgccgtagcgtctggatgagggggtatcggccctggttgcaggagaagccggcaaagtcatttaagtccagtgcccagaccatggccccgcccagncccttctgcttcagatagctgaccttggttttgaagctctccacatcatcaaagcccacccactggttgtcccggaagatgtagggcaccttctgatcctggattctctgtttggtggcccccttccaagagcagactatataataggccaacatcccttctttcttggtgaaggggcctggagggccacaccctgtgtgtggggccccctactcttggttcttatgaggcggcagggagaaggagccccccgaggtggcctcccc

SEQ ID NO: 218 - DNA, 534 bp, unknown
  >sr|IMAGp998B171824.3|cl|IMAGp998B171824|gb| BX115855|oid|234566085 BX115855 Soares_testis_NHT Homo sapiens cDNA clone IMAGp998B171824 ; IMAGE742576, mRNA sequence.
ttggcagacatgaggcaacgattgctcccgtccgtcaccagccttctccttgtggccctgctgtttccaggatcgtctcaagccagacatgtgaaccactcagccactgaggctctcggagaactcagggaaagagcccctgggcaaggcacaaacgggtttcagctgctacgccacgcagtgaaacgggacctcttaccaccgcgcaccccaccttaccaagggacaggccagcagcacagacaacgctgtggataaggagagtagagacttcctcttcctgcctcctgtcttctggagttgtgaccgcagggtggcccaggtggatcggcttcagaggccaggaggcagctctctgcagccgaagagcaggagcctcacccagggtctgtggctctgactaagcctggactgcctctcggctgtgctccgtggactggctcctcagggatccatgtgagagaccggagtatgatcctcagtgcgaggacaaataaaagtagtgattatgtccaccccaaaaaaaaaaaaaaa

SEQ ID NO: 219 - DNA, 741 bp, unknown
  >sr|IMAGp998L24923.2|cl|IMAGp998L24923|gb| BX096399|oid|234527172 BX096399 Soares pineal gland 3NbHPG Homo sapiens cDNA clone IMAGp998L24923 ; IMAGE396839, mRNA sequence.
ttagtccctaattctagaccttacccattttacacacactattagctaaatcgtgttcctgaggcatggctctgattactttactctggctttagccttggcctcatccgttaaattgaaactgttcaccagacctgtatgcattaaaaatttttttattagaaaaatcagtgtcttgctagccatgtatgatagctatcaaattgatcattctgtaaatggtagcaataagtgtatcaacactttgacctcctctgtgtctagtaacatttgcatatagtacaactgagcacatagcaccagaatattggaacagatgcttctccaaagccaagggttgccttacatttagagtgggaaaagaggaaaatgagttagcagaggagcagcggggaggggggtaaattacaggacataatttgcttctaaaaatttgtgtttcgtgttgtagttatataattaacttttctattgtactgattgacagatttggcacagacatcctctcattgtgaatatatatgaatatatcagagttttggactctggggtatatttacataaacacaagtattctgaaagcattgataaccacactggtgtctcttaattttgtctataagatgaccttataaaatccttcaagaattatttaaatcaagtgtacgaagctctgcagttttttgntatgtctgaaatattcattaaaaagnttattaaaacctgaaaaaaaaaaaaaaa

SEQ ID NO: 220 - DNA, 393 bp, unknown
  >sr|IMAGp998E135802.2|cl|IMAGp998E135802|gb| BX096812|oid|234527996 BX096812 NCI_CGAP_Lu24 Homo sapiens cDNA clone IMAGp998E135802 ; IMAGE2338548, mRNA sequence.
cccctcttcccaaagatgtggtgacttaagaggctctctaagcacactgttgactccaaaacatccgcaggtcagagccaggtgggaaggtggtccgtgcaggatgtgccaggccctgtggcaggtcttgccccatgagtccatatgccagtgggcagtttccagtacacaggtgatggcagccagagcgcggccccaagtgcaacacgttctttggttattggctcagaagcctggaaacaggagcttctgcggaccttcacgcacgtgttattcccaaaatgccactagatggcagcagagaagaacttttccctgatttaagcgctctgcgagcagccactcagcattacactaagaattcagacccctggtactcaggggtcttcgaga

SEQ ID NO: 221 - DNA, 620 bp, unknown
  >sr|IMAGp998D171153.1|cl|IMAGp998D171153|gb| BX098506|oid|234531385 BX098506 Soares_pregnant_uterus_NbHPU Homo sapiens cDNA clone IMAGp998D171153 ; IMAGE484960, mRNA sequence.
cgaagggaccctgggcgtctgaggccctgatccgggcgggccttcaggatgttagcagtccctgtccggttgaaggtaggaagccgaaaacccgagtgggggacgaacagacttacctcctgtcccgcaaaagatccactagatcgtcgtctccaaaacctgagagaccgcgaacgggttccagagccgcagcgttctctcagacctggcgttcaggaagattctagagagcacgggcaggtcccggaggtctcagacccgcaggcctgcggggcgggggcggggtggcgcccaggacgactccccgggctcagcttggctgcctgcctccttctcaaacaatgagtacctactgtgtgtagaagcagcaggacagcctgtcatcactgggaaagaggccagaagagccgtttggccagggtctccaatttaggctttcaacattatctctaaagaaggttatacattatgtcggctccactggctgatgggatcaaagaccctccttgtatatccttaaacctgtactctattaccaggccactgtgcctgtggagcactgtaagttcctaccttttaataaattttataattatcttgtgagcaaaaaaaaaaaaaaa

SEQ ID NO: 222 - DNA, 696 bp, unknown
  >sr|IMAGp998F17593.1|cl|IMAGp998F17593|gb| BX117945|oid|234570266 BX117945 Soares melanocyte 2NbHM Homo sapiens cDNA clone IMAGp998F17593 ; IMAGE269968, mRNA sequence.
ctactgtaccctggcctgggaacacattgccctccccacacaaaactggagggcttggcttctgcgtgtgagaaatcaacatttttaaagcacttgccttctaccaaccccagcttgcaatcactgggccttcccctcctatccaaggggttggaggggccccttggctctccttttggcaggaggagcctgcttcattacaccaatgactctgccatccccctccctggccctagaccccaaacacatctccctctacccaatttactcttctcgccccacctagggacagattccccctgctctttttgtcctagaaaccccgctagtttgggatggtagcgtctggggtggggagggcttccccttccccactcgagggtgcgggtggggaagggggggtggggtggagacagccctggggcagggaggatggtctctccactgtagaaagtagagtaggattgtggtcagacttaatttgaggcatctagtgaagacacgtacaaatccaccaaggaaaaagatttcaaaagcaaaataaaagcgggaaataaaacagacccaagaataatcaagtcaaagtgatgttgcacaaaatgcagagaaaccaagaagggggagggttaatgtattaaatgtgctattaagaacttaattttattaaaagtactattacttaaaaaaaaaaaaaaa

SEQ ID NO: 223 - DNA, 215 bp, unknown
  >gi|917103|sr|IMAGp998N11360|cl| IMAGp998N11360|gb|H41051|H41051 yp52c06.s1 Homo sapiens cDNA clone 191050 3′.
tccaacccgcggggancaggtgtccccggactgggntnctgttnggaagatggncgaccccgggcatgagctggcagcagcactattacggcggctcgcggccaaattcgcgccctcgccggnccaccgcacagctggctgggcacagcattgactacagccaggagatncacctgaaaataagcaagaaaatcgcccagctcaaccaagnttaa

SEQ ID NO: 224 - DNA, 473 bp, unknown
  >sr|IMAGp998I152328.2|cl|IMAGp998I152328|gb| BX095210|oid|234524789 BX095210 NCI_CGAP_Co3 Homo sapiens cDNA clone IMAGp998I152328 ; IMAGE936278, mRNA sequence.
atgacctctttagactcagctcattttcactgctacatcttcaagccattctcctgcctcttggcaacctctagtggcccagcttctgcctcacagcagcctctccatgcatgcctagctcctgcctctttagggaacttacaggcctaaaactttcttaatttgggcttctcaggcccagctcctgccttctgttggcctctacaggcctggcatcatcctttcaacagcctctttaggcccggcctctccaggaccaaaacatccttaagtcaacctcaccaggcccggctcctgtctccttgcggcctccagaggccgagcttttgcctgccaatggcctctctagccccagcttttgcctgccaatggcctctctagccccagctcctgcctctcagcaccctctttaggcccagctcctgccttaataaatttgaataaattattgttatgtgaaaaaaaaaaaaaaa

SEQ ID NO: 225 - DNA, 279 bp, unknown
  >sr|IMAGp998I112449.2|cl|IMAGp998I112449|gb| BX105875|oid|234546131 BX105875 NCI_CGAP_Pr12 Homo sapiens cDNA clone IMAGp998I112449 ; IMAGE982738, mRNA sequence
aattcgcggccgcgtcgactttttttttttttatcgttttgttttaaatataaagagctattttagctctcttgaatctaaaaaagcaacttaagtttgtcggttactgctattgggattaccttatgaaatcataacataatgttgtttttaaatatgaaatcattttcctacgaatttcccaatttgtgtttcataaacattccagtaagagaaaatgattgattacaaagcagaatatcaaaggtttttaataaattgtcgacgcggccgcgaatt

SEQ ID NO: 226 - DNA, 547 bp, unknown
  >sr|IMAGp998A17282.1|cl|IMAGp998A17282|gb| BX091082|oid|234516531 BX091082 Soares infant brain 1NIB Homo sapiens cDNA clone IMAGp998A17282 ; IMAGE50008, mRNA sequence.
ttttttttttttttaacacttcttaaatataaagttttgcaacaaacctttgatttatatcaaatataattaaaaattaacaaatgaatacattgtatcaaaaagtacgaagatatacattatcatgcacatacttgtatatatgtatatatgtatatatgttacatgcacacataacttgttcaaactacagccactgcttgactggttccagatgcagacaactgggtgatgcacttcaaatttggcagctcactatgagattcaggggacatatatgatagccggagaagacacagtgagaaacaaaacccaaagtcaggaaaacaagacataatgtctcctttccccgctccctgtagttttcatcaattccaatgcctagaagagtctagacaagccagctaggctgtgtgggtttagaaaagtggattctcgacccaacactgcctggggaaatggctgtggttctgcatggaatggacacattcgacacccaagataacatgggctgggggctggggaacaagagacaaaacgtcagtgt

SEQ ID NO: 227 - DNA, 328 bp, unknown
  >gi|3246373|gb|AI026885|sr|IMAGp998J014173.1| cl|IMAGp998J014173|AI026885 ov92c01.x1 Soares_testis_NHT Homo sapiens cDNA clone IMAGE1644768 3′, mRNA sequence.
ttttgtattcactagacttttatttcgttccagggcttatgacagagcccaagagagtcagagaagctttctacaaattcagctgtcgcattcattttcttacacactagagttgccacttaaaaatactaaaataaaataaaaaattaatgttgaccatctaacaggaatacttgactcagctactaagttacaaatttgcttcatttttggaagacatctagcatctgagaattggacttttcccagaagttcacttggtaacctcaggtaagtcctggttcatcctggagaggatccgaggggaaagcctgatgggacccacggc

SEQ ID NO: 228 - DNA, 262 bp, unknown
  >gi|4892849|gb|AI682667|sr|IMAGp998O235762.1| cl|IMAGp998O235762|AI682667 wc64e12.x1 NCI_CGAP_Pan1 Homo sapiens cDNA clone IMAGE2323438 3′, mRNA sequence.
tttttttttttttttttttttttttttttttttttttttttttttttttaaacaattaaaaaatttatgggcttccgggggccccccatcagcgggcaaaaaggcccctttaagcaaaaggttttcagttttcccgggccccatttggtgaacaggaacccatccctggggaaaattctttttaaaaaaactgggcaaaaaccccttgggccgggaagggggatttttttttttacttttccttccccccattttttccttt

SEQ ID NO: 229 - DNA, 477 bp, unknown
  >sr|IMAGp998C01743.1|cl|IMAGp998C01743|gb| BX099107|oid|234532590 BX099107 Soares_fetal_heart_NbHH19W Homo sapiens cDNA clone IMAGp998C01743 ; IMAGE327480, mRNA sequence.
gcaaggctagaaagatgctacgttgggccttcagtcacctgatcagcagagagactctcagctgtggtactgccctgtaagaacctgcccccgcaaaactctggagtccctgggacacaccctatccaagaagacccaggggtggaacagcggctgctgttgctcctggcctcatcagcctccaaactcaaccacaaccagctgcctctgcagttggacaagacttggcccccggacaagactcgcccagcacttgcggctgggcccggggagcagtgagtggaaatcccccacgagggtctagctctaccacattcaggaggcctcaggaggccagcctgccatgagagcacatgtcctctggccaggagtagtggctgagctctgtgatcgctgtgatgtggacccagcctccagggagcagagtgtcggggatggaggggcccagcctggactgactgctacttccngtctctg

SEQ ID NO: 230 - DNA, 468 bp, unknown
  >gi|4081545|gb|AI344339|sr|IMAGp998D175081.1| cl|IMAGp998D175081|AI344339 tc03g09.x1 NCI_CGAP_Co16 Homo sapiens cDNA clone IMAGE2062816 3′ similar to contains TAR1.t3 TAR1 repetitive element ;, mRNA sequence.
gcggccgccaaactccgtaaggatggcgtcggccgcccggcctgcccctccctgggccccgctgccctgcacctccgccttcagccataggcgctggaggttgtgctgacgctcgaaggtccagaccagggcctcctcgcgactgcgggcaaaggtgcaccggtttcggtggcgccggcagccgagcccagggctgtagtagtggcagacttggtagcacaggggccttgggaaggtgggccggcagcccaccacgcgccagaccttgctcttggtggcctgcttaaagcgggccagcaggatctcgcgggagcagtcatgctccaccctacggaggacgtaggtgctttcattgagccgctgggtgcaacgggaggcagcccagacccaggtccaccagggcacacagccgggccagggacgggcctctggtggccgcggggctgttgggcagcagggtggaccctg

SEQ ID NO: 231 - DNA, 367 bp, unknown
  >sr|IMAGp998B2474.1|cl|IMAGp998B2474|gb| BX089396|oid|234513140 BX089396 Soares infant brain 1NIB Homo sapiens cDNA clone IMAGp998B2474 ; IMAGE23877, mRNA sequence.
ttttttttttttttaagtaatggtaaagtttatttcatttttaataacaattagnaggacaaaatgtttaaaatttgcagtttaaaacatgcacattcacaaaaggccaatgacacataacactgcatagaaataatattactcaattttaataactataaaacacagtgcatcaaacatcaagataaacaaacctgagaaaactactataaccacagattcaatactctccactcatgcagcttcacaatttctacagcagtttcagcaggaatggtttttcagggagctgaaaatactactttatctttaacgcaaaactgcagttttctgtagtagctgccttccaaggctgccctgtcttttc

SEQ ID NO: 232 - DNA, 498 bp, unknown
  >sr|IMAGp998I094494.2|cl|IMAGp998I094494|gb| BX095909|oid|234526189 BX095909 Soares_testis_NHT Homo sapiens cDNA clone IMAGp998I094494 ; IMAGE1837520, mRNA sequence
gcaacctctagtggcccagcttctgcctcacagcagcctctccatgcatgcctagctcctgcctctttagggaacttacaggcctaaaactttcttaatttgggcttctcaggcccagctcctgccttctgttggcctctacaggcctggcatcatcctttcaacagcctctttaggcccggcctctccaggaccaaaacatccttaagtcaacctcaccaggcccggctcctgtctccttgcggcctccagaggccgagcttttgcctgccaatggcctctctagccccagcttttgcctgccaatggcctctctagccccagcttctgcctttcatcggtctctccaggcttagctcctttctcttcacggcctctgcaggcctaaaacttcctcaatttggcatctccaggcccagctcctgcctccaggccgcctctgcaggcctatctcaagccttacaacagcctctttacccccagctcctttctccgact

SEQ ID NO: 233 - DNA, 591 bp, unknown
  >sr|IMAGp998B08733.1|cl|IMAGp998B08733|gb| BX102114|oid|234538608 BX102114 Soares_senescent_fibroblasts_NbHSF Homo sapiens cDNA clone IMAGp998B08733 ; IMAGE323623, mRNA sequence.
ataactgatacttagctaacaattgtccataacttgtttaagtgtaagaaataccatttcaggaatgaaaaagaaggaatactactttctaagaaagaagatcttataatagacatatttagtaggttaaactactcctttgaaagaataagttttggttcaaagtcaataatgaagacgagatttgtttttctcttctctggctgatttttaaggatattatgtagttcatttagttaacagaattgaaatgtttgatatggcaagataggtatggtaatttcaaagtgaattgggaattcctctggctcatagaacccttttttttttcctttaagtattcttgagatacaaaaaaaaaaagtaaataaaatttcaaaaaaaaagttccggatctgtttttaagctccatctggtcctcataacctgcaagatttttcttaaaacctttcagctgaaagngggggtaaaggtggagtaanctgtggattcgcttctgttgtcttttaaaangtcaaatatataacatgtaatttttttaaaaaccaccagatacanaaatgngctttaacatcagcctgaaacctaaat

SEQ ID NO: 234 - DNA, 8483 bp, unknown
  >Homo sapiens|chromosomeNCBI3441552793691552878511
ataggtaaaacaggatgtaaagtttatatacaagaatataatgtttatctgaaatatttacagtgttggttaaagcaatatttttacaacttttaaaggtaaactactatgtatattacaggtaagctacaatgggtttaatttgcaaaagttaagtaagaaatgttttaaacaaggcttaaagtactcaagtcaattataaaatttatatcttttgccttttacttgaagaaatcatgctatagaaatggttaatgtgcttctaataaatggaagtattgtagctggaatgtgatacatgtaacagtttaagttcccattgaaggtataaaatgatgaattgttgtaagacttagacactgagtctcagtctggagctgatgaagatgttgagataacagccagctttatctcaacagggtttgtgacccacaagtttgggccacagagaaaattgaagcaatttgcatgttatgacaacctcagtgggaagtgaaaatcagctgactcaaaacaacaaacaacaaccaaccagacccaagtcacagttgcacctattcaaaactagctttaaagtgagctatttttaaacttcataaaaatattcatgattttattagtttgaatatttctacaagattcgggtgggcttttcctttaggtgaaaacagctatccactcctgtggccttataactcaggaaatgctggggatgcaaacgtgcaaaaggcagggggaagctgcccaggctgagactggagcagctaggagtgtgcttggggaacgggagctgagatcccggagcagaaatggtcagccgtgctctggagcaggcctgtcggagccatcaggatgccgggactagcactgcagcttgcggatgctgcgggagatgcgcttgaactctctctgccccttctgccaccgcttcaccgaggtgatcaccagctccccaccctgtttctgtcccatgaccagatagggcgcgttgatgtcgttcatctcctcacaggtgcactgcaagctgtctttgagccacagcaccgatttcttcaggtccctttcggacacaccgttcagcttgtaaatggtcttgctcttggtctccaggatgattttggtatctcggttgatgtaggttatctccttcacttttattttcagtgctatgagggagggcagaaagagtcatgccagttataatgagggaatacagtatacctccccccattctgccaactcgtgtgacttggcctggatgaggaaagagaggcttcctagagctaatgggaccctcagaggctgcagatcgggggttgggagctaggggcagcaggtggctccactgctaaagcctcaaagcatttgccaggcacttggtttccaatcagccattttttcaaaactctctctttgcgggactcaattaacttttcttctcctttcctcactcacccttgtgtttgtaagaaagttgcatgtggtgtgtgctctctccaacaaatgacttcctctgttggaaacttaagcatgaaaagaaatgcctgacttttaggtgaaatatgatccctctgtggccgtaaagtttaaactcaatttattatgcaagaggtttttggacaaacctgtaaggaaactgtgtcacgcaatcattgttttctaaagatcagattacagggaaggagaattgatcttacagctgcatttgatttggtaaagcaggagaaacaatatgcaagtctctttttaacagcacaacgtggttagtcataaaagtgtttttctgaagacctcatccttatatgctctcatttgccagtccaaggccaaggcgtttacggatcatatatgtactgtttccaaaatgtatatgtagactcgttagaatgcgaagaccaaaagtaagtggtttactataaaggacacgttttatagtcatgaacagtatgctctgggaaagaaagccctacaaacgtgtatgctggaattaaatttcaagactccatgaaagcaggaaaagaatagaattttaaggttagaagggatcatcccattaaaattgcttgttaatacacaaagaatcttaaatccagagaagtttagtgacttgcatgaggtcacccagtttaggttgtttgctagaactccagttgccaaacctcctcctgtaataccactgtggtgtcttgaggcaatgtccatgtttttgtagtggttgcagcataaacttgcccagctgcaatgtgtcctaagaccctgtgtgcccctgacagctattctgtatagatgatgtgtaagtatgacttcctgactttgatggagtttaatctatgtttaaattagtaatagaggaagatttttctgtgttgcgtacaactgtttttgagattttcttttttttttttttaaatatcagaatgggaatttgactgagtatggctttctagtcaaccaggaaattatagaatctctagcaggtaaatcaaaaatgctatgacttggccaaccatggttttgtctggtatgatatgcatctgaactgtggtactgaattctcagtttttgagactcaagtcgtggaataagccatacttgtggaaatgtttcatgcttttgtgtcctctgcattgggagtagggtgttggatagacctcaccatggtgggagaaatggactgattacactttacctgggagccttgtctgtagtacaggtagggcatatataagatttcacctggccaaatgaaacattctcaagctccaaacatcagaaaaaaccatcacttttaaccaggaggaagattttttagtcatctttgtctagctctaaaataactaattttgcaaatgtttatcatgagaaaacattagggattttctccctggattttcagctaggcccaaacagagagaattgaatcccccacataacctttcctgtcctggaggatgtgactgaggatgcaggatgtgatcgcaggcagaggtgatgatactttctggtcctacaataggttgtatatataggaaggtagttctgaaccattttttattcaccagtacttgcaccactgaatttcccttgtacttcaggagcacagtagggaagaggtgatgccttcccagagtagtcaagtcaaataaatcagatttctatgcttgttcctcctacttttccattattcacaaagaacagaggcaagatggaatagtgcttataaaccaataatcttatgatgcagttcttaaaattctgcttcattatccctctatgggtcaaaaagagtgaagaaatagaaaacaaagcttgtgaggtatctatgcatgtgataccttaagtgtgataacttctgtgttgttttgaaaggctttctctctgtaaggagggagcacactaaggcacacaggcatccatgttttggctcatgtcttgatagggcatccttagaaagggggtcttctgtaaggtcagccacaaagcaagcaagtaataggtgtgtgtgcatgtttcccaaagccaattgttatggagctccacagtgtaacaatcaacttttcatggaagtcaggaaggatctgttttatgtgcagtgcaaaggatataaactgccagaagaacatgatattttataggctttttaatagaaattttataaaatgatatttttctatcttgcccttaatgaagtgatgtagcatgggggctgagaactgtgttttggtaaggctctggtgatttggaattatctgctgctaacatgcctggctcttagccagtttgagggttgtatacatttgaaaacaggcatatgaaaccccgtcagccccatttgtggctcctcctgaggggaaaagttgctaaggactttgagctacctcccttctttctctttcctcagcagtgtgaagcctttcccaacaccgggactatggtagttctgggaccagtcagagttaaacccaggacactttctggaagagacctggagttgtcaaagagcagggattggatggttccagagttttagggacctgaccaggcctgagatttctgggatggcaaggtaaggagagtgagagctggagtgatttccctcagtctcgaggactatgttgaatatgtagaagtgttaatatgggtctattagactccattattgattgacagagcagggaacatatctgcttatgagtaagttcagccttgtgcctgaataaatacttttttaaaaggtaccaataattccaacacaccaaaaggtacctgcccttggaaaaggataaaagcaagaatgcctctgggtgaagttcctggtcattcccaactctgaatttagcattgatcctgtgtctagctgggctcctcttctgacctgagagccctgaagtcagcagatgaggtgctggtagagttccatgtagacaaagccccagctctctttccccttataatcctgtcttcaggggagtgacttgcaacttatggaacatatcacatgtgttttttattgtccccaaacaccatgtgatctatcatgtatcttgctaaagtaaatttctaagctttttttttcctttttaaaactattttcgaaactgtggcaaatgtgtctagacttagtcatacaaaatttacaaaaaccttttaaaagggattttaagatggtgttttaaggttgttaggtgttccatgttaggtgtcttctcagtaagccactcttctttttcaacttaaggaacccctccttgacaaattattaatcagtgccttggatatattcactaatggtagaacacagaggtttaaaatcattatgggttataaagaaagtaaaaatatttttatttgtggccaatttaaagccttgaatctgtttggctggggctaatatttacacttccatccctggtgcacatgttaagtaatcaaaagaaaatagaactcgggattatgcctgtcggcaaaaaaaaaaaaaaaaaaaaagggatttaaaatgtgccactggaaattgataatctgaataattttcattatttgattattggcttaacagaacaatgagtctcacctaacaagatacaactcaatgctagacagttacataaggaggaaactaggatagatcaagttagatgattttgggatacatgagctataatggtgttttacccaacctttatctagcaacatcataatgatttgcaccttgaagttttactggtaaataaaacttctgaatgtgaaatctgttgttgttgttgtattacttaccaaaatcatttttacaaagcgtttccattatgtcgttgtcatcatcatttttatttttgcaggcttcacatacctttggagctagaaatgtgaaaaacagtctttagtaagtttgcttaaaagagaacagtaggtggcgtttgtaaacaactcaaagggacttatccagacaaaagtagcaattgtcctatttaacctcagcttctgcctcctctataaaagcaggaacactcaatcctaagaaagttttcccatgcttcccaatttttttgggggggaggggtggtgggagcaaaatgagaatgcctatatgttgttgtgtttaataaattatgactctcaaaagggagttgtgggcagagtatacaagtgtatgtagttatagttaactgagacagggtgcagcttttcagctcatcctcccaacctctgggaaattcagtctgaaaaagttattcccaaggccttttgaaggagaaagcacccaaagaaccaaagatacttgaggaatttgggaaaactggtaactagaaaatatagacaaaagattgtctttctcagccacaggacaccagtgtagttggctctactcggcagagctctgtatctcaggggggccaagtctggactgggaggggtgactggctagcgttccacatgtgggctgtttccaccttgaaaacacatctcactaattacaacagcctgctctaagtggttctgtgtcaccttgtgcctctgccaagattgtgcttgagttaggcagcattcatttcattgttatctgcaagctccctggtacaggttcttcttgcccttctgtgcctgtttcaaactctcaaaacaaacaaataaataaacctgtttctgaagctaatcaaagacgaagcaaaaatggtttagtggtctgatcaaacttagggattgtatactagcacaattatcagaagacctgctaaaagctcttctaagagtttgatttcttgggagaagtaaaaaaaaaaaaaaaagaacaattatattgcccttctctcatttataaatgtgttaattatgtctttaaaatttaggtgaaatgagattttcactaatgtgaaatttaatgttcatagaaaactatgtgacttaatagaagcgttatttatcaaattagaaaaatcacaaagctggaattttgaagaacataaacttttattttgaaggagaaaagtggctttttggagaaatgtaagatgcgtaatgttgctgctgatagtgggaagaaataaaagcaacatacataaaactgcccttaagtcccctcacccaaacaaaatcaaacatttccaattaggagctctcaggatgtatataaaatattactaacctgtattggttggtctatggtacatgttggacaaatgtttgaaaaatgccaaacatttgcaataaattagaaatctaaccaataagatgattgcaaactcatggtccctgccaggaaagattgctaaagaactttgcatttgctttaactaatgatgtctgtaggaaaactactagagattgaaagggagaggctgctgaagccctgaaagggattaggaagctcccttttccaacccaggggacccaaatcagctcagaggcattgtatatgttttcctttcagaaaaaatattgagagcccatgaggttttcattgctttgatggtttttgcacaggaactctccctccgttttctaaaaacttgggaatctcttttgcatgattttctccagctaatcaaaaccactcctaagttaaaaactgatagcaatagaaaagtgatttccagagattccctttatgccctcttcctcaagtatctgagcagttacaaccaatgttcaattttccacacactccgtgccagtcccggttttatttccaaaatctaaattgacaaaggacaaaaagacccttaaaaggttatctacttgtgcatcctggttttcctttctgtacaggaaatgttcctgtgactagaatcctgcccagtggtaggaaggtgcagaatgaggctgagcgaggggcctctggccagaatgctcaccacaaactcctccaccaaacaccacttccaacgctctgcgcagctccagtgctagcatctttaacaataatcgtgtatctatttttttttaaaccgccgttcagttcttcgtttttttagttttgttgtttttgtttgtttgttttatttttaaactgcccttaagtatagctggtacaactcgggaggagaatgcgcgagacttgggcggcgtgggaggggtgcctcaaatttgatacaggcttgttgtgatgacagaccaggtcaggcagaacttctgcccttcccgctactggcaccccaagcagggatgcactgggatgcgtggcaggggcgggatctcctgggagcgtctcagcccagcagggagtggggaagcaagagggaaggcttaccttcctcggtggctggcaggaggtggtcgctgctagcgagggggatgcaaaggtcgttgtcctgggggaaacggtcgcactcaagcatgtcgggccaggggaagccgaaggcggacatgaccggggcgcagcggtccttcacctgcacgcagagcgagtggcatggctggatggtctcgtctaggtcatcgaggcagacgggggcgaagagcgagcacaggaacttcttggtgtccgggtggcactgcttcatgaccagcgggatccaagcgccggcctgctccagcacctccttcatggtctcgtggcccagcaggttgggcagccgcatgttctggtattcgatgccgtggcacagctgcaggttggcagggatgggcttgcaattgctgcgcttgtaggagaagtcgggctggccaaagaggaagagcccgcgcgccgagcccaggcagcagtgcgaggcgaggaagagcagcagcagcgagccagggccctgcagcatcgtgggcgcgcgaccccgagggggcagagggagcggagccggggaagggcgaggcggccggagttcgagcttgtcccgggcccgctctcttcgctgggtgcgactcggggccccgaaaagctggcagccggcggctggggcgcggagaagcgggacaccgggaggacagcgcgggcgaggcgctgcaagcccgcgcgcagctccggggggctccgacccgggggagcagaatgagcc

SEQ ID NO: 235 - DNA, 153 bp, unknown
  >gi|2714120|gb|AA704202|sr|IMAGp998K211090.1| cl|IMAGp998K211090|AA704202 zj77e11.s1 Soares_fetal_liver_spleen_1NFLS_S1 Homo sapiens cDNA clone IMAGE460940 3′, mRNA sequence.
aaatacttttactgggatactcagtggtagaaaatggtacttggacagttgaactgatttttagcagtaaccaagatctgtcagctctgagcttggggaacatcattctgtttggtacccaaagcaacaagtgactattgagcaggggttacc

SEQ ID NO: 236 - DNA, 382 bp, unknown
  >gi|2186431|gb|AA461311|sr|IMAGp998A181964.1| cl|IMAGp998A181964|AA461311 zx65b09.s1 Soares_total_fetus_Nb2HF8_9w Homo sapiens cDNA clone IMAGE796313 3′, mRNA sequence.
ttacaattttactctttcttagcaccaactccatcatgcaagactgacagggaagaatcacaacatgtaaaagctaaaagaaaccccttatacagtccagtgcttacaacctttaaaacacattaaaagcttgaggaatttgttaacatgcaaattcccaggcctaaccacagagacactgatttagatgatatggggtagatcccagtaaagcagcccaggtgtttttgaaatagatggatcatgaaccaaactttaggaaataacaatatactgcctcacttttagattaagaaatttaaactcagagagcttagtgattttcctgaggccatgtaattagctaggggcaaagaagacattagactagtagcctcctatc

SEQ ID NO: 237 - DNA, 672 bp, unknown
  >sr|IMAGp998H04617.1|cl|IMAGp998H04617|gb| BX100150|oid|234534676 BX100150 Soares_multiple_sclerosis_2NbHMSP Homo sapiens cDNA clone IMAGp998H04617 ; IMAGE279219, mRNA sequence.
atacctgatacgcgcttctgggctttgtctgtggctgccaggtggtcagcgtgtttacggaggaagaggacagctttgatttcattggtggatttgatccatttcctctctaccatgtcaatgaaaagccatccagtctcttgtccaagcaggtgtacttgcctgcgtaagtgaggaaacagctgatcctgctcctgtggcctccagcctcagcgaccgaccagtgacaatgacaggagctcccaggccttgggacgcgcccccacccagcaccccccaggcggccggcagcacctgccctgggttctaagtactggacaccagccagggcggcagggcagtgccacggctggctgcagcgtcaagagagtttgtaatttcctttctcttaaaaaaaaaaaagaaaagaaaacatacaaaagaaaaggcaaaaccccacatgcccacctcctctggcaacatgggggtcacagctctgcccccaggctgtcgtctcgtcgaggagcccctccctcaggtgcccacctggggctgctggaccctcgggctgcaagcactgctgctgggatgcagccctccccaggaagncaangcgaggcccganacccctcaagcggngangncccctgntgaacatggagggnntcctaacccccaaacncnngccanaan

SEQ ID NO: 238 - DNA, 551 bp, unknown
  >sr|IMAGp998I12670.3|cl|IMAGp998I12670|gb| BX089595|oid|234513538 BX089595 Soares_fetal_lung_NbHL19W Homo sapiens cDNA clone IMAGp998I12670 ; IMAGE299603, mRNA sequence
gaggagaagaaatagaagaaagttctgcaacaaccatgtctgagaacttccagtattaatgtcagacaccaaaccaaagatccaggaagctccgagaacaccaggcagaataaatgccaacaacctacacttggacatataattttcaaactatatgaaataaaagataaaggaaaactctgaaagaaaccagaggtggggcagaaaacaccttacctacagagacacaaagataagaactgcattcaacattgcagaaactgtgaaagcaagaagacagtgaaatgaaaaattcaaaatgttgacagaaaaaaacccaccaacctaagtttctgtacccactgaaaacacccttcaaaagtgaaggagaattaaggccttcctcagaaaaataaaaattcaagaaacttgttgccaggagacctgtcttgcaagaaattttaaatgaaattctttagggaaacaaaagatatgtaactgaaacctggatcaacatttttttaaaaagagcattaaagaaagaattgtggtacaataaaaaaaaaaaaaaa

SEQ ID NO: 239 - DNA, 239 bp, unknown
  >gi|5445722|gb|AI825133|sr|IMAGp998D145711.1| cl|IMAGp998D145711|AI825133 wb04h07.x1 NCI_CGAP_GC6 Homo sapiens cDNA clone IMAGE2304733 3′, mRNA sequence.
ttttttttttttttttttttttttttttttttcaaaaatggaaaaattttatgttttttatataaccattccctagggttaacagattttatttttatgatacatatctttaagtgggggcccactggggacatagcaaatttgcaataaatccttattaagctgaagggaactaacaaaaaccattaattaccccttttttaacacccaaaccagttttggtttttaagggtttgggg

SEQ ID NO: 240 - DNA, 525 bp, unknown
  >sr|IMAGp998L06178.1|cl|IMAGp998L06178|gb| BX113044|oid|234560465 BX113044 Soares infant brain 1NIB Homo sapiens cDNA clone IMAGp998L06178 ; IMAGE38015, mRNA sequence.
nacaacactgagnactcacgataaaacagacggcncatgtctacaactatatccccccacctgtgggtcagatgtgccaaaaccccatctacatgcagaaggaaggagacccagtagcctattaccgaaacctgcaagagttcagctatagcaacctggaggagaaaaaagaagagccagccacacctgcttacacaataagtgccactgagctgctagaaaagcaggccacaccaagagagcctgagctgctgtattaaaatattgctgagcgagtcaaggaacttcccagcgcaggcctagtccactataacttttgtaccttacctaaaaggcagtttgccccttcctatgaatctcgacgccaaaaccaagacagaatcaataaaaccgttttatatggaactcccaggaaatgctttgtggggcagtcaaaacccaaccaccctttactgcaagctaagccgcaatcagaaccggactacctcgaagttctggaaaaacaaactgcaatcagtcagctgt

SEQ ID NO: 241 - DNA, 364 bp, unknown
  >sr|IMAGp998I135799.2|cl|IMAGp998I135799|gb| BX100717|oid|234535810 BX100717 NCI_CGAP_Lu24 Homo sapiens cDNA clone IMAGp998I135799 ; IMAGE2337492, mRNA sequence.
tttttttttacatcgacagttgtattagagaaaagcttaaagacaaggaaaaattaaggaaatgaggcacaaaacgtagagtagatacaaccaacaaaaccaaattggattctttgagaagattagtacacttgagaaagccctggtacactagtcagagaaaagagagcagggacaaattacaatgtcagaatctacgaacccttcagacataaaaacttgaggctgtctcaaaagtcagtgggaaacgttaaatgcaattgacaaattactaggaaaaaactgctatcagaagaaatagaacatgttactagctcctgtctttcagaaatcttacctatatcttgtcaaaaaaaaaaaaaaa

SEQ ID NO: 242 - DNA, 493 bp, unknown
  >sr|IMAGp998D154762.2|cl|IMAGp998D154762|gb| BX103241|oid|234540862 BX103241 Soares_total_fetus_Nb2HF8_9w Homo sapiens cDNA clone IMAGp998D154762 ; IMAGE1940318, mRNA sequence.
aggtttttccagaattagtctggatcagtgcctaacaaagctgttattaattctccacacatgagtctggctaactgggccagctctcatgttttactcctgtctggctacaaatagcactgtgcatagtagccctgtttccccatggtgttggactgtggtaaagtatgttgtgacatactggaattctaataatcctgttgtaagtatgtacagaatctatgtaacttattgttgacatacagaggtttgggaagtagtctaatggacattccttacaatagaatggctgggaagggaaagaaaaaaaaaaaaaaactacctaactccaatcttaatgttgtagttttatagcaaacaaaaattgtcagctttttgtattgaaaggtcagtggtggtaagacaaggtgtcttgtaaattaagattttaagaagtgcattaaaatgttttagaattactctgggaattctgtatatcacactaaaattttta

SEQ ID NO: 243 - DNA, 751 bp, unknown
  >sr|IMAGp998P20366.1|cl|IMAGp998P20366|gb| BX097677|oid|234529727 BX097677 Soares fetal liver spleen 1NFLS Homo sapiens cDNA clone IMAGp998P20366 ; IMAGE193411, mRNA sequence.
caccctgcagccccagcactgcctggctccacgtgcctcctggtctcagtatggcgctgtcctgggttcttacagtcctgagcctcctacctctgctggaagcccagatcccattgtgtgccaacctagtaccggtgcccatcaccaacgccaccctggaccggatcactggcaagtggttttatatcgcatcggcctttcgaaacgaggagtacaataagtcggttcaggagatccaagcaaccttcttttactttacccccaacaagacagaggacacgatctttctcagagagtaccagacccgccagaaccagtgcttctataactccagttacctgaatgtccagcgggagaatgggaccgtctccagatacgagggaggccgagaacatgttgctcacctgctgttccttagggacaccaagaccttgatgtttggttcctacctggacgatgagaagaactgggggctgtctttctatgctgacaagccagagacgaccaaggagcaactgggagagttctacgaagctctcgactgcttgtgcattcccaggtcagatgtcatgtacaccgactggaaaaaggataagtgtgagccactggagaagcagcacgagaaggagaggaaacaggaggagggggaatcctagcaggacacagccttggatcaggacagagacttgggggccatcctgcccctccaacccgacatgtgtacctcagctttttccctcacttgcatcaa

SEQ ID NO: 244 - DNA, 276 bp, unknown
  >gi|3785069|gb|AI216028|sr|IMAGp998C034512.1| cl|IMAGp998C034512|AI216028 qh10e02.x1 Soares_NFL_T_GBC_S1 Homo sapiens cDNA clone IMAGE1844282 3′, mRNA sequence.
tttgtgtgatcgcacttatttaacatttattttgagttagggtgtgagtgctggggcaggtgactaaccatcccctgccctctgtggtggcctgagagagaagcaggaatgccccgtgcaggttggggatcagcaaacagtctagaccagcttctctcctgtatcccgtttggagagtccctgggtcaggggccagtccatcctgcagcattgaagggcgtgaggaatgagcagggctgtggggctcacagagcaggcagctctggggagaagcgg

SEQ ID NO: 245 - DNA, 468 bp, unknown
  >sr|IMAGp998G01115.1|cl|IMAGp998G01115|gb| BX111632|oid|234557641 BX111632 Soares fetal liver spleen 1NFLS Homo sapiens cDNA clone IMAGp998G01115 ; IMAGE122136, mRNA sequence.
acagttctccctgcccctagtatgcagcagcatttgtttccccttggtaatacacccgagtcagtaaaccccttatttgcttgttggttcagtgcccagaagggcccaaaacagcaggtgagcctcaatgtacaattcagtgagctgttgtcttgtgttccccggaagcattcctcctgtaaaaaccaagccctccaaaccagtagaactccagttgaggggacaggtgagaggaaccactcctacttctatcccttgattcctggacccgtgaatcctggttataggagagacaacacatgcaaagcagaaccttgggggcagcacccaaaagttgtctcccaagtggtgctgtaacggagtcttcacgaggccgtttcactctaatgtttgtccagctgcttcctcctgctgagatacgtggtatgactgtgattacctggtcacaagcccattgcctcactttct

SEQ ID NO: 246 - DNA, 752 bp, unknown
  >sr|IMAGp998B22787.1|cl|IMAGp998B22787|gb| BX109149|oid|234552677 BX109149 Soares_fetal_heart_NbHH19W Homo sapiens cDNA clone IMAGp998B22787 ; IMAGE344373, mRNA sequence.
aggctagtggctggacctgcactcagatggcaattcaggtccagttcatgagatcttggactgttttgacaaatgatttggggaaagggatagggagagagttgtcagtaccgatgctgagactgtgagagccatttctgagtggaggtgaggaaaaacggagcgatgaccgtgagggtctttgcgtggcgcctcgagaccacacctgggaacggtgactttggaactggctgacgtgtttatttggtgtgaggtcaactacagctctgagggcaataacatgatcctcataaaacagaccccccaggctgggcctcagcccgcttgctgccaagcaggggtggtggcctaataagtgagacattgctatcaactctctgtaaatggtgggttcattcactctgtggatgacatagcctgttctttgcccttctgtgtcagccccattgattgctttttaaccaccttcttgctttgtgctgcttctttcagatgaaaaggaagggaaaagagacttcatttccatacccactaaccatgccaacagaaggctttgtgaagaaggaagtaaaagctatcggtaggagttagaaagaaacatttctagttacaagaacagcagaaaaagacctgaacttcagagtggaccacaaatgaatatgaattaataacacagatctcttttcaaaagccaacacaaaatgaggatgtgttttttgcaaaagcagttgttcttgccactgggcttatgc

SEQ ID NO: 247 - DNA, 505 bp, unknown
  >sr|IMAGp998J134174.2|cl|IMAGp998J134174|gb| BX106851|oid|234548083 BX106851 Soares_testis_NHT Homo sapiens cDNA clone IMAGp998J134174 ; IMAGE1645164, mRNA sequence.
accaccagnaatgtgccttttccttatcaaaaagaattgctgccccttacaggacaggaggactccaattgtgaccaagttggagcagccttgagccacaactcagacttattttcattctctgctcattggccattaggaacccgatccttatgacctgcagtgacctgctgctaccagaactgactcgcttcaaagtgacggatgaaacccatatggagatattcctggggtgggtaggccagttggaagacagagtgtttacacctgagagaggacttgagaattttactcagtcacgttgctcactgctgcgcactcctgcagacgcatcccagtggactctgcgctgcatccccagagtacagcgagtttcccgcctcccgttagagatccaacacatattttttttgaaagaagaaactgatggaagacaggaagcaaaaaggagaggaaggaaagggataggaaggaggggaaggaaagggaaaggaaggaaaagaaa

SEQ ID NO: 248 - DNA, 327 bp, unknown
  >gi|711665|sr|IMAGp998D04101|cl|IMAGp998D04101| gb|T83377|T83377 ye03h02.s1 Homo sapiens cDNA clone 116691 3′.
ttttaacataaaacagggctatttatttttattattaaacatgagtacttctgaaatatgacatttcatggnacataaaatgatgtgtatgacaaaatctgttgctaactccaaatattaacttaatatagcttagaaaaatgacataatatactctgcatttttctgaccattctatttttacaagtactccagttatatttagcattcaatttaaaagacaagtgcactgaaggantttgaggttataatataattaacccaaatattatataacnatacacacttaaatgnctggttgtggtttattggatttggcngggtatc

SEQ ID NO: 249 - DNA, 327 bp, unknown; identical to SEQ ID NO: 248
  >gi|711665|sr|IMAGp998D04101|cl|IMAGp998D04101| gb|T83377|T83377 ye03h02.s1 Homo sapiens cDNA clone 116691 3′.

SEQ ID NO: 250 - DNA, 334 bp, unknown
  >sr|IMAGp998O0470.2|cl|IMAGp998O0470|gb| BX101214|oid|234536804 BX101214 Soares infant brain 1NIB Homo sapiens cDNA clone IMAGp998O0470 ; IMAGE22040, mRNA sequence.
ttttttttttttttaaaggttagagaatccaagtttattagaaacactccaacaaaaaacaaaggtgagaagagagggcccagcccacctccactcctccctttcctccagaacagaataccagtttgtatccggcaaactggctccttccccagggttggtcccagnggggatttacatggcactgccaaagcaggacgggagccctagtcctcagggcactgcaggatgtcataggtcacgtagcccacttggtccacctggttcaactcactccgggaactcacgcgccagtagaagcggtcctggcagaaataggctttctctcggtact

SEQ ID NO: 251 - DNA, 474 bp, unknown
  >sr|IMAGp998A125385.2|cl|IMAGp998A125385|gb| BX113574|oid|234561525 BX113574 NCI_CGAP_Ut2 Homo sapiens cDNA clone IMAGp998A125385 ; IMAGE2179475, mRNA sequence.
ttttttttttttttcttgtgcaaatgctttactgagggagtactttctggagaaacctgtgagggaaccaggaaaactggataggccaaggggtcagaccaagcagagaggtggttccaggagaggcctggcctcagcccgatcccaggaacaatttgagagtgtcattcaacccagacaatcctgcacagacacgggggagctggcaattgtaccctccatggtgtcactctcctggccatgagtgacaggtggcctcttggtgggagaggtggggcagggggaaggcagctcccactagaccagggcagtatttaggggaaggaggcagattggagcagttggctgccaacacctggcctcactcagggagaggggccttgggcctggatgatatctgggtgaggtacccacaccatctgctgcaacctctgtgtatccattatgggctcacgccattcacaggcatgaatt

SEQ ID NO: 252 - DNA, 406 bp, unknown
  >sr|IMAGp998D194160.2|cl|IMAGp998D194160|gb| BX098870|oid|234532114 BX098870 Soares_testis_NHT Homo sapiens cDNA clone IMAGp998D194160 ; IMAGE1639650, mRNA sequence.
caggttgtagtgacctagcctgcaaaaatgagctccagnacacccctgagcaaggttaaaagaaaaaaaacaaattcctttactgtctctcctgtactgaactattagttatgactatgtttgccaatgcttatatttagtaaaatttataaattcctgtttttctttcaacgtagctggaaggtcactagctatacaaggccacaagttatgccaagtcaacagttatgccataaattacatgacttgtaatttatgtaattaccgcctttgttttgctcttgtatactagcctatataagctaaactctgtctttgtttggggctcaggtttttaaatgtgaatccactgagccagtgcgtaccttaaaataaatatcctcctatattcaaaaaaaaaaaaaaa

SEQ ID NO: 253 - DNA, 486 bp, unknown
  >gi|2191401|gb|AA465234|sr|IMAGp998L132010.1| cl|IMAGp998L132010|AA465234 aa24g07.s1 NCI_CGAP_GCB1 Homo sapiens cDNA clone IMAGE814236 3′, mRNA sequence.
acattttcaatcattttaatgaaaagggcgacacagccatggtgtgttcagatactcagcatgacaccgcctgcaagctctattgtctttcgctgattaaactgggctggttgagaattacatttctgcacccatggttccgctgtggtgaacaccgggagtaagcaagctcttattcaggtcccaagcaagccacagccagcatgtcagaaagcaagattcaagtcagggtccccttggcttcgactggctctactggatcaaacagctccagcggggacagaggcagcaggaaagagcttcctcaataaggaaattgaagagcaggaggaaggaaaaaataacctgaaactcagatgggcaagattaggggtggggagtgagagccagagatcatgtgtcagcacccagagcggaggagaaagaattctgattccagtccacaccaccctagactgcagcattgagcaccccagggctggcgtt

SEQ ID NO: 254 - DNA, 591 bp, unknown
  >gi|2186490|gb|AA461370|sr|IMAGp998G081965.1| cl|IMAGp998G081965|AA461370 zx70f04.r1 Soares_total_fetus_Nb2HF8_9w Homo sapiens cDNA clone IMAGE796831 5′ similar to SWECE1_BOVIN P42891 ENDOTHELIN-CONVERTING ENZYME 1 ;, mRNA sequence.
aaatattgctgataatggaggcctgcgggaagcttttagggcttacaggaaatggataaatgacagaaggcagggacttgaggagcctcttctaccaggcatcacattcaccaacaaccagctcttcttcctgagttatgctcatgtgaggtgcaattcctacagaccagaagctgcccgagaacaagtccaaattggtgctcacagtccccctcagtttagggtcaatggtgcaattagtaactttgaagaattccagaaagcttttaactgtccacccaattccacgatgaacagaggcatggactcctgccgactctggtagctgggacgctggtttatggcatcctgagacagttgcacagtgccagcggaggcttgcactgagccttcatcgcccattgctttaggcctggagactttcatttttagtgcattttcattatttgggtaggtgacctgcttggatctagacagcatctgttcaaagttgtagggcttataaagtggaatataagaatgaactaagtatgtttctttagaaaatcaaaccaacaaaaataaatccctaggctacttttgttaaaatgc

SEQ ID NO: 255 - DNA, 693 bp, unknown
  >sr|IMAGp998B072455.2|cl|IMAGp998B072455|gb| BX118394|oid|234571164 BX118394 NCI_CGAP_Pr21 Homo sapiens cDNA clone IMAGp998B072455 ; IMAGE984870, mRNA sequence.
tgaaattcccggcgctcacgcactactggcccctgatccggttcttggtgcccctgggcatcaccaacatagccctcgacttcggggagcagccatctcctttttatatttacaagaggtcacaagaggacagttaaaactgatgacgcctaaggacagatcacacgtaatagaagagctgccgcagccggttcccattagaagggcctccaagaacaacacacacaggaactcgcctgctcttctcctgatggtccacagtcacccctgccctttgagcaggaggagcgtgacctcctgccacagctacagccacaattattgaaattgtggtcaacattccatttaaagcattaaagtctgttgttttagtcctgactaagcagtaggcttctatggattgggtatttttttttttttacccacccaaatgtgttttatgttgagatgtttcgagtttttttggggggttgagggtggtgcaggtcgtgaggggcaagagctgctgcataggagataattctctttaactgattaatgtttttgctttattcaacagctattgaattaggcagtctgtacctttagtgttttccagaacaaatgtgttgaagtggcagaagggtggaagaattgaaaattattcttgcacagttgcacctgagttgtattattgctccatgtctccttatg

(19) United States
(12) Patent Application Publication
(10) Pub. No.: US 2009/0298053 A1
(43) Pub. Date: Dec. 3, 2009

(54) Use of novel biomarkers for detection of testicular carcinoma in situ and derived cancers in human samples

(75) Inventors: Kristian Almstrup, Roskilde (DK); Christina E. Høi-Hansen, Virum (DK); Ewa Rajpert-De Meyts, Greve (DK); Ute Wirkner, Eufenbach (DE); Wilhelm Ansorge, Gaiberg (DE); Henrik Leffers, Copenhagen (DK); Niels Erik Skakkebæk, Farum (DK); Christian Schwager, Limburgerhof (DE); Jonathon Blake, Hofheim (DE)

Correspondence Address: KNOBBE MARTENS OLSON & BEAR LLP, 2040 MAIN STREET, FOURTEENTH FLOOR, IRVINE, CA, 92614, US

(73) Assignee: University of Copenhagen, Copenhagen N (DK)

(21) Appl. No.: 11/587,336

(22) PCT Filed: Apr. 21, 2005
(86) PCT No.: PCT/DK05/00280

§ 371 Date: Nov. 12, 2008

(30) Foreign Application Priority Data
Apr. 23, 2004 (DK) PA2004 00647
Dec. 30, 2004 (DK) PA2004 02032

(51) Int. Cl. C12Q 1/68; C12Q 1/02; G01N 33/574

(52) U.S. Cl. 435/6; 435/29; 435/7.23

(57) ABSTRACT

The present invention relates to methods and kits for identification of testicular carcinoma in situ (CIS), gonadoblastoma (a CIS-like pre-cancerous lesion found in dysgenetic gonads) and CIS-derived cancers based on at least one of the biomarkers included in the invention. It also relates to diagnosis of a subject's status of the testicular carcinoma in situ and the derived cancers based on the measurement of a relative abundance of one of the biomarkers.

### FIELD OF THE INVENTION

The present invention provides biomarkers that characterize testicular carcinoma in situ (CIS), which is the precursor for classical testicular seminoma and non-seminomas. The same biomarkers may also be used to characterize gonadoblastoma, a CIS-like pre-cancerous lesion found in dysgenetic gonads.

### BACKGROUND OF THE INVENTION

Testicular carcinoma in situ (CIS) is the common precursor of nearly all testicular germ cell tumors (TGCTs) that occur in young adults. The incidence of TGCTs has increased markedly over the past several decades, and these tumors are now the most common type of cancer in young men.

CIS cells may transform into either a seminoma or a non-seminoma. The seminoma retains a germ-cell-like phenotype, whereas the tumour known as non-seminoma or teratoma retains embryonic stem cell features; pluripotency and the ability to differentiate into virtually all somatic tissues. Non-seminomas comprise embryonal carcinoma (EC), various mixtures of differentiated teratomatous tissue components and extraembryonic tissues, such as yolk sac tumour and choriocarcinoma. In addition to CIS, gonadoblastoma, a CIS-like lesion occurs in dysgenetic testes and intersex gonads, which frequently may contain some ovarian structures.

The only difference between CIS and gonadoblastoma concerns the overall architecture of the lesion in the surrounding gonad (CIS is found usually in single rows along the basement membrane and there is a single layer of Sertoli cells between CIS cells and the lumen of seminiferous tubules, while gonadoblastoma contains nests (clumps) of CIS-like cells surrounded by small somatic granulosa-like cells and sometimes spermatogonia-like cells). The morphology and gene expression patterns of CIS cells and gonadoblastoma cells are indistinguishable (Jørgensen et al. Histopathology 1997), therefore, further in this application, the present inventors use the term CIS for both precursor lesions.

The most commonly used marker for CIS in clinical practice is placental-like alkaline phosphatase (ALPP or PLAP) (Manivel, J C, et al. Am J Surg Pathol 1987, 11:21-9), which is also a known marker of primordial germ cells. Epidemiological evidence and evidence [symbol] germ cells indicate that CIS is an inborn lesion, probably arising from gonocytes in early fetal life and progress to an overt TGCT after puberty. The precise nature of the molecular events underlying the initiation of malignant transformation from the gonocyte to the CIS cell has not yet been elucidated, and the following progression into overt tumors remains largely unknown.

In order to identify new markers for early detection of CIS, the present inventors analyzed gene expression in CIS cells and tumors in a systematic manner, by employing both a differential display (DD) methodology and by using a cDNA microarray covering the entire human transcriptome.

### SUMMARY OF THE INVENTION

In the microarray studies the present inventors have circumvented detecting changes of gene expression not related to CIS (as CIS cells constitute only a small percentage of cells in a typical tissue sample) by using testis tissues containing different amounts of tubules with CIS and searched for genes regulated in a manner proportional to the content of CIS in the sample.

Selected candidates for CIS and tumor markers from both the DD and the microarray study have been verified by semi-quantitative reverse transcriptase PCR (RT-PCR) and the expressing cell type determined by in situ hybridization. Furthermore, the present inventors use antibodies against the some promising candidates and used immunohistochemistry to identify the expressing cell types in tissues with CIS/gonadoblastoma and in tumors derived from CIS.

The present inventors also investigated the gene expression profile (using the same large cDNA microarray) of testicular seminoma and embryonic carcinoma (EC, an undifferentiated component of non-seminomas) and compared that to the expression profile of CIS.

This facilitated identification of genes that mark the progression of CIS into either the seminoma or the non-seminoma. Results were confirmed by RT-PCR and markers were localized to tumor cells by in situ hybridization as well as for selected markers at the protein level by immunohistochemistry.

Carcincoma in situ (CIS) is the common precursor of histologically heterogeneous testicular germ cell tumors, which have markedly increased in frequency in the recent decades and [symbol] expression profiling, the present inventors identified more than 200 genes highly expressed in testicular CIS, including many never reported in testicular neoplasms. Expression was further verified by semi-quantitative RT-PCR and in situ hybridization, and by immunohistochemistry for a few protein products, for which antibodies were available.

### DESCRIPTION OF THE INVENTION

The biomarkers disclosed herein can be used for the detection of CIS and gonadoblastoma but also biomarkers for the detection of the seminomas and non-seminomas derived from CIS and thus for the progression of CIS is included in the present invention.

The biomarkers were identified using differential display and genome-wide cDNA microarray analysis and subsequent verified by other methods, including in situ hybridization, which showed that the CIS markers were expressed in CIS cells.

For some biomarkers localization to CIS cells and some CIS-derived overt tumours was also done at the protein level by immunohistochemistry.

The present invention thus relates to the use of the identified biomarkers to identify CIS and the status of CIS derived cancers in human samples such as semen samples, blood, testicular biopsies, and biopsies from any other human tissue.

In one aspect, the present invention relates to a method for determining the presence of precursors of germ cell tumours in an individual. Such a diagnostic method would comprise determining the expression profile of a group of markers in a sample, and concluding from said expression profile whether or not the sample contains precursors of germ cell tumours, wherein the group of markers consists of markers selected independently from the markers listed in table 1, whereby the number of markers in the group is between one and the total number of markers listed in table 1.

Specifically, the method comprises the following steps:
a) determining the expression profile of a group of markers in said sample, wherein the group of markers consists of at least one marker selected independently from the markers listed in table 1;
b) comparing said expression profile with a reference expression profile;
c) identifying whether the expression profile is different from said reference expression profile and
   evaluating whether the sample contains precursors of germ cell tumours if the expression profile is different from the reference expression profile.

In the present context the term “precursors of germ cell tumours” relates to the commonly accepted fact that CIS is a common precursor for all germ cell tumours of the adolescents and young adults. CIS is also known as intratubular germ cell neoplasia of the unclassified type, testicular intraepithelial neoplasia and gonocytoma in situ, with the last term probably the most accurate from the biological point of view.

In the present context the term “expression profile” relates to assaying the level of transcripts, such as mRNA, either relative to a reference value, relatively to another transcript or sample and/or absolutely values. In example cDNA micro arrays measure the abundance of a transcript in two different samples relative to each other, whereas e.g. real-time PCR measures the absolute copy number of transcripts in the sample. Both relative and absolute measurements enables a person skilled in the art to make a diagnostic decision based on the data presented.

As known to the skilled addressee, an expression profile of a transcript could also be obtained from e.g. amplified RNA, aRNA, and/or complementary DNA, cDNA, by various techniques. Thus, the present invention also relates to any methods using such manipulations. The genes listed in table 1 are in no particular order, and thus both the listed and the complementary strand is an object of the present inventions.

The sequences listed in table 1, may represent only a part of the sequence of the gene listed, e.g. an EST sequence, fragment of sequence or similar. However, the full genomic sequence, the coding sequence, untranslated regions, and promoter regions is an object of the present invention.

In general, assaying the level of transcripts, and thus determining the expression profile of a group of markers can be made by several technical approaches, such as, but not limited to, micro arrays, filter arrays, DNA chips, gene chips, differential display, SAGE, northern blot, RT-PCR, quantitative PCR, PCR, real-time PCR or any other known technique known to the skilled addressee

When assaying levels of expression of the sequences presented in table 1, the levels refers [symbol] nucleic acid is determined by methods well known in the art. “Differentially expressed” or “changes in the level of expression” may refer to an increase or decrease in the measurable expression level of a given nucleic acid and “Differentially expressed” when referring to microarray analysis means the ratio of the expression level of a given nucleic acid in one sample and the expression level of the given nucleic acid in another sample is not equal to 1.0. “Differentially expressed” when referring to microarray analysis according to the invention also means the ratio of the expression level of a given nucleic acid in one sample and the expression level of the given nucleic acid in another sample is greater than or less than 1.0 and includes greater than 1.2 and less than 0.7, as well as greater than 1.5 and less than 0.5. A nucleic acid also is said to be differentially expressed in two samples if one of the two samples contains no detectable expression of the nucleic acid.

Absolute quantification of the level of expression of a nucleic acid can be accomplished by including known concentration(s) of one or more control nucleic acid species, generating a standard curve based on the amount of the control nucleic acid and extrapolating the expression level of the “unknown” nucleic acid species from the hybridization intensities of the unknown with respect to the standard curve. The level of expression is measured by hybridization analysis using labeled target nucleic acids according to methods well known in the art. The label on the target nucleic acid can be a luminescent label, an enzymatic label, a radioactive label, a chemical label or a physical label. Preferably, target nucleic acids are labeled with a fluorescent molecule. Preferred fluorescent labels include, but are not limited to, fluorescein, amino coumarin acetic acid, tetramethylrhodamine isothiocyanate (TRITC), Texas Red, Cy3 and Cy5.

Concluding from said expression profile whether or not the sample contains precursors of germ cell tumours can be obtained by assaying the expression profile and determine if the markers are either present and/or up-regulated and/or down-regulated. Depending on the experimental set-up the marker genes may appear up-regulated but could also appear down-regulated, if e.g. the ratio were switched. The settings in the present application focus on the up-regulated markers.

Depending on the assay method and set-up the measurement of the marker genes may be done in reference to either another transcript, a sample or measured absolutely.

The group of markers consists of markers selected independently from the markers listed in table 1, the number of markers in the group is between one and the total number of markers listed in table 1, such as 1 marker, 2 markers, 3 markers, such as 4 markers, such as 5 markers, e.g. 6 markers, 7 markers, 8 markers, such as 9 markers, e.g. 10 [symbol] markers, such as 16 markers, such as 17 markers, e.g. 18 markers, 19 markers, e.g. 20 markers, such as 21 markers, 22 markers, 23 markers, e.g. 24 markers, such as 25 markers, such as 30 markers, such as 40 markers, e.g. 50 markers, such as 60 markers, such as 70 markers, e.g. 80 markers, e.g. 90 markers, such as 100 markers, such as 120 markers, e.g. 140 markers, such as 150 markers, e.g. 170 markers, e.g. 200 markers, e.g. 220 markers, e.g. 240 markers, such as 255 markers.

The term “sample” could be a semen sample, a blood sample, a serum sample, a urine sample, a testicular biopsy sample, or a biopsy from any other human tissue. The marker genes described here may be used to identify CIS or seminoma or non-seminoma cells in other clinical samples than those related to the testis and semen samples, i.e. in extra-gonadal brain tumors or blood samples from patients with disseminated seminoma.

In a presently preferred embodiment the sample is a semen sample.

The general scope of the present invention relates to detection of precursors of germ cell tumours in various samples as described above. It is obvious to the skilled addressee that the methods described herein enables a person skilled in the art to detect the presence of and/or the development of even very small amounts of precursors of germ cell tumours in the selected samples. In a presently preferred embodiment the methods described herein enables detection of at least one cell which is a precursors of germ cell tumours.

The expression profiles and as described below the intensity values are indicative of precursors of germ cell tumours since these data are found significantly more often in patients with a precursors of germ cell tumours than in patients without the precursors of germ cell tumours.

As used herein, “expression pattern” or “expression profile” comprises the pattern (i.e., qualitatively and/or quantitatively) of expression of one or more expressed nucleic acid sequences where one or more members of the set are differentially expressed.

As the skilled addressee would recognise the present invention enables a person skilled in the art in both diagnosing and/or monitoring precursors of germ cell tumours in a patient by detecting the expression level(s) of one or more genes described in table 1.

The present invention also relates to use of any of the sequences/genes/mRNAs listed in the present application for diagnostics of precursors of germ cell tumours located in the testis or in all other locations, using any method that can detect the products on the mRNA [symbol] Western blot, protein-microarrays etc.) in any human samples including semen samples, blood, tissue samples, urine, etc. in patients or healthy individuals, the sequences selected from the group consisting of SEQ ID NO 1-255 are of particularly value.

In other words, the present invention relates to a method for diagnosing precursors of germ cell tumours, the method comprising the steps of assaying an expression level for each of a predetermined number of markers of precursors of germ cell tumours in a sample; and, identifying said precursors of germ cell tumours if at least one of said expression levels is greater than a reference expression level.

Alternatively, the method for determining the presence of precursors of germ cell tumours in a sample may be based on measurements of the polypetides expressed by the nucleotide sequences comprising the markers selected from the group of SEQ ID NO: 1 to SEQ ID NO:255.

Thus, another aspect of the present invention relates to a method for determining the presence of precursors of germ cell tumours in an individual comprising determining the intensity signal(s) of a group of markers in a patient sample, and concluding from said intensity signal(s) whether the patient sample contains of precursors of germ cell tumours, wherein the group of markers consists of markers selected independently from the markers listed in table 1, whereby the number of markers in the group is between one and the total number of markers listed in table 1.

Such a method comprises the following steps:
a) determining the intensity signal of the proteins encoded by each of the members of the group of markers in said sample, wherein the group of markers consists of at least one marker selected independently from the markers listed in table 1
b) comparing each individual intensity signal with a corresponding reference intensity signal;
c) identifying whether each individual intensity signal is different from said corresponding reference intensity signal and
   evaluating whether the sample contains precursors of germ cell tumours if the intensity signal is different from the corresponding reference intensity signal.

The term “intensity signal” relates to assaying the protein level of the proteins encoded by the transcripts from table 1. Any immuno-assay is able to perform such measurement e.g. ELISA, RIA, Western blots, or dot blots. Also other methods capable of measuring the amount of a protein quantitatively or relatively. Such techniques include, but are not limited to; mass spectrometry base measurements, protein microarrays, ProteinChip® (Ciphergen) and derived methods.

As exemplified below, the present inventors are generating antibodies against the gene products of the present inventions. FIG. 6 displays an example of one such antibody used in an immuno-staining of an tissue section comprising CIS cells.

Antibodies generated against the gene products of the present invention may enable monitoring of metastasis and invasion of germ cell tumours.

As shown in table 1, several markers are up-regulated in highly abundant CIS cells, thus in one embodiment, the present invention can be performed on such up-regulated markers. Such specific markers are the markers selected from the group consisting of SEQ ID NO 1-157.

A presently preferred embodiment of the present invention further relates to a method according to the invention, wherein the group of markers consists of at least one marker which in a sample with CIS in 100% of the seminiferous tubules is up-regulated more than 7 fold when compared to a reference sample, such as 7.1 fold, e.g. 7.2 fold, such as 7.5 fold, e.g. 8 fold, e.g. 9 fold, such as 10 fold, e.g. 11 fold, e.g. 12 fold, such as 13 fold, 14 fold, 15 fold, e.g. 16 fold, such as 17 fold, 18 fold, 19 fold, e.g. 20 fold, such as 25 fold, e.g. 30 fold, 35 fold, 40 fold, e.g. 45 fold, such as 50 fold, e.g. 55 fold, e.g. 60 fold, such as 65 fold, e.g. 70 fold, such as 75 fold, e.g. 80 fold, such as 85 fold, e.g. 90 fold, e.g. 95 fold, such as 100 fold.

In another embodiment of the present invention some markers are, in an sample with CIS in 100% of the seminiferous tubules, up-regulated more than 4.3 fold but less than 7 fold. Such markers are presently considered as medium-potential markers, thus the applicability of these markers are considered to be less useful than the markers with higher abundance in CIS cells.

In yet another embodiment of the present invention some markers are, in a sample with CIS in 100% of the semiferous tubules, up-regulated more than 3 fold but less than 4.3 fold. Such markers are presently considered as markers of lesser potential, thus the [symbol] markers with higher abundance in CIS cells, however still capable of adding valuable information for the purposes of the present invention.

Another presently preferred embodiment of the present invention relates to a method according to the invention, wherein the expression profile distinguishes between classical testicular seminoma and testicular non-seminoma in gonads or in extra-gonadal localizations.

When the present inventors compared tissue samples comprising non-seminoma (EC-cells) versus CIS cells and seminoma cells, the markers defined in SEQ ID NO 158 to SEQ ID NO 202 showed an up-regulation of more than 5 fold, whereas the CIS cells and seminoma cells showed an up-regulation of less than 2 fold.

Thus, the markers defined in SEQ ID NO 158 to SEQ ID NO 202 enable a person skilled in the art to distinguish non-seminoma from CIS and seminoma, since these markers are all upregulated in non-seminoma as described in table 1 and the examples below, when compared to samples containing CIS and/or seminoma.

When the present inventors compared tissue comprising seminoma cells versus CIS cells and non-seminoma cells, the markers defined in SEQ ID NO 203 to SEQ ID NO 255 showed an up-regulation of more than 3 fold, whereas the CIS cells and non-seminoma cells showed an up-regulation of less than 2 fold.

Thus, the markers defined in SEQ ID NO 203 to SEQ ID NO 255 enable a person skilled in the art to distinguish classical seminoma from CIS and/or non-seminoma, since these markers are all upregulated in seminoma as described in table 1 and the examples below, when compared to samples containing CIS and/or non-seminoma.

One presently preferred embodiment relates to a method which can distinguish seminoma from non-seminoma.

Another presently preferred embodiment relates to a method which can distinguish seminoma from CIS.

Yet another presently preferred embodiment relates to a method which can distinguish non-seminoma from CIS.

Therefore, one aspect of the present invention relates to a method for monitoring [symbol] comprising determining the expression profile and/or the intensity signal(s) of a group of markers in a sample and concluding from expression profile and/or the intensity signal(s), whether the sample contains precursors of germ cell tumours, wherein the group of markers consists of markers selected independently from the markers listed in table 1, whereby the number of markers in the group is between one and the total number of markers listed in table 1.

In one detailed embodiment the method comprises the following steps:
a) determining the expression profile of a group of markers in said sample, wherein the group of markers consists of at least one marker selected independently from the markers listed in table 1;
b) comparing said expression profile with a reference expression profile;
c) identifying whether the expression profile is different from said reference expression profile and
   evaluating the development stage of precursors of germ cell tumours to overt cancer cells if the expression profile is different from the reference expression profile.

Alternatively, the method comprises the following steps:
a) determining the intensity signal of the proteins encoded by each of the members of the group of markers in said sample, wherein the group of markers consists of at least one marker selected independently from the markers listed in table 1
b) comparing each individual intensity signal with a corresponding reference intensity signal;
c) identifying whether each individual intensity signal is different from said corresponding reference intensity signal and
   evaluating the development stage of precursors of germ cell tumours to overt cancer cells if the intensity signal is different from the corresponding reference intensity signal.

In yet another aspect, the present invention relates to a method for identifying precursors of germ cell tumours in a sample from a mammal, the method comprising
assaying said sample by a quantitative detection assay and determining the expression profile and/or the intensity signal(s) of at least one marker selected from the group consisting of

SEQ ID NO: 1 to SEQ ID NO: 255
comparing said expression profile and/or the intensity signal(s) with reference value(s)
identifying whether the expression profile and/or the intensity signal(s) of at least one marker from the sample is significantly different from the reference value, and
   evaluating whether the sample contains precursors of germ cell tumours if the expression profile and/or the intensity signal(s) is different from the reference value.

As the skilled addressee would recognise the present invention is useful as a method for determining the presence of precursors of germ cell tumours in an individual comprising detecting the presence or absence of at least one expression product, wherein said at least one expression product comprise a nucleotide sequence selected from the group consisting of SEQ ID 1-255 in a sample isolated from the individual.

In another aspect, the present invention also relates to a method of assessing the efficacy of a therapy for inhibiting precursors of germ cell tumours in a subject, the method comprising
comparing the expression profile of a group of markers in a first sample obtained from the subject prior to providing at least a portion of the therapy to the subject and the expression profile of a group of markers in a second sample obtained from the subject following provision of the portion of the therapy, wherein a significantly altered expression profile of the marker(s) in the second sample, relative to the first sample, is an indication that the therapy is efficacious for inhibiting precursors of germ cell tumours in the subject
   wherein the group of markers consists of markers selected independently from the markers listed In table 1 and whereby the number of markers in the group is between one and the total number of markers listed in table 1.

Alternatively, the above method aspect can also be assessed by comparing the intensity signals of the proteins encoded by each of the member of the group of markers instead of comparing the expression profiles.

The method of assessing the efficacy of a therapy of the present invention also relates to methods for identifying, evaluating, and monitoring drug candidates for the treatment of precursors of germ cell tumours and of course any of the later stages. According to the invention, a candidate drug or therapy is assayed for its ability to decrease the expression of one or more markers of precursors of germ cell tumours. In one embodiment, a specific drug or therapy may reduce the expression of markers for a specific type or subclass of precursors of germ cell tumours described herein. Alternatively, a preferred drug may have a general effect on precursors of germ cell tumours and decrease the expression of different markers characteristic of different types or classes of precursors of germ cell tumours. In one embodiment, a preferred drug decreases the expression of a precursors of germ cell tumours marker by killing precursors of germ cell tumour cells or by interfering with their replication.

Current trends in stem cell research enable a person skilled in the art of various treatments comprising transplanting or regeneration of tissue by the use of e.g. embryonal stem cells.

In the present application the present inventors have identified that CIS cells have many features, which are embryonal stem cell like, see FIG. 4 and FIG. 5 and the examples below.

CIS cells thus both are embryonal stem cell like and cancer precursor cells. Thus, by use of the present markers a person skilled in the art would be able to judge, if the e.g. tissue derived from embryonal stem cells contains cells, which are likely to become cancer precursor cells, and thus represent valuable information for consideration before transplanting e.g. an embryonal stem cells.

In another aspect, the present invention also relates to a methods for analysing the presence of undifferentiated stem cells highly likely to progress to precursors of germ cell tumours and/or cancer in an individual.

Such methods comprise the following steps in one embodiment:
a) determining the expression profile of a group of markers in said sample, wherein the group of markers consists of at least one marker selected independently from the markers listed in table 1;
b) comparing said expression profile with a reference expression profile;
c) identifying whether the expression profile is different from said reference expression profile and
   evaluating whether the sample contains undifferentiated stem cells highly likely to progress to precursors of germ cell tumours and/or cancer in the individual if the expression profile is different from the reference expression profile.

Alternatively, such methods comprise the following steps in a second embodiment:
a) determining the intensity signal of the proteins encoded by each of the members of the group of markers in said sample, wherein the group of markers consists of at least one marker selected independently from the markers listed in table 1
b) comparing each individual intensity signal with a corresponding reference intensity signal;
c) identifying whether each individual intensity signal is different from said corresponding reference intensity signal and
   evaluating whether the sample contains undifferentiated stem cells highly likely to progress to precursors of germ cell tumours and/or cancer in the individual if the intensity signal is different from the reference expression profile.

Testicular carcinoma in situ (CIS) is the common precursor of nearly all testicular germ cell tumors (TGCTS) that occur in young adults. The incidence of TGCTs has increased markedly over the past several decades, and these tumors are now the most common type of cancer in young men, thus representing an increasing health problem. There is a current need in the art of potential screening methods to identify the patients in an early non-invasive stage for the purpose of e.g. treatment strategy.

Thus, in another aspect, the present invention also relates to a method for screening for the presence of precursors of germ cell tumours in an individual of a population, said method comprising determining the expression profile and/or the intensity signal(s) of a group of markers in a sample obtained from said individual, and concluding from the expression profile and/or the intensity signal(s) whether the sample contains at least one precursor cell of germ cell tumours, wherein the group of markers consists of markers selected independently from the markers listed in table 1, whereby the number of markers in the group is between one and the total number of markers listed in table 1.

### Tested Individual Markers

A range of the genes listed in table 1 has been further investigated at the protein level. Initially the genes from FIG. 1 which are highly expressed at the RNA level were studied at the protein level on tissue sections containing CIS using immunohistochemical detection. The antibodies which stained only CIS or tumour-cells in testicular tissue sections were further used to stain semen samples spreads (cytospins) on a microscope slide.

One embodiment of the present invention relates to a method according to the present invention, wherein the marker is selected from the group consisting of AP-2γ, NANOG, TCL1A, PIM-2, and E-cadherin.

AP-2γ

AP-2γ is encoded by the TFAP2C gene mapped on chromosome 20q13.2 and is a member of a family of DNA-binding transcription factors, which includes AP-2α, AP-2β, AP-2γ, TFAP-2δ and TFAP-2ε. During vertebrate embryogenesis, these proteins play important roles in the development and differentiation of the neural tube, neural crest derivatives, skin, heart and urogenital tissues and show overlapping, but distinct, patterns of expression within these tissues.

AP-2γ is required within the extra-embryonic lineages for early post-implantation development, but apparently does not play an autonomous role within the embryo proper. Mice lacking AP-2γ die at day 7.5-8.5 post coitum due to malformation of extra-embryonic tissues.

In the above genome-wide gene expression profiling study of CIS cells, we have identified a number of genes expressed also in embryonic stem cells and foetal gonocotes but not in adult germ cells, thus providing many possible new markers for CIS. One of such genes was TFAP2C (mapped to chromosome 20q13.2), which encodes the transcription factor activator protein-2 (AP-2γ).

By immunhistochemical detection of the AP-2γ protein the present inventors investigated the protein expression in a large range of tissues including fetal and malignant tissues. The present inventors found that AP-2γ protein expression was very restricted and only found in early gonocytes and other not fully differentiated cells. Also it was found to be very solid nuclear marker of testicular CIS cells and derived neoplastic cells (FIG. 6).

The present inventors disclose AP-2γ as a novel marker for foetal gonocytes and neoplastic germ cells, including testicular CIS, with a role in pathways regulating cell differentiation and a possible involvement in oncogenesis.

The fact that AP-2γ protein was not expressed in normal adult reproductive tract but was abundant in nuclei of CIS and tumour cells, which are better protected than the cytoplasm from degradation in semen due to a structural strength, prompted us to analyse the value of AP-2γ for detection of CIS and/or tumour cells in ejaculates in a series of patients and controls.

In an investigation of 104 andrological patients, presented in Example 10, cells positive for AP-2γ were found only in semen samples from patients diagnosed a priori with testicular neoplasms and, surprisingly, in a 23-year-old control subject with oligozoospermia and no symptoms of a germ cell tumour (FIG. 7).

Testicular biopsies performed during the follow-up of the 23-year-old control subject revealed widespread CIS in one testis, thus proving a diagnostic value of the new assay. Optimisation of the method is in progress, but a preliminary assessment suggests that the method may have a place in screening of germ cell neoplasia in at-risk patients. The local ethics committee approved the studies of AP-2γ expression and participants gave written informed consent.

The number of patients that we have screened for the presence of immunoreactive AP-2γ is continuously increasing as we are testing patients as they appear in our clinic. So far we have tested 347 andrological patients (including the 104 mentioned above) and found CIS-like cells positive for AP-2γ in semen samples of 3 patients, including the first case described above (a 23-year-old subfertile control subject with widespread CIS confirmed by testicular biopsies). Two other patients are currently under clinical follow-up and being considered for testicular biopses.

Thus, in a particular preferred embodiment, the present invention relates to a method according to the present invention, wherein the marker is AP-2γ.

### TCL-1A

TCL1A is a powerful oncogene that, when overexpressed in both B and T cells, predominantly yields mature B-cell lymphomas. TCL1A both in vitro and in vivo, increase AKT kinase activity and as a consequence enhances substrate phosphorylation. In vivo, TCL1A stabilizes the mitochondrial transmembrane potential and enhances cell proliferation and survival. TCL1 forms trimers, which associate with AKT in vivo and TCL1A is thus an AKT kinase coactivator that promotes AKT-induced cell survival and proliferation.

Expression of the TCL1A protein in testicular CIS as detected by immunohistochemical staining is shown in FIG. 8.

In another particular preferred embodiment, the present invention relates to a method according to the present invention, wherein the marker is TCL-1A.

### NANOG

Nanog-deficient Mouse inner cell mass (ICM) fail to generate epiblast and only produce parietal endoderm-like cells. Mouse Nanog-deficient embryonic stem (ES) cells loose pluripotency and differentiate into extraembryonic endoderm lineage. Nanog is thus a critical factor in maintaining pluripotency in both ICM and ES cells.

Elevated Nanog expression from transgene constructs is sufficient for clonal expansion of ES cells, bypassing Stat3 and maintaining POU5F1 (Oct4) levels.

NANOG was found to be highly expressed in CIS both at the RNA and protein level. Immunohistochemical detection of NANOG is shown in FIG. 9 and at the RNA level in FIGS. 1, 2, and 3.

In another particular preferred embodiment, the present invention relates to a method according to the present invention, wherein the marker is NANOG.

### PIM-2 and E-cadherin

Similar to AP-2γ, TCL1A, and NANOG, the proteins encoded by the E-cadherin gene and the PIM-2 oncogene were also found highly expressed in CIS cells. Immunohistochemical detection of E-cadherin is shown in FIG. 10 and PIM-2 in FIG. 11.

In another particular preferred embodiment, the present invention relates to a method according to the present invention, wherein the marker is PIM-2 and/or E-cadherin.

Antibodies directed against the proteins encoded by the genes in table 1 were used for immunohistochemical investigations on sections from different tissues including testicular CIS.

The present application furthermore particularly relates to a method according to the present invention, wherein the intensity signal is determined by immunocytological detection (immuno staining).

As the skilled person will know, any of the methods of the present invention may be used for screening for the presence of precursors of germ cell tumours in individual of a population, either alone or in any combination.

In another aspect, the present invention also relates to a kit for use in an assay or method as defined in the present application.

In one embodiment, such kit may be a diagnostic array comprising: a solid support; and a plurality of diagnostic agents coupled to said solid support, wherein each of said agents is used to assay the expression level of at least one of the markers shown in table 1, wherein each of said diagnostic agents is selected from the group consisting of PNA, DNA, and RNA molecules that specifically hybridize to a transcript from a marker of precursor of germ cell tumours or wherein each of said diagnostic agents is an antibody that specifically binds to a protein expression product of a marker of precursor of germ cell tumours.

In another embodiment, the present invention further provides databases of marker genes and information about the marker genes, including the expression levels that are characteristic of precursor of germ cell tumours. According to the invention, marker gene information is preferably stored in a memory in a computer system. Alternatively, the information is stored in a removable data medium such as a magnetic disk, a CDROM, a tape, or an optical disk. In a further embodiment, the input/output of the computer system can be attached to a network and the information about the marker genes can be transmitted across the network.

Preferred information includes the identity of a predetermined number of marker genes the expression of which correlates with a particular type of precursor of germ cell tumours. In addition, threshold expression levels of one or more marker genes may be stored in a [symbol] expression level is a level of expression of the marker gene that is indicative of the presence of a particular type or class precursor of germ cell tumours.

In a preferred embodiment, a computer system or removable data medium includes the identity and expression information about a plurality of marker genes for several types or classes of precursor of germ cell tumours disclosed herein. In addition, information about marker genes for normal testis tissue may be included. Information stored on a computer system or data medium as described above is useful as a reference for comparison with expression data generated in an assay of testis tissue of unknown disease status

It should be understood that any feature and/or aspect discussed above in connection with the methods according to the invention apply by analogy to the uses according to the invention.

All patent and non-patent references cited in the present application, are hereby incorporated by reference In their entirety.

As will be apparent, preferred features and characteristics of one aspect of the invention may be applicable to other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention will hereinafter be described by way of the following non-limiting Figures and Examples.

### FIGURE LEGENDS

FIG. 1

Hierarchical clustering of genes found by filtering data so only genes 4 fold or more up-regulated in the sample with CIS in 100% of the tubules and 2 fold in the samples with 50 and 75% tubules with CIS. Hierarchical clustering was done using Euclidean distances and average linkage. Some redundancy between entries is seen and implies consistency in the results. The image clone number (Unique ID on the array) is reported in front of the gene names and can be used to search the German Resource Center for Genome Research for

[symbol]

FIG. 2

Further analysis of selected genes that in microarray study were found up-regulated in CIS. A) Examples of the semi-quantitative RT-PCR on a spectrum of testicular tissue samples. The gene name is on the right side of the panes. In the gels of NANOG, and POU5F1 the beta-actin-marker band of 800 bp is visible. Representative DNA fragments from all RT-PCR amplifications were excised, cloned, and sequence verified. 129142 is an Image clone number. B) Corresponding quantifications of NANOG and POU5F1 and the ratio between the two. Quantification was done by digitalizing the image of the agarose gel. M=100 base pair marker, SEM=seminoma, TER=teratoma, EC=embryonal carcinoma, CIS=testicular tissue showing 100% tubules with carcinoma in situ, NOR=normal testicular tissue, CIS A=CIS amplified for microarray analysis, H2O=control. The CIS sample (to the right) is the same sample used in the microarray analysis.

FIG. 3

Analysis of the cellular expression in tubules with CIS by in situ hybridization of NANOG, SLC7A3, TFAP2C and ALPL showing that the expression was confined to CIS cells (arrows). CIS cells are seen as a stained ring with a stained nucleus in the center, while the rest appears empty, due to large amounts of glycogen washed out during the tissue processing. Staining was in addition seen in Sertoli cells nuclei, probably due to over-staining necessary to visualize low abundance transcripts (arrows), since this was also seen with the sense probe. The sense control is from neighbouring sections of the same tubule as the anti-sense and inserted in the lower right corner. The scale bar corresponds to 50 μm.

FIG. 4

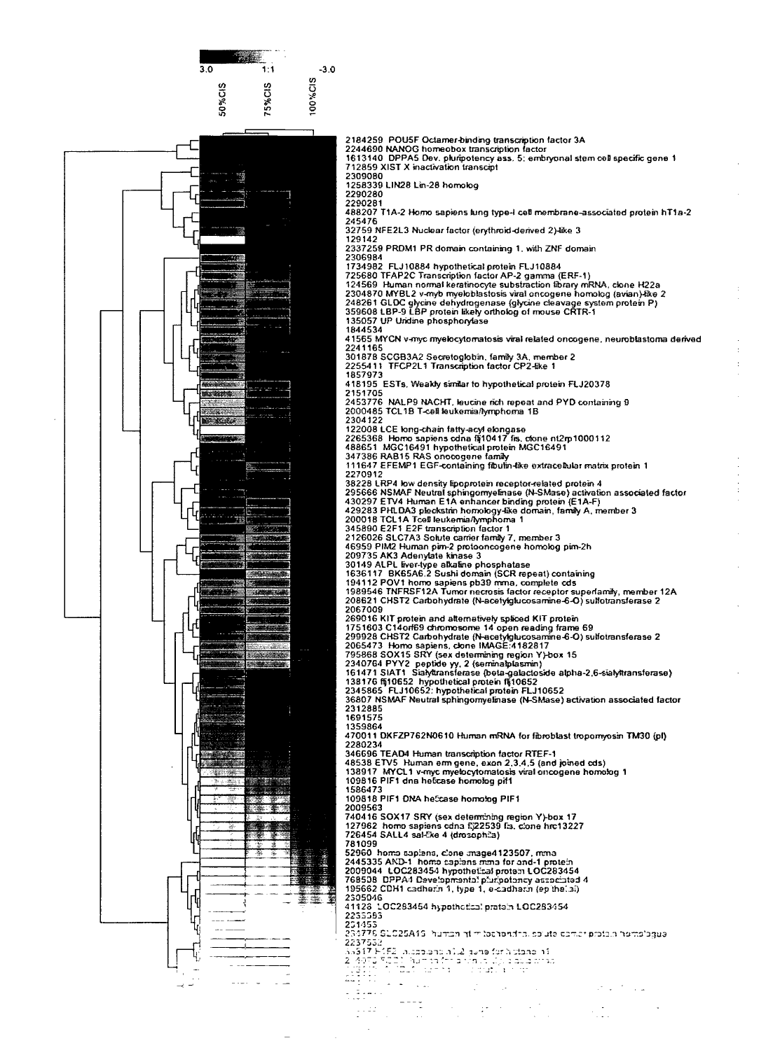

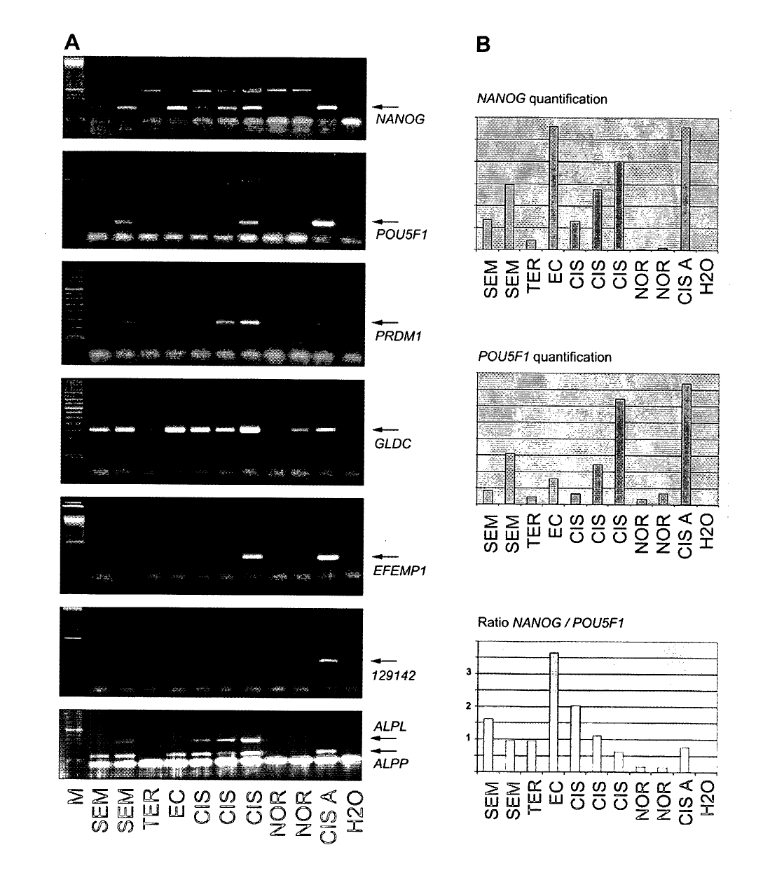

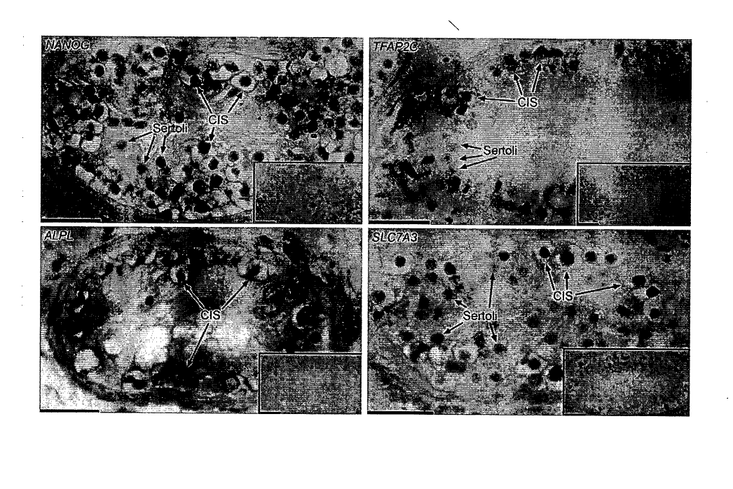

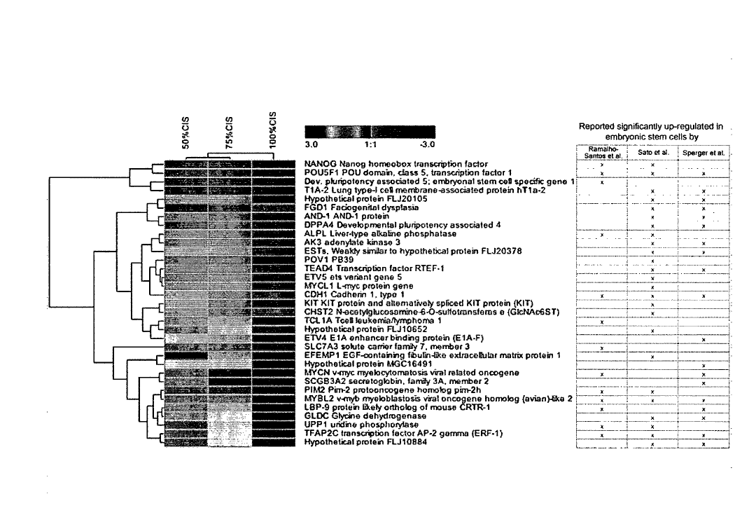

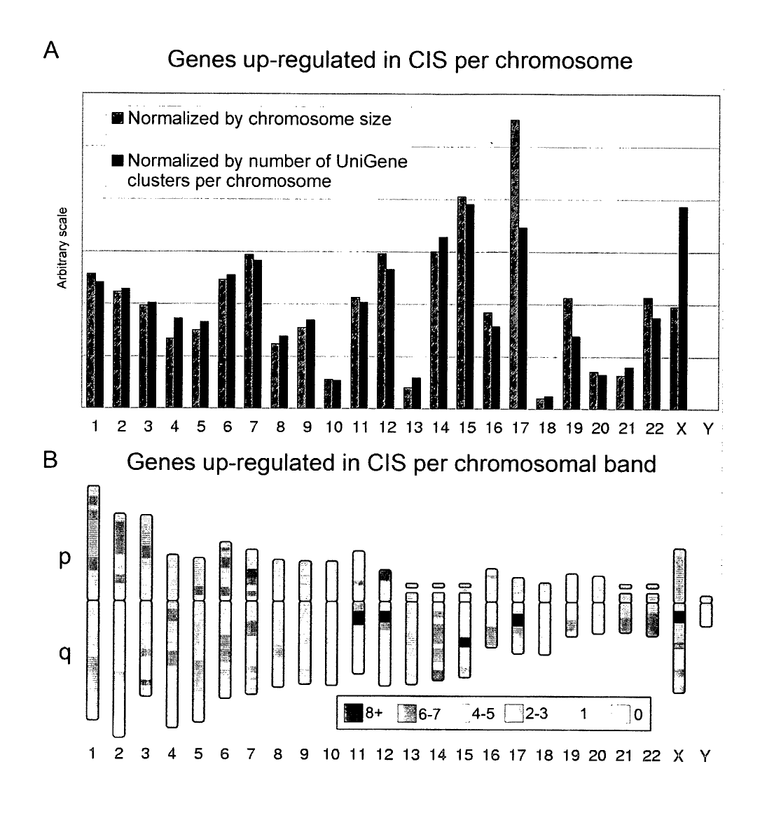

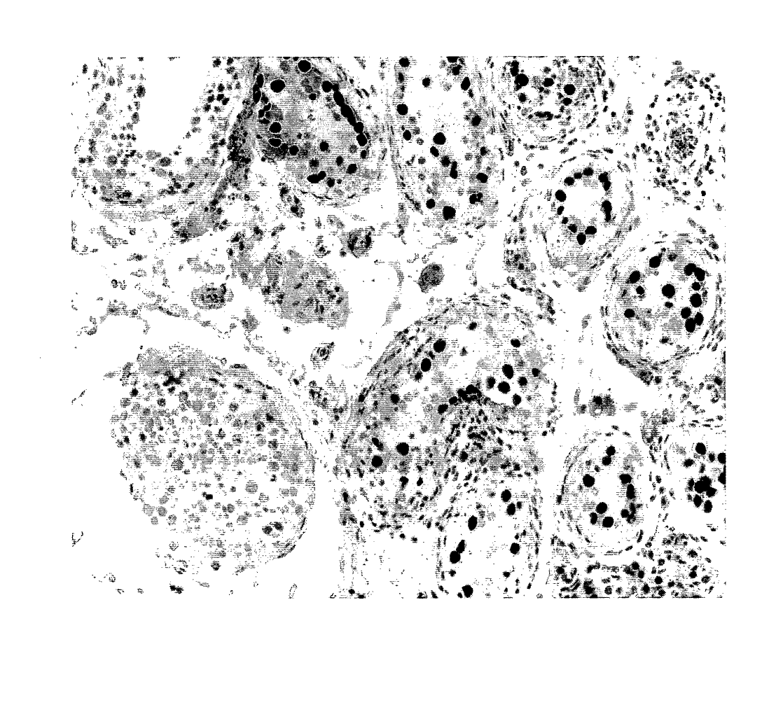

Genes from FIG. 1 reported to be up regulated in ESC. Data on mouse ESC are from Ramalho-Santos et al. (Ramalho-Santos, M, et al. Science 2002, 298:597-600) whereas data on human ESC are from Sato et al. (Sato, N, et al. Dev Biol 2003, 260:404-13) and Sperger et al. (Sperger, J M, et al. Proc Natl Acad Sci USA 2003, 100:13350-5). The overlap between genes reported in these studies and genes identified in CIS (FIG. 1) amount to roughly 35% but may be even greater due to differences in gene annotation and RNA references used.

FIG. 5

Chromosomal distribution of genes more than 2 fold up-regulated in the sample with 100% tubules having CIS. A) Distribution per chromosome and normalized by the size of the chromosomes (blue bars) or by the number of UniGene clusters on each chromosome (red bars). Chromosomal size was acquired from Ensembl, http://www.ensembl.org/, and number of UniGene clusters (Build 34 version 3) from NCBI, http://www.ncbi.nlm.nih.gov/. B) Detailed distribution of the number of up-regulated genes in each chromosomal band. The color-coding is outline in the inserted box.

FIG. 6.

Immuno-staining of transcription factor AP-2γ (TFAP2C) on a cross-section of testicular tissue containing both normal seminiferous tubules without CIS and tubules with CIS lesion. The CIS cells stain darkly and the signal is located to the nucleus of the CIS cells.

FIG. 7

A. Semen sample from a control patient spiked with CIS cells that are strongly stained with AP-2γ.

B1. AP-2γ-positive CIS cells in the semen sample of the 23-years-old subfertile control subject diagnosed with CIS by this assay.

B2, B3. Examples of AP-2γ stained cells in the 23-years-old subfertile control subject diagnosed with CIS by this assay, higher magnification.

C1, C2. Examples of AP-2γ stained cells in a patient known a priori to harbour testicular cancer.

D. Histological features of the biopsy of the left testis of the 23-years-old subfertile control subject with AP-2γ staining.

E. placental alkaline phosphatase (PLAP) staining showing CIS cells both adjacent to the tubular membrane and in the lumen. F. Semen sample from a control patient, with no AP-2γ-positive cells.

[symbol]

FIG. 8

Immuno-staining of TCL1A on a cross-section of testicular tissue containing tubules with CIS lesion. The CIS cells stain highly red and the signal is located to the nucleus of the CIS cells.

FIG. 9

Immuno-staining of homeobox protein NANOG on a cross-section of testicular tissue containing both normal seminiferous tubules without CIS and tubules with CIS lesion. The CIS cells stain darkly and the signal is located to the nucleus of the CIS cells.

FIG. 10

Immuno-staining of E-cadherin on a cross-section of testicular tissue containing tubules with CIS lesion. The CIS cells stain dark red and the signal is located to the cytoplasm or cellular membrane of the CIS cells.

FIG. 11

Immuno-staining of PIM-2 on a cross-section of testicular tissue containing tubules with CIS lesion. The CIS cells stain dark red and the signal is located to the nucleus of the CIS cells.

TABLE I

| | | IMAGE | |
|---|---|---|---|
| | [symbol] Entry [RZPD] | clone | Feature description |
| [symbol] ID No: | IMAGp998N195554; IMAGp971M1993 | 2244690 | Nanog homeobox |
| [symbol] | | | |
| [symbol] ID No: | IMAGp998G08223; IMAGp971H0410 | 147103 | hXBP-1 transcription factor |
| [symbol] | | | |
| [symbol] ID No: | IMAGp998F08222; IMAGp971F0410 | 146695 | |
| [symbol] | | | |
| [symbol] ID No: | IMAGp998A06220; IMAGp971B0610 | 145805 | TMF1 TATA element modulatory factor 1 |
| [symbol] | | | |
| [symbol] ID No: | IMAGp998D203167; IMAGp971C1959 | 1258339 | LIN28 lin-28 homolog (C. elegans) |
| [symbol] | | | |
| [symbol] ID No: | IMAGp998D054091; IMAGp971C0466 | 1613140 | developmental pluripotency associated 5; embryonal |
| [symbol] | | | stem cell specific gene 1 |
| [symbol] ID No: | IMAGp998K241161; IMAGp971D2341 | 488207 | Homo sapiens lung type-I cell membrane- |
| [symbol] | | | associated protein hT1a-2 (hT1a-2) mRNA |
| [symbol] ID No: | IMAGp998J095673; IMAGp971I09130 | 2290280 | |
| [symbol] | | | |
| [symbol] ID No: | IMAGp998I175722; IMAGp971I1796 | 2309080 | |
| [symbol] | | | |
| [symbol] ID No: | IMAGp998J105673; IMAGp971E17130 | 2290281 | |
| [symbol] | | | |
| [symbol] ID No: | IMAGp998N115245; IMAGp971I0390 | 2126026 | SLC7A3 solute carrier family 7 (cationic |
| [symbol] | | | amino acid transporter, y+ system), |
| | | | member 3 |
| [symbol] ID No: | IMAGp998J05529; IMAGp971I0522 | 245476 | |
| [symbol] | | | |
| [symbol] ID No: | IMAGp998A03577; IMAGp971A0324 | 263690 | |
| [symbol] | | | |
| [symbol] ID No: | IMAGp998H234408; IMAGp971G2474 | 1734982 | FLJ10884 hypothetical protein FLJ10884 |
| [symbol] | | | |
| [symbol] ID No: | IMAGp998G13226; IMAGp971F0610 | 148260 | |
| [symbol] | | | |
| [symbol] ID No: | IMAGp998J23134; IMAGp971H236 | 129142 | |
| [symbol] | | | |
| [symbol] ID No: | IMAGp998K225545; IMAGp971L2192 | 2241165 | |
| [symbol] | | | |
| [symbol] ID No: | IMAGp998N121162; IMAGp971O1341 | 488651 | MGC16491 hypothetical protein MGC16491 |
| [symbol] | | | |
| [symbol] ID No: | IMAGp998H07676; IMAGp971G1031 | 301878 | SCGB3A2 secretoglobin, family 3A, |
| [symbol] | | | member 2 |
| [symbol] ID No: | IMAGp998B171780; IMAGp971G1744 | 725680 | TFAP2C transcription factor AP-2 gamma |
| [symbol] | | | (activating enhancer binding protein 2 |
| | | | gamma) |
| [symbol] ID No: | IMAGp998A2488; IMAGp971B232 | 111647 | EFEMP1 EGF-containing fibulin-like |
| [symbol] | | | extracellular matrix protein 1 |
| [symbol] ID No: | IMAGp998H01154; IMAGp971C01113 | 32759 | NFE2L3 nuclear factor (erythroid-derived |
| [symbol] | | | 2)-like 3 |
| [symbol] ID No: | IMAGp998J02274; IMAGp971K01117 | 46959 | Human pim-2 protooncogene homolog |
| [symbol] | | | pim-2h mRNA, complete cds |
| [symbol] ID No: | IMAGp998L10121; IMAGp971M185 | 124569 | Human normal keratinocyte substraction library |
| [symbol] | | | mRNA, clone H22a, complete sequence |
| [symbol] ID No: | IMAGp998J075711; IMAGp971J1294 | 2304870 | MYBL2 v-myb myeloblastosis viral |
| [symbol] | | | oncogene homolog (avian)-like 2 |
| [symbol] ID No: | IMAGp998N06536; IMAGp971P1522 | 248261 | glycine dehydrogenase (glycine cleavage |
| [symbol] | | | system protein P) |
| [symbol] ID No: | IMAGp998M17826; IMAGp971N1034 | 359608 | LBP protein likely ortholog of mouse CRTR-1 |
| [symbol] | | | |
| [symbol] ID No: | IMAGp998P11794; IMAGp971O0935 | 347386 | RAB15 RAB15, member RAS onocogene |
| [symbol] | | | family |
| [symbol] ID No: | IMAGp998O08148; IMAGp971M05112 | 30149 | Human mRNA for liver-type alkaline |
| [symbol] | | | phosphatase |
| [symbol] ID No: | IMAGp998C095623; IMAGp971I0993 | 2270912 | |
| [symbol] | | | |
| [symbol] ID No: | IMAGp998O205798; IMAGp971P1197 | 2337259 | PRDM1 PR domain containing 1, with ZNF |
| [symbol] | | | domain |
| [symbol] ID No: | IMAGp998A10192; IMAGp971B108 | 135057 | UP uridine phosphorylase |
| [symbol] | | | |
| [symbol] ID No: | IMAGp998F20979; IMAGp971E2038 | 418195 | ESTs, Weakly similar to hypothetical protein FLJ20378 [Homo |
| [symbol] | | | sapiens] |
| [symbol] ID No: | IMAGp998B095717; IMAGp971C1895 | 2306984 | |
| [symbol] | | | |
| [symbol] ID No: | IMAGp998M125582; IMAGp971H07127 | 2255411 | transcription factor CP2-like 1 |
| [symbol] | | | |
| [symbol] ID No: | IMAGp998M154512; IMAGp971N1778 | 1844534 | |
| [symbol] | | | |
| [symbol] ID No: | IMAGp998M144547; IMAGp971G0981 | 1857973 | |
| [symbol] | | | |
| [symbol] ID No: | IMAGp998L105312; IMAGp971P0590 | 2151705 | HLXB9 homeo box HB9 |
| [symbol] | | | |
| [symbol] ID No: | IMAGp998D201008; IMAGp971D2339 | 429283 | pleckstrin homology-like domain, family A, |
| [symbol] | | | member 3 |
| [symbol] ID No: | IMAGp998H065732; IMAGp971C0596 | 2312885 | |
| [symbol] | | | |
| [symbol] ID No: | IMAGp998H084295; IMAGp971G0772 | 1691575 | COL22A1 collagen, type XXII, alpha 1 |
| [symbol] | | | |
| [symbol] ID No: | IMAGp998F176102; IMAGp971E15132 | 2453776 | NACHT, leucine rich repeat and PYD |
| [symbol] | | | containing 9 |
| [symbol] ID No: | IMAGp998K013431; IMAGp971J01106 | 1359864 | |
| [symbol] | | | |
| [symbol] ID No: | IMAGp998M23174; IMAGp971P24114 | 41558 | |
| [symbol] | | | |
| [symbol] ID No: | IMAGp998C17793; IMAGp971B1734 | 346696 | Human transcription factor RTEF-1 (RTEF1) |
| [symbol] | | | mRNA, complete cds |
| [symbol] ID No: | IMAGp998O144918; IMAGp971P13122 | 2000485 | T-cell leukemia/lymphoma 1B or TCL6 T- |
| [symbol] | | | cell leukemia/lymphoma 6 |
| [symbol] ID No: | IMAGp998O021010; IMAGp971O0139 | 430297 | Human E1A enhancer binding protein |
| [symbol] | | | (E1A-F) mRNA, partial cds |
| [symbol] ID No: | IMAGp998E201114; IMAGp971J1941 | 470011 | Human mRNA for fibroblast tropomyosin |
| [symbol] | | | TM30 (pl) |
| [symbol] ID No: | IMAGp998H224299; IMAGp971D2472 | 1693125 | |
| [symbol] | | | |
| [symbol] ID No: | IMAGp998G195647; IMAGp971J18129 | 2280234 | |
| [symbol] | | | |
| [symbol] ID No: | IMAGp998K06166; IMAGp971K05114 | 38228 | low density lipoprotein receptor-related |
| [symbol] | | | protein 4 |
| [symbol] ID No: | IMAGp998D03384; IMAGp971E0415 | 200018 | H. sapiens mRNA for Tcell |
| [symbol] | | | leukemia/lymphoma 1 |
| [symbol] ID No: | IMAGp998E01223; IMAGp971D0610 | 147048 | Homo sapiens endothelial cell protein |
| [symbol] | | | C/APC receptor (EPCR) mRNA, complete |
| | | | cds |
| [symbol] ID No: | IMAGp998D054941; IMAGp971D0184 | 2009044 | |
| [symbol] | | | |
| [symbol] ID No: | IMAGp998A17115; IMAGp971B104 | 122008 | long-chain fatty-acyl elongase |
| [symbol] | | | |
| [symbol] ID No: | IMAGp998K035709; IMAGp971J0394 | 2304122 | |
| [symbol] | | | |
| [symbol] ID No: | IMAGp998F13278; IMAGp971K05119 | 48538 | ETV5 ets variant gene 5 (ets-related |
| [symbol] | | | molecule) |
| [symbol] ID No: | IMAGp998B06202; IMAGp971A069 | 138917 | Human L-myc protein gene, complete cds |
| [symbol] | | | |
| [symbol] ID No: | IMAGp998E1783; IMAGp971E141 | 109816 | DNA helicase homolog PIF1 |
| [symbol] | | | |
| [symbol] ID No: | IMAGp998L095608; IMAGp971N09128 | 2265368 | Homo sapiens cDNA FLJ10417 fis, clone |
| [symbol] | | | NT2RP1000112 |
| [symbol] ID No: | IMAGp998E11660; IMAGp971E0330 | 295666 | |
| [symbol] | | | |
| [symbol] ID No: | IMAGp998K051891; IMAGp971K0650 | 768508 | developmental pluripotency associated 4 |
| [symbol] | | | |
| [symbol] ID No: | IMAGp998J14406; IMAGp971J1117 | 208621 | |
| [symbol] | | | |
| [symbol] ID No: | IMAGp998B231782; IMAGp971B2344 | 726454 | sal-like 4 (Drosophila) |
| [symbol] | | | |
| [symbol] ID No: | IMAGp998M024021; IMAGp971P0165 | 1586473 | |
| [symbol] | | | |
| [symbol] ID No: | IMAGp998C105092; IMAGp971B0286 | 2067009 | |
| [symbol] | | | |
| [symbol] ID No: | IMAGp998I19131; IMAGp971E206 | 127962 | Homo sapiens cDNA FLJ22539 fis, clone |
| [symbol] | | | |
| [symbol] ID No: | IMAGp998F246080; IMAGp971E22131 | 2445335 | Homo sapiens mRNA for AND-1 protein |
| [symbol] | | | |
| [symbol] ID No: | IMAGp998B03791; IMAGp971F1134 | 345890 | E2F transcription factor 1 |
| [symbol] | | | |
| [symbol] ID No: | IMAGp998N15372; IMAGp971M1615 | 195662 | cadherin 1, type 1, E-cadherin (epithelial) |
| [symbol] | | | |
| [symbol] ID No: | IMAGp998G201924; IMAGp971G2151 | 781099 | |
| [symbol] | | | |
| [symbol] ID No: | IMAGp998E1983; IMAGp971E212 | 109818 | |
| [symbol] | | | |
| [symbol] ID No: | IMAGp998C09200; IMAGp971D099 | 138176 | hypothetical protein FLJ10652 |
| [symbol] | | | |
| [symbol] ID No: | IMAGp998I204942; IMAGp971J2084 | 2009563 | |
| [symbol] | | | |
| [symbol] ID No: | IMAGp998H171818; IMAGp971I1747 | 740416 | SRY (sex determining region Y)-box 17 |
| [symbol] | | | |
| [symbol] ID No: | IMAGp998P175394; IMAGp971N18124 | 2183296 | Homo sapiens cDNA FLJ12742 fis, clone |
| [symbol] | | | |
| [symbol] ID No: | IMAGp998F105821; IMAGp971B1898 | 2345865 | |
| [symbol] | | | |
| [symbol] ID No: | IMAGp998G01671; IMAGp971H0330 | 299928 | Homo sapiens GN6ST mRNA for N- |
| [symbol] | | | acetylglucosamine-6-O-sulfotransferase |
| | | | (GlcNAc6ST) |
| [symbol] ID No: | IMAGp998D125539; IMAGp971F1691 | 2238683 | |
| [symbol] | | | |
| [symbol] ID No: | IMAGp998C22571; IMAGp971D2123 | 261453 | |
| [symbol] | | | |
| [symbol] ID No: | IMAGp998M24260; IMAGp971I2312 | 161471 | Sialyltransferase (CMP-N- |
| [symbol] | | | acetylneuraminate-beta-galactoside alpha- |
| | | | 2,6-sialyltransferase) |
| [symbol] ID No: | IMAGp998N11579; IMAGp971K1823 | 264778 | Solute carrier family 25 (mitochondrial |
| [symbol] | | | carrier; Graves disease autoantigen), |
| | | | member 16 |
| [symbol] ID No: | IMAGp998E15164; IMAGp971H10113 | 36807 | Homo sapiens neutral sphingomyelinase |
| [symbol] | | | (N-SMase) activation associated factor |
| [symbol] ID No: | IMAGp998N06288; IMAGp971L03118 | 52960 | Homo sapiens, clone IMAGE4123507, mRNA |
| [symbol] | | | |
| [symbol] ID No: | IMAGp998M18194; IMAGp971N068 | 136121 | Homo sapiens cDNA FLJ10247 fis, clone |
| [symbol] | | | HEMBB1000705 |
| [symbol] ID No: | IMAGp998J155722; IMAGp971K1295 | 2309102 | |
| [symbol] | | | |
| [symbol] ID No: | IMAGp998B21790; IMAGp971E1334 | 345524 | integral membrane protein 2C |
| [symbol] | | | |
| [symbol] ID No: | IMAGp998J09792; IMAGp971C04134 | 346472 | Homo sapiens, clone IMAGE4123507, mRNA |
| [symbol] | | | |
| [symbol] ID No: | IMAGp998H175550; IMAGp971H2092 | 2243008 | |
| [symbol] | | | |
| [symbol] ID No: | IMAGp998E24312; IMAGp971F2412 | 172415 | Solute carrier family 35 (UDP-galactose |
| [symbol] | | | transporter), member A2 |
| [symbol] ID No: | IMAGp998J081153; IMAGp971J1040 | 485095 | LOC388610 (LOC388610), mRNA |
| [symbol] | | | |
| [symbol] ID No: | IMAGp998G18196; IMAGp971H148 | 136745 | hypothetical protein FLJ20171 |
| [symbol] | | | |
| [symbol] ID No: | IMAGp998F241862; IMAGp971F2248 | 757271 | |
| [symbol] | | | |
| [symbol] ID No: | IMAGp998B09229; IMAGp971B0410 | 149288 | Nedd4 family interacting protein 2 |
| [symbol] | | | |
| [symbol] ID No: | IMAGp998E11127; IMAGp971H145 | 126322 | Caspase 4, apoptosis-related cysteine |
| [symbol] | | | protease |
| [symbol] ID No: | IMAGp998N02421; IMAGp971N0518 | 214465 | Oxysterol binding protein-like 3 |
| [symbol] | | | |
| [symbol] ID No: | IMAGp998B145536; IMAGp971B1592 | 2237485 | |
| [symbol] | | | |
| [symbol] ID No: | IMAGp998C144120; IMAGp971D1066 | 1624261 | |
| [symbol] | | | |
| [symbol] ID No: | IMAGp998M232973; IMAGp971E1958 | 1184062 | |
| [symbol] | | | |
| [symbol] ID No: | IMAGp998D051943; IMAGp971D0552 | 788308 | Cell cycle progression 8 protein |
| [symbol] | | | |
| [symbol] ID No: | IMAGp998M031831; IMAGp971M0448 | 745514 | |
| [symbol] | | | |
| [symbol] ID No: | IMAGp998K243513; IMAGp971L2358 | 1391375 | |
| [symbol] | | | |
| [symbol] ID No: | IMAGp998B165468; IMAGp971J04132 | 2211375 | Annexin A3 |
| [symbol] | | | |
| [symbol] ID No: | IMAGp998G024171; IMAGp971L0469 | 1643929 | |
| [symbol] | | | |
| [symbol] ID No: | IMAGp998K15174; IMAGp971L16114 | 41539 | Homo sapiens mRNA for KIAA0644 protein, |
| [symbol] | | | complete cds |
| [symbol] ID No: | IMAGp998I04624; IMAGp971H0926 | 281931 | cytoplasmic polyadenylation element |
| [symbol] | | | binding protein |
| [symbol] ID No: | IMAGp998M24235; IMAGp971N2411 | 1518711 | hypothetical protein FLJ22662 |
| [symbol] | | | |
| [symbol] ID No: | IMAGp998F16280; IMAGp971H22117 | 49499 | Dedicator of cytokinesis 11 |
| [symbol] | | | |
| [symbol] ID No: | IMAGp998B23677; IMAGp971A2431 | 302134 | H. sapiens mRNA for SOX-4 protein |
| [symbol] | | | |
| [symbol] ID No: | IMAGp998N204778; IMAGp971F1184 | 1946707 | ESTs, Weakly similar to C Chain C, Human Activated Protein C |
| [symbol] | | | [H. sapiens] |
| [symbol] ID No: | IMAGp998E163300; IMAGp971F12104 | 1309431 | |
| [symbol] | | | |
| [symbol] ID No: | IMAGp998P192044; IMAGp971O2055 | 827394 | ash2 (absent, small, or homeotic)-like |
| [symbol] | | | (Drosophila) |
| [symbol] ID No: | IMAGp998I144113; IMAGp971I1366 | 1621717 | hypothetical protein LOC130576 |
| [symbol] | | | |
| [symbol] ID No: | IMAGp998N045486; IMAGp971L02126 | 2218563 | |
| [symbol] | | | |
| [symbol] ID No: | IMAGp998I16328; IMAGp971J1413 | 178647 | Myosin VA (heavy polypeptide 12, myoxin) |
| [symbol] | | | |
| [symbol] ID No: | IMAGp998C14658; IMAGp971A1330 | 294853 | UDP-glucose ceramide glucosyltransferase |
| [symbol] | | | |
| [symbol] ID No: | IMAGp998M174328; IMAGp971I1573 | 1704376 | |
| [symbol] | | | |
| [symbol] ID No: | IMAGp998D065777; IMAGp971B0496 | 2328917 | Vacuolar protein sorting 13A (yeast |
| [symbol] | | | |
| [symbol] ID No: | IMAGp998D01196; IMAGp971D199 | 136656 | Sorting nexin 10 |
| [symbol] | | | |
| [symbol] ID No: | IMAGp998B2070; IMAGp971A13110 | 22170 | Homo sapiens family with sequence |
| [symbol] | | | similarity 16, member A, X-linked |
| [symbol] ID No: | IMAGp998I17258; IMAGp971G1812 | 160600 | Human mRNA for complement control |
| [symbol] | | | protein factor I |
| [symbol] ID No: | IMAGp998M04179; IMAGp971M107 | 130347 | Dysferlin, limb girdle muscular dystrophy |
| [symbol] | | | 2B (autosomal recessive) |
| [symbol] ID No: | IMAGp998K175764; IMAGp971N22120 | 2324104 | |
| [symbol] | | | |
| [symbol] ID No: | IMAGp998J11128; IMAGp971H045 | 126826 | |
| [symbol] | | | |
| [symbol] ID No: | IMAGp998H195650; IMAGp971K18129 | 2281410 | |
| [symbol] | | | |
| [symbol] ID No: | IMAGp998P025858; IMAGp971N0499 | 2360305 | |
| [symbol] | | | |
| [symbol] ID No: | IMAGp998M03208; IMAGp971N109 | 141482 | Solute carrier family 38, member 1 |
| [symbol] | | | |
| [symbol] ID No: | IMAGp998H07795; IMAGp971C0535 | 347574 | Full length insert cDNA clone ZD86G04 |
| [symbol] | | | |
| [symbol] ID No: | IMAGp998A112011; IMAGp971D15103 | 814354 | |
| [symbol] | | | |
| [symbol] ID No: | IMAGp998A205772; IMAGp971F2296 | 2326939 | Timeless homolog (Drosophila) |
| [symbol] | | | |
| [symbol] ID No: | IMAGp998I09280; IMAGp971J02117 | 49203 | DNA-damage-inducible transcript 4-like |
| [symbol] | | | |
| [symbol] ID No: | IMAGp998J24653; IMAGp971I1829 | 293111 | Human cDNA for uracil-DNA glycosylase |
| [symbol] | | | |
| [symbol] ID No: | IMAGp998N161887; IMAGp971I2350 | 767055 | Sema domain, immunoglobulin domain |
| [symbol] | | | (Ig), short basic domain, secreted, |
| | | | (semaphorin) 3A |
| [symbol] genes | | | |
| [symbol] ID No: 134 | | | THIOREDOXIN INTERACTING PROTEIN; |
| | | | UPREGULATED BY 1,25- |
| | | | DIHYDROXYVITAMIN D-3 |
| [symbol] ID No: 135 | | | CALDESMON (CDM). |
| [symbol] ID No: 136 | | | HLA CLASS II HISTOCOMPATIBILITY |
| | | | ANTIGEN, GAMMA CHAIN (HLA-DR |
| | | | ANTIGENS ASSOCIATED INVARIANT |
| | | | CHAIN) (IA ANTIGEN-ASSOCIATED |
| | | | INVARIANT CHAIN) (II) (P33) (CD74 |
| | | | ANTIGEN). |
| [symbol] ID No: 137 | | | COMPLEMENT COMPONENT C7 |
| | | | PRECURSOR |
| [symbol] ID No: 138 | | | SMALL INDUCIBLE CYTOKINE B16 |
| | | | (TRANSMEMBRANE CHEMOKINE CXCL16) |
| | | | (SR-PSOX) (SCAVENGER RECEPTOR FOR |
| | | | PHOSPHATIDYLSERINE AND OXIDIZED |
| | | | LOW DENSITY LIPOPROTEIN) |
| [symbol] ID No: 139 | | | CYTOCHROME B REDUCTASE 1; |
| | | | DUODENAL CYTOCHROME B |
| [symbol] ID No: 140 | | | INTERFERON-INDUCED TRANSMEMBRANE |
| | | | PROTEIN 1 (INTERFERON-INDUCED |
| | | | PROTEIN 17) (INTERFERON-INDUCIBLE |
| | | | PROTEIN 9-27) (LEU-13 ANTIGEN) (CD225 |
| | | | ANTIGEN). |
| [symbol] ID No: 141 | | | SAL-LIKE PROTEIN 4 (ZINC FINGER |
| | | | PROTEIN SALL4). |
| [symbol] ID No: 142 | | | ENDOPLASMIN PRECURSOR (94 KDA |
| | | | GLUCOSE-REGULATED PROTEIN) (GRP94) |
| | | | (GP96 HOMOLOG) (TUMOR REJECTION |
| | | | ANTIGEN 1). |
| [symbol] ID No: 143 | | | ALPHA 2 TYPE I COLLAGEN; COLLAGEN I, |
| | | | ALPHA-2 POLYPEPTIDE; COLLAGEN OF |
| | | | SKIN, TENDON AND BONE, ALPHA-2 |
| | | | CHAIN |
| [symbol] ID No: 144 | | | HISTONE DEACETYLASE 3 (HD3) (RPD3- |
| | | | 2). |
| [symbol] ID No: 145 | | | HLA CLASS II HISTOCOMPATIBILITY |
| | | | ANTIGEN, DR ALPHA CHAIN PRECURSOR |
| [symbol] ID No: 146 | | | DYNAMIN-LIKE 120 KDA PROTEIN, |
| | | | MITOCHONDRIAL PRECURSOR (OPTIC |
| | | | ATROPHY 1 GENE PROTEIN). |
| [symbol] ID No: 147 | | | BULLOUS PEMPHIGOID ANTIGEN 1 |
| | | | ISOFORMS 1/2/3/4/5/8 (230 KDA |
| | | | BULLOUS PEMPHIGOID ANTIGEN) (BPA) |
| | | | (HEMIDESMOSOMAL PLAQUE PROTEIN) |
| | | | (DYSTONIA MUSCULORUM PROTEIN) |
| [symbol] ID No: 148 | | | DECORIN PRECURSOR (BONE |
| | | | PROTEOGLYCAN II) (PG-S2) (PG40) |
| [symbol] ID No: 149 | | | LEUKOCYTE SURFACE ANTIGEN CD47 |
| | | | PRECURSOR (ANTIGENIC SURFACE |
| | | | DETERMINANT PROTEIN OA3) (INTEGRIN |
| | | | ASSOCIATED PROTEIN) (IAP) (MER6). |
| [symbol] ID No: 150 | | | b2-microglobulin |
| [symbol] ID No: 151 | | | 60S RIBOSOMAL PROTEIN L27 |
| [symbol] ID No: 152 | | | 40S RIBOSOMAL PROTEIN S16 |
| [symbol] ID No: 153 | | | UNCHARACTERIZED HYPOTHALAMUS PROTEIN |
| | | | HCDASE NM_018479 |
| [symbol] ID No: 154 | | | Overexpressed in CIS |
| [symbol] ID No: 155 | | | Ensambl EST ENSESTT00000023592 |
| [symbol] ID No: 156 | | | Chromosome 16 clone |
| [symbol] ID No: 157 | | | Chromosome-1 clone hypothetical protein XP_211586 |
| [symbol] genes | | | |
| [symbol] ID No: | IMAGp998F065401; IMAGp971I06125 | 2185733 | S100A14 S100 calcium binding protein A14 |
| [symbol] | | | |
| [symbol] ID No: | IMAGp998J065499; IMAGp971I22120 | 2223461 | transmembrane 4 superfamily member 3 |
| [symbol] | | | |
| [symbol] ID No: | IMAGp998A05535; IMAGp971B0621 | 247564 | Homo sapiens transcribed sequence with moderate |
| [symbol] | | | similarity to protein ref: NP_060190.1 (H. sapiens) |
| | | | hypothetical protein FLJ20234 [Homo |
| [symbol] ID No: | IMAGp998B19790; IMAGp971B0934 | 345522 | SEC13-like 1 (S. cerevisiae) |
| [symbol] | | | |
| [symbol] ID No: | IMAGp998M105514; IMAGp971K10120 | 2229297 | |
| [symbol] | | | |
| [symbol] ID No: | IMAGp998O095541; IMAGp971P0592 | 2239712 | Homo sapiens transcribed sequence with weak similarity to |
| [symbol] | | | protein ref: NP_038602.1 (M. musculus) L1 repeat, |
| | | | Tf subfamily, member 18 [Mus musculus] |
| [symbol] ID No: | IMAGp998D121896; IMAGp971D1150 | 770267 | |
| [symbol] | | | |
| [symbol] ID No: | IMAGp998H235725; IMAGp971H1195 | 2310214 | |
| [symbol] | | | |
| [symbol] ID No: | IMAGp998P03372; IMAGp971O0415 | 195698 | Human fibrinogen beta chain gene, |
| [symbol] | | | complete mRNA |
| [symbol] ID No: | IMAGp998B071870; IMAGp971C0749 | 760230 | Homo sapiens secreted cement gland |
| [symbol] | | | protein XAG-2 homolog (hAG-2/R) mRNA, |
| | | | complete cds |
| [symbol] ID No: | IMAGp998D08822; IMAGp971C0735 | 357847 | coiled-coil domain containing 2 |
| [symbol] | | | |
| [symbol] ID No: | IMAGp998I08822; IMAGP971C0635 | 357967 | KIAA1712 protein |
| [symbol] | | | |
| [symbol] ID No: | IMAGp998D09822; IMAGp971C0935 | 357848 | Homo sapiens similar to hypothetical protein |
| [symbol] | | | (LOC285177), mRNA |
| [symbol] ID No: | IMAGp998K045001; IMAGp971H0285 | 2032251 | chromosome 4 open reading frame 7 |
| [symbol] | | | |
| [symbol] ID No: | IMAGp998K125400; IMAGp971C19125 | 2185475 | |
| [symbol] | | | |
| [symbol] ID No: | IMAGp998K151776; IMAGp971P2043 | 724358 | keratin 19 |
| [symbol] | | | |
| [symbol] ID No: | IMAGp998I164581; IMAGp971L1480 | 1870935 | fibronectin 1 |
| [symbol] | | | |
| [symbol] ID No: | IMAGp998F044825; IMAGp971I03121 | 1964547 | |
| [symbol] | | | |
| [symbol] ID No: | IMAGp998L206082; IMAGp971L19131 | 2446243 | |
| [symbol] | | | |
| [symbol] ID No: | IMAGp998D085368; IMAGp971A0792 | 2173015 | Homo sapiens caudal-type homeobox gene |
| [symbol] | | | 2 (CDX2) sequence |
| [symbol] ID No: | IMAGp998D20611; IMAGp971E2025 | 276835 | TPA regulated locus |
| [symbol] | | | |
| [symbol] ID No: | IMAGp998B14742; IMAGp971C2433 | 327085 | discs, large (Drosophila) homolog- |
| [symbol] | | | associated protein 1 |
| [symbol] ID No: | IMAGp998B07407; IMAGp971E0417 | 208806 | Human serum prealbumin gene, complete |
| [symbol] | | | cds |
| [symbol] ID No: | IMAGp998C16522; IMAGp971C1521 | 242631 | Human mRNA for apolipoprotein AII |
| [symbol] | | | precursor |
| [symbol] ID No: | IMAGp998O055540; IMAGp971O0692 | 2239324 | cholecystokinin B receptor |
| [symbol] | | | |
| [symbol] ID No: | IMAGp998A244944; IMAGp971B2484 | 2010143 | |
| [symbol] | | | |
| [symbol] ID No: | IMAGp998I15843; IMAGp971H1436 | 366038 | SERUM ALBUMIN PRECURSOR |
| [symbol] | | | |
| [symbol] ID No: | IMAGp998K105256; IMAGp971F02120 | 2130177 | |
| [symbol] | | | |
| [symbol] ID No: | IMAGp998B094453; IMAGp971B2475 | 1752104 | |
| [symbol] | | | |
| [symbol] ID No: | IMAGp998G193982; IMAGp971E1864 | 1571370 | Homo sapiens transcribed sequence with moderate similarity to |
| [symbol] | | | protein pir: S30392 (H. sapiens) S30392 phospholipase A2 |
| | | | homolog (clone AF-5) - human retrotransposon |
| [symbol] ID No: | IMAGp998P194992; IMAGp971N1785 | 2028930 | ESTs, Moderately similar to JE0065 retroviral proteinase-like |
| [symbol] | | | protein [H. sapiens] |
| [symbol] ID No: | IMAGp998F015873; IMAGp971H0199 | 2365824 | |
| [symbol] | | | |
| [symbol] ID No: | IMAGp998A054888; IMAGp971B04121 | 1988620 | |
| [symbol] | | | |
| [symbol] ID No: | IMAGp998A04528; IMAGp971A0222 | 244875 | Human cystathionine-beta-synthase (CBS) |
| [symbol] | | | mRNA |
| [symbol] ID No: | IMAGp998H22207; IMAGp971I2210 | 140997 | Growth differentiation factor 15 |
| [symbol] | | | |
| [symbol] ID No: | IMAGp998P151066; IMAGp971M0340 | 451838 | |
| [symbol] | | | |
| [symbol] ID No: | IMAGp998K055550; IMAGp971D0892 | 2243068 | |
| [symbol] | | | |
| [symbol] ID No: | IMAGp998A065260; IMAGp971A0890 | 2131469 | ETS2 v-ets erythroblastosis virus E26 |
| [symbol] | | | oncogene homolog 2 (avian) |
| [symbol] ID No: | IMAGp998J076121; IMAGp971M04102 | 2461158 | Inhibitor of DNA binding 3, dominant |
| [symbol] | | | negative helix-loop-helix protein |
| [symbol] ID No: | IMAGp998L10662; IMAGp971G0530 | 296601 | H. sapiens mRNA for M130 antigen |
| [symbol] | | | cytoplasmic variant 2 |
| [symbol] ID No: | IMAGp998D0595; IMAGp971C043 | 114388 | |
| [symbol] | | | |
| [symbol] ID No: | IMAGp998G06783; IMAGp971L0333 | 342941 | APLN apelin |
| [symbol] | | | |
| [symbol] ID No: | IMAGp998K08276; IMAGp971K12119 | 47615 | Human MHC HLA-B7 class I cell surface |
| [symbol] | | | glycoprotein heavy chain mRNA, complete |
| | | | cds |
| [symbol] ID No: | IMAGp998F152230; IMAGp971F1655 | 898574 | |
| [symbol] | | | |
| [symbol] ID No: | IMAGp998O075514; IMAGp971L03120 | 2229342 | FLJ10808 hypothetical protein FLJ10808 |
| [symbol] | | | |
| [symbol] na genes | | | |
| [symbol] ID No: | IMAGp998A022575; IMAGp971C0257 | 1030921 | |
| [symbol] | | | |
| [symbol] ID No: | IMAGp998E122300; IMAGp971F1455 | 925427 | |
| [symbol] | | | |
| [symbol] ID No: | IMAGp998C12278; IMAGp971H11117 | 48417 | FRCP2 likely ortholog of mouse fibronectin |
| [symbol] | | | type III repeat containing protein 2 |
| [symbol] ID No: | IMAGp998E191782; IMAGp971A1744 | 726522 | Homo sapiens similar to Envoplakin (210 kDa paraneoplastic |
| [symbol] | | | pemphigus antigen) (p210) (210 kDa cornified envelope |
| | | | precursor) (LOC339237), mRNA |
| [symbol] ID No: | IMAGp998O124335; IMAGp971P1173 | 1707107 | Human chitotriosidase precursor mRNA, |
| [symbol] | | | complete cds |
| [symbol] ID No: | IMAGp998K102640; IMAGp971L1957 | 1056129 | |
| [symbol] | | | |
| [symbol] ID No: | IMAGp998F035711; IMAGp971E1095 | 2304770 | |
| [symbol] | | | |
| [symbol] ID No: | IMAGp998O184348; IMAGp971P2373 | 1712105 | ALL1 fused gene from 5q31 |
| [symbol] | | | |
| [symbol] ID No: | IMAGp998B201153; IMAGp971E1741 | 484915 | RNA binding motif protein 5 |
| [symbol] | | | |
| [symbol] ID No: | IMAGp998P08567; IMAGp971P0523 | 260215 | Homo sapiens cDNA FLJ31733 fis, clone |
| [symbol] | | | NT2RI2006943 |
| [symbol] ID No: | IMAGp998A191006; IMAGp971B1539 | 428442 | tachykinin 3 (neuromedin K, neurokinin |
| [symbol] | | | beta) |
| [symbol] ID No: | IMAGp998B02152; IMAGp971I04112 | 31971 | Human mRNA for KIAA0363 gene, partial |
| [symbol] | | | cds |
| [symbol] ID No: | IMAGp998M23288; IMAGp971N21118 | 52650 | Human erythroblastosis virus oncogene homolog 2 (ets-2) |
| [symbol] | | | |
| [symbol] ID No: | IMAGp998I12605; IMAGp971E1525 | 274643 | |
| [symbol] | | | |
| [symbol] ID No: | IMAGp998F015343; IMAGp971E0391 | 2163456 | Chitinase 1 (chitotriosidase) |
| [symbol] | | | |
| [symbol] ID No: | IMAGp998B171824; IMAGp971E1447 | 742576 | H. sapiens HE2 mRNA |
| [symbol] | | | |
| [symbol] ID No: | IMAGp998L24923; IMAGp971O2438 | 396839 | |
| [symbol] | | | |
| [symbol] ID No: | IMAGp998E135802; IMAGp971F1597 | 2338548 | chitinase 1 (chitotriosidase) |
| [symbol] | | | |
| [symbol] ID No: | IMAGp998D171153; IMAGp971F2341 | 484960 | hypothetical protein BC007899 |
| [symbol] | | | |
| [symbol] ID No: | IMAGp998F17593; IMAGp971B1524 | 269968 | Transcribed sequence with strong similarity to protein 1 |
| [symbol] | | | pdb: 14GS (H. sapiens) B Chain B, Glutathione S- |
| | | | Transferase P1-1 Apo Form 1 |
| [symbol] ID No: | IMAGp998N11360; IMAGp971O1414 | 191050 | |
| [symbol] | | | |
| [symbol] ID No: | IMAGp998I152328; IMAGp971I2156 | 936278 | Transcribed sequence with moderate similarity to protein |
| [symbol] | | | pir: S52491 (H. sapiens) S52491 polyadenylate binding |
| | | | protein II - human |
| [symbol] ID No: | IMAGp998I112449; IMAGp971G05108 | 982738 | |
| [symbol] | | | |
| [symbol] ID No: | IMAGp998A17282; IMAGp971B18117 | 50008 | Calcium channel, voltage-dependent, L |
| [symbol] | | | type, alpha 1B subunit |
| [symbol] ID No: | IMAGp998J014173; IMAGp971J0469 | 1644768 | Golgi autoantigen, golgin subfamily a, |
| [symbol] | | | member 6 |
| [symbol] ID No: | IMAGp998O235762; IMAGp971O22130 | 2323438 | |
| [symbol] | | | |
| [symbol] ID No: | IMAGp998C01743; IMAGp971D0132 | 327480 | Collagen, type XXIII, alpha 1 |
| [symbol] | | | |
| [symbol] ID No: | IMAGp998D175081; IMAGp971F1286 | 2062816 | hypothetical protein LOC200213 |
| [symbol] | | | |
| [symbol] ID No: | IMAGp998B2474; IMAGp971D23110 | 23877 | ATPase, aminophospholipid transporter |
| [symbol] | | | (APLT), Class I, type 8A, member 1 |
| [symbol] ID No: | IMAGp998I094494; IMAGp971I0978 | 1837520 | Homo sapiens transcribed sequence with moderate similarity to |
| [symbol] | | | protein pir: S52491 (H. sapiens) S52491 polyadenylate binding |
| | | | protein II - human |
| [symbol] ID No: | IMAGp998B08733; IMAGp971A0333 | 323623 | DKFZP564O1863 protein |
| [symbol] | | | |
| [symbol] ID No: | IMAGp998I11564; IMAGp971K1323 | 258898 | Homo sapiens secreted frizzled-related |
| [symbol] | | | protein 2 (SFRP2), mRNA |
| [symbol] ID No: | IMAGp998K211090; IMAGp971I2040 | 460940 | |
| [symbol] | | | |
| [symbol] ID No: | IMAGp998A181964; IMAGp971E0253 | 796313 | |
| [symbol] | | | |
| [symbol] ID No: | IMAGp998H04617; IMAGp971G0326 | 279219 | chromosome 20 open reading frame 58 |
| [symbol] | | | |
| [symbol] ID No: | IMAGp998I12670; IMAGp971L0430 | 299603 | |
| [symbol] | | | |
| [symbol] ID No: | IMAGp998D145711; IMAGp971D2194 | 2304733 | |
| [symbol] | | | |
| [symbol] ID No: | IMAGp998L06178; IMAGp971H02115 | 38015 | Slit and trk like gene 2 |
| [symbol] | | | |
| [symbol] ID No: | IMAGp998I135799; IMAGp971C2497 | 2337492 | Hypothetical gene supported by AK126852 |
| [symbol] | | | (LOC400516), mRNA |
| [symbol] ID No: | IMAGp998D154762; IMAGp971D1183 | 1940318 | disco-interacting protein 2 (Drosophila) |
| [symbol] | | | homolog |
| [symbol] ID No: | IMAGp998P20366; IMAGp971O1915 | 193411 | Human mRNA for alpha1-acid glycoprotein |
| [symbol] | | | (orosomucoid) |
| [symbol] ID No: | IMAGp998C034512; IMAGp971C0279 | 1844282 | transglutaminase 3 (E polypeptide, |
| [symbol] | | | protein-glutamine-gamma- |
| | | | glutamyltransferase) |
| [symbol] ID No: | IMAGp998G01115; IMAGp971E01103 | 122136 | |
| [symbol] | | | |
| [symbol] ID No: | IMAGp998B22787; IMAGp971A2234 | 344373 | |
| [symbol] | | | |
| [symbol] ID No: | IMAGp998J134174; IMAGp971J1669 | 1645164 | hypothetical protein LOC340094 |
| [symbol] | | | |
| [symbol] ID No: | IMAGp998D04101; IMAGp971A034 | 116691 | |
| [symbol] | | | |
| [symbol] ID No: | IMAGp998D04101; IMAGp971A034 | 116691 | |
| [symbol] | | | |
| [symbol] ID No: | IMAGp998O0470; IMAGp971O03110 | 22040 | Human type IV collagenase mRNA, |
| [symbol] | | | complete cds |
| [symbol] ID No: | IMAGp998A125385; IMAGp971D14124 | 2179475 | phospholipase A2, group IID |
| [symbol] | | | |
| [symbol] ID No: | IMAGp998D194160; IMAGp971F1767 | 1639650 | |
| [symbol] | | | |
| [symbol] ID No: | IMAGp998L132010; IMAGp971K1355 | 814236 | |
| [symbol] | | | |
| [symbol] ID No: | IMAGp998G081965; IMAGp971G1553 | 796831 | Human metalloendopeptidase homolog |
| [symbol] | | | (PEX) mRNA, complete sequence |
| [symbol] ID No: | IMAGp998B072455; IMAGp971B0956 | 984870 | Similar to ankylosis, progressive homolog |
| [symbol] | | | |

| | | | 50% | 75% | 100% | | | Seminom/ |
|---|---|---|---|---|---|---|---|---|
| | Genename | Chromosome | CIS | CIS | CIS | Seminoma | EC | EC |
| [symbol] ID No: | NANOG | 12p13.2 | 10.3 | 15.4 | 33.2 | | | |
| [symbol] | | | | | | | | |
| [symbol] ID No: | hXBP1 | | 2.0 | 30.0 | 25.5 | | | |
| [symbol] | | | | | | | | |
| [symbol] ID No: | | | 23.8 | 14.7 | 24.6 | | | |
| [symbol] | | | | | | | | |
| [symbol] ID No: | TMF1 | | 24.8 | 27.6 | 16.6 | | | |
| [symbol] | | | | | | | | |
| [symbol] ID No: | LIN28 | 1p35.3 | 3.6 | 4.7 | 15.0 | | | |
| [symbol] | | | | | | | | |
| [symbol] ID No: | | 11p15.4 | 10.1 | 14.0 | 15.0 | | | |
| [symbol] | | | | | | | | |
| [symbol] ID No: | T1A-2 | 1p36 | 6.6 | 7.3 | 14.8 | | | |
| [symbol] | | | | | | | | |
| [symbol] ID No: | | | 2.9 | 3.2 | 14.2 | | | |
| [symbol] | | | | | | | | |
| [symbol] ID No: | | | 3.3 | 8.7 | 14.2 | | | |
| [symbol] | | | | | | | | |
| [symbol] ID No: | | | 2.5 | 3.8 | 12.9 | | | |
| [symbol] | | | | | | | | |
| [symbol] ID No: | SLC7A3 | Xq12 | 2.6 | 2.1 | 12.3 | | | |
| [symbol] | | | | | | | | |
| [symbol] ID No: | | 14 | 6.7 | 4.5 | 11.4 | | | |
| [symbol] | | | | | | | | |
| [symbol] ID No: | | 6 | NA | 2.7 | 10.0 | | | |
| [symbol] | | | | | | | | |
| [symbol] ID No: | | 1p32.1 | 2.3 | 3.7 | 10.0 | | | |
| [symbol] | | | | | | | | |
| [symbol] ID No: | | | 19.4 | 16.4 | 9.9 | | | |
| [symbol] | | | | | | | | |
| [symbol] ID No: | | 7 | NA | 5.8 | 9.1 | | | |
| [symbol] | | | | | | | | |
| [symbol] ID No: | | | 2.9 | 4.8 | 9.0 | | | |
| [symbol] | | | | | | | | |
| [symbol] ID No: | | 1p35.3 | 3.8 | 3.8 | 8.6 | | | |
| [symbol] | | | | | | | | |
| [symbol] ID No: | SCGB3A2 | 5q32 | 2.7 | 5.2 | 8.3 | | | |
| [symbol] | | | | | | | | |
| [symbol] ID No: | TFAP2C | 20q13.2 | 2.4 | 3.8 | 8.2 | | | |
| [symbol] | | | | | | | | |
| [symbol] ID No: | EFEMP1 | 2p16 | 5.6 | 3.1 | 8.2 | | | |
| [symbol] | | | | | | | | |
| [symbol] ID No: | NFE2L3 | 7p15-p14 | 3.5 | 6.1 | 8.1 | | | |
| [symbol] | | | | | | | | |
| [symbol] ID No: | PIM2 | Xp11.23 | 2.5 | 2.5 | 8.0 | | | |
| [symbol] | | | | | | | | |
| [symbol] ID No: | | 2 | 2.9 | 2.9 | 8.0 | | | |
| [symbol] | | | | | | | | |
| [symbol] ID No: | MYBL2 | | 2.7 | 2.8 | 7.7 | | | |
| [symbol] | | | | | | | | |
| [symbol] ID No: | GLDC | 9p22 | 2.8 | 3.7 | 7.5 | | | |
| [symbol] | | | | | | | | |
| [symbol] ID No: | LBP-9 | 2q14 | 2.8 | 3.2 | 7.4 | | | |
| [symbol] | | | | | | | | |
| [symbol] ID No: | RAB15 | 14 | 3.4 | 4.3 | 7.4 | | | |
| [symbol] | | | | | | | | |
| [symbol] ID No: | ALPL | 1p36.1-p34 | 1.9 | 2.4 | 7.3 | | | |
| [symbol] | | | | | | | | |
| [symbol] ID No: | | | 5.1 | 3.7 | 7.3 | | | |
| [symbol] | | | | | | | | |
| [symbol] ID No: | PRDM1 | 6 | 6.0 | 6.4 | 7.1 | | | |
| [symbol] | | | | | | | | |
| [symbol] ID No: | UP | | 2.3 | 3.3 | 7.1 | | | |
| [symbol] | | | | | | | | |
| [symbol] ID No: | | | 1.5 | 3.0 | 6.9 | | | |
| [symbol] | | | | | | | | |
| [symbol] ID No: | | | 2.0 | 6.9 | 6.7 | | | |
| [symbol] | | | | | | | | |
| [symbol] ID No: | TFCP2L1 | | 2.5 | 4.9 | 6.7 | | | |
| [symbol] | | | | | | | | |
| [symbol] ID No: | | | 2.5 | 3.5 | 6.6 | | | |
| [symbol] | | | | | | | | |
| [symbol] ID No: | | | 2.4 | 4.3 | 6.4 | | | |
| [symbol] | | | | | | | | |
| [symbol] ID No: | HLXB9 | | 1.7 | 3.1 | 6.3 | | | |
| [symbol] | | | | | | | | |
| [symbol] ID No: | PHLDA3 | 1q31 | 3.1 | 2.7 | 6.2 | | | |
| [symbol] | | | | | | | | |
| [symbol] ID No: | | | 2.2 | 3.1 | 6.1 | | | |
| [symbol] | | | | | | | | |
| [symbol] ID No: | COL22A1 | | 2.0 | 3.3 | 6.1 | | | |
| [symbol] | | | | | | | | |
| [symbol] ID No: | NALP9 | | 1.3 | 4.3 | 6.1 | | | |
| [symbol] | | | | | | | | |
| [symbol] ID No: | | | 2.4 | 3.5 | 6.0 | | | |
| [symbol] | | | | | | | | |
| [symbol] ID No: | | 1 | 6.0 | 5.9 | 6.0 | | | |
| [symbol] | | | | | | | | |
| [symbol] ID No: | TEAD4 | 12p13.2-p13.3 | 2.0 | 2.8 | 5.9 | | | |
| [symbol] | | | | | | | | |
| [symbol] ID No: | TCL1B, | 14q32.1 | 1.7 | 4.0 | 5.9 | | | |
| [symbol] | TCL6 | | | | | | | |
| [symbol] ID No: | ETV4 | 17q21 | 3.8 | 3.1 | 5.8 | | | |
| [symbol] | | | | | | | | |
| [symbol] ID No: | DKFZP76 | 1p36.13 | 2.2 | 3.3 | 5.7 | | | |
| [symbol] | 2N0610 | | | | | | | |
| [symbol] ID No: | | | 2.1 | 2.8 | 5.6 | | | |
| [symbol] | | | | | | | | |
| [symbol] ID No: | | | 2.3 | 3.0 | 5.5 | | | |
| [symbol] | | | | | | | | |
| [symbol] ID No: | LRP4 | 11p11.2-p12 | 4.2 | 4.5 | 5.5 | | | |
| [symbol] | | | | | | | | |
| [symbol] ID No: | TCL1A | 14q32.1 | 2.7 | 3.1 | 5.5 | | | |
| [symbol] | | | | | | | | |
| [symbol] ID No: | PROCR | 20 | 14.1 | 7.6 | 5.4 | | | |
| [symbol] | | | | | | | | |
| [symbol] ID No: | | | 1.3 | 2.3 | 5.3 | | | |
| [symbol] | | | | | | | | |
| [symbol] ID No: | LCE | 4q25 | NA | 5.2 | 5.3 | | | |
| [symbol] | | | | | | | | |
| [symbol] ID No: | | | 1.8 | 3.7 | 5.3 | | | |
| [symbol] | | | | | | | | |
| [symbol] ID No: | ETV5 | 3q28 | 2.1 | 2.8 | 5.3 | | | |
| [symbol] | | | | | | | | |
| [symbol] ID No: | | 1 | 1.9 | 2.8 | 5.2 | | | |
| [symbol] | | | | | | | | |
| [symbol] ID No: | PIF1 | 15q22.1 | 1.9 | 2.5 | 5.1 | | | |
| [symbol] | | | | | | | | |
| [symbol] ID No: | | 7 | 2.0 | 4.7 | 5.1 | | | |
| [symbol] | | | | | | | | |
| [symbol] ID No: | | | 3.2 | 4.0 | 5.1 | | | |
| [symbol] | | | | | | | | |
| [symbol] ID No: | DPPA4 | | 1.2 | 2.4 | 4.9 | | | |
| [symbol] | | | | | | | | |
| [symbol] ID No: | E2IG2 | | 2.5 | 2.1 | 4.8 | | | |
| [symbol] | | | | | | | | |
| [symbol] ID No: | SALL4 | 20q13.13-q13.2 | 2.1 | 2.8 | 4.8 | | | |
| [symbol] | | | | | | | | |
| [symbol] ID No: | | | 1.9 | 2.6 | 4.8 | | | |
| [symbol] | | | | | | | | |
| [symbol] ID No: | | | 2.2 | 2.2 | 4.7 | | | |
| [symbol] | | | | | | | | |
| [symbol] ID No: | HRC13227 | 4 | 2.2 | 3.2 | 4.7 | | | |
| [symbol] | | | | | | | | |
| [symbol] ID No: | AND-1 | 14q22.1 | 0.9 | 2.1 | 4.7 | | | |
| [symbol] | | | | | | | | |
| [symbol] ID No: | E2F1 | | 4.6 | 2.8 | 4.7 | | | |
| [symbol] | | | | | | | | |
| [symbol] ID No: | CDH1 | 16q22.1 | 1.7 | 2.1 | 4.7 | | | |
| [symbol] | | | | | | | | |
| [symbol] ID No: | | | 2.2 | 2.6 | 4.6 | | | |
| [symbol] | | | | | | | | |
| [symbol] ID No: | | | 2.1 | 2.3 | 4.5 | | | |
| [symbol] | | | | | | | | |
| [symbol] ID No: | FLJ10652 | 12p12.1 | 2.3 | 3.5 | 4.5 | | | |
| [symbol] | | | | | | | | |
| [symbol] ID No: | | | 2.1 | 2.5 | 4.4 | | | |
| [symbol] | | | | | | | | |
| [symbol] ID No: | SOX17 | 8q11.22 | 2.0 | 2.2 | 4.4 | | | |
| [symbol] | | | | | | | | |
| [symbol] ID No: | NT2RP2000644 | | NA | 10.5 | 4.3 | | | |
| [symbol] | | | | | | | | |
| [symbol] ID No: | | | 2.3 | 3.7 | 4.4 | | | |
| [symbol] | | | | | | | | |
| [symbol] ID No: | CHST2 | 3q24 | 2.8 | 2.1 | 4.2 | | | |
| [symbol] | | | | | | | | |
| [symbol] ID No: | | | 1.4 | 2.5 | 4.2 | | | |
| [symbol] | | | | | | | | |
| [symbol] ID No: | | | 1.8 | 2.4 | 4.1 | | | |
| [symbol] | | | | | | | | |
| [symbol] ID No: | SIAT1 | 3q27-q28 | 2.7 | 3.0 | 4.1 | | | |
| [symbol] | | | | | | | | |
| [symbol] ID No: | SLC25A16 | 10q21.3-q22.1 | 1.6 | 2.3 | 4.1 | | | |
| [symbol] | | | | | | | | |
| [symbol] ID No: | NSMAF | | 2.4 | 3.7 | 4.0 | | | |
| [symbol] | | | | | | | | |
| [symbol] ID No: | | | 2.0 | 2.8 | 4.0 | | | |
| [symbol] | | | | | | | | |
| [symbol] ID No: | | 14 | 4.8 | 2.4 | 4.0 | | | |
| [symbol] | | | | | | | | |
| [symbol] ID No: | | | 2.6 | 2.4 | 4.0 | | | |
| [symbol] | | | | | | | | |
| [symbol] ID No: | ITM2C | 2q37 | 2.8 | 2.3 | 4.0 | | | |
| [symbol] | | | | | | | | |
| [symbol] ID No: | | | 2.2 | 2.9 | 3.9 | | | |
| [symbol] | | | | | | | | |
| [symbol] ID No: | | 3 | 2.1 | 2.2 | 3.9 | | | |
| [symbol] | | | | | | | | |
| [symbol] ID No: | SLC35A2 | Xp11.23-p11.22 | 2.1 | 2.6 | 3.9 | | | |
| [symbol] | | | | | | | | |
| [symbol] ID No: | | | 2.2 | 2.2 | 3.8 | | | |
| [symbol] | | | | | | | | |
| [symbol] ID No: | FLJ20171 | 8q21.3 | 3.2 | 2.4 | 3.8 | | | |
| [symbol] | | | | | | | | |
| [symbol] ID No: | | | 2.5 | 2.8 | 3.8 | | | |
| [symbol] | | | | | | | | |
| [symbol] ID No: | NDFIP2 | 13q22.2 | 5.7 | 4.9 | 3.7 | | | |
| [symbol] | | | | | | | | |
| [symbol] ID No: | CASP4 | | 5.1 | 2.6 | 3.7 | | | |
| [symbol] | | | | | | | | |
| [symbol] ID No: | OSBPL3 | 7p15 | 4.2 | 4.0 | 3.6 | | | |
| [symbol] | | | | | | | | |
| [symbol] ID No: | | | 1.1 | 2.2 | 3.6 | | | |
| [symbol] | | | | | | | | |
| [symbol] ID No: | | | 1.5 | 2.7 | 3.6 | | | |
| [symbol] | | | | | | | | |
| [symbol] ID No: | | | 2.5 | 3.2 | 3.6 | | | |
| [symbol] | | | | | | | | |
| [symbol] ID No: | CPR8 | 15q21.1 | 2.0 | 2.2 | 3.6 | | | |
| [symbol] | | | | | | | | |
| [symbol] ID No: | | | 4.1 | 3.3 | 3.5 | | | |
| [symbol] | | | | | | | | |
| [symbol] ID No: | | | 1.3 | 3.3 | 3.5 | | | |
| [symbol] | | | | | | | | |
| [symbol] ID No: | ANXA3 | 4q13-q22 | 2.6 | 2.7 | 3.5 | | | |
| [symbol] | | | | | | | | |
| [symbol] ID No: | | | 1.4 | 4.0 | 3.5 | | | |
| [symbol] | | | | | | | | |
| [symbol] ID No: | KIAA0644 | 7p21.1 | 1.8 | 3.6 | 3.4 | | | |
| [symbol] | | | | | | | | |
| [symbol] ID No: | CPEB1 | 15q24.2 | 1.4 | 2.6 | 3.4 | | | |
| [symbol] | | | | | | | | |
| [symbol] ID No: | FLJ22662 | 12p13.31 | 2.6 | 2.2 | 3.4 | | | |
| [symbol] | | | | | | | | |
| [symbol] ID No: | DOCK11 | | 2.3 | 2.8 | 3.4 | | | |
| [symbol] | | | | | | | | |
| [symbol] ID No: | SOX4 | 6p23 | 2.0 | 2.4 | 3.4 | | | |
| [symbol] | | | | | | | | |
| [symbol] ID No: | | | 2.5 | 3.4 | 3.4 | | | |
| [symbol] | | | | | | | | |
| [symbol] ID No: | | | 2.4 | 2.9 | 3.3 | | | |
| [symbol] | | | | | | | | |
| [symbol] ID No: | ASH2L | | 2.3 | 2.6 | 3.3 | | | |
| [symbol] | | | | | | | | |
| [symbol] ID No: | | | 0.6 | 3.5 | 3.3 | | | |
| [symbol] | | | | | | | | |
| [symbol] ID No: | | | 1.4 | 2.2 | 3.3 | | | |
| [symbol] | | | | | | | | |
| [symbol] ID No: | MYO5A | 15 | 2.1 | 2.0 | 3.3 | | | |
| [symbol] | | | | | | | | |
| [symbol] ID No: | UGCG | 9 | 1.8 | 2.5 | 3.2 | | | |
| [symbol] | | | | | | | | |
| [symbol] ID No: | | | 2.2 | 2.5 | 3.2 | | | |
| [symbol] | | | | | | | | |
| [symbol] ID No: | VPS13A | | 2.0 | 2.7 | 3.2 | | | |
| [symbol] | | | | | | | | |
| [symbol] ID No: | SNX10 | 7p21.1 | 1.6 | 4.9 | 3.2 | | | |
| [symbol] | | | | | | | | |
| [symbol] ID No: | FAM16AX | Xp22.32 | 2.0 | 2.1 | 3.2 | | | |
| [symbol] | | | | | | | | |
| [symbol] ID No: | IF | 4q25 | 6.5 | 2.3 | 3.2 | | | |
| [symbol] | | | | | | | | |
| [symbol] ID No: | DYSF | 2p13.3-p13.1 | 2.9 | 2.3 | 3.2 | | | |
| [symbol] | | | | | | | | |
| [symbol] ID No: | | | 1.8 | 2.8 | 3.2 | | | |
| [symbol] | | | | | | | | |
| [symbol] ID No: | | | 0.8 | 2.7 | 3.2 | | | |
| [symbol] | | | | | | | | |
| [symbol] ID No: | | | 1.3 | 2.3 | 3.1 | | | |
| [symbol] | | | | | | | | |
| [symbol] ID No: | | | 1.5 | 2.6 | 3.1 | | | |
| [symbol] | | | | | | | | |
| [symbol] ID No: | SLC38A1 | | 1.9 | 2.5 | 3.1 | | | |
| [symbol] | | | | | | | | |
| [symbol] ID No: | | | 2.1 | 2.6 | 3.1 | | | |
| [symbol] | | | | | | | | |
| [symbol] ID No: | | | 1.3 | 2.9 | 3.1 | | | |
| [symbol] | | | | | | | | |
| [symbol] ID No: | TIMELESS | | 1.5 | 2.1 | 3.1 | | | |
| [symbol] | | | | | | | | |
| [symbol] ID No: | DDIT4L | 4q23 | 2.9 | 5.5 | 3.1 | | | |
| [symbol] | | | | | | | | |
| [symbol] ID No: | UNG | 12 | 1.8 | 2.7 | 3.0 | | | |
| [symbol] | | | | | | | | |
| [symbol] ID No: | SEMA3A | | 1.9 | 2.3 | 3.0 | | | |
| [symbol] | | | | | | | | |
| [symbol] genes | | | | | | | | |
| [symbol] ID No: 134 | TXNIP | | | | | | | |
| [symbol] ID No: 135 | CALD1 | | | | | | | |
| [symbol] ID No: 136 | CD74 | | | | | | | |
| [symbol] ID No: 137 | C7 | | | | | | | |
| [symbol] ID No: 138 | CXCL16 | | | | | | | |
| [symbol] ID No: 139 | CYBRD1 | | | | | | | |
| [symbol] ID No: 140 | FITM 1 | | | | | | | |
| [symbol] ID No: 141 | SALL4 | | | | | | | |
| [symbol] ID No: 142 | TRA1 | | | | | | | |
| [symbol] ID No: 143 | COL1A2 | | | | | | | |
| [symbol] ID No: 144 | HDAC3 | | | | | | | |
| [symbol] ID No: 145 | HLA-DRA | | | | | | | |
| [symbol] ID No: 146 | OPA1 | | | | | | | |
| [symbol] ID No: 147 | BPAG1 | | | | | | | |
| [symbol] ID No: 148 | DCN | | | | | | | |
| [symbol] ID No: 149 | CD47 | | | | | | | |
| [symbol] ID No: 150 | | | | | | | | |
| [symbol] ID No: 151 | RPL27 | | | | | | | |
| [symbol] ID No: 152 | RPS16 | | | | | | | |
| [symbol] ID No: 153 | | | | | | | | |
| [symbol] ID No: 154 | OIC1 | | | | | | | |
| [symbol] ID No: 155 | | | | | | | | |
| [symbol] ID No: 156 | | | | | | | | |
| [symbol] ID No: 157 | | | | | | | | |
| [symbol] genes | | | | | | | | |
| [symbol] ID No: | S100A14 | | 1.1575 | 1.335 | 1.685 | 0.6 | 24.36 | 0.105 |
| [symbol] | | | | | | | | |
| [symbol] ID No: | TM4SF3 | | 3.165 | 0.88 | 0.9625 | 0.6 | 17.62 | 0.05 |
| [symbol] | | | | | | | | |
| [symbol] ID No: | | 10 | 1.1025 | 1.15 | 1.4575 | 1.29 | 15.53 | NA |
| [symbol] | | | | | | | | |
| [symbol] ID No: | | | 2.3325 | 0.845 | 0.5625 | 0.61 | 15.035 | 0.065 |
| [symbol] | | | | | | | | |
| [symbol] ID No: | | | 2.3675 | 0.87 | 0.5475 | 0.715 | 14.865 | 0.085 |
| [symbol] | | | | | | | | |
| [symbol] ID No: | | | 1.21 | 1.23 | 0.95 | 0.93 | 13.86 | 0.06 |
| [symbol] | | | | | | | | |
| [symbol] ID No: | | | 0.79 | 0.705 | 0.5075 | 0.695 | 13.475 | 0.04 |
| [symbol] | | | | | | | | |
| [symbol] ID No: | | | 0.8525 | 0.61 | 0.835 | 1.05 | 13.38 | 0.06 |
| [symbol] | | | | | | | | |
| [symbol] ID No: | FGB | 4q28 | 1.205 | 1.36 | 1.105 | 0.8 | 11.695 | 0.08 |
| [symbol] | | | | | | | | |
| [symbol] ID No: | AGR2 | 7p21.3 | 0.78 | 0.725 | 0.71 | 1.2 | 11.05 | 0.06 |
| [symbol] | | | | | | | | |
| [symbol] ID No: | CCDC2 | | 2.14 | 0.85 | 0.56 | 0.67 | 9.57 | 0.105 |
| [symbol] | | | | | | | | |
| [symbol] ID No: | KIAA1712 | | 2.145 | 0.865 | 0.58 | 0.89 | 9.5 | 0.065 |
| [symbol] | | | | | | | | |
| [symbol] ID No: | FLJ20958 | | 1.955 | 0.875 | 0.605 | 0.655 | 9.415 | 0.095 |
| [symbol] | | | | | | | | |
| [symbol] ID No: | C4orf7 | | 0.795 | 1.015 | 1.055 | 1.405 | 9.35 | 0.095 |
| [symbol] | | | | | | | | |
| [symbol] ID No: | | | 0.665 | 0.82 | 1.165 | 1.02 | 9.3 | 0.08 |
| [symbol] | | | | | | | | |
| [symbol] ID No: | KRT19 | 15 | 0.88 | 0.91 | 0.77 | 0.56 | 8.88 | 0.065 |
| [symbol] | | | | | | | | |
| [symbol] ID No: | FN1 | | 1.7425 | 0.8 | 0.685 | 0.755 | 8.875 | 0.09 |
| [symbol] | | | | | | | | |
| [symbol] ID No: | | | 0.8275 | 1.325 | 0.955 | 0.915 | 8.615 | 0.105 |
| [symbol] | | | | | | | | |
| [symbol] ID No: | | | 0.8725 | 0.79 | 0.9425 | 1.185 | 8.475 | 0.15 |
| [symbol] | | | | | | | | |
| [symbol] ID No: | CDX2 | 13q12.3 | 1.185 | 0.97 | 0.985 | 1.34 | 8.415 | 0.165 |
| [symbol] | | | | | | | | |
| [symbol] ID No: | TPARL | 4q12 | 1.06 | 1.46 | 0.8625 | 1.125 | 8.395 | 0.1 |
| [symbol] | | | | | | | | |
| [symbol] ID No: | DLGAP1 | 8 | 2.6125 | 0.98 | 0.755 | 0.695 | 7.83 | 0.095 |
| [symbol] | | | | | | | | |
| [symbol] ID No: | TTR | 18q12.1 | 0.8325 | 0.965 | 0.95 | 0.925 | 7.675 | 0.175 |
| [symbol] | | | | | | | | |
| [symbol] ID No: | APOA2 | 1q21-q23 | 0.615 | 0.85 | 1.065 | 1.12 | 7.51 | 0.28 |
| [symbol] | | | | | | | | |
| [symbol] ID No: | CCKBR | | 1.115 | 0.855 | 1.45 | 0.995 | 7.25 | 0.11 |
| [symbol] | | | | | | | | |
| [symbol] ID No: | | | 0.85 | 1.15 | 1.785 | 1.21 | 7.175 | 0.12 |
| [symbol] | | | | | | | | |
| [symbol] ID No: | Q8IUK7 | | 0.64 | 1.28 | 1.01 | 0.86 | 7.01 | 0.24 |
| [symbol] | | | | | | | | |
| [symbol] ID No: | | | 0.56 | 0.88 | 0.8925 | 0.79 | 6.835 | 0.31 |
| [symbol] | | | | | | | | |
| [symbol] ID No: | | | 1.2275 | 0.855 | 0.77 | 0.63 | 6.765 | 0.26 |
| [symbol] | | | | | | | | |
| [symbol] ID No: | | | 1.2325 | 1.335 | 1.49 | 1.82 | 6.69 | 0.11 |
| [symbol] | | | | | | | | |
| [symbol] ID No: | | | 1.4825 | 1.23 | 1.7875 | 1.33 | 6.685 | 0.15 |
| [symbol] | | | | | | | | |
| [symbol] ID No: | | | 3.56 | 1.845 | 0.87 | 1.83 | 6.505 | 0.305 |
| [symbol] | | | | | | | | |
| [symbol] ID No: | | | 1.5625 | 0.88 | 1.02 | 0.925 | 6.45 | 0.125 |
| [symbol] | | | | | | | | |
| [symbol] ID No: | CBS | 21q22.3 | 1.3275 | 0.925 | 0.795 | 1.735 | 6.33 | 0.085 |
| [symbol] | | | | | | | | |
| [symbol] ID No: | GDF15 | 19p13.1-13.2 | 1.095 | 1.18 | 1.0975 | 1.515 | 6.23 | 0.14 |
| [symbol] | | | | | | | | |
| [symbol] ID No: | | | 2.61 | 1.385 | 0.7125 | 0.915 | 5.995 | 0.115 |
| [symbol] | | | | | | | | |
| [symbol] ID No: | | 2 | 1.825 | 2.145 | 1.45 | 1.49 | 5.965 | 0.27 |
| [symbol] | | | | | | | | |
| [symbol] ID No: | ETS2 | | 3.2925 | 1.38 | 1.58 | 0.695 | 5.965 | 0.22 |
| [symbol] | | | | | | | | |
| [symbol] ID No: | ID3 | | 1.7475 | 0.93 | 1.0025 | 0.85 | 5.85 | 0.13 |
| [symbol] | | | | | | | | |
| [symbol] ID No: | CD163 | 12p13.3 | 4.7225 | 1.315 | 0.565 | 1.79 | 5.77 | 0.115 |
| [symbol] | | | | | | | | |
| [symbol] ID No: | | | 3.07 | 2.04 | 1.7 | 0.775 | 5.765 | 0.165 |
| [symbol] | | | | | | | | |
| [symbol] ID No: | APLN | Xq25-26.3 | 1.4975 | 3.02 | 1.8725 | 0.905 | 5.75 | 0.13 |
| [symbol] | | | | | | | | |
| [symbol] ID No: | HLA-B | 6p21.3 | NA | 1.735 | 0.74 | 1.925 | 5.71 | 0.27 |
| [symbol] | | | | | | | | |
| [symbol] ID No: | | | 1.545 | 0.95 | 1.065 | 1.68 | 5.7 | 0.15 |
| [symbol] | | | | | | | | |
| [symbol] ID No: | FLJ10808 | | 3.1825 | 2.86 | 1.83 | 0.515 | 5.69 | 0.155 |
| [symbol] | | | | | | | | |
| [symbol] na genes | | | | | | | | |
| [symbol] ID No: | | | 0.755 | 1.165 | 0.81 | 5.92 | 0.59 | 19.32 |
| [symbol] | | | | | | | | |
| [symbol] ID No: | | | 0.765 | 1.1 | 0.8375 | 7.37 | 0.47 | 11.74 |
| [symbol] | | | | | | | | |
| [symbol] ID No: | FRCP2 | | 1.4325 | 1.48 | 3.045 | 3.135 | 0.135 | 10.505 |
| [symbol] | | | | | | | | |
| [symbol] ID No: | | | 0.975 | 1.125 | 0.8925 | 5.3 | 0.895 | 7.96 |
| [symbol] | | | | | | | | |
| [symbol] ID No: | CHIT1 | 1q31-q32 | 1.4275 | 1.065 | 0.82 | 13.945 | 0.92 | 7.67 |
| [symbol] | | | | | | | | |
| [symbol] ID No: | | | 1.05 | 0.68 | 1.265 | 5.195 | 0.17 | 7.165 |
| [symbol] | | | | | | | | |
| [symbol] ID No: | | | 1.51 | 1.23 | 1.85 | 8.875 | 0.705 | 5.34 |
| [symbol] | | | | | | | | |
| [symbol] ID No: | AF5Q31 | 5q31 | 0.9475 | 0.795 | 0.97 | 6.575 | 0.585 | 4.925 |
| [symbol] | | | | | | | | |
| [symbol] ID No: | RBM5 | 3p21.3 | 1.4275 | 1.15 | 1.485 | 4.95 | 0.92 | 4.665 |
| [symbol] | | | | | | | | |
| [symbol] ID No: | | 12 | 0.9425 | 0.955 | 1.2775 | 3.575 | 0.68 | 4.6 |
| [symbol] | | | | | | | | |
| [symbol] ID No: | TAC3 | 12q13-q21 | 0.6375 | 1.045 | 0.8875 | 7.06 | 0.45 | 4.41 |
| [symbol] | | | | | | | | |
| [symbol] ID No: | KIAA0363 | 7p15.1 | 1.5125 | 1.515 | 1.78 | 3.735 | 0.32 | 4.3 |
| [symbol] | | | | | | | | |
| [symbol] ID No: | | | 0.365 | 0.635 | 0.3925 | 3.1 | 0.335 | 4.14 |
| [symbol] | | | | | | | | |
| [symbol] ID No: | | | NA | NA | NA | 4.11 | 0.41 | 4.115 |
| [symbol] | | | | | | | | |
| [symbol] ID No: | CHIT1 | | 0.85 | 1.205 | 0.9575 | 4.56 | 0.675 | 3.98 |
| [symbol] | | | | | | | | |
| [symbol] ID No: | SPAG11 | 8 | 1.005 | 1.11 | 0.855 | 3.635 | 1.78 | 3.9 |
| [symbol] | | | | | | | | |
| [symbol] ID No: | | 13 | 1.1 | 0.845 | 0.7575 | 3.235 | 0.5 | 3.89 |
| [symbol] | | | | | | | | |
| [symbol] ID No: | CHIT1 | | 1.165 | 1.05 | 1.015 | 3.89 | 0.935 | 3.65 |
| [symbol] | | | | | | | | |
| [symbol] ID No: | LOC148898 | 1p36.11 | 0.5775 | 0.765 | 0.55 | 4.18 | 1.28 | 3.595 |
| [symbol] | | | | | | | | |
| [symbol] ID No: | | | 1.495 | 1.645 | 4.11 | 5.04 | 0.51 | 3.49 |
| [symbol] | | | | | | | | |
| [symbol] ID No: | | | 0.89 | 0.95 | 0.9675 | 6.375 | 0.77 | 3.47 |
| [symbol] | | | | | | | | |
| [symbol] ID No: | | | 0.525 | 0.76 | 0.7275 | 3.06 | 0.37 | 3.425 |
| [symbol] | | | | | | | | |
| [symbol] ID No: | | | 1.74 | 1.31 | 0.855 | 5.835 | 0.97 | 3.3 |
| [symbol] | | | | | | | | |
| [symbol] ID No: | CACNA1B | 9 | 1.1675 | 1.35 | 1.155 | 3.475 | 0.6 | 3.285 |
| [symbol] | | | | | | | | |
| [symbol] ID No: | GOLGA6 | | 3.2825 | 1.34 | 0.91 | 12.645 | 1.23 | 3.26 |
| [symbol] | | | | | | | | |
| [symbol] ID No: | | | 0.875 | 1.355 | 1.1025 | 3.445 | 0.465 | 3.24 |
| [symbol] | | | | | | | | |
| [symbol] ID No: | COL23A1 | 5 | 1.195 | 1.345 | 1.61 | 3.25 | 0.495 | 3.05 |
| [symbol] | | | | | | | | |
| [symbol] ID No: | LOC200213 | | 1.4125 | 1.425 | 1.7625 | 3.165 | 0.48 | 3.025 |
| [symbol] | | | | | | | | |
| [symbol] ID No: | ATP8A1 | 4 | 2.2925 | 1.815 | 1.565 | 3.275 | 0.58 | 3.015 |
| [symbol] | | | | | | | | |
| [symbol] ID No: | | | 0.6 | 0.89 | 0.7725 | 3.37 | 0.28 | 3.005 |
| [symbol] | | | | | | | | |
| [symbol] ID No: | DKFZP564O1863 | 12p12.3 | 1.155 | 1.335 | 0.95 | 5.315 | 1.005 | 2.985 |
| [symbol] | | | | | | | | |
| [symbol] ID No: | SFRP2 | | 1.9175 | 1.6 | 1.4625 | 5.425 | 0.595 | 2.86 |
| [symbol] | | | | | | | | |
| [symbol] ID No: | | | 0.865 | 1.26 | 1.445 | 5.265 | 0.545 | 2.86 |
| [symbol] | | | | | | | | |
| [symbol] ID No: | | | 1.41 | 1.25 | 3.07 | 4.805 | 1.27 | 2.85 |
| [symbol] | | | | | | | | |
| [symbol] ID No: | C20orf58 | 20 | 1.2025 | 1.075 | 1.0825 | 3.935 | 0.495 | 2.845 |
| [symbol] | | | | | | | | |
| [symbol] ID No: | | | 1.35 | 2.66 | 1.9625 | 4.195 | 1.03 | 2.81 |
| [symbol] | | | | | | | | |
| [symbol] ID No: | | | 1.045 | 0.615 | 1.2575 | 4.38 | 1.46 | 2.78 |
| [symbol] | | | | | | | | |
| [symbol] ID No: | SLITRK2 | | 1.055 | 1.42 | 1.2175 | 4.23 | 0.615 | 2.78 |
| [symbol] | | | | | | | | |
| [symbol] ID No: | | | 1.905 | 0.87 | 0.76 | 3.9 | 0.995 | 2.645 |
| [symbol] | | | | | | | | |
| [symbol] ID No: | DIP2 | 21q22.3 | 1.0925 | 1.245 | 1.325 | 3.34 | 0.65 | 2.52 |
| [symbol] | | | | | | | | |
| [symbol] ID No: | ORM1, ORM2 | 9q31-q32 | 0.865 | 0.81 | 1 | 4.54 | 0.675 | 2.435 |
| [symbol] | | | | | | | | |
| [symbol] ID No: | TGM3 | | 0.69 | 1.465 | 0.54 | 3.72 | 0.705 | 2.415 |
| [symbol] | | | | | | | | |
| [symbol] ID No: | | 2 | 0.7625 | 1.05 | 1.045 | 4.26 | 0.51 | 2.315 |
| [symbol] | | | | | | | | |
| [symbol] ID No: | | 9 | 0.5275 | 0.99 | 2.345 | 5.42 | 0.505 | 2.305 |
| [symbol] | | | | | | | | |
| [symbol] ID No: | | | 1.265 | 0.94 | 0.7825 | 4.99 | 0.93 | 2.26 |
| [symbol] | | | | | | | | |
| [symbol] ID No: | | | 1.05 | 1.035 | 1.165 | 6.91 | 0.79 | 2.23 |
| [symbol] | | | | | | | | |
| [symbol] ID No: | | | 1.05 | 1.035 | 1.165 | 6.91 | 0.79 | 2.23 |
| [symbol] | | | | | | | | |
| [symbol] ID No: | MMP9 | 20q11.2-q13.1 | 1.0825 | 1.05 | 0.9175 | 6.705 | 2.12 | 2.095 |
| [symbol] | | | | | | | | |
| [symbol] ID No: | PLA2G2D | 1p36.12 | 1.355 | 1.02 | 0.8925 | 4.54 | 1.095 | 2.05 |
| [symbol] | | | | | | | | |
| [symbol] ID No: | | | 1.485 | 1.225 | 1.395 | 3.85 | 0.62 | 2.03 |
| [symbol] | | | | | | | | |
| [symbol] ID No: | | | 0.635 | 1.06 | 0.685 | 4.8 | 0.735 | 2.025 |
| [symbol] | | | | | | | | |
| [symbol] ID No: | PHEX | Xp22.2-p22.1 | 1.4175 | 1.31 | 1.135 | 3.235 | 0.74 | 2 |
| [symbol] | | | | | | | | |
| [symbol] ID No: | | | 1.3475 | 1.025 | 0.99 | 85.23 | 1.39 | 1.12 |
| [symbol] | | | | | | | | |

[symbol]

indicates data missing or illegible when filed

[symbol]

lists features descriptions of all the sequences. In most instnaces the RZPD (German Resource Center for Genome Research) and [symbol] clone numbers are reported. In addition to that, also the best applicable feature description and gene name as well as chromosomal [symbol] is reported. In the last 6 columns results from the microarray study are reported. 50%, 75%, and 100% CIS reflects analysis on [symbol] with CIS in 50%, 75, and 100% of the tubules in a given sample. Seminoma reflects a sample with classical seminoma and EC [symbol] a sample with embryonal carcinoma. Seminoma/EC reflects an individual experiment with seminoma vs. EC. [symbol] and evaluation of the results of the AP-2γ staining of semen samples from the various patient categories. The staining was classified using an arbitrary [symbol] after a systematical and independent evaluation by two investigators (CHH and ERM), who had no prior knowledge of the diagnosis. The group with [symbol] neoplasms included patients with the following tumours: 5 seminomas, 4 non-seminomas, 1 mixed GCT and 2 unilateral CIS, which had not proceeded [symbol] tumour, and the subfertile patient diagnosed with CIS in this study. The group of patients with other diseases comprised: 1 benign testis tumour [symbol] adenoma), 1 malignant lymphoma, 2 Hodgkin's disease, 1 glioblastoma, 1 Wegeners granulomatosis, 1 Crohn's disease, 1 bone tumour, 1 torsio testis, [symbol] tumour, 1 Klinefelter, 1 XYY male. The healthy controls were recruited among apparently healthy young men attending our department for other [symbol] and 59 subfertile men, of whom 16 had normospermia and 43 had oligospermia. Statistical analysis of data were performed using the Confidence Interval [symbol] programme, version 2.0, developed by D.G. Altman, D. Machin, T.N. Bryant and M.J. Gardner.

TABLE II

| | | Mean age | AP-2γ-positive | |
|---|---|---|---|---|
| Studied groups | N | (years) | N | (%, 95% CI) |
| Testicular tumour or CIS before | 13^{#} | 33 | 6^{#} | (46.2%, CI 23-71%) |
| operation/irradiation | | | | |
| Testicular cancer after operation, | 7 | 32 | 0 | (0%, CI 0-35%) |
| [symbol] contralateral CIS | | | | |
| Other diseases | 12 | 29 | 0 | (0%, CI 0-24%) |
| Healthy controls | 14 | 32 | 0 | (0%, CI 0-22%) |
| subfertile patients with normospermia | 16 | 32 | 0 | (0%, CI 0-19%) |
| subfertile patients with oligospermia | 43^{#} | 34 | 1^{#} | (2.3%, CI 0.4-12%) |

[symbol]

indicates data missing or illegible when filed

TABLE III
Summary of the Immunohistochemical staining for AP-2γ.

| | | | | |
|---|---|---|---|---|
| | | | AP-2γ (staining intensity | |
| | | | and approximate % of | |
| [symbol] | N | Histology/Diagnosis | positive cells in a section) | Remarks |

[symbol]

testes:

| | | | | |
|---|---|---|---|---|
| 10.22 wg | 11 | Normal fetal testes | − to ++ (75-100%) | 3 samples negative |
| [symbol] wg | 4 | Normal fetal testes | +/− to + (5-50%) | |
| [symbol] wg | 2 | Normal fetal testes | +/− (<10%) | |
| [symbol] months | 2 | Normal infantile testes | − to + (25%) | |
| [symbol] months | 5 | Normal infantile testes | +/− (single cells or <10%) | |
| [symbol] years | 3 | Normal infantile testes | − | |
| [symbol] years | 7 | Normal pre-pubertal testes | − | |
| [symbol] years | 1 | Normal testes with complete spermatogenesis | − | |

[symbol]

ovaries:

| | | | | |
|---|---|---|---|---|
| [symbol] | 1 | Normal fetal ovary | + (single cells) | Oogonia+ |
| [symbol] wg | 5 | Normal fetal ovaries | +/− to + | Oogonia+, oocytes− |
| [symbol] wg | 7 | Normal fetal ovaries | +/− to ++ | Oogonia+, oocytes− |
| [symbol] wg | 9 | Normal fetal ovaries | + to ++ | Oogonia+, oocytes− |
| [symbol] | 1 | Normal adult ovary | − | |

[symbol]

and intersex gonads:

| | | | | |
|---|---|---|---|---|
| [symbol] wg | 1 | Fetal testis (mosaic isochromosomeY) | − | AMH+, Oct4- |
| [symbol] | 2 | Fetal testes (AIS, 46, XY females) | + (10%) | |
| [symbol] months | 1 | Infantile testis (AIS, 46, XY females) | + (5%) | |
| [symbol] months | 1 | Ovotestis (46 XX hermaphrodite) | +/− | Gonocytes positive, |
| | | | | oocytes negative |
| [symbol] months - 6 | 3 | Pre-pubertal testes (AIS, 46 XY females) | + to +++ (2-10%) | Gonocytes with CIS |
| [symbol] | | | | characteristics, |
| | | | | PLAP+ or −. |
| [symbol] | 6 | Pre-pubertal testes (AIS, 46 XY females) | − | |
| [symbol] years | 1 | Infantile testis with decreased number of germ | − | |
| | | cell (idiopathic genital ambiguity) | | |
| [symbol] years | 1 | Pre-pubertal testis (Prader-Willy's syndrome) | − | |
| [symbol] years | 1 | Leydig-cell hyperplasia (adrenogenital | − | |
| | | syndrome) | | |
| [symbol] years | 1 | 46, XY female, 17βHD mutation | − | |
| [symbol] years | 1 | Dysgenetic testis with CIS and gonadoblastoma | ++ (90-100%) | |
| | | (45, X/46XY male) | | |
| [symbol] years | 1 | Klinefelter, bilateral testicular cryptorchidism | − | |
| [symbol] years | 9 | Adult testes with TDS (undifferentiated tubules, | − | |
| | | microliths) but without CIS or TGCT | | |
| [symbol] years | 3 | Adult testes with TDS (undifferentiated tubules, | − | |
| | | microliths) and contralateral TGCT | | |

[symbol]

carcinoma in situ and tumour samples:

| | | | | |
|---|---|---|---|---|
| [symbol] | 5 | CIS adjacent to seminomas | + to +++ (90-100%) | |
| [symbol] | 5 | CIS adjacent to non-seminomas | + to +++ (90-100%) | |
| [symbol] | 2 | CIS adjacent to mixed TGCT | ++ (90-100%) | |
| [symbol] | 2 | CIS without overt TGCT | +++ (90-100%) | |
| [symbol] | 12 | Classical, homogenous seminomas | + to +++ (90-100%) | |
| [symbol] | 12 | Non-seminomas | +/− to ++ | Various epithelial |
| | | | | and stromal |
| | | | | elements positive |
| [symbol] | 2 | Spermatocytic seminomas | − | |
| [symbol] /6 years | 3 | Leydig cell tumors | − to + | Cytoplasmic, background? |
| [symbol] years | 1 | Gonadoblastoma | +++ | |
| [symbol] | 1 | Testicular B-cell lymphoma | − | |

[symbol]

issue samples:

| | | | | |
|---|---|---|---|---|
| [symbol] | 1 of | Liver, heart, thymus, thyroid gland, kidney and | − | |
| [symbol] wg | each | adrenal gland | | |
| [symbol] | 11 | Normal breast tissues | + to ++ (90-100%) | Subpopulation of basally |
| | | | | situated duct cells |
| [symbol] | 47 | Infiltrating ductal breast adenocarcinomas | 7 samples +/− (<10%) | |
| | | | 7 samples + (50%) | |
| | | | 33 samples − | |
| [symbol] | 1 | Breast mucinous carcinoma | − | |
| [symbol] | 4 | Breast medullary carcinomas | 2 samples +/− (90-100%) | |
| [symbol] | 3 | Ductal breast carcinoma in situ | +/− | Single basal cells |
| [symbol] | 2 | Tubular breast cancers | − | |
| [symbol] | 1 | Infiltrating nodular breast adenocarcinoma | − | |
| [symbol] | 4 | Lung | + | Epithelial pneumocytes |
| | | | | positive |
| [symbol] | 7 | Skin | +/− (25%) | Basal cells |
| [symbol] | 1 | Kidney | +/− (50%) | Single cells in canaliculi, |
| | | | | glomeruli− |
| [symbol] | 1-3 | Skeletal muscle, heart muscle, stomach, | − | |
| | of | esophagus, small intestine, colon, liver, spleen, | | |
| | each | pancreas, salivary gland, pituitary gland, adrenal | | |
| | | gland, thyroid gland, parathyroid gland, thymus | | |
| | | gland, tonsil, epididymis, uterus, cervix, ovarian | | |
| | | serous adenocarcinoma, prostate gland, | | |
| | | prostatic cancer, omentum, peripheral nerve, | | |
| | | cerebral cortex, cerebellum | | |

[symbol]

ll lines

| | | | |
|---|---|---|---|
| [symbol] cells | Day 0, 3, 15 of retinoic acid treatment | − to + | In all samples a |
| | | | subpopulation of cells |
| | | | were stained. |
| [symbol] | Day 0, 1, 3, 7, 10 of retinoic acid treatment | ++ | In all samples most cells |
| | | | were stained. |
| [symbol] | Exposed to ICI-182, 780, ethanol or 1 nM | − to + | In all samples a |
| | estradiol for 24 or 48 h. | | subpopulation of cells |
| | | | were stained. |

Abbreviations: N = number; wg = weeks of gestation; AIS = androgen insensitivity syndrome; AMH = anti-Müllerian hormone; TDS = testicular dysgenesis syndrome; TGCT = germ cell tumor; CIS = carcinoma in situ.
Staining intensity: +++: strong staining, ++: moderate staining, +: weak staining, +/−: very weak staining, −: no positive cells detected. [symbol]
indicates data missing or illegible when filed

### EXAMPLES

### Materials and Methods

### Tissue Samples

The testicular tissue samples were obtained directly after orchidectomy and macroscopic pathological evaluation. The use of the orchidectomy samples was approved by the Regional Committee for Medical Research Ethics in Denmark. Samples of homogeneous overt tumors (seminoma or EC) and of testicular parenchyma preserved in the vicinity of a tumor were excised. Each testicular sample was divided into several tissue fragments: 2-3 fragments were snap-frozen at −80° C. for nucleic acid extraction, several adjacent fragments were fixed overnight at 4° C. in Stieve's fluid or paraformaldehyde (PFA), and embedded in paraffin. Fixed sections were subsequently stained with haematoxylin and eosin (HE) or with an antibody against PLAP for histological evaluation. Most samples of testicular parenchyma contained variable numbers of tubules with CIS cells. Samples where no tubules with CIS were found and complete spermatogenesis was present, were used as a normal reference. In an attempt to limit the uncertainty about cellular differences between fragments from the same patient, the present inventors also made a direct print of the frozen fragment that specifically was used for RNA isolation. After staining with a PLAP antibody, the present inventors could roughly confirm the approximate proportion of tubules with CIS as established by histology in adjacent tissue fragments. Samples of tissue containing approximately 50%, 75%, and 100% tubules with CIS cells were chosen for microarray analysis along with the above-mentioned samples of overt tumours and the normal testicular tissue (serving as the common reference). Additional samples of teratomas and embryonal carcinomas were also used in the differential display screening and in the RT-PCR.

### Labeling of Testicular RNA and Hybridization to Microarrays

Total RNA was purified using the Macherey & Nagel kit, DNase digested, and the RNA quality evaluated using the Agilent Bioanalyzer 2100 (Agilent Technologies Inc., CA, USA), 5 μg of total RNA was linearly amplified using the RiboAmp RNA amplification kit (Arcturus GmbH, Germany) yielding a 400-700 fold amplification of the mRNA. The amplified mRNA was reevaluated on the Bioanalyzer 2100 and 5 μg was then subjected to in-direct labeling with Cy3 and Cy5 (Amersham Biosciences) using the Atlas Glass Fluorescent labeling kit (3D Biosciences Clonetech, CA, USA).

The labeled RNA probes were combined and hybridized at 42° C. over night to a 52.000 element cDNA microarray representing the complete Unigene database. The microarray consists of two slides, which are hybridized on top of each other with the labeled probe inbetween. Production and handling of the microarray is described elsewhere (http://emblh3r.embl.de/). Hybridized slides were washed at 42° C. for 5 min in 2×SSC, 0.1% SDS and 10 min in 0.2×SSC, 0.1% SDS, rinsed, dipped in isopropanol, and dried.

Slides were scanned on a GenePix 4000B scanner (Axon Instruments Inc., Union City, Calif., USA) using settings where overall intensity of each channel was equal. The generated images were then analyzed in ChipSkipper (http://chipskipper.embl.de/) and quantified using a histogram segmentation. Flagged spots and controls were taken out and quantified spots were normalized using a framed median ratio centering (frame=200 genes). The average of dye-swaps was used and spots that did not dye-swap or showed a big standard deviation were sorted out. Data was then imported into software Genesis where hierarchical clustering was done using average linkage and Euclidian distances.

### Differential Display Competitive PCR Screening

Total RNA was prepared using the RNeasy kit from Qiagen as described by the manufacturer (Qiagen, Hilden, Germany). RNA samples were double DNAse digested, first “on-column” and subsequently after the RNA had been purified. cDNA was prepared using one-base-anchored AAGCT11V (V=A, C or G) downstream primers. DDRT-PCR (Liang and Pardee, 1992) was subsequently performed using either one- or two-base-anchored (AAGCT11VN; N=A, C, G or T) primers in combination with different of upstream primers. Bands of interest were cut from the DDRT-PCR gels and reamplified using the up- and downstream primers that displayed the bands, but the downstream primer was extended at the 5′-end with a T7-promotor sequence (taatacgactcactatagggAAGCT11V; T7 promotor sequence in small letters), allowing direct sequencing on ALFexpress sequencers (Amersham-Pharmacia Biotech, Uppsala, Sweden), using a Cy5-labelled T7-promotor complementary primer. The DDRT-PCR method has been described in detail previously (Jorgensen, M, et al. Electrophoresis 1999, 20:230-40).

### Verification by RT-PCR

The expression of a selected set of the regulated genes identified in the microarray analysts was verified with RT-PCR as described previously. In brief, cDNA was synthesized using a [symbol] primer. Specific primers targeting each mRNA were designed spanning [symbol] (final concentrations): 12 mM Tris-HCl, pH 8.3; 50 mM KCl; 1.9 mM MgCl2; 0.1% Triton X-100; 0.005% Gelatine; 250 μM dNTP; and 30 pmol of each primer. H2O was used as a negative control and β-actin was used as an internal control in all PCR reactions, resulting in an approximately 800 base pair actin fragment. Cycle conditions: 1 cycle of 2 min. at 95° C.; 30-40 cycles (depending on the intensity of bands) of: 30 sec. at 95° C., 1 min. at 62° C., 1 min. at 72° C. and finally 1 cycle of 5 min. at 72° C. PCR products were resolved on 1.5% agarose gels and visualized by ethidium bromide staining. Digital images of the agarose gels were quantified by the STORM phosphor imager software (ImageQuant, Amersham Biosciences). In a few of the RT-PCR analyses of less abundant transcripts, no bands were detectable after PCR and nested primers were designed. One μl from the first PCR reaction was transferred to a new reaction containing the nested primers and analyzed as above.

### Preparation of Biotin-Labeled Probes and In Situ Hybridization

In situ hybridization was performed to confirm that the identified transcripts were indeed expressed in CIS cells. Probes for in situ hybridization were prepared by re-amplification of the PCR products using nested primers specific to the individual products and with an added T7- or T3-promotor sequence. PCR conditions were: 5 min. 95° C.; 5 cycles of: 30 sec. 95° C., 1 min. 45° C., 1 min. 72° C.; 20 cycles of 30 sec. 95° C., 1 min. 65° C., 1 min. 72° C. and finally 5 min. 72° C. The resulting PCR product was purified on a 2% low melting point agarose gel and sequenced from both ends, using Cy5-labelled primers complementary to the added T3 and T7 tags. Aliquots of about 200 ng were used for in vitro transcription labeling, using the MEGAscript-T3 (sense) or MEGAscript-T7 (anti-sense) kits, as described by the manufacturer (Ambion, Houston, Tex., USA). The composition of the 10×-nucleotide mix was: 7.5 mM ATP, GTP and CTP, 3.75 mM UTP, 1.5 mM biotin-labeled UTP. To estimate quantity and labeling efficiencies, aliquots of the labeled RNA product were analyzed by agarose gel electrophoresis, and dotted onto nitrocellulose filters and developed as described below.

In situ hybridization was performed essentially as described previously (Nielsen, J E, et al. Eur J Histochem 2003, 47:215-22), except for one additional step needed to remove mercury from sections fixed in Stieve fixative. After deparaffination, mercury was removed by 15 min. treatment with potassium iodide (KI/I ratio 2/1), followed by three washes in DEPC water. The iodine was subsequently removed by incubation in sodium thiosulfate pentahydrate (5% w/v, 10 min.) followed by washing (4× DEPC water). Subsequently, the procedure was, in brief, as follows: sections were re-fixed in 4% PFA, treated with, proteinase K (Sigma, St. Louis Mo. USA) (1.0-5.0 μg/ml), post-fixed in PFA, pre-hybridized

[symbol]

Visualization was performed with streptavidin conjugated with alkaline phosphatase (1:1000) (Roche Diagnostics GmbH, Mannheim, Germany) followed by a development with BCIP/NBT (Nielsen, J E, et al. Eur J Histochem 2003, 47:215-22).

### Example 1

### Genes Identified by Microarray

By analyzing testicular samples containing increasing amounts of tubules with CIS the present inventors identified differentially expressed genes as compared to the normal reference with complete spermatogenesis and no CIS cells present.

From normalized scatter plots of gene expression in CIS-containing-tissue versus normal testis tissue, the present inventors observed that approximately 200-300 genes were significantly up-regulated in CIS-containing tissues. Data was filtered so that only genes that were up-regulated in the CIS samples and had low standard deviation were considered for further analysis. The up-regulated genes were a heterogeneous group including transcription factors, such as TFAP2C (ERF-1), LBP-9, TEAD4 (RTEF-1), NFE2L3 (NRF3) and POU5F1, solute carriers e.g. SLC25A16, SLC7A3, oncogenes e.g. RAB-15, TCL1B (TML1), MYBL2 (B-MYB), PIM2 and many more genes, which are listed in Table X. Some of these genes were previously described as CIS markers, e.g. KIT (c-KIT), POV1 (PB39), MYCN (N-MYC) and POU5F1 (OCT-3/4), but the majority were novel, not previously characterized genes.

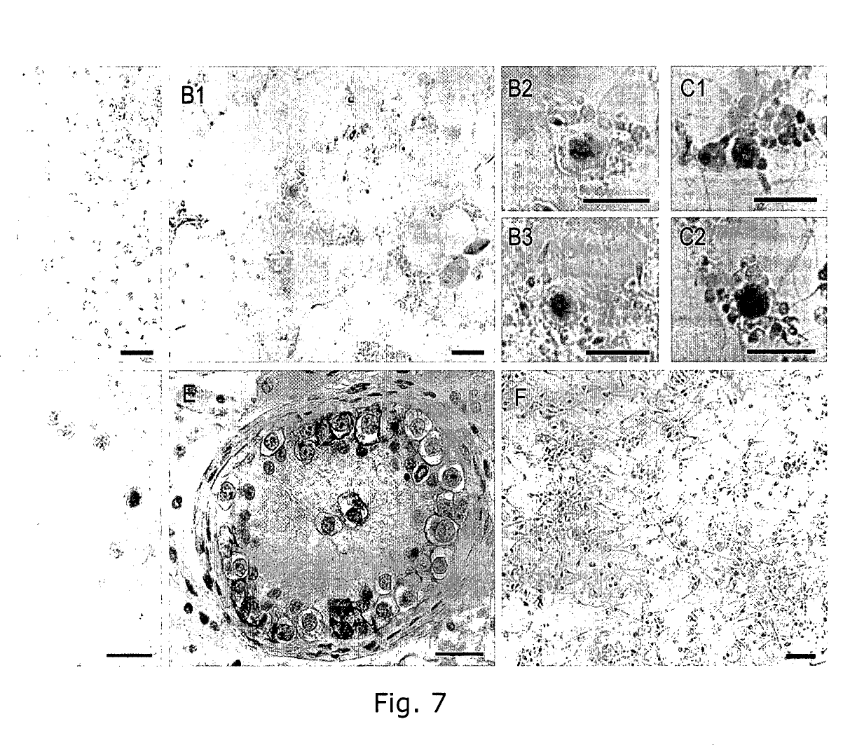

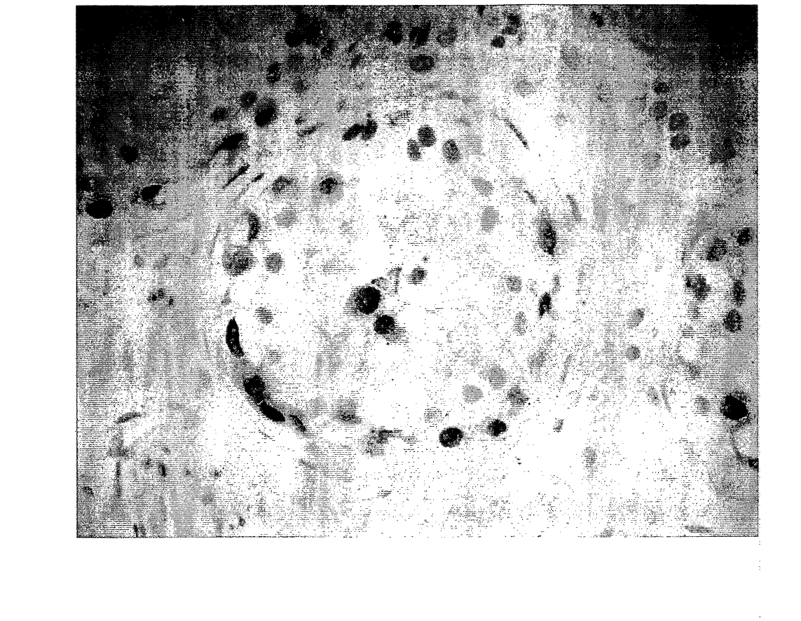

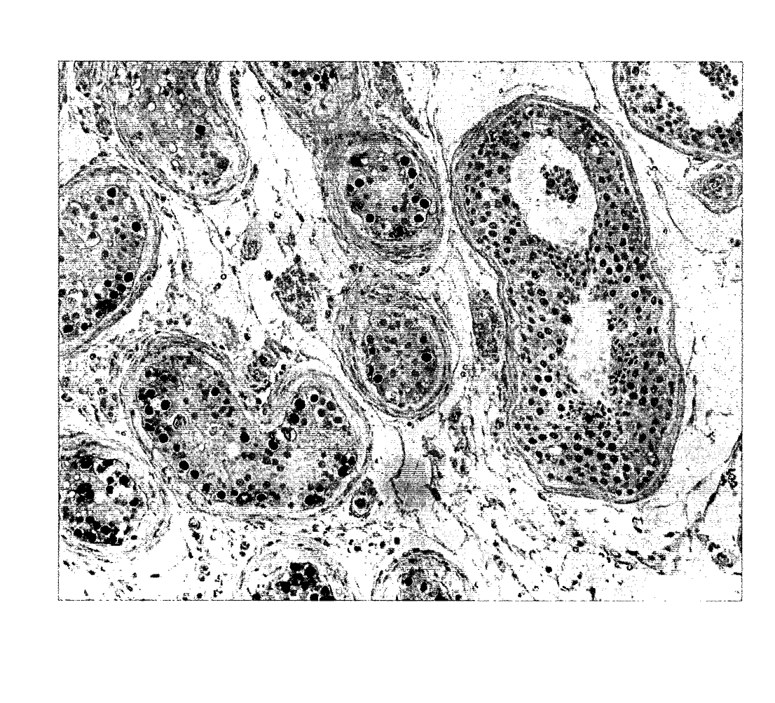

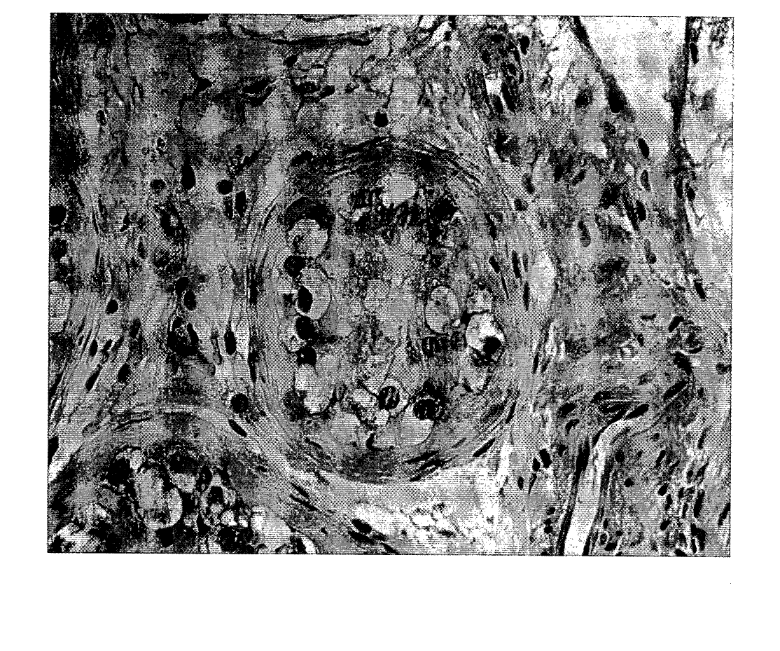

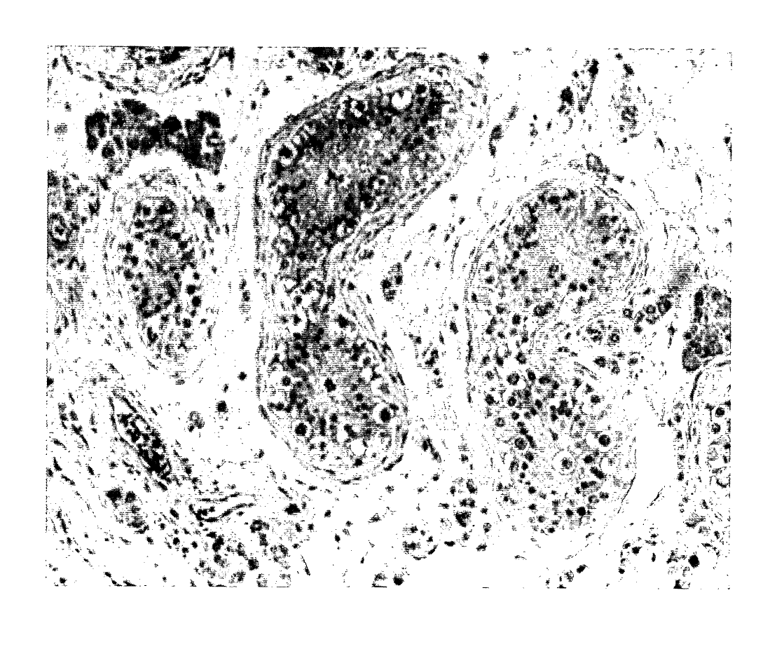

A hierarchical cluster analysis with average linkage of the top most up-regulated genes (minimum 4 fold increase in the sample with CIS in 100% of the tubules and more than 2 fold in the other samples) is shown in FIG. 1. For most of the genes, a gradual increase in expression was observed with increase in the percent of tubules with CIS. This strongly indicates that expression of these genes is linked to the CIS phenotype and indeed to the actual amount of tubules with CIS in the testis. To verify that, the present inventors reanalyzed the expression of selected genes by RT-PCR, in a panel of samples covering a broad spectrum of different testicular neoplasms and normal testicular tissue (FIG. 2A). We also investigated the expression of the non-tissue specific alkaline phosphatase (ALPL) simultaneously with the previously known CIS marker PLAP and found that they were expressed in a similar manner (FIG. 2A).

Both the microarray and the RT-PCR analysis indicated that the observed up-regulation of genes in the samples with CIS were linked to the CIS phenotype and thus to CIS cells. [symbol] TCL1A, TCL1B), may be due to the presence of infiltrating lymphocytes, which are commonly seen in patients with CIS, but not present in the normal testis (Jahnukainen, K, et al. Int J Androl 1995, 18:313-20). In addition, some transcripts up-regulated in the tissues with CIS may originate from immature Sertoli cells, which are sometimes present in testes with CIS, but such dysgenetic changes are relatively rare, so this possibility is less likely.

To confirm the cellular localization, the present inventors performed in situ hybridization for some of the identified genes on tissue sections with CIS. All transcripts tested (NANOG, ALPL, TFAP2C, and SLC7A3) were localized to CIS cells. The present inventors also observed in some cases variable staining of Sertoli cell nuclei, which was most probably unspecific, since similar, but lighter staining was observed for the control sense probe. However, in all cases, the strongest signal was from CIS cells.

### Example 2

### Genes Identified by Differential Display

To obtain knowledge on gene expression changes between the different stages in the development from non-malignant testicular tissue through CIS to the malignant stage of a germ cell cancer, the present inventors performed a systematic comparison by the DDRT-PCR method (Jorgensen, M, et al. Electrophoresis 1999, 20:230-40). For the initial screening the present inventors analysed six testicular biopsy samples: two with homogenous classical seminoma, two containing almost exclusively CIS tubules, of which one was from an intraabdominal CIS and two with normal spermatogenesis. The undescended testis with CIS was of our interest, because in the hypothesis of Testicular Dysgenesis Syndrome, both testicular cancer and undescended testes are symptoms of maldeveloped genitalia.

We performed 402 primer combinations, and assuming that each competitive PCR reaction gives rise to approximately 100 bands on a DDRT gel and that each band corresponds to one mRNA, this investigation allowed comparison of about 40,000 mRNAs. We excised and sequenced 56 bands, which showed overexpression (ranging from 1-10 fold) in at least one of the CIS samples. Several mRNAs were detected with more than one primer combination; for instance, overexpression of CCND2 was detected with three different primer combinations. The RT-PCR analysis verified overexpression of 24 known genes, 4 transcripts corresponding to ESTs and 1 sequence not annotated as a gene.

### Example 3

### Antibodies Against the Genes

Several vendors of commercial antibodies were searched for antibodies to the proteins encoded by the identified genes highly expressed in testicular CIS. In addition, new antibodies against protein product of the newly identified CIS markers genes are going to be generated.

### Example 4

### Identification of Markers for CIS Progression into Seminoma or Non-seminoma

Besides identification of highly specific CIS markers the present inventors also identified markers for the progression of CIS either into seminoma or the non-seminoma. Currently it is not known why some CIS cells give rise to a seminoma and others non-seminomas. It is however speculated that CIS may be predisposed to the one or the other. By identification of the markers for CIS progression together with the CIS markers, the present inventors will be able to: 1) identify CIS cells in a subject 2) predict whether the subject is predisposed to either a seminoma or a non-seminoma, or may develop a combined tumour 3) evaluate to what degree the CIS in the subject has evolved into a seminoma or a non-seminoma 4) monitor during a possible subsequent treatment whether or not all CIS cells or derived tumours have been successfully eradicated.

### Example 5

### Kit Development

A diagnostic kit for screening for a panel of the marker genes can be provided. Such a kit may be in different forms, depending whether the gene expression in a sample will be screened at the RNA or at the protein level. The most obvious form would be a kit based on reverse transcriptase polymerase chain reaction (RT-PCR). By picking out a battery of the most promising CIS marker genes, RNA purified from a human sample can be tested for the presence of CIS marker genes at the transcript level. The present inventors can also take advantage of the high throughput microarray technique for assaying the expression of hundreds of genes simultaneously. This could be in the format of a microarray focused on genes identified here as CIS markers. Additional microarrays e.g. containing markers for the progression into seminoma or nonseminoma can also be developed.

At the protein level, the present inventors can also develop two approaches: either select a limited number of best markers and make an ELISA-based assay using robust and well validated specific antibodies. The present inventors can also develop a protein-microarray using a large number of antibodies against a broader panel of markers, including those previously known, spotted on a glass slide.

### Example 6

### New Nuclear Markers

One very important finding from the study of CIS markers at the protein level was to find proteins localized to the nucleus of the CIS cells. This is important because CIS cells shed to seminal fluid are exposed to different pH and high concentrations of proteases that may damage the cell membrane (and the proteins located within, e.g. receptors) before the ejaculation takes place. A nuclear marker will be better retained than a cell surface marker and thus will increase the possibility for detection in semen samples.

### Example 7

### Human Samples for Detection of CIS

The most convenient and least invasive biological sample to detect CIS cells will be semen (ejaculate). In our earlier studies the present inventors established that some CIS cells are present in semen samples in patients with CIS or overt germ cell tumours in the testes. However, in some cases of early invasion/metastasis, it may also be possible to detect CIS/early tumour cells in a blood sample. Testicular tissue in the form of i.e. biopsies, is nowadays used to detect CIS, and the novel markers can be used to confirm the results of e.g. a CIS array screen, before the final treatment (e.g. orchidectomy) takes place. Yet, other human samples e.g. urine may perhaps be used in the future, e.g. in very young boys, but as only single cells are expected to be detected in urine, careful testing and validation has to be performed.

### Example 8

### CIS Cells are Embryonic Stem Cell-like

Among the genes highly expressed in CIS, the present inventors noticed a range of genes known to be expressed primarily in embryonic stem cells (ESC) and/or primordial germ cells (PGC), such as POU5F1 DPPA4, DPPA5, and NANOG. The present inventors therefore performed a systematic comparison of genes highly expressed in CIS with genes recently [symbol] 13; Sperger, J M, et al. Proc Natl Acad Sci USA 2003, 100: 13350-5). As shown in FIG. 4, many of the genes found in ESC were also highly expressed in our CIS samples. Among the 100 highest expressed CIS marker genes (listed in FIG. 1), 34 genes were reported in ESC. The overlap might even be greater, as many of the genes included on the array are yet uncharacterized and thus may be differently annotated on the microarrays used to expression profile ESC. Excluding genes with no functional description (gene name) leads to an approximately 47% overlap. Furthermore, studies where a relative level of expression in ESC cell compared to a reference was reported (Ramalho-Santos, M, et al. Science 2002, 298:597-600;Sperger, J M, et al. Proc Natl Acad Sci USA 2003, 100:13350-5), the ranking of the expression level seemed to correlate between CIS and ESC, though with some exceptions. The studies the present inventors compared with, in addition all have used different reference samples to explore ESC specific genes and therefore not always agree on the expression in ESC (FIG. 4).

Furthermore, the chromosomal distribution of the over-expressed genes in CIS may be associated with a non-random genomic amplification in certain “hot-spot” areas. The present inventors therefore created a map of the chromosomal distribution of the genes highly expressed in CIS. Searching for the chromosomal localization of these genes and normalizing with the length of each chromosome revealed that there was an over-representation of up-regulated genes on chromosomes 7, 12, 14, 15, and especially chromosome 17 (FIG. 5) Plotting the density of up-regulated genes in each chromosomal band allowed us to narrow down hot-spots to smaller regions, with 17q as the most pronounced region. Recurrent genomic gain of chromosomes 17q and 12 was recently discovered in cultured human ESCs (Draper, J S, et al. Nat Biotechnol 2004, 22:53-4; Pera, M F Nat Biotechnol 2004, 22:42-3). It was postulated that cultured ESC might gain extra chromosome material in these areas after freezing and thawing, which could inhibit apoptosis and increase their self-renewal in culture etc. (Draper, J S, et al. Nat Biotechnol 2004, 22:53-4). This again indicates that CIS cells may have strong similarities to embryonic stem cells and the identified CIS markers may thus be used to identify transplanted embryonic stem cells how have evolved into CIS cells leading to uncontrolled growth of the embryonic stem cells.

### Example 9

### Transcription Factor AP-2γ is a Developmentally Regulated Marker of Testicular Carcinoma In Situ and Germ Cell Tumors

### Purpose

Transcription Factor Activator Protein-2γ (TFAP2C, AP-2γ) was previously reported in extraembryonic ectoderm and breast carcinomas, but not in the testis. the above gene expression study we detected AP-2γ in carcinoma in situ testis (CIS, or intratubular germ cell neoplasia, IGCN), precursor of testicular germ cell tumors (TGCT). In this example we aimed to investigate the expression pattern of AP-2γ and to shed light on this factor in germ cell differentiation and the pathogenesis of germ cell neoplasia.

### Experimental Design

We analyzed expression pattern of AP-2γ at the RNA and protein level in normal human tissues and a panel of tumors and tumor-derived cell lines. In the gonads, we established the ontogeny of expression of AP-2γ in normal and dysgenetic samples. We also investigated the regulation of AP-2γ by steroids and retinoic acid.

### Results

We detected abundant AP-2γ in testicular CIS and in TGCTs of young adults, and confirmed differential expression of AP-2γ in somatic tumors. We found that AP-2γ expression was regulated by retinoic acid in an embryonal carcinoma cell-line (NT2). The investigation of ontogeny of AP-2γ protein expression in fetal gonads revealed that it was confined to oogonia/gonocytes and was down-regulated with germ cell differentiation. In some pre-pubertal intersex cases, AP-2γ was detected outside of the normal window of expression, probably marking neoplastic transformation of germ cells.

### Conclusions

AP-2γ is developmentally regulated and associated with the undifferentiated phenotype in germ cells. This transcription factor may be involved in self-renewal and survival of immature germ cells and tissue-specific stem cells. AP-2γ is a novel marker of testicular CIS and CIS-derived tumors.

The aim of this study was to explore the possible role of AP-2γ in the pathogenesis of reproductive tumors, especially in the testis, and to further investigate the timing of neoplastic transformation of germ cells. To that end, we determined a comprehensive expression profile of AP-2γ in a large panel of normal tissues, tumors and tumo-derived cell lines, established the ontogeny of AP-2γ in normal and dysgenetic human gonads and studied regulation of the AP-2γ expression in selected cell lines.

### MATERIALS AND METHODS

### Tissue Samples

The Regional Committee for Medical Research Ethics in Denmark approved the use of human tissue samples for the studies of novel genes expressed in germ cell cancer.

The tissue samples from adults with testicular neoplasms were obtained directly after orchidectomy and macroscopic pathological evaluation. Each testicular sample was divided into several tissue fragments, which were either snap-frozen at −80° C. for nucleic acid extraction, or fixed overnight at 4° C. in Stieve's fluid, buffered formalin or paraformaldehyde (PFA), and subsequently embedded in paraffin.

Tissue sections were stained with haematoxylin and eosin (HE) or with antibodies against placental alkaline phosphatase (PLAP or ALPP) for histological evaluation (Giwercman, A, et al. APMIS 1991, 99:586-94).

A series of 31 of overt testicular tumors were analyzed by immunohistochemistry, including classical seminomas, various non-seminomatous tumor components, spermatocytic seminomas, Leydig cell tumors and a testicular B-cell lymphoma (listed in Table III). Moreover 14 samples of testicular CIS were analyzed: twelve, which were from tissue adjacent to a TGCT, and two, which had not progressed to an overt tumor, but only had signs of micro-invasion. Furthermore, we included 8 samples containing normal testicular tissue from either patients with prostate cancer or from tissue surrounding a GCT. A representative panel of tissues adjacent to the microscopically examined samples (seminomas, nonseminomatous tumors and specimens with CIS) was analyzed by RT-PCR and ISH. RNAs for RT-PCR were isolated from homogeneous tumors and from specimens containing the largest possible number of tubules with CIS, as judged by microscopical examination of adjacent tissue fragments.

The series for immunohistochemical analysis of the ontogeny of expression of AP-2γ in normal and dysgenetic gonads included paraffin embedded specimens from tissue archives of the Rigshospitalet, Copenhagen University Hospital. The 40 normal fetal tissue samples (19 testicular and 21 ovarian specimens) were obtained after induced or spontaneous abortions and stillbirths, mainly due to placental or maternal problems. The developmental age was calculated from the date of the last menstrual bleeding, supported by the foot size of the fetus. Normal postnatal testicular samples (N=16) were obtained either from autopsies of infants who died suddenly of causes unrelated to the reproductive system or [symbol] individuals with intersex disorders or other components of the testicular dysgenesis syndrome (TDS)(Skakkebaek, N E, et al. Hum Reprod 2001, 16:972-8), and 12 testicular biopsies with mild dysgenetic features (e.g. undifferentiated tubules and/or microliths) with or without CIS, performed for diagnostic reasons from young adult men with sub-fertility or with contralateral germ cell tumors.

In addition 130 samples from extragonadal tissues were examined. Panels of normal tissues and mammary gland tissues with or without neoplastic lesions were investigated using commercial tissue arrays (MaxArray Human Breast Carcinoma and Human Normal Tissue Microarray slides, Zymed, S. San Francisco, Calif., USA). All specimens are listed in Table III.

### Studies in Cell Lines

The following established cancer cell lines were grown in standard conditions (5% CO₂ 37° C.): embryonal carcinoma NT2 and 2102EP lines, and a mammary tumor-derived MCF7 line. Total RNA was isolated for RT-PCR as described below, while for the immunohistochemical staining of the protein, the cells were spun in a cyto-centrifuge or directly grown on microscopic slides.

For analysis of the effect of retinoic acid (RA) on the AP-2γ expression (Andrews, P W Dev Biol 1984, 103:285-93), NT2 cells were grown in DMEM medium (10% FBS, 2 mM L-glutamine, 25 IU/ml penicillin, 25 μg/ml streptomycin) and stimulated with 10 μM RA (Sigma-Aldrich, St. Louis, Mo.) for 0-15 days to induce differentiation; 2102Ep cells were grown in DMEM medium with added 100 μM β-mercaptoethanol, and stimulated with 10 μM RA for 0-10 days. A possible estrogen regulation of AP-2γ was analyzed in MCF7 cells, which were grown in steroid-depleted DMEM medium (5% dextran-charcoal stripped FBS, 1× non-essential amino acids, 1 nM insulin, 2 mM L-glutamine, 25 IU/ml penicillin, 25 μg/ml streptomycin) for 6 days and then exposed to 1 nM estradiol, ICI-182,780 (anti-estrogen), or ethanol (vehicle) for 24 or 48 hours.

### RT-PCR

Total RNA was purified using the NucleoSpin RNAII kit as described by the manufacturer (Macherey-Nagel, Düren, Germany). RNA samples were DNAse digested, and cDNA was synthesized using a dT₂₀ primer. Specific primers were designed for TFAP2C (TFAP2C-ex6: ATCTTGGAGGACGAAATGAGAT, TFAP2C-ex7: CAGATGGCCTGGCTGCCAA), spanning intron-exon boundaries. RT-PCR was performed in duplex in 30 μl of (final concentrations): 12 [symbol] expression of the marker gene ACTB was analyzed with the following primers (ACTB-ex4: ACCCACACTGTGCCCATCTA, ACTB-ex6: ATCAAAGTCCTCGGCCACATT). Cycle conditions for all PCR reactions: 1 cycle of 2 min. at 95° C.; 40 cycles of: 30 sec. at 95° C., 1 min. at 62° C., 1 min. at 72° C. and finally 1 cycle of 5 min. at 72° C. PCR products were run on 1.5% agarose gels and visualized by ethidium bromide staining. Fragment size was 205 base pairs for TFAP2C and 815 for ACTB. Representative bands were excised, cloned (with TOPO® Cloning Invitrogen, Carlsbad, Calif., USA) and sequenced for verification.

### In Situ Hybridization (ISH)

Probes for ISH were prepared by use of specific primers (1^{st} primer combination: AAGAGTTTGTTACCTACCTTACT, CATCAATTGACATTTCAATGGC, 2^{nd} primer combination AATTAACCCTCACTAAAGGGTTAAAGAGCCTTCACT,

TAATACGACTCACTATAGGGCTAAGTGTGTGG) containing an added T3- or T7-promotor sequence, respectively (promotor sequences underlined). PCR conditions were: 5 min. 95° C.; 5 cycles of: 30 sec. 95° C., 1 min. 45° C., 1 min. 72° C.; 20 cycles of 30 sec. 95° C., 1 min. 65° C., 1 min. 72° C. and finally 5 min. 72° C. The resulting PCR product was purified on a 2% low melting point agarose gel and sequenced from both ends, using Cy5-labelled primers complementary to the added T3 and T7 tags. Aliquots of about 200 ng were used for in vitro transcription labeling, using the MEGAscript-T3 (sense) or MEGAscript-T7 (anti-sense) kits, as described by the manufacturer (Ambion, Houston, Tex., USA). The composition of the 10×-nucleotide mix was: 7.5 mM ATP, GTP and CTP, 3.75 mM UTP, 1.5 mM biotin-labeled UTP. To estimate quantity and labeling efficiencies, aliquots of the labeled RNA product were analyzed by agarose gel electrophoresis, dotted onto nitrocellulose filters and developed. ISH was performed on 3-6 different samples as described previously (Hoei-Hansen, C E, et al. Mol Hum Reprod 2004, 10:423-31).

### Immunohistochemistry

A commercially available monoclonal anti-AP-2γ antibody (6E4/4:sc-12762, kindly provided for testing by Santa Cruz Biotechnology Inc., Santa Cruz, Calif., USA) was used. The antibody was validated by western blots by the manufacturer. In addition, several other antibodies were used as controls, in particular to detect CIS cells (anti-PLAP, a monoclonal antibody from BioGenex, San Ramon, Calif., USA; anti-OCT3/4, C-20, sc 8629, Santa Cruz Biotechnology), and to control for integrity of fetal and infantile testis samples obtained at autopsies (anti-AMH, a monoclonal antibody kindly provided by R. Cate, Biogen). The immunohistochemical staining was performed using a standard indirect peroxidase method, as previously described for other antibodies (Giwercman, A, et al. APMIS 1991, [symbol] fixed in formalin or PFA were heated in a 5% urea, pH 8.5, whereas sections fixed in Stieve's fluid were heated in TEG buffer, pH 9.0 (TRIS 6.06 g/5L, EGTA 0.95 g/5L). Subsequently, the sections were incubated with 0.5% H₂O₂ to inhibit the endogenous peroxidase, followed by diluted non-immune goat serum (Zymed, S. San Francisco, Calif., USA) to block unspecific binding sites. The incubation with the primary anti-AP-2γ antibody diluted 1:25-1:100 was carried out overnight at 4° C. For tissues fixed in Stieve's fluid the primary antibody was diluted in Background Reducing antibody diluent (DakoCytomation, Glostrup, Denmark). For negative control, a serial section from each block was incubated with a dilution buffer. Subsequently, a secondary biotinylated goat-anti-mouse link antibody was applied (Zymed, S. San Francisco, Calif., USA), followed by the horseradish peroxidase-streptavidin complex (Zymed, S. San Francisco, Calif., USA). Between all steps the sections were thoroughly washed. The bound antibody was visualized using aminoethyl carbazole substrate (Zymed, S. San Francisco, Calif., USA). Most sections were lightly counterstained with Mayer's haematoxylin to mark unstained nuclei.

The sections were examined under a light microscope (Zeiss) and scored systematically by two investigators (CHH and ERM). The staining was assessed using an arbitrary semi-quantitative score of the percentage of cells stained in a section and their staining intensity: +++: strong staining, ++: moderate staining, +: weak staining, +/−: very weak staining, neg: no positive cells detected.

### RESULTS

### AP-2γ is a New Marker for Testicular Carcinoma In Situ and Derived Germ Cell Tumors

After the discovery of the high expression of AP-2γ in testicular tissues harboring CIS cells (5), we extensively mapped the expression at the mRNA level by reverse transcriptase PCR (RT-PCR) in a panel of testicular neoplasms and tumor-derived cell lines. Subsequently, we determined cellular localization of AP-2γ transcripts by in situ hybridization (ISH), and at the protein level by immunohistochemistry in a comprehensive panel of normal and neoplastic tissues.

RT-PCR showed a high expression of AP-2γ in samples of seminoma, teratoma, embryonal carcinoma and CIS as compared to normal testicular tissue, which showed only a trace of reaction in one biopsy from a patient with an adjacent TGCT, and could encompass a few CIS or micro-invasive tumor cells (FIG. 8). Expression in CIS cells was approximately equivalent in tissue containing CIS adjacent to overt tumors and in CIS at the micro-invasive stage. For a few CIS and tumor samples the expression was confirmed in the [symbol] al. Mol Hum Reprod 2004, 10:423-31)(results not shown). ISH revealed cytoplasmic staining in CIS cells, seminoma, embryonal carcinoma and to lesser extent in some components of teratomas (FIG. 9). ISH in normal testicular tissue was not completely conclusive; in contrast to IHC, which did not show any detectable protein in normal germ cells, a reaction was observed in some spermatocytes and spermatids (results not shown). The presence of TFAP2C transcripts in the normal germ cells with a simultaneous lack of the protein product cannot, therefore, be excluded. This could be due to translational regulation of AP-2γ in the testis. A similar discrepancy between expression at the mRNA and the protein level was previously described for AP-2α in colon cancer (Ropponen, K M, et al. J Clin Pathol 2001, 54:533-8). Immunohistochemistry demonstrated a high abundance of AP-2γ protein in CIS. CIS cells adjacent to TGCTs were uniformly strongly positive for AP-2γ regardless of the tumor type, and equally strong in CIS present in the testis and as an isolated pre-malignant lesion. Gonadoblastoma, a CIS-like pre-malignant lesion of severely dysgenetic gonads was also strongly positive (FIG. 10). Differential expression was however observed in the overt TGCTs: seminomas were strongly positive, whereas a heterogeneous pattern was detected in the non-seminomas (FIG. 9). Embryonal carcinoma demonstrated strongest staining, teratomas displayed staining in some epithelial and stromal elements but many highly differentiated components were negative. In all TGCT specimens staining was present exclusively in the nucleus. No expression was detected in spermatocytic seminoma. In non-germ-cell-derived testicular tumors no expression was detected in a B-cell lymphoma and two Leydig cell tumors (one testicular and one adrenal). One Leydig-Sertoli cell tumor was unexpectedly positive, however, as the staining was confined to the cytoplasm, this may be an unspecific reaction, which is quite frequently observed for various antibodies in the protein and glycoprotein-filled Leydig cells. A similar but much weaker unspecific staining in Leydig cell cytoplasm was also observed in a few specimens of testicular parenchyma. In general, the intensity of staining varied somewhat depending on the fixative used, with stronger staining in tissues fixed with PFA and formalin compared to those fixed in Stieve's and Bouin. In addition, in samples fixed in Stieve's fluid or in high-percentage formalin, there was a tendency to an unspecific staining of erythrocytes. The complete results of the immunohistochemical staining are listed in Table III and representative examples are shown in FIGS. 9, 10 and 11.

### The expression of AP-2γ is Developmentally Regulated in Human Gonads

To investigate whether the high expression of AP-2γ in CIS cells is linked to their gonocyte-like phenotype, as is the case for a number of other markers for CIS cells (Rajpert-De [symbol] of expression of AP-2γ by immunohistochemistry in a panel of specimens of normal human fetal testis and ovaries during development.

The AP-2γ protein was detected in almost all gonocytes in testes from 10 weeks of gestation (earlier samples were not available) until approximately 22 weeks of gestation (FIG. 10), although a few of our analyzed tissue samples failed to show expression. Expression was gradually reduced after week 22, but persisted in a few cells until approximately 4 months of postnatal age. Afterwards, AP-2γ protein was not detected in spermatogonia in normal pre-pubertal, post-pubertal or adult testes.

In fetal ovaries AP-2γ expression was seen in oogonia in high amounts in early gestational ages and to a lesser extent in third trimester. The number of positive oogonia was decreasing with the increasing number of oocytes that entered the meiotic prophase, which were AP-2γ negative. No expression was seen in adult ovary, but only few samples were analyzed.

### Changes of the Expression Pattern of AP-2γ in Intersex and Dysgenetic Gonads

Subsequently, we asked the question whether or not the normal developmental pattern of the expression of AP-2γ is changed in testicular dysgenesis and disorders of sex differentiation, conditions with a markedly increased risk of germ cell neoplasia. Expression of AP-2γ in dysgenetic gonads was analyzed only by immunohistochemistry, because the specimens were available only as paraffin-embedded blocks from tissue archives. The pattern of expression in these specimens was in general similar to that in the normal testes, but in some samples expression deviated from the observed pattern. Expression of AP-2γ was not observed in a sample from a male fetus gestational age 15 weeks with mosaic isochromosome Y, similar to previously observed lack of OCT3/4 (Rajpert-De Meyts, E, et al. Hum Reprod 2004). Staining for AMH was positive, suggesting that the integrity of proteins should be preserved in the sample. Prolonged expression of AP-2γ was seen in cells resembling gonocytes in testicular cords in an ovotestis from a 14-month old XX hermaphrodite, but not in the oocytes present in the ovarian part. In a few of the dysgenetic samples neoplastic, pre-invasive CIS or gonadoblastoma was present and these showed a high expression of AP-2γ. In three cases staining was observed in foci of germ cells that morphologically were classified as somewhere in between gonocytes and CIS (see FIG. 10). In two of the cases (girls with AIS of age 9 months and 6 years, respectively) PLAP was also positive. The third was from a testis removed from a 2-year old girl with AIS, where a very small focus of AP-2γ positive cells was detected. We were not able to locate this focus morphologically in adjacent sections cut before and after, and stainings with PLAP and OCT3/4 were accordingly negative.

### Expression of AP-2γ is Associated with Cell Differentiation or Proliferation and is Cell Type Dependent

In order to define the extent of expression and a possible link to hormonal stimulation, expression of AP-2γ was analyzed in some tissues that are endocrinologically activated and in a panel of normal somatic tissues. In normal female mammary tissue expression was seen in a subpopulation of basally situated epithelial duct cells, and expression was also detected in some, but not all, premalignant or manifest malignant changes in the female breast (FIG. 11, Table III). In samples from skin, the basal layer of keratinocytes was positive, and weaker staining could be seen in the adjacent layers of epidermis (FIG. 11), which is in concert with the previously reported pattern. In normal adult kidney AP-2γ expression was seen in a subset of cells in proximal tubules. Finally, AP-2γ expression was present in lung tissue, in pneumocytes lining alveoles. All other analyzed tissue samples were negative (Table III). We cannot exclude that the AP-2γ expression may be developmentally regulated in some tissues, in analogy to the testis. We could not gain access to a full spectrum of human fetal tissues at different developmental stages, but we have examined and found no AP-2γ expression in the following fetal tissues from the first or second trimester of gestation: epididymis, liver, heart, thymus, thyroid gland, kidney and adrenal gland.

Finally, regulation of AP-2γ expression was studied in vitro in three cell lines. We showed by RT-PCR that AP-2γ was RA-regulated in NT2 cells in a biphasic manner. Strong expression was observed in cells without RA-induced differentiation, after 3 days of RA treatment (which at that point induced cell proliferation) the expression was somewhat stronger and then declined after 15 days when the cells have differentiated (FIG. 8). A heterogeneous staining for AP-2γ protein was seen in NT2 cells by immunocytochemistry in all analyzed samples, regardless of the length of RA treatment. It was not possible to establish the pattern quantitatively, because of differences in Intensity from cell to cell. In contrast, in the 2102Ep cells, which do not differentiate with RA treatment, expression was unaffected by RA treatment at the RNA level, and immunocyto-chemical staining was uniformly strong in all analyzed cells. In MCF7 cells, expression was similar in cells exposed to estraciol, the arti-estrogen ICI, or ethanol, both at the RNA level, and at the protein level, where expression was seen in a subset of cells in all analyzed cells, regardless of treatment ( [symbol] ). For examples of RT-PCR results, see FIG. 8.

### DISCUSSION

In the present study we comprehensively investigated the pattern of expression of the transcription factor AP-2γ, and established AP-2γ as a novel marker for neoplastic germ cells, including testicular carcinoma in situ, the common precursor of TGCTs. Furthermore, we discovered that AP-2γ was developmentally regulated in human gonads, with marked differences between the testes and ovaries.

Our investigations of the pattern of expression of AP-2γ in extra-gonadal normal and neoplastic tissues were largely in concert with previous studies, thus confirming the specificity of the antibody. The transcription factor was preferentially expressed in proliferating immature cells, which retained tissue-specific stem cell characteristics, suggesting a putative oncogenic function of AP-2γ.

In the testis, we found that the AP-2γ protein was expressed in early gonocytes but not in any cell type in the pre- or post-pubertal testis. The expression rapidly declined from approximately 22 weeks of gestation, and was gradually lost while gonocytes differentiated into infantile spermatogonia up to about 4 months of postnatal age, when the final stage of this differentiation takes place, probably stimulated by the hormonal surge at the “mini-puberty” (Andersson, A M, et al. J Clin Endocrinol Metab 1998, 83:675-81;Hadziselimovic, F, et al. J Urol 1986, 136:274-6). Interestingly, in a few pre-pubertal intersex cases, AP-2γ was observed in a subset of gonocytes outside the normal window of expression. This prolonged expression may reflect a delay in differentiation but may perhaps be a marker of the initiation of neoplastic transformation of these immature germ cells into “pre-CIS” cells, which then may further transform to a typical CIS pattern at the onset of puberty. In the ovary, AP-2γ protein was confined to oogonia and was riot detectable after meiotic entry in oocytes. This pattern of expression suggests that the protein plays a role exclusively in undifferentiated germ cells. Analyses in cell lines confirmed that AP-2γ was most abundant in undifferentiated cells, as expression of the transcript was down-regulated after a transient induction in AP-2γpositive NT-2 cells treated with RA for up to 15 days. In cells that do not differentiate (2102Ep) expression was constantly high. Oulad-Abdelghani and colleagues (Oulad-Abdelghani, M, et al. Exp Cell Res 1996, 225:338-47) reported a transient rise in AP-2γ levels with a peak after 12 hours of RA treatment in embryonal carcinoma derived P19 cells, but did not analyze levels beyond 24 hours. An association of AP-2γ with an immature state of germ cells was also observed in overt TGCTs, where classical seminoma and embryonal carcinoma were strongly positive, while highly differentiated somatic elements of teratomas were negative. Spermatocytic seminoma, which is not derived from CIS but from mature spermatogonia, was also AP-2γ-negative. negative. Overall, the pattern of expression of AP-2γ in TGCTs resembles that of KIT and CCT-4, which are also associated with an undifferentiated state and pluripotency, typical for cells retaining some stem cell like features. The high expression of AP-2γ in CIS [symbol] cell. In adult testes AP-2γ was only seen either in CIS or in overt TGCTs, or in rare specimens harboring dysgenetic features, and in the latter only in gonadoblastoma and CIS cells. Thus, AP-2γ can be added to a list of markers for germ cell neoplasms that are also present in gonocytes but not detectable in the normal adult testis, such as PLAP, KIT or OCT-4 (Rajpert-De Meyts, E, et al. APMIS 2003, 111:267-78; discussion 278-9).

Besides an inverse association with cell differentiation, which was also noted by others in other cell systems (Jager, R, et al. Mol Cancer Res 2003, 1:921-9), very little is known concerning the biological function and the mechanism of action of AP-2γ, especially in germ cells. The most interesting question is what are the gene targets for AP-2γ in germ cells, and with which other proteins does this transcription factor interact. In other cell types AP-2γ has been previously shown to interact with a number of genes that are also highly expressed in CIS cells, e.g. IGF-binding protein 5 (Jager, R, et al. Mol Cancer Res 2003, 1:921-9) or KIT (Rajpert-De Meyts, E, et al. Int J Androl 1994, 17:85-92). KIT appears to be of particular interest, as it was reported to be down-regulated in several types of tumors derived from tissues that express AP-2γ, e.g. breast cancer, colorectal cancer or melanoma (Ropponen, K M, et al. J Clin Pathol 2001, 54:533-8).

Estrogen signaling is another possible candidate pathway which could be regulated by AP-2γ, as is suggested by its expression in the epithelium of milk ducts and the association with some forms of breast cancer. While the classic estrogen receptor (ERα) is not expressed in the testis (neither in the fetal life or adulthood), the ERβ is expressed in human fetal testis in the second trimester, and gonocytes are potential targets for estrogen action. This is of interest because it has been suggested that inappropriate exposure to estrogens during fetal life may have an impact on the development of CIS and TGCTs (Skakkebaek, N E, et al. Hum Reprod 2001, 16:972-8). Furthermore, the progression from CIS into an overt TGCT occurs after the endocrinological surge of puberty, when the intra-testicular levels of steroid hormones rise markedly. In the current study, we have analyzed the expression of AP-2γ in a breast tumor-derived cell line, MCF7, which is estrogen dependent. We have not observed any changes in expression, neither after treatment with estradiol, nor an anti-estrogen. This is in disagreement with a recent study (Orso, F, et al. Biochem J 2004, 377:429-38), which showed induction of AP-2γ by estrogens in several breast tumor cell lines. Whether AP-2γ is indeed involved in estrogen signaling remains an unresolved issue, since existing reports are partially contradictory and will require further investigations. We need also to keep in mind that the function of AP-2γ may be cell-type specific, as was demonstrated recently for the transcriptional activation of the ERBB2 gene (Vernimmen, D, et al. 3rd Cancer 2003, 89:899-906) and

[symbol]

In summary, our investigations suggest a role for AP-2γ in pathways regulating cell differentiation, including in fetal gonocytes, which was not previously known. Frequent expression of AP-2γ in various malignancies suggests that the pathways may be involved in oncogenesis. Finally, developmental expression of this transcription factor in the testis makes AP-2γ a very promising new marker for diagnosis of early stages of germ cell neoplasia.

### Example 10

### Early Diagnosis of Pre-invasive Testicular Germ Cell Neoplasia by Immunocytological Detection of AP-2γ-positive Cells in Semen Samples

In the above genome-wide gene expression profiling study of CIS cells, we have identified a number of genes expressed also in embryonic stem cells and foetal gonocytes but not in adult germ cells, thus providing many possible new markers for CIS.

One of such genes was TFAP2C (mapped to chromosome 20q13.2), which encodes the transcription factor activator protein-2 (AP-2γ). We established AP-2γ as a novel marker for foetal gonocytes and neoplastic germ cells, including testicular CIS, with a role in pathways regulating cell differentiation and a possible involvement in oncogenesis. The fact that AP-2γ protein was not expressed in normal adult reproductive tract but was abundant in nuclei of CIS and tumour cells, which are better protected than the cytoplasm from degradation in semen due to a structural strength, prompted us to analyse the value of AP-2γ for detection of CIS and/or tumour cells in ejaculates in a series of patients and controls. The local ethics committee approved the study and participants gave written informed consent.

The present example presents a new non-invasive method capable of detecting testicular cancer at the pre-invasive carcinoma in situ (CIS) in a semen sample. The assay is based on immunocytological staining of semen samples for transcription factor AP-2γ, previously identified as a marker for neoplastic germ cells.

In an investigation of 104 andrological patients, cells positive for AP-2γ were found only in semen samples from patients diagnosed a priori with testicular neoplasms and in a 23-year-old control subject with oligozoospermia and no symptoms of a germ cell tumour (FIG. 7). Testicular biopsies performed during the follow-up of this patient revealed widespread CIS in one testis, thus proving a diagnostic value of the new assay. Optimisation of the method is in progress, but a preliminary assessment gives promise that the method may have a place in screening of germ cell neoplasia in at-risk patients.

The number of patients that we have screened for the presence of immunoreactive AP-2γ is [symbol] have tested 347 andrological patients (including the 104 mentioned above) and found CIS-like cells positive for AP-2γ in semen samples of 3 patients, including the first case described above (a 23-year-old subfertile control subject with widespread CIS confirmed by testicular biopsies). Two other patients are currently under clinical follow-up and being considered for testicular biopses.

### Materials and Methods

A semen sample from each patient was diluted if necessary, divided in portions of 100 μl, spun down on a microscope slide with 400 μl PBS buffer, dried, fixed 10 min in formalin, washed and dried again. The immunocytochemical staining was performed with the monoclonal anti-AP-2γ antibody (6E4/4:sc-12762, Santa Cruz Biotechnology Inc., Santa Cruz, Calif., USA) using a standard indirect peroxidase method, as previously described (Almstrup, K, et al. Cancer Res 2004, 64:4736-43), except for small modifications tested using control semen samples spiked with AP-2γ-positive seminoma cells (FIG. 7A). For negative control, another cytospin was incubated with a dilution buffer, and for positive control AP-2γ-positive seminoma cells were used. Staining was scored using an arbitrary 0-5 scale based on the intensity and morphological resemblance of stained elements to CIS or tumour cells: Score 0—no staining, 1—negative with some unspecific particles stained, 2—trace reaction in small fragments of nucleus-resembling structures, 3—clear staining in a part of a nucleus, with correct morphology, 4—clear staining in a whole nucleus, with correct morphology, 5—two or more cells with score 4. Scores 0-2 were classified as negative, while 3-5 were classified as positive.

### Results

At the evaluation of one of the first series of semen samples, we detected six AP-2γ-positive cells in a sample from one of the control participants (FIG. 7B). He was a 23-year-old man that presented for routine semen analysis due to the infertility problem in the couple. The presence of one small testis and a very poor semen quality in combination with the finding of AP-2γ stained cells constituted a well-justified indication for bilateral open testicular biopsies, which were performed in agreement with the patient. The right-sided biopsy demonstrated normal testicular tissue, whereas the left-sided biopsy revealed numerous tubules containing CIS, which was confirmed with PLAP and AP-2γ staining (FIG. 7D, E). The patient was advised to undergo unilateral orchidectomy, prior to which semen was cryopreserved.

A preliminary assessment of the value of the AP-2γ immunoassay was subsequently done after analysis of semen samples from 104 patients: 12 patients with CIS or testicular [symbol] testicular cancer patients after operation (none had contralateral CIS), 12 patients with other diseases (e.g. referred for semen cryopreservation before treatment for non-testicular cancers and diseases) and 73 healthy or subfertile control patients.

The results are summarised in Table II and examples are shown in FIG. 7. We detected AP-2γ-positive cells in 5 of 12 patients harbouring a priori diagnosed testicular neoplasia, giving a sensitivity of our assay of 42%. However, after addition of the newly diagnosed control patient with CIS, the sensitivity is 46% (95% CI, 23 to 71%). It is especially noteworthy that we did not detect false positive staining among the healthy controls and patients with other cancers, suggesting a high specificity of the assay.

The large proportion of negative results in the testicular cancer patients could be due to a small volume of the semen sample used for the test (50-300 μl out of total normal volume 3-5 ml, depending on concentration and number of samples), thus stressing the need for either repeated analysis or larger volume analysed. Negative results could also be due to compression from the tumour causing obstruction of seminiferous tubules resulting in few or no CIS cells shed into semen. However, considering the variability of the number of CIS cells present in the tissue adjacent to a tumour, the sensitivity of our assay is reasonably good, as nearly half of the patients harbouring testicular germ cell neoplasm have been detected in this preliminary study.

The relatively low sensitivity nevertheless leaves room for improvement and further optimisation of the method is currently in progress. The results of planned studies in a wider panel of patients will confirm whether this assay should be offered widely to patients at risk for TGCT as a method of non-invasive screening for CIS. Such screening could be performed, for example, at andrology and fertility clinics in young men with atrophic testes, history of cryptorchidism or in need for an assisted reproduction technique. If diagnosis of testicular cancer is made at the pre-invasive CIS stage, the patient can be offered the most gentle and optimal treatment.

### REFERENCES

Almstrup, K, Hoei-Hansen, C E, Wirkner, U, Blake, 3, Schwager, C, Ansorge, W, Nielsen, J E, Skakkebaek, N E, Rajpert-De Meyts, E and Leffers, H, 2004, Embryonic stem cell-like features of testicular carcinoma in situ revealed by genome-wide gene expression profiling Cancer Res 64:4736-43
Andersson, A M, Toppari, J, Haavisto, A M, Petersen, J H, Simell, T, Simell, O and Skakkebaek, N E, 1998, Longitudinal reproductive hormone profiles in infants: peak of inhibin B levels in infant boys exceeds levels in adult men J Clin Endocrinol Metab 83:675-81
Andrews, P W, 1984, Retinoic acid induces neuronal differentiation of a cloned human embryonal carcinoma cell line in vitro Dev Biol 103:285-93
Draper, J S, Smith, K, Gokhale, P, Moore, H D, Maltby, E, Johnson, J, Meisner, L, Zwaka, T P, Thomson, J A and Andrews, P W, 2004, Recurrent gain of chromosomes 17q and 12 in cultured human embryonic stem cells Nat Biotechnol 22:53-4
Giwercman, A, Cantell, L and Marks, A, 1991, Placental-like alkaline phosphatase as a marker of carcinoma-in-situ of the testis. Comparison with monoclonal antibodies M2A and 43-9F APMIS 99:586-94
Hadziselimovic, F, Thommen, L, Girard, J and Herzog, B, 1986, The significance of postnatal gonadotropin surge for testicular development in normal and cryptorchid testes J Urol 136:274-6
Hoei-Hansen, C E, Nielsen, J E, Almstrup, K, Hansen, M A, Skakkebaek, N E, Rajpert-DeMeyts, E and Leffers, H, 2004, Identification of genes differentially expressed in testes containing carcinoma in situ Mol Hum Reprod 10:423-31
Jager, R, Werling, U, Rimpf, S, Jacob, A and Schorle, H, 2003, Transcription factor AP-2gamma stimulates proliferation and apoptosis and impairs differentiation in a transgenic model Mol Cancer Res 1:921-9
Jahnukainen, K, Jorgensen N, Pollanen, P, Giwercman, A and Skakkebaek, N E, 1995, Incidence of testicular mononuclear cell infiltrates in normal human males and in patients with germ cell neoplasia Int J Androl 18:313-20
Jorgensen, M, Bevort, M, Kledal, T S, Hansen, B V, Dalgaard, M and Leffers, H, 1999, Differential display competitive polymerase chain reaction: an optimal tool for assaying gene expression Electrophoresis 20:230-40
Manivel, J C, Jessurun, J, Wick, M R and Dehner, L P, 1987, Placental alkaline phosphatase immunoreactivity in testicular germ-cell neoplasms Am J Surg Pathol 11:21-9
Nielsen, J E, Hansen, M A, Jorgensen, M, Tanaka, M, Almstrup, K, Skakkebaek, N E and Leffers, H, 2003, Germ cell differentiation-dependent and stage-specific expression of LANCL1 in rodent testis Eur J Histochem 47:215-22
Orso, F, Cottone, E, Hasleton, M D, Ibbitt, J C, Sismondi, P, Hurst, H C and De Bortoli, M, 2004, Activator protein-2gamma (AP-2gamma) expression is specifically induced by oestrogens through binding of the oestrogen receptor to a canonical element within the 5′-untranslated region Biochem J 377:429-38 [symbol]
Pera, M F, 2004, Unnatural selection of cultured human ES cells? Nat Biotechnol 22:42-3
Rajpert-De Meyts, E, Bartkova, J, Samson, M, Hoei-Hansen, C E, Frydelund-Larsen, L, Bartek, J and Skakkebaek, N E, 2003, The emerging phenotype of the testicular carcinoma in situ germ cell APMIS 111:267-78; discussion 278-9
Rajpert-De Meyts, E, Hanstein, R, Jorgensen, N, Graem, N, Vogt, P H and Skakkebaek, N E, 2004, Developmental expression of POU5F1 (OCT-3/4) in normal and dysgenetic human gonads Hum Reprod
Rajpert-De Meyts, E and Skakkebaek, N E, 1994, Expression of the c-kit protein product in carcinoma-in-situ and invasive testicular germ cell tumours Int J Androl 17:85-92
Ramalho-Santos, M, Yoon, S, Matsuzaki, Y, Mulligan, R C and Melton, D A, 2002, “Stemness”: transcriptional profiling of embryonic and adult stem cells Science 298:597-600
Ropponen, K M, Kellokoski, J K, Pirinen, R T, Moisio, K I, Eskelinen, M J, Alhava, E M and Kosma, V M, 2001, Expression of transcription factor AP-2 in colorectal adenomas and adenocarcinomas; comparison of immunohistochemistry and in situ hybridisation J Clin Pathol 54:533-8
Sato, N, Sanjuan, I M, Heke, M, Uchida, M, Naef, F and Brivanlou, A H, 2003, Molecular signature of human embryonic stem cells and its comparison with the mouse Dev Biol 260:404-13
Skakkebaek, N E, Rajpert-De Meyts, E and Main, K M, 2001, Testicular dysgenesis syndrome: an increasingly common developmental disorder with environmental aspects Hum Reprod 16:972-8
Sperger, J M, Chen, X, Draper, J S, Antosiewicz, J E, Chon, C H, Jones, S B, Brooks, J D, Andrews, P W, Brown, P O and Thomson, J A, 2003, Gene expression patterns in human embryonic stem cells and human pluripotent germ cell tumors Proc Natl Acad Sci USA 100:13350-5
Vernimmen, D, Gueders, M, Pisvin, S, Delvenne, P and Winkler, R, 2003, Different mechanisms are implicated in ERBB2 gene overexpression in breast and in other cancers Br J Cancer 89:899-906

1. A method for determining the presence of precursors of germ cell tumours in a sample, said method comprising:
a) determining the expression profile of a group of markers in said sample, wherein the group of markers consists of at least one marker selected independently from the markers listed in table 1;
b) comparing said expression profile with a reference expression profile;
c) identifying whether the expression profile is different from said reference expression profile; and
evaluating whether the sample contains precursors of germ cell tumours if the expression profile is different from the reference expression profile.

2. A method for determining the presence of precursors of germ cell tumours in a sample, said method comprising:
a) determining the intensity signal of the proteins encoded by each of the members of the group of markers in said sample, wherein the group of markers consists of at least one marker selected independently from the markers listed in table 1;
b) comparing each individual intensity signal with a corresponding reference intensity signal;
c) identifying whether each individual intensity signal is different from said corresponding reference intensity signal and
evaluating whether the sample contains precursors of germ cell tumours if the intensity signal is different from the corresponding reference intensity signal.

3. The method according to claim 1, wherein the group of markers is any one of SEQ ID NO 1-156, or SEQ ID NO 157.

4. The method according to claim 1, wherein the group of markers consists of at least one marker, which in a sample with CIS in 100% of the seminiferous tubules, is up-regulated more than 7 fold when compared to a reference sample.

5. The method according to claim 1, wherein the expression profile distinguishes between classical seminoma and non-seminoma, in gonads or in extra-gonadal localizations.

6. A method for monitoring progression from precursors of germ cell tumours to overt cancer cells in an individual, comprising:
a) determining the expression profile of a group of markers in said sample, wherein the group of markers consists of at least one marker selected independently from the markers listed in table 1;
b) comparing said expression profile with a reference expression profile;
c) identifying whether the expression profile is different from said reference expression profile; and
evaluating the development stage of precursors of germ cell tumours to overt cancer cells if the expression profile is different from the reference expression profile.

7. A method for monitoring progression from precursors of germ cell tumours to overt cancer including in an individual, comprising:
a) determining the intensity signal of the proteins encoded by each of the members of the group of markers in said sample, wherein the group of markers consists of at least one marker selected independently from the markers listed in table 1;
b) comparing each individual intensity signal with a corresponding reference intensity signal;
c) identifying whether each individual intensity signal is different from said corresponding reference intensity signal; and
evaluating the development stage of precursors of germ cell tumours to overt cancer cells if the intensity signal is different from the corresponding reference intensity signal.

8. The method according to claim 6, wherein said method distinguishes seminoma from non-seminoma.

9. The method according to claim 6, wherein said method distinguishes seminoma from CIS.

10. The method according to claim 6, wherein said method distinguishes non-seminoma from CIS.

11. A method of identifying precursors of germ cell tumours in a sample from a mammal, the method comprising:
assaying said sample by a quantitative detection assay and determining the expression profile or the intensity signal(s) of at least one marker selected from
SEQ ID NO:1 SEQ ID NO 254, or SEQ ID NO 255,
comparing said expression profile or the intensity signal(s) with reference value(s);
identifying whether the expression profile or the intensity signal(s) of at least one marker from the sample is significantly different from the reference value; and
evaluating whether the sample contains precursors of germ cell tumours if the expression profile or the intensity signal(s) is different from the reference value.

12. A method for determining the presence of precursors of germ cell tumours in an individual comprising detecting the presence or absence of at least one expression product, wherein said at least one expression product comprises a nucleotide sequence selected from SEQ ID NO 1-254 or SEQ ID NO 255 in a sample isolated from the individual.

13. A method of assessing the efficacy of a therapy for inhibiting precursors of germ cell tumours in a subject, the method comprising:
comparing the expression profile of a group of markers in a first sample obtained from the subject prior to providing at least a portion of the therapy to the subject and the expression profile of a group of markers in a second sample obtained from the subject following provision of the portion of the therapy, wherein a significantly altered expression profile of the marker(s) in the second sample, relative to the first sample, is an indication that the therapy is efficacious for inhibiting precursors of germ cell tumours in the subject,
wherein the group of markers consists of markers selected independently from the markers listed in table 1 and whereby the number of markers in the group is between 1 and 255.

14. A method of assessing the efficacy of a therapy for inhibiting precursors of germ cell tumours in a subject, the method comprising:
comparing the intensity signal of the proteins encoded by a group of markers in a first sample obtained from the subject prior to providing at least a portion of the therapy to the subject and the intensity signal of the proteins encoded by a group of markers in a second sample obtained from the subject following provision of the portion of the therapy, wherein a significantly altered intensity signal of the proteins encoded by the marker(s) in the second sample, relative to the first sample, is an indication that the therapy is efficacious for inhibiting precursors of germ cell tumours in the subject,
wherein the group of markers consists of markers selected independently from the markers listed in table 1 and whereby the number of markers in the group is between 1 and 255.

15. A method for analysing the presence of undifferentiated stem cells highly likely to progress to precursors of germ cell tumours or cancer in an individual, said method comprising
a) determining the expression profile of a group of markers in said sample, wherein the group of markers consists of at least one marker selected independently from the markers listed in table 1;
b) comparing said expression profile with a reference expression profile;
c) identifying whether the expression profile is different from said reference expression profiles; and
evaluating whether the sample contains undifferentiated stem cells highly likely to progress to precursors of germ cell tumours or cancer in the individual if the expression profile is different from the reference expression profile.

16. A method for analysing the presence of undifferentiated stem cells highly likely to progress to precursors of germ cell tumours or cancer in an individual, said method comprising:
a) determining the intensity signal of the proteins encoded by each of the members of the group of markers in said sample, wherein the group of markers consists of at least one marker selected independently from the markers listed in table 1;
b) comparing each individual intensity signal with a corresponding reference intensity signal;
c) identifying whether each individual intensity signal is different from said corresponding reference intensity signal; and
evaluating whether the sample contains undifferentiated stem cells highly likely to progress to precursors of germ cell tumours or cancer in the individual if the intensity signal is different from the reference expression profile.

17. The method according to claim 1, wherein the marker is selected from the group consisting of AP-2γ, PIM-2, TCL1A, NANOG, and E-cadherin.

18-22. (canceled)

23. The method according to claim 16, wherein the intensity signal is determined by immunocytological detection.

24. (canceled)

25. A kit comprising a means to detect a marker listed in table 1.
